(19) United States
(12) Patent Application Publication
(10) Pub. No.: US 2024/0209086 A1
(43) Pub. Date: Jun. 27, 2024

(54) THERAPEUTIC ANTIBODIES

(71) Applicant: PETMEDIX LTD., Cambridge (GB)

(72) Inventors: Meng Amy LI, Cambridge (GB); Roberto BANDIERA, Cambridge (GB); Yasmin Zoe PATERSON, Cambridge (GB); Ayesha BRADLEY, Cambridge (GB)

(21) Appl. No.: 18/524,083

(22) Filed: Nov. 30, 2023

(30) Foreign Application Priority Data
Nov. 30, 2022 (GB) 2217993.1

(51) Int. Cl. C07K 16/28; A61K 39/00; A61K 47/68; A61K 47/69; A61K 51/10; A61P 35/00; G01N 33/68

(52) U.S. Cl. CPC C07K 16/2809; A61K 47/6803; A61K 47/6849; A61K 47/6913; A61K 47/6929; A61K 51/1027; A61K 51/1042; A61P 35/00; C07K 16/2887; G01N 33/6872; A61K 2039/505; C07K 2317/31; C07K 2317/622; C07K 2317/92; G01N 2333/7051; G01N 2333/70596

(57) ABSTRACT

This disclosure provides a bispecific canine antigen-binding molecule comprising a first antigen binding domain or antigen-binding portion thereof that specifically binds canine CD3, and a second antigen binding domain or antigen-binding portion thereof that specifically binds canine CD20, compositions comprising the same, and methods of their use. The disclosure also provides a canine antibody or antigen-binding portion thereof that binds canine CD3, compositions comprising the same, and methods of their use. The disclosure also provides a canine antibody or antigen-binding portion thereof that binds canine CD20, compositions comprising the same, and methods of their use.

### CROSS REFERENCE TO RELATED APPLICATIONS

This application claims the benefit of and priority from United Kingdom Patent Application No. 2217993.1, filed Nov. 30 2022, the content of which is incorporated herein by reference in its entirety.

### SEQUENCE LISTING

The instant application contains a Sequence Listing which has been submitted electronically in XML format and is hereby incorporated by reference in its entirety. Said XML copy, created on Mar. 7, 2024, is named ZP000483A_SL.xml and is 1,077,674 bytes in size.

### INTRODUCTION

Cancer is one of the leading causes of death in companion animals. Common cancers include squamous cell carcinoma, mammary, prostate, connective tissues, melanoma, mouth and throat and lymphoma.

In the human field, cancer immunotherapy has attracted a huge amount of attention in the last three decades, as several therapeutic approaches have shown good results. One such approach is the use of bispecific antibodies which are capable of redirecting potent effector cells such as cytotoxic T cells and NK cells to mediate tumour lysis.

Cytotoxic T cells, as part of the adaptive immunity, are excellent effector cells to mediate killing, as they are relatively abundant, have the capacity to proliferate upon activation and have potent killing efficacy. In physiological conditions, T cells only direct their cytotoxic activity towards cells expressing major histocompatibility class (MHC) molecules loaded with epitopes they recognise through the T cell receptor (TCR). The TCR is a protein complex composed of either the α/β or γ/δ heterodimer in association with CD3 molecules, generally one CD3εγ heterodimer, one CD3εδ heterodimer and two CD3ζ chains. Bispecific T cell engagers can bypass the normal TCR-MHC interaction requirement to trigger T cell activation through one arm binding to the T cell/CD3 complex to elicit a polyclonal T cell response against a target antigen dictated by the second, target-recognising arm.

The application of bispecific T cell engagers has been growing tremendously in the human fields in the past decade after the FDA approval of the anti-CD19/anti-CD3 bispecific drug, Blinatumumab. So far, there are 43 CD3 based bispecific T cell engager antibodies targeting haematological and solid tumours in clinical phase development (Labrijn et al Nat Rev Drug Discov. 2021 18: 585-608). This class of antibody therapies is seen as the next generation human cancer immunotherapy.

There remains a need for anti-canine CD3 antibodies.

Building bispecific T cell engager therapeutic molecules for canine field is highly restricted. Unlike the human field, the canine antibody field has, until very recently, lacked several of the fundamental technologies required to enable fully canine bispecific antibodies such as how to heterodimerise canine heavy chains, how to restrict light chain mispairing, as well as purification methodologies tailored for canine bispecific molecules. In addition, the canine field also lacks research tools, protocols and models for T cell biology including the more specific T cell engager bispecific antibody biology. Overcoming these technical challenges would significantly benefit the discovery and development of the canine T cell engager bispecific molecules.

More importantly, such a class of molecules will potentially serve the very much unmet need in both haematological as well as solid tumours in the canine field, which unlike human, chemotherapy as the current standard treatment does not offer extended overall survival benefit.

Canine lymphomas are among the most common cancers diagnosed in dogs, representing around 7-14% of all cancers. As in humans, there are many different types of canine lymphoma and vary from rapidly progressing cancer to chronic disease.

CD20 is a cell-surface protein thought to be involved in regulation of B-cell proliferation and differentiation. The antigen comprises four transmembrane spanning regions and is present on the surface of almost all B-cells, both normal and malignant.

Human antibodies which recognise human CD20, such as rituximab, are used to treat human diseases characterized by excessive numbers of B-cells, or overactive or dysfunctional B-cells. These antibodies destroy B-cells. Rituximab is viewed as a revolutionary advance in the treatment of B-cell lymphoma.

Since the development of antibodies such as rituximab for humans over two decades ago, a number of antibodies which recognise canine CD20 have been reported in the literature. However, none are currently in common clinical use. Therefore, there still remains a need for different therapies. The invention is aimed at addressing this need.

Beyond anti-canine CD20 monospecific antibody therapy, T cell engager based bispecific antibodies could offer significant advantages, drawing from the experiences in human fields. Although Rituximab works as a monotherapy or in combination with chemotherapeutics, there are significant numbers of relapsed/refractory lymphoma patients in clinical setting. This has led to discovery and development of second generation of human anti-CD20 monospecific antibody drugs such as Ofatumumab and Obinutuzumab, which has much enhanced tumour killing CDC and ADCC activities respectively compared to Rituximab (Oflazoglu & Audoly 2010 mAbs, 2: 14-19). Despite this success, the field has moved into the next generation anti-CD3 and CD20 bispecific T cell engagers, with one FDA approved drug several entered into phase III clinical trials. Such bispecific molecules induce durable complete responses in patients with relapsed or refractory B-cell lymphoma who has received at least one prior therapy.

Therefore, there is a need for the discovery and development of the next generation CD3/CD20 bispecific antibodies to continuing the combat with canine B cell lymphoma.

Beyond CD3 based T cell engagers, anti-CD3 activating monospecific antibodies have been widely used in human fields for many decades as a tool to study T cell biology. In addition, such antibodies have been demonstrated to be efficacious T cell immunosuppressant in managing rejection after organ transplant and preventing graft-versus-host disease (Wunderlich M et al 2014 Blood 123 (24): e134-e144.). Although highly effective in immunomodulation, the severe side effect of cytokine release syndrome due to the strong initial T cell activation, led to the withdrawal of anti-human CD3 drug, Orthoclone OKT3. Second generation design with the use of effector function deficient OKT3 circumvents the high cytokine release problem and has led to the FDA approval of Teplizumab in 2022 for treatment of patients recently diagnosed with T1 D. This approval has reignited the interest in this class as therapeutic drugs in modulating immunological tolerance.

These pioneering human studies have paved the way for anti-CD3 therapy to realise its full therapeutic potential. Identification of anti-canine CD3 antibodies therefore benefit the canine field in terms of basic research on T cell biology as well as therapeutic opportunities in canine autoimmune diseases. For example, type 1 diabetes in dogs is prevalent, approximately 0.2%-1.0% of dogs develop T1D and this incident rate is expected to grow.

### SUMMARY

In a first aspect, the invention relates to a canine antibody, antigen binding domain or antigen-binding portion thereof that binds canine CD3. The antibody may comprise three heavy chain variable region (HCVR) complementarity determining region (CDR)s and/or 3 light chain variable region (LCVR) CDRs as described herein. The antibody, antigen binding domain or antigen binding portion thereof may comprise the HCVR and/or LCVR s as described herein. The invention also relates to an immunoconjugate comprising such canine antibody, antigen binding domain or antigen-binding portion thereof as well as pharmaceutical composition comprising such an antibody, antigen binding domain or antigen-binding portion thereof. Further aspects relate to the treatment of disease comprising administering such a canine antibody, antigen binding domain or antigen-binding portion thereof. In particular, the disease may be cancer and to a method for increasing an immune response in a subject comprising administering such a canine antibody, antigen binding domain or antigen-binding portion thereof.

In another aspect, the invention relates to a bispecific antibody comprising a canine antibody, antigen binding domain or antigen-binding portion thereof that binds canine CD3 and a canine antibody, antigen binding domain or antigen-binding portion thereof that binds a second canine antigen target.

In another aspect, the invention relates to a kit comprising a canine antibody, antigen binding domain or antigen-binding portion thereof that binds canine CD3 or a pharmaceutical composition as described herein.

In another aspect, the invention relates to a nucleic acid sequence that encodes an antibody, antigen binding domain or antigen-binding portion thereof that binds canine CD3 as described herein.

In another aspect, the invention relates to a vector comprising a nucleic acid sequence that encodes an antibody, antigen binding domain or antibody antigen-binding portion thereof that binds canine CD3 as described herein.

In another aspect, the invention relates to a host cell comprising such a nucleic acid sequence or vector.

In another aspect, the invention relates to a method for making a canine antibody or antigen binding domain that binds CD3 comprising culturing the isolated host cell and recovering said antibody.

In another aspect, the invention relates to a method for making a canine antibody or antigen binding domain that binds CD3 comprising the steps of
a) immunising a transgenic mouse that expresses a nucleic acid construct comprising canine heavy chain V genes and canine light chain V genes with CD3 antigen,
b) generating a library of antibodies from said mouse and
c) isolating an antibody from said library.

In another aspect, the invention relates to a method for detecting a CD3 protein or an extracellular domain of a CD3 protein in a biological sample from a canine subject, comprising contacting a biological sample with the antibody, antigen binding domain or antigen-binding portion thereof wherein said antibody, antigen binding domain or antigen-binding portion thereof is linked to a detectable label.

In another aspect, the invention relates to a combination therapy comprising a canine antibody, antigen binding domain or antigen-binding portion thereof that binds canine CD3 or a pharmaceutical composition comprising a canine antibody, antigen binding domain or antigen-binding portion thereof that binds canine CD3.

In another aspect, the invention relates to a bispecific canine antigen-binding molecule comprising a first antibody, antigen binding domain or antigen-binding portion thereof that specifically binds canine CD3, and a second antibody, antigen binding domain or antigen-binding portion thereof that specifically binds canine CD20. The first antibody, antigen binding domain or antigen-binding portion thereof that specifically binds canine CD3 may be as described herein. The second first antibody, antigen binding domain or antigen-binding portion thereof that specifically binds canine CD20 may be as described herein.

In another aspect, the invention relates to a pharmaceutical composition comprising such a bispecific antigen-binding molecule that binds canine CD3 and canine CD20. The invention also relates to method of treating cancer or a condition mediated by B-cells in a canine subject in need thereof/method for increasing an immune response in a subject comprising administering an effective amount of the bispecific canine antigen-binding molecule.

The invention also relates to a kit comprising such bispecific canine antigen-binding molecule or a pharmaceutical composition as described herein.

The invention also relates to a nucleic acid sequence that encodes such a bispecific canine antigen-binding molecule.

The invention also relates to a vector comprising such a nucleic acid sequence.

The invention also relates to a host cell comprising such a nucleic acid sequence.

The invention also relates to a method for making a bispecific antigen-binding molecule comprising culturing the isolated host cell as described herein and recovering said antibody.

The invention also relates to a method for detecting a CD3 protein and a CD20 protein in a biological sample from a canine subject, comprising contacting a biological sample with the bispecific antigen-binding molecule as described herein wherein said antibody, antigen binding domain or antigen-binding portion thereof is linked to a detectable label.

The invention also relates to a canine antibody, antigen binding domain or antigen-binding portion thereof which binds canine CD20 wherein said antibody comprises In one embodiment, the canine antibody or antigen-binding fragment portion that binds canine CD20 comprises the complementarity determining regions (CDRs) of a heavy chain variable region (HCVR) having an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto.

In another aspect, the invention relates to a pharmaceutical composition comprising a canine antibody, antigen binding domain or antigen-binding portion thereof which binds canine CD20 as described above.

The invention also relates to method of treating cancer or a condition mediated by B-cells in a canine subject in need thereof/method for increasing an immune response in a subject comprising administering an effective amount of a canine antibody, antigen binding domain or antigen-binding portion thereof which binds canine CD20 as described above.

The invention also relates to a kit comprising a canine antibody, antigen binding domain or antigen-binding portion that binds CD20 thereof as described above or a pharmaceutical composition as above.

The invention also relates to a nucleic acid sequence that encodes such a canine antibody, antigen binding domain or antigen-binding portion that binds CD20 thereof as described above.

The invention also relates to a vector comprising such a nucleic acid sequence.

The invention also relates to a host cell comprising such a nucleic acid sequence.

The invention also relates to a method for making a canine antibody or antigen binding domain that binds CD20 as described above comprising culturing the isolated host cell as described above and recovering said antibody.

The invention also relates to a method for making a canine antibody or antigen binding domain that binds CD20 as described above comprising the steps of
a) immunising a transgenic mouse that expresses a nucleic acid construct comprising canine heavy chain V genes and canine light chain V genes with CD20 antigen,
b) generating a library of antibodies from said mouse and
c) isolating an antibody from said library.

The invention also relates to a method for detecting a CD20 protein or an extracellular domain of a CD20 protein in a biological sample from a canine subject, comprising contacting a biological sample with the antibody, antigen binding domain or antigen-binding portion thereof as described above wherein said antibody, antigen binding domain or antigen-binding portion thereof is linked to a detectable label.

The invention also relates to a method of inhibiting tumour growth or metastasis comprising contacting a tumour cell with an effective amount of the antibody, antigen binding domain or antigen-binding portion thereof as described above.

The invention also relates to a method of killing a tumour cell expressing CD20, comprising contacting the cell with the antibody, antigen binding domain or pharmaceutical composition as described above, such that killing of the cell expressing CD20 occurs.

### BRIEF DESCRIPTION OF THE DRAWINGS

The invention is further described in the following non-limiting figures.

FIGS. 1A-1B. CD3 immunised Ky9 mice serum titre. Serum antibody titre measured using flow cytometry after staining HEK cells expressing canine CD3εδ heterodimers at the cell surface. FIG. 1A. Serum collected from mice 10 days after the first boost. FIG. 1B. Serum collected from mice 10 days after the second boost. At both time points a dose dependent increase in binding was detected, indicating the presence of specific anti-CD3 antibodies in serum.

FIG. 2. Schematic of a four-chain bispecific screening format for anti-CD3 candidates. CD3 candidate sequences were synthesised in a bispecific format for screening. The antibody includes a candidate CD3 arm and an anti-human CD20 arm composed of Rituximab variable sequences. To enhance the heterodimerisation, the human Fc comprised knob-in-hole mutations were used.

FIG. 3. CD3 cell binding capacity. The graph shows the difference in GeoMean intensity of signal obtained by flow cytometry of CD3εδ-expressing HEK293 cells or the parental line stained with candidate CD3 antibodies and subsequently with a PE labelled secondary antibody recognising the human Fc. A candidate antibody is considered a binder when the signal obtained from staining CD3εδ-expressing HEK293 cells is higher than that obtained from staining wild type HEK cells.

FIGS. 4A-4B. CD3εδ binding measured by SPR. Example sensograms showing the single cycle binding kinetics of CD3 candidate antibodies in bispecific format to canine CD3εδ-Fc. FIG. 4A. PMX157, PMX158, PMX159, PMX160. FIG. 4B. PMX161, PMX162, PMX163, PMX164. Tested candidates all demonstrated the ability to bind to canine CD3εδ-Fc, although to a different degree. Lines with no legend are non-binders, served as negative controls.

FIGS. 5A-5B. Bispecific killing screening. Comparison of bispecific killing activity and IFN-γ release for CD3 candidates in bispecific format. FIG. 5A. IFN-γ release following 48 hours of effector: target incubation at an antibody concentration of 1 ug/ml. Effector cells were PBMCs and target cells were MDCK II cells expressing hCD20+GFP or dCD20+GFP. All IFN-γ curves are baseline subtracted from the signal obtained from no cell control and are plotted as the concentration of IFN-γ releases in picograms per millilitre. FIG. 5B. CD3 bispecific antibodies, with one canine CD3 arm and one Rituximab arm (FIG. 2), have the ability to induce bispecific killing of canine cells expressing human CD20 at an antibody concentration of 1 ug/ml. Data are plotted as percentage of killing where 100% means all cells were killed and 0% means signal was identical to what obtained in control cells (no antibody added).

FIG. 6. Bispecific killing dose response measurement. Comparison of the ability of CD3 candidates in bispecific format, with one canine CD3 arm and one Rituximab arm (FIG. 2), to induce bispecific killing of canine cells expressing human CD20 using PBMCs at different antibody concentrations. Data are plotted as percentage of killing where 100% means all cells were killed and 0% means signal was identical to what obtained in control cells (no antibody added). On the same plots as the bispecific killing, representative curves showing the IFN-γ release following 48 hours of effector: target incubation at different antibody concentrations are also shown. All IFN-γ curves are baseline subtracted from the signal obtained from no cell control and are plotted as the concentration of IFN-γ releases in picograms per millilitre.

FIG. 7. CD3 affinity determination in bispecific format. Direct comparison of shortlisted candidate CD3 bispecific antibodies, with one canine CD3 arm and one Rituximab arm (FIG. 2), for their ability to bind to canine CD3εδ-Fc and the affinity is determined by SPR.

FIGS. 8A-8E. Ex vivo T cell activation with monospecific CD3 antibodies. FIG. 8A. Brightfield images of canine PBMC cultured for 72 h in tissue culture plates pre coated with the indicated antibodies. The presence of large cell clusters is proportional to the extent of T cell activation. FIG. 8B. ELISA quantification of IFN-γ in supernatants of canine PBMC cultured in tissue culture plates pre coated with the indicated antibodies. Supernatants were collected after 3 or 4 days of culturing. Tested CD3 candidate antibodies were able to activate T cells to different degrees, making them the perfect tool sets to achieve different levels of T cell activation. FIG. 8C. Proportion of CD25 and CD5 double positive canine T cells in the presence or absence of PMX159 together with a co-stimulatory anti-canine CD28. FIG. 8D. Proportion of PD-1 and CD5 double positive canine T cells in the presence or absence of PMX159 together with a co-stimulatory anti-canine CD28. FIG. 8E. Proportion of Ki67% positive proliferative CD5+ T cells in the presence or absence of PMX159 together with a co-stimulatory anti-canine CD28.

FIG. 9. CD20 binding capacity of monospecific anti-CD20 candidates. Single-dose cell binding capacity of candidate monospecific CD20 antibodies at 10 μg/ml. The graph shows the difference in mean fluorescence intensity (MFI) of signal obtained by flow cytometry of CD20-expressing HEK293 cells stained with candidate CD20 antibodies and subsequently with a FITC labelled secondary antibody recognising the canine Fc. All candidates shown, with exception of PMX250 and PMX251, demonstrated strong binding capacity to CD20 expressing cells.

FIG. 10. Functional screening of monospecific CD20 candidates for CDC capacity. Single-dose complement dependent cytotoxicity (CDC), showing the percentage of cells killed in the assay by each candidate antibody used at 1 μg/ml.

FIG. 11. Functional screening of monospecific CD20 candidates for ADCC capacity. Single dose antibody-dependent cellular cytotoxicity (ADCC), showing the percentage of cells killed in the assay by each candidate antibody used at 0.01 μg/ml.

FIGS. 12A-12B. Comparing the CD3 binding capacity of different direct assembled canine CD20 and CD3 heavy chains with either CD20 or CD3 light chains. FIG. 12A. Graphic representation of bispecific assembly composed of either CD3 light chain or CD20 light chain assembled with the heavy chain CD3/CD20 heterodimer using human KiH Fc. FIG. 12B. Single dose cell binding of direct assembled CD3/CD20 bispecific antibodies at 1 μg/ml, with either a CD3 or CD20 VL chain. The image shows the flow cytometry profile of CD3εδ-expressing HEK293 cells stained with the direct assembled bispecific antibodies and subsequently with a PE labelled secondary antibody recognising the human Fc. Secondary only staining is served as a non-binding control.

FIGS. 13A-13F: Exploration of the compatibility of CD3 and CD20 VLs with different CD20 VHs. FIG. 13A. Graphic representation of bispecific assembly composed of a single CD3 light chain candidate PMX172VH, with either PMX172VL (CD3VL) or different CD20 VH and VL candidates using human KiH Fc for heterodimerisation. FIG. 13B. Single dose cell-based CD3 binding of direct assembled CD3/CD20 bispecific antibodies at 1 μg/ml, with a single CD3 VL-PMX172VL or cognate CD20 VL, CD3VH-PMX172VH, and differing CD20 VHs. Flow cytometry profiles of CD3εδ-expressing HEK293 cells stained with the direct assembled bispecific antibodies and subsequently with a PE labelled secondary antibody recognising the human Fc. Secondary only staining is served as a non-binding control. FIG. 13C. Single dose cell based CD20 binding of direct assembled CD3/CD20 bispecific antibodies at 1 μg/ml, with CD3 VL PMX172VL. Flow cytometry profiles of CD20-expressing MDCK cells stained with the direct assembled bispecific antibodies and subsequently with a PE labelled secondary antibody recognising the human Fc. Secondary only staining is served as a non-binding control. FIG. 13D. Fluorescent mean intensity summary of flow profile shown in FIG. 13C. FIG. 13E. Three-chain bispecific assemblies of VH and VL of CD3 candidate PMX172 together with indicated anti-CD20 VHs to induce bispecific killing of canine cells expressing human CD20 at different antibody concentrations. Data are plotted as percentage of killing where 100% means all cells were killed and 0% means signal was identical to what obtained in control cells (no antibody added). FIG. 13F. Percentage of B cell depletion following addition of different concentrations of three chain bispecific assemblies as described in FIG. 13E. Data are plotted as percentage of B cell depletion, where 0% means signal was identical to what obtained in the no antibody control. FIG. 13G. Frequency of activated CD8 T cells, as measured by CD25 activation, following addition of different concentrations of three chain bispecific assemblies as described in FIG. 13E.

FIGS. 14A-14B. Identifying new CD20 VHs for use in the bispecific assembly. FIG. 14A. CD20 binding assay using 25 ug/ml, 8.3 ug/ml, 2.8 ug/ml and 0.92 ug/ml of bispecific candidates with anti-CD3 PMX172 VH together with the differing VH CD20 binders as indicated and PMX172 VL. Human KiH was used for Fc human heterodimerisation. The graph shows the mean fluorescence intensity (MFI) of signal obtained by flow cytometry of CD20-expressing MDCK cells stained with three chain bispecific antibodies as described and subsequently with a PE labelled secondary antibody recognising the human Fc. FIG. 14B. Multiple sequence alignment of the anti-CD20 VH sequences in strong binder and weak binder groups with CDRs indicated. All sequences were aligned to germline V3-5. Grey shades indicate the sequence differences to the germline sequence.

FIG. 15. Further screening of canine CD20 VHs using a bispecific killing assay. Dose dependent bispecific cell killing of bispecific assemblies with anti-CD3 PMX172 VH together with the differing VH CD20 binders as indicated with PMX172 common light chain with human KiH Fc for heterodimerisation. Target cells: canine cells expressing human CD20. Effector: Canine PBMC. Data are plotted as percentage of killing where 100% means all cells were killed and 0% means signal was identical to what obtained in control cells (no antibody added).

FIGS. 16A-16B. Further screening of canine CD20 VHs using an endogenous B cell depletion assay. FIG. 16A. Percentage of B cell depletion following addition of different concentrations of bispecific assemblies with anti-CD3 PMX172 VH together with the differing VH CD20 binders, PMX172 VL as the common light chain and human KiH Fc for heterodimerisation. Data are plotted as percentage of B cell depletion, where 0% means signal was identical to what obtained in the no antibody control. FIG. 16B. Proportion of activated CD8 T cells, as measured by CD25 activation, following addition of different concentrations of bispecific assemblies.

FIGS. 17A-17B. Naïve PBMC B cell killing assay of fully canine CD3/CD20 bispecific candidates. FIG. 17A. Percentage of B cell depletion following addition of 1.25 ug/ml of candidate fully canine 3-chain assemblies. Data are plotted as percentage of B cell depletion, where 0% means signal was identical to what obtained in the no antibody control. FIG. 17B. Proportion of activated CD8 T cells, as measured by CD25 activation, following addition of 1.25 ug/ml of candidate fully canine 3-chain assemblies compared to a no antibody control.

FIGS. 18A-18B. Whole blood B cell killing assay of fully canine CD3/CD20 bispecific candidates. FIG. 18A. Percentage of B cell depletion following addition of 1.25 ug/ml of candidate fully canine 3-chain assemblies. Data are plotted as percentage of B cell depletion, where 0% means signal was identical to what obtained in the no antibody control. FIG. 18B. Proportion of activated CD8 T cells, as measured by CD25 activation, following addition of 1.25 ug/ml of candidate fully canine 3-chain assemblies compared to a no antibody control.

FIGS. 19A-19B. Design for canine CD20 and CD3 knock in mice. FIG. 19A. Graphic representation of the canine CD20 knock in design to replace the genomic region encoding all coding exons of mouse CD20 with the canine corresponding genomic region. FIG. 19B. Graphic representation of the canine CD3 knock in (KI) design to replace the mouse genomic region encoding the leader and extracellular domain with canine corresponding genomic region.

FIG. 20. Dose dependent B cell depletion of CD3/CD20 bispecific mAb candidates in canine CD3/CD20 KI mice. Percentage of B cells detected on day 6 in peripheral blood following addition of 0.01, 0.05, 0.1, 0.5 and 1 mg/kg of bispecific candidates. Data are plotted as percentage of mouse CD19 expressing cells, where 0% means signal was identical to what obtained in the unstained control. Significant differences between sample means are indicated: *, P<0.05; **, P<0.01 as determined using one-way ANOVA.

FIGS. 21A-21B. Dose dependent T cell activation of CD3/CD20 bispecific mAb candidates in canine CD3/CD20 KI mice. FIG. 21A. Percentage of activated T cells detected on day 6 in peripheral blood following addition of 0.01, 0.05, 0.1, 0.5 and 1 mg/kg of candidate bispecific mAbs. Data are plotted as percentage of CD8+ T cells expressing activation markers PD1 and TIM3, where 0% means signal was identical to that obtained in the unstained control. Significant differences between sample means are indicated: *, P<0.05; **, P<0.01 as determined using one-way ANOVA. FIG. 21B. Correlation plot shows the relationship between the percentage of activated CD8+ T cells (CD8+ve, PD1+ve and TIM3+ve) and percentage of B cells (CD19+ve). Correlation analysis was performed with Spearman's correlation test and a p value<0.05 was considered statistically significant.

FIGS. 22A-22B. Dose dependent effector memory activation of CD3/CD20 bispecific mAb candidates in canine CD3/CD20 KI mice. FIG. 22A. Percentage of effector memory T cells detected on day 6 in peripheral blood following addition of 0.01, 0.05, 0.1, 0.5 and 1 mg/kg of candidate bispecific mAbs. Data are plotted as a percentage of CD8+ T cells negative for naïve T cell markers CD45RA and CD62L. Significant differences between sample means are indicated: *, P<0.05; **, P<0.01 as determined using one-way ANOVA. FIG. 22B. Correlation plot shows the relationship between the percentage of effector memory CD8+ T cells (CD8+ve, CD45RA-ve and CD62L-ve) and the percentage of activated CD8+ T cells (CD8+ve, PD1+ve and TIM3+ve). Correlation analysis was performed with Spearman's correlation test and a p value<0.05 was considered statistically significant.

FIG. 23. Cytokine release following bispecific candidate mAbs treatment in canine CD3/CD20 KI mice. IL-2, IL-6, IFN-γ and TNF-α levels as determined using LEGENDplex™ Mouse Th Cytokine bead based multiplex immunoassay, from serum samples of mice (n=5) treated with 0.5 mg/kg of candidate mAbs compared to vehicle control.

FIGS. 24A-24B. Time course of B cell depletion in peripheral blood and spleen of CD3/CD20 bispecific mAb candidates in canine CD3/CD20 KI mice. FIG. 24A. Percentage of B cells identified in spleen by CD19 positive staining over a 21 day time period following treatment with 0.5 mg/kg candidate mAbs. FIG. 24B. Percentage of B cells detected in peripheral blood over a 21 day time period following treatment with 0.5 mg/kg candidate mAbs. Data are plotted as percentage of positive CD19 expressing cells, where 0% means signal was identical to that obtained in the unstained control. Significant differences between sample means are indicated: *, P<0.05; **, P<0.01 as determined using one-way ANOVA.

FIGS. 25A-25B. Time course of cytotoxic T cell activation in peripheral blood and spleen of CD3/CD20 bispecific mAb candidates in canine CD3/CD20 KI mice. FIG. 25A. Percentage of activated CD8+ T cells identified by PD1+ TIM3+ over a 21 day time period in peripheral blood following addition 0.5 mg/kg of candidate fully canine 3-chain assemblies. FIG. 25B. Percentage of activated CD8+ T cells detected over a 21 day time period in spleen following addition 0.5 mg/kg of candidate bispecific mAbs. Data are plotted as percentage of CD8+ T cells positive for activation markers PD1 and TIM3, where 0% means signal was identical to that obtained in the unstained control. Significant differences between sample means are indicated: *, P<0.05; **, P<0.01 as determined using one-way ANOVA.

FIGS. 26A-26B. CD8+ T cell counts in peripheral blood and spleen of CD3/CD20 bispecific mAb candidates in canine CD3/CD20 KI mice. FIG. 26A. Percentage of CD8+ T cells detected in spleen over a 21 day time period following treatment with 0.5 mg/kg candidate bispecific mAbs. FIG. 26B. Percentage of CD8+ T cells detected in peripheral blood over a 21 day time period following treatment with 0.5 mg/kg candidate bispecific mAbs. Data are plotted as percentage of positive CD8a expressing cells, where 0% means signal was identical to that obtained in the unstained control. Significant differences between sample means are indicated: *, P<0.05; **, P<0.01 as determined using one-way ANOVA.

FIGS. 27A-27B. Evaluation of canine CD20 expression in A20 tumour cells in a syngeneic mouse model. FIG. 27A. Cells isolated from tumours, generated using canine A20 overexpressing cells and wildtype A20 control, subcutaneously injected into recipient canine CD3/CD20 KI mice were stained with PE-canine CD20. Dot plots show sideward scatter signal plotted against PE signal. Positive PE signal was determined against an unstained control. A clear population shift is observed in the canine overexpressing tumours compared the wildtype control with over 90% being PE positive. FIG. 27B. Percentage of infiltrating immune cell types present in cCD20 overexpressing A20 generated tumours (n=6). Populations investigated include effector T cells (CD3+ve, CD4+ve), cytotoxic T cells (CD3+ve, CD8a+ve), NK cell markers (CD3−ve, CD49b+ve), PMC-MDSC markers (CD11 b+ve, Ly6C low, Ly6G+ve), and monocytic MDSC markers (CD11b+ve, Ly6C high, Ly6G−ve).

FIGS. 28A-28C. Pharmacokinetics and pharmacodynamics of canine CD3 and CD20 bispecific candidates in healthy beagles. FIG. 28A. Percentage of B-cell (CD21+cells) baselined to the percentage of B cells pre-dosing (Pre-bleed) in peripheral blood over time. Dashed lines correspond to the 24 hour timepoint post each dosing event (0.01 mg/kg (Day 0), 0.05 mg/kg (Day 8) and 0.5 mg/kg (Day15). Statistical analysis was performed in GraphPad prism, where *=p<0.05 and **=P<0.001 based on one-way ANOVA. FIG. 28B. Percentage of activated T cells (CD8+, PD1+) detected at 24 hr and 6 days post each dosing event of candidate fully canine 3-chain assemblies (0.01 mg/kg (Day 0), 0.05 mg/kg (Day 8) and 0.5 mg/kg (Day15) in peripheral blood. FIG. 28C. Percentage of effector memory T cells (CD45RA-CD62L−) detected at 24 hr and 6 days post each dosing event of candidate fully canine 3-chain assemblies (0.01 mg/kg (Day 0), 0.05 mg/kg (Day 8) and 0.5 mg/kg (Day15) in peripheral blood.

FIG. 29. CD3/CD20 bispecific antibody combination treatment with chemo and monospecific anti-CD20 therapy. Combination therapies are a successful way of ensuring maximum tumour cell clearance whilst minimising safety concerns and adverse events. This will be assessed by comparing the current standard of care for lymphoma, CHOP, as well as what is described as r-CHOP in the human field (CHOP+anti-CD20 monotherapy) with either monoclonal therapy alone or as a combination therapy, where the monoclonal CD20 will be dosed in order to allow for the debulking of B cells followed the CD3/CD20 bispecific in order to allow for deep tissue penetration. All parameters described in Example 14 will be conducted as previously described to assess both B and T cell population dynamics over time.

FIGS. 30A-30B. Cell binding capacity of monospecific CD3 mAbs. The graph shows the difference in GeoMean intensity of fluorescent signal obtained by flow cytometry of CD3εδ-expressing (FIG. 30A) or CD3εγ (FIG. 30B)-expressing HEK293 cells or the parental line stained with candidate CD3 antibodies and subsequently with a PE labelled secondary antibody recognising the human Fc. A candidate antibody is considered a binder when the signal obtained from staining CD3εδ or εγ-expressing HEK293 cells is higher than that obtained from staining wild type HEK cells.

### DETAILED DESCRIPTION

The embodiments of the invention will now be further described. In the following passages, different embodiments are described. Each aspect so defined may be combined with any other aspect or aspects unless clearly indicated to the contrary.

Generally, nomenclatures used in connection with, and techniques of, cell and tissue culture, pathology, oncology, molecular biology, immunology, microbiology, genetics and protein and nucleic acid chemistry and hybridization described herein are those well-known and commonly used in the art. The methods and techniques of the present disclosure are generally performed according to conventional methods well-known in the art and as described in various general and more specific references that are cited and discussed throughout the present specification unless otherwise indicated. See, e.g., Green and Sambrook et al., Molecular Cloning: A Laboratory Manual, 4th ed., Cold Spring Harbor Laboratory Press, Cold Spring Harbor, N.Y. (2012); Therapeutic Monoclonal Antibodies: From Bench to Clinic, Zhiqiang An (Editor), Wiley, (2009); and Antibody Engineering, 2nd Ed., Vols 1 and 2, Ontermann and Dübel, eds., Springer-Verlag, Heidelberg (2010), Handbook of Therapeutic Antibodies, Dübel and Janice M. Reichert, Wiley, (2014).

Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

The inventors have developed fully canine antibodies that bind specifically to canine CD3. These antibodies were generated in transgenic rodents expressing canine V, D, J genes. Therefore, the antibodies are less likely to be immunogenic for administration to canine subjects than caninized antibodies or chimeric antibodies. Furthermore, as these antibodies can be used directly, with no further modifications to their variable regions, there is no risk of reducing the affinity or otherwise compromising the antibody. Other technologies risk introducing development or efficacy liabilities through the ex vivo combination of antibody sequences of canine origin with that from another species, typically rodent. Thus, the invention relates to a canine antibody or antigen-binding portion thereof which binds canine CD3.

The invention further relates to a bispecific canine antigen-binding molecule comprising a first antibody, antigen binding domain or antigen-binding portion thereof that specifically binds canine CD3, and a second antibody, antigen binding domain or antigen-binding portion thereof that specifically binds another canine antigen, for example CD20.

The properties of the antibodies, antigen binding domains and antigen binding portions thereof of the invention can be exploited in therapeutic methods and uses as well as in pharmaceutical formulations as described herein.

The term CD3 refers to antigen cluster of differentiation 3. CD3 is a multimeric protein complex, known historically as the T3 complex, and is composed of four distinct polypeptide chains; epsilon (s), gamma (γ), delta (6) and zeta (ζ), that assemble and function as three pairs of dimers (εγ, εδ, ζζ). The CD3 complex serves as a T cell co-receptor that associates noncovalently with the T cell receptor (TCR). Unless otherwise stated, the term CD3 as used herein refers to canine CD3.

The antibodies, antigen binding domains and antigen binding portions thereof bind specifically to wild type canine CD3, in particular the CD3εδ dimer. Nucleic acid and amino acid sequences of the wild type canine CD3 subunits are shown in Table 1. The amino acid sequence of wild type CD3ε is SEQ ID NO: 4 and the amino acid sequence of wild type CD35 is SEQ ID NO: 5. Unless otherwise stated, the term CD3 as used herein refers to canine CD3.

The terms “CD3 antigen binding domain”, “CD3 binding molecule/protein/polypeptide/agent/moiety”, “CD3 antigen binding molecule molecule/protein/polypeptide/agent/moiety”, “anti-CD3 antibody”, “anti-CD3 antibody or antigen binding portion thereof” all refer to a molecule capable of specifically binding to the canine CD3 antigen. The binding reaction may be shown by standard methods, for example with reference to a negative control test using an antibody of unrelated specificity.

The term CD20 refers to the B-lymphocyte antigen CD20. The antibodies, antigen binding domains and antigen binding portions thereof bind specifically to wild type canine CD20 as defined in SEQ ID NO: 22 (nucleotide sequence) and SEQ ID NO: 24 (amino acid sequence) and shown in Table 1. Unless otherwise stated, the term CD20 as used herein refers to canine CD20. B-lymphocyte antigen CD20 or CD20 is expressed on the surface of all B-cells beginning at the pro-B phase (CD45R+, CD117+) and progressively increasing in concentration until maturity. In humans and canines, CD20 is encoded by the MS4A1 gene.

The terms “CD20 antigen binding domain”, “CD20 binding molecule/protein/polypeptide/agent/moiety”, “CD20 antigen binding molecule molecule/protein/polypeptide/agent/moiety”, “anti-CD20 antibody”, “anti-CD20 antibody or antigen binding portion thereof” all refer to a molecule capable of specifically binding to the canine CD20 antigen. The binding reaction may be shown by standard methods, for example with reference to a negative control test using an antibody of unrelated specificity.

An antibody, antigen binding domain or antigen binding portion thereof of the invention, including a multispecific, e.g. bispecific or trispecific, binding agent described herein, “which binds” or is “capable of binding” an antigen of interest, that is canine CD3, CD3 and CD20 or CD20 is one that binds the antigen with sufficient affinity such that the antibody, antigen binding domain or antigen binding portion thereof is useful as a therapeutic agent in targeting a cell or tissue expressing the respective antigen as described herein.

An antibody, antigen binding domains or antigen binding portion thereof described herein binds specifically to canine CD3. In other words, binding to the canine CD3 antigen is measurably different from a non-specific interaction. In particular, the antibodies described herein do not cross react with mouse CD3.

Also described are antibodies, antigen binding domains or antigen binding portions thereof that bind specifically to canine CD20. In other words, binding to the canine CD20 antigen is measurably different from a non-specific interaction. In particular, the antibodies described herein do not cross react with mouse CD20.

The term “specific binding” or “specifically binds to” or is “specific for” a particular polypeptide or an epitope on a particular polypeptide target as used herein can be exhibited, for example, by a molecule having a KD for the target of at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or lower. In one embodiment, the KD is at least about 10⁻⁸ M to about 10⁻⁹ M, e.g. In one embodiment, the KD is in the nanomolar range. In one embodiment, the term “specific binding” refers to binding where a molecule binds to a particular polypeptide or epitope on a particular polypeptide without substantially binding to any other polypeptide or polypeptide epitope. The terms KD and K_{D} are used interchangeably herein. Further binding affinities are set out elsewhere herein.

As used herein, the term “antigen-binding molecule” refers to a protein, polypeptide or molecular complex comprising at least one complementarity determining region (CDR) that alone, or in combination with one or more additional CDRs and/or framework regions (FRs), specifically binds to a particular antigen. In certain embodiments, an antigen-binding molecule is an antibody or a portion of an antibody, as those terms are defined elsewhere herein. In some embodiments, the antigen-binding domain specifically binds to the canine CD3 antigen. In some embodiments, the antigen-binding domain specifically binds to the canine CD20 antigen. The term “antigen-binding molecule” includes antibodies and antigen-binding portions of antibodies, including, e.g., bispecific antibodies.

As used herein, the term “bispecific antigen-binding molecule” refers to a protein, polypeptide or molecular complex comprising at least a “first antigen-binding domain” and a “second antigen-binding domain”. Each antigen-binding domain within the bispecific antigen-binding molecule comprises at least one CDR that alone, or in combination with one or more additional CDRs and/or FRs, specifically binds to a particular antigen. In the context of the present invention, the first antigen-binding domain specifically binds a first distinct antigen (e.g., canine CD3), and the second antigen-binding domain specifically binds a second distinct antigen (e.g., canine CD20).

The term “antibody” as used herein broadly refers to any immunoglobulin (Ig) molecule, or antigen binding portion thereof, comprised of four polypeptide chains, two heavy (H) chains and two light (L) chains, or any functional fragment, mutant, variant, or derivation thereof, which retains the essential epitope binding features of an Ig molecule.

In a full-length antibody, each heavy chain is comprised of a heavy chain variable region or domain (abbreviated herein as HCVR) and a heavy chain constant region. The heavy chain constant region is comprised of three domains, C_{H}1, C_{H}2 and C_{H}3. Each light chain is comprised of a light chain variable region or domain (abbreviated herein as LCVR) and a light chain constant region. The light chain constant region is comprised of one domain, CL.

The heavy chain and light chain variable regions can be further subdivided into regions of hypervariability, termed complementarity determining regions (CDR), interspersed with regions that are more conserved, termed framework regions (FR). Each heavy chain and light chain variable region is composed of three CDRs and four FRs, arranged from amino-terminus to carboxy-terminus in the following order: FR1, CDR1, FR2, CDR2, FR3, CDR3, FR4.

Immunoglobulin molecules can generally be of any isotype, class or subclass. The C_{H}3 domain according to the various aspects of the invention is a C_{H}3 domain of the canine IgG subtype, for example IgG-A, IgG-B, IgG-C, and IgG-D.

In canine, there are four IgG heavy chains referred to as A, B, C, and D. These heavy chains represent four different subclasses of dog IgG, which are referred to as IgG-A, IgG-B, IgG-C and IgG-D. The DNA and amino acid sequences of these four heavy chains were first identified by Tang et al. (Vet. Immunol. Immunopathol. 80: 259-270 (2001)). The amino acid and DNA sequences for these heavy chains are also available from the GenBank data bases (IgGA: accession number AAL35301.1, IgGB: accession number AAL35302.1, IgGC: accession number AAL35303.1, IgGD: accession number AAL35304.1). Canine antibodies also contain two types of light chains, kappa and lambda (GenBank accession number kappa light chain amino acid sequence ABY 57289.1, GenBank accession number ABY 55569.1). The antibodies herein may have a lambda or kappa light chain. In one embodiment, the light chain is a lambda light chain.

The term “CDR” refers to the complementarity-determining region within antibody variable sequences. There are three CDRs in each of the variable regions of the heavy chain and the light chain, which are designated CDR1, CDR2 and CDR3, for each of the variable regions. The term “CDR set” refers to a group of three CDRs that occur in a single variable region capable of binding the antigen. The exact boundaries of these CDRs can be defined differently according to different systems known in the art.

The Kabat Complementarity Determining Regions (CDRs) are based on sequence variability and are the most commonly used (Kabat et al., (1971) Ann. NY Acad. Sci. 190:382-391 and Kabat, et al., (1991) Sequences of Proteins of Immunological Interest, Fifth Edition, U.S. Department of Health and Human Services, NIH Publication No. 91-3242). Chothia refers instead to the location of the structural loops (Chothia and Lesk J. Mol. Biol. 196:901-917 (1987)). The Kabat numbering system is generally used when referring to a residue in the variable domain (approximately residues 1-107 of the light chain and residues 1-113 of the heavy chain). Another system is the ImMunoGeneTics (IMGT) numbering scheme (Lefranc et al., Dev. Comp. Immunol., 29, 185-203 (2005)). With the emergence of large-scale single cell VDJ sequencing data, enclone, a system for computing clonotypes from single cell data becomes a useful tool for streamline analysis (Jaffe et al. enclone: precision clonotyping and analysis of immune receptors. biRxiv Jul. 9, 2022. https://doi.org/10.1101/2022.04.21.489084). enclone is a computational tool available from https://10xgenomics.github.io/enclone/ which adopts the Adaptive Immune Receptor Repertoire (AIRR) numbering and CDR definitions (Heiden et al. front Immunol. 2018; 9:2206), and is used herein in respect of numbering and CDR definitions unless otherwise specified.

A chimeric antibody is a recombinant protein that contains the variable domains including the complementarity determining regions (CDRs) of an antibody derived from one species, preferably a rodent or human antibody, while the constant domains of the antibody molecule are derived from those of a canine antibody.

As used herein, the term “caninized antibody” refers to forms of recombinant antibodies that contain sequences from both canine and non-canine (e.g., murine) antibodies. In general, a caninized antibody will comprise substantially all of at least one or more typically, two variable domains in which all or substantially all of the hypervariable loops correspond to those of a non-canine immunoglobulin, and all or substantially all of the framework (FR) regions (and typically all or substantially all of the remaining frame) are those of a canine immunoglobulin sequence. A caninized antibody may comprise both the three heavy chain CDRs and the three light chain CDRS from a murine or human antibody together with a canine frame or a modified canine frame. A modified canine frame comprises one or more amino acids changes that can further optimize the effectiveness of the caninized antibody, e.g., to increase its binding to its target. The non-canine sequences, e.g., of the hypervariable loops, may further be compared to canine sequences and as many residues changed to be as similar to authentic canine sequences as possible.

In contrast, fully canine antibodies as preferred according to the present invention have canine variable regions and do not include full or partial CDRs or FRs from another species. Advantageously, fully canine antibodies as described herein have been obtained from transgenic mice comprising canine immunoglobulin sequences. Antibodies produced in these immunised mice are developed through in vivo B cell signalling and development to allow for natural affinity maturation including in vivo V(D)J recombination, in vivo junctional diversification, in vivo pairing of heavy and light chains and in vivo hypermutation. Fully canine antibodies produced in this way generate antibodies with optimal properties for developability, minimizing lengthy lead optimization prior to production at scale. Advantageously, such fully canine antibodies present the lowest possible risk of immunogenicity when introduced into a patient animal which, in turn, facilitates a repeated dosing regimen. Given that ex vivo mAb engineering runs the risk of introducing development liabilities, immunogenicity, and reduced affinity (as outlined above), fully canine antibodies of the present invention are, therefore, most likely to be efficacious therapies in a clinical context. Thus, in an embodiment, the term canine antibody refers to a fully canine antibody.

The term “monoclonal antibody” as used herein refers to an antibody obtained from a population of substantially homogeneous antibodies, i.e., the individual antibodies comprising the population are identical except for possible naturally occurring mutations and/or post-translation modifications (e.g., isomerizations, amidations, carbohydrate addition) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen.

The term “antigen binding site” refers to the part of the antibody or antibody fragment that comprises the area that specifically binds to an antigen. An antigen binding site may be provided by one or more antibody variable domains. An antigen binding site is typically comprised within the associated VH and VL of an antibody or antibody fragment.

The term “epitope” or “antigenic determinant” refers to a site on the surface of an antigen to which an immunoglobulin, antibody or antibody fragment, specifically binds. Generally, an antigen has several or many different epitopes and reacts with many different antibodies. The term specifically includes linear epitopes and conformational epitopes. Epitopes within protein antigens can be formed both from contiguous amino acids (usually a linear epitope) or non-contiguous amino acids juxtaposed by tertiary folding of the protein (usually a conformational epitope). Epitopes formed from contiguous amino acids are typically, but not always, retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods for determining what epitopes are bound by a given antibody or antibody fragment (i.e., epitope mapping by alanine-scanning mutagenesis or Pepscan) are well known in the art and include, for example, immunoblotting and immunoprecipitation assays, wherein overlapping or contiguous peptides are tested for reactivity with a given antibody or antibody fragment. An antibody binds “essentially the same epitope” as a reference antibody, when the two antibodies recognize identical or sterically overlapping epitopes. The most widely used and rapid methods for determining whether two epitopes bind to identical or sterically overlapping epitopes are competition assays, which can be configured in different formats, using either labelled antigen or labelled antibody.

In one embodiment, the epitope mays determined by site directed mutagenesis, for example using alanine scanning. In one embodiment, the epitope is a linear epitope. In one embodiment, the epitope is a conformational epitope.

The invention also relates to an antibody, antigen binding domain or antigen binding portion thereof that competes with an antibody, antigen binding domain or antigen binding portion thereof according to the invention.

Proteolytic digestion of antibodies releases different fragments termed Fv (Fragment variable), Fab (Fragment antigen binding) and Fc (Fragment crystallisation). The Fc fragment comprises the carboxy-terminal portions of both H chains held together by disulfides. The constant domains of the Fc fragment are responsible for mediating the effector functions of an antibody.

The invention extends to antigen binding portions or antigen binding fragments of the antibody. The terms “binding portion” and “fragment” are used interchangeably herein. An antibody fragment/portion is a portion of an antibody, for example a F(ab′)₂, Fab, Fv, scFv, heavy chain, light chain, variable heavy (VH), variable light (VL) chain domain and the like. Functional fragments of a full-length antibody retain the target specificity of a full antibody. Recombinant functional antibody fragments, such as Fab (Fragment, antibody), scFv (single chain variable chain fragments) and single domain antibodies (dAbs) have therefore been used to develop therapeutics as an alternative to therapeutics based on mAbs.

The invention also extends to antibody mimetics that comprise a sequence of the invention.

An “Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments scFv fragments (˜25 kDa) that consist of the two variable domains, V_{H} and V_{L} connected into a single polypeptide chain. Naturally, V_{H} and V_{L} domains are non-covalently associated via hydrophobic interactions and tend to dissociate. However, stable fragments can be engineered by linking the domains with a hydrophilic flexible linker to create a single chain Fv (scFv).

The smallest antigen binding fragment is the single variable fragment, namely the variable heavy (V_{H}) or variable light (V_{L}) chain domain. V_{H} and V_{L} domains respectively are capable of binding to an antigen. Binding to a light chain/heavy chain partner respectively or indeed the presence of other parts of the full antibody is not required for target binding. The antigen-binding entity of an antibody, reduced in size to one single domain (corresponding to the V_{H} or V_{L} domain), is generally referred to as a “single domain antibody” or “immunoglobulin single variable domain”. A single domain antibody (˜12 to 15 kDa) has thus either the V_{H} or V_{L} domain, i.e. it does not have other parts of a full antibody. The term “dAb” for “domain antibodies” generally refers to a single immunoglobulin variable domain (V_{H}, V_{HH} or V_{L}) polypeptide that specifically binds antigen.

The antibodies, antigen binding domains and antigen-binding portions or fragments thereof according to the invention are preferably isolated.

The term “isolated” refers to a moiety that is isolated from its natural environment. For example, the term “isolated” refers to an antibody or fragment thereof that is substantially free of other antibodies, antibodies or antibody fragments. Moreover, an isolated antibody may be substantially free of other cellular material and/or chemicals.

As used herein, the term “homology” or “identity” generally refers to the percentage of amino acid residues in a sequence that are identical with the residues of the reference polypeptide with which it is compared, after aligning the sequences and in some embodiments after introducing gaps, if necessary, to achieve the maximum percent homology, and not considering any conservative substitutions as part of the sequence identity. Thus, the percent homology between two amino acid sequences is equivalent to the percent identity between the two sequences. Neither N- or C-terminal extensions, tags or insertions shall be construed as reducing identity or homology. Methods and computer programs for the alignment are well known. The percent identity between two amino acid sequences can be determined using well known mathematical algorithms.

By “amino acid” herein is meant one of the 20 naturally occurring amino acids or any non-natural analogues that may be present at a specific, defined position. Amino acid encompasses both naturally occurring and synthetic amino acids. Although in most cases, when the protein is to be produced recombinantly, only naturally occurring amino acids are used.

As used herein, a “substitution of an amino acid residue” with another amino acid residue in an amino acid sequence of protein or polypeptide as described herein (an antibody for example), is equivalent to “replacing an amino acid residue” with another amino acid residue and denotes that a particular amino acid residue at a specific position in the original (e.g. wild type/germline) amino acid sequence has been replaced by (or substituted for) by a different amino acid residue. This can be done using standard techniques available to the skilled person, e.g. using recombinant DNA technology. The amino acids are changed relative to the native (wild type/germline) sequence as found in nature in the wild type (wt), but may be made in IgG molecules that contain other changes relative to the native sequence. By “wild type” or “WT” or “native” herein is meant an amino acid sequence or a nucleotide sequence that is found in nature, including allelic variations. A WT protein, polypeptide, antibody or immunoglobulin has an amino acid sequence or a nucleotide sequence that has not been intentionally modified.

Canine Antibody or Antigen-Binding Portion Thereof that Binds Canine CD3εδ

In one aspect, the invention relates to a canine antibody, antigen binding domain or antigen-binding portion thereof that binds canine CD3, in particular CD3F6.

The canine antibody, antigen binding domain or antigen-binding portion thereof according to the invention that binds canine CD3, in particular CD3 εδ, has one or more of the following properties:
a) binds specifically to canine CD3, in particular CD3 εδ;
b) binds to canine CD3, in particular CD3εδ, in an agonistic fashion and activates the canine T cell receptor, for example as demonstrated by mediating cell killing in a bispecific format where one arm recognises CD3, in particular CD3 εδ, and the other arm recognises CD20 for example, as shown in Example 4;
c) activates canine T cell receptor and leads to T cell activation ex vivo or in vivo, for example as determined by the concentration of interferon gamma, IL-2 or other T cell cytokines secreted upon activation as shown in the Examples;
d) activates canine T cells and leads to the upregulation of surface marker expression, such as CD25, CD69, PD-1 and/or other known markers for T cell activation as shown in the examples.
e) activates canine T cells and induces morphological changes such as T cell and/or PBMC clustering as shown in the Examples and/or
f) activates canine T cells and induces T cell proliferation demonstrated by markers such as Ki67 as shown in the Examples.

One possible application of the agonistic anti-CD3 antibodies, antigen binding domains or portions thereof of the invention is for the ex vivo activation and expansion of canine T cells. This can be achieved by using the anti-CD3 antibodies alone in combination with anti-CD28 antibodies and/or other T cell stimulatory factors (such as IL-2 for instance).

Another possible application of the agonistic mono-specific anti-CD3 antibodies or antigen binding domains is the immunological tolerance of T cells in treating auto-immune diseases, such as T1 D and host graft rejection diseases. Effector function deficient canine Fc is used for this application. The mono-specific anti-CD3 antibodies or antigen binding domains may be capable of inducing T cell anergy. T cell anergy is a long-term state of hypo/non-responsiveness. It is induced by the stimulation of T cells via TCR in the absence of co-stimulatory signals e.g., CD28. Inducing T cell anergy leads to a state of immunosuppression which can be used to treat autoimmune diseases, such as type I diabetes. Measures of T cell activation are known in the art and include surface markers, proliferation of T cells and cytokine release.

In one embodiment, the canine antibody, antigen binding domain or antigen-binding portion thereof that binds canine CD3 according to the invention has one or more of the following properties:
a) binds canine CD3, in particular CD3εδ, with a binding dissociation equilibrium constant (KD) of 100 nM-1000 nM;
b) binds to canine CD3, in particular CD3εδ, in an agonistic fashion and activates the canine T cell receptor, and mediates target specific cell killing in a bispecific format where one arm recognises CD3, in particular CD3εδ and the other arm recognises CD20 for example, as shown in Example 4 in vitro and ex vivo;
c) triggers the T cell surface upregulation of markers such as CD25, CD69 upon target mediated cell killing in a bispecific format where one arm recognises CD3, in particular CD3εδ and the other arm recognises a target antigen such as CD20 in vitro, ex vivo and in vivo, for example as demonstrated in the Examples;
d) activates canine T cells with low, e.g. minimal, secretion of cytokines such as IFN-γ in vitro and/or
e) mediates in vivo target specific cell killing in a bispecific format where one arm recognises CD3, in particular CD3εδ and the other arm recognises for example CD20, with minimal cytokine release, for example, IFN-γ, IL-2, IL-6 and/or TNF-α release.

An antibody, antigen binding domain or antigen-binding portion thereof that exhibits the properties a) to e) as in the forgoing paragraph is particularly useful for use in bispecific antibody molecules with an anti-CD3 arm for T cell engagement and a target cell specific arm, such as directed to the antigen CD20, CD19, BCMA, CD123, CD33, CD38.

In yet a further embodiment, the canine antibody, antigen binding domain or antigen-binding portion thereof binds canine CD3, in particular CD3εδ with a monovalent binding dissociation equilibrium constant (KD) of less than 100 nM or 100 nM-1 uM. An antibody, antigen binding domain or antigen-binding portion thereof that exhibits such kinetic property is particularly useful for use in a monovalent format and can be used as an agonistic antibody to activate T cells.

These properties above can be measured by methods known in the art, such as the methods disclosed in the Examples, including in vivo studies in mouse models or in dogs.

In one embodiment, the canine antibody, antigen binding domain or antigen-binding fragment portion that binds canine CD3 comprises the complementarity determining regions (CDRs) of a heavy chain variable region (HCVR) having an amino acid sequence as set forth in Table 2 or a sequence with at least 70%, 75%, 80%, 85%, 90% or 95% sequence identity thereto. The complementarity determining regions (CDRs) refer to the three CDRs, i.e. CDR1, 2 and 3.

In one embodiment, the canine antibody, antigen binding domain or antigen-binding fragment portion thereof comprises: (a) the complementarity determining regions (CDRs) of a heavy chain variable region (HCVR) having an amino acid sequence as set forth in Table 2 or a sequence with at least 70%, 75%, 80%, 85%, 90% or 95% sequence identity thereto; and (b) the CDRs of a light chain variable region (LCVR) having an amino acid sequence as set forth in Table 2 or a sequence with at least 70%, 75%, 80%, 85%, 90% or 95% sequence identity thereto. In other words, in one embodiment, the canine antibody, antigen binding domain or antigen-binding fragment portion thereof comprises: (a) the HCVR CDRs as set out for one of the PMX molecules in Table 2 or a sequence with at least 70%, 75%, 80%, 85%, 90% or 95% sequence identity thereto; and (b) the LCVR CDRs as set out for one of the PMX molecules in Table 2 or a sequence with at least 70%, 75%, 80%, 85%, 90% or 95% sequence identity thereto. In one embodiment, the canine antibody, antigen binding domain or antigen-binding fragment portion thereof comprises the HCVR CDRs and the LCVR CDRs of PMX157, PMX158, PMX160, PMX190, PMX162, PMX163, PMX189, PMX165, PMX167, PMX168, PMX169, PMX170, PMX171, PMX172, PMX173, PMX174, PMX175, PMX176, PMX177, PMX178, PMX179, PMX180, PMX181, PMX182, PMX183, PMX184, PMX185, PMX186, PMX187, PMX188, PMX190, PMX192, PMX193, PMX194, PMX195, PMX196, PMX197, PMX198, PMX200, PMX272, PMX273, PMX285 or PMX286 as shown in Table 2.

In one embodiment, the invention relates to an isolated canine antibody, antigen binding domain or antigen-binding portion thereof which binds canine CD3 wherein said antibody comprises
a) a HCVR CDR1 sequence comprising or consisting of a SEQ ID No. as shown for a PMX molecule in Table 2 or an amino acid sequence which has at least 70%, 75%, 80%, 85%, 90% or 95% sequence identity thereto,
b) a HCVR CDR2 sequence comprising or consisting of a SEQ ID No. as shown for the respective PMX molecule in Table 2 or an amino acid sequence which has at least 70%, 75%, 80%, 85%, 90% or 95% sequence identity thereto,
c) a HCVR CDR3 sequence comprising or consisting of a SEQ ID No. as shown for the respective PMX molecule in Table 2 or an amino acid sequence which has at least 70%, 75%, 80%, 85%, 90% or 95% sequence identity thereto,
d) a LCVR CDR1 sequence comprising or consisting of a SEQ ID No. as shown for the respective PMX molecule in Table 2 or an amino acid sequence which has at least 60%, 70%, 80% or 90% sequence identity thereto,
e) a LCVR CDR2 sequence comprising or consisting of a SEQ ID No. as shown for the respective PMX molecule in Table 2 or an amino acid sequence which has at least 70%, 75%, 80%, 85%, 90% or 95% % sequence identity thereto and
f) a LCVR CDR3 sequence comprising or consisting of a SEQ ID No. as shown for the respective PMX molecule in Table 2 or an amino acid sequence which has at least 70%, 75%, 80%, 85%, 90% or 95% sequence identity thereto.

In one embodiment, the isolated canine antibody, antigen binding domain or antigen-binding portion thereof which binds canine CD3 wherein said antibody, antigen binding domain or antigen-binding portion thereof comprises
a) a HCVR CDR1 sequence comprising or consisting of a SEQ ID No. as shown for a PMX molecule in Table 2,
b) a HCVR CDR2 sequence comprising or consisting of SEQ ID No. as shown for the respective PMX molecule in Table 2;
c) a HCVR CDR3 sequence comprising or consisting of SEQ ID No. as shown for the respective PMX molecule in Table 2,
d) a LCVR CDR1 sequence comprising or consisting of SEQ ID No. as shown for the respective PMX molecule in Table 2,
e) a LCVR CDR2 sequence comprising or consisting of SEQ ID No. as shown for the respective PMX molecule in Table 2 and
f) a LCVR CDR3 sequence comprising or consisting of SEQ ID No. as shown for the respective PMX molecule in Table 2
or an isolated canine antibody, antigen binding domain or antigen-binding portion thereof with a CDR as described above but which has one or more CDR which has 1, 2, 3, 4 or 5 amino acid substitutions in one or more LCVR or HCVR CDR compared to the reference CDR sequence.

In one embodiment, the canine antibody, antigen binding domain or antigen-binding fragment portion comprises: (a) a heavy chain variable region (HCVR) having an amino acid sequence as set forth in Table 2 or an amino acid sequence which has at least 70%, 75%, 80%, 85%, 90% or 95% sequence identity thereto; and (b) a light chain variable region (LCVR) having an amino acid sequence as set forth in Table 2 or an amino acid sequence which has at least 70%, 75%, 80%, 85%, 90% or 95% sequence identity thereto. In one embodiment, the canine antibody, antigen binding domain or antigen-binding fragment portion thereof comprises a HC CDRs and a LC CDRs of a PMX molecule as shown in Table 2.

In one embodiment, the canine antibody, antigen binding domain or antigen-binding fragment portion thereof comprises the HCVR and the LCVR of PMX157, PMX158, PMX160, PMX190, PMX162, PMX163, PMX189, PMX165, PMX167, PMX168, PMX169, PMX170, PMX171, PMX172, PMX173, PMX174, PMX175, PMX176, PMX177, PMX178, PMX179, PMX180, PMX181, PMX182, PMX183, PMX184, PMX185, PMX186, PMX187, PMX188, PMX190, PMX192, PMX193, PMX194, PMX195, PMX196, PMX197, PMX198, PMX200, PMX272, PMX273, PMX285 or PMX286 as shown in Table 2.

In one embodiment, the antibody, antigen binding domain or antigen-binding portion thereof comprises
a) a HCVR sequence comprising or consisting of a SEQ ID No. as shown for a PMX molecule in Table 2 or a sequence with 1, 2, 3, 4, 5, 6, 7, 8 9, or 10 amino acid modifications (e.g. a deletion, addition or substitution) in the HCVR framework region compared to the reference HCVR and
b) a LCVR sequence comprising or consisting of SEQ ID No. as shown for the respective PMX molecule in Table 2 or a sequence with 1, 2, 3, 4, 5, 6, 7, 8 9, or 10 amino acid modifications (e.g. a deletion, addition or substitution) in the LCVR framework region compared to the reference LCVR.

In one embodiment, the antigen binding domain or antigen-binding portion thereof is a F(ab′)2, Fab, Fv, scFv, heavy chain, light chain, variable heavy (V_{H}) domain or variable light (V_{L}).

In one embodiment, the antigen binding domain or antigen-binding portion is a heavy chain and comprises the HCVR CDRs as set out for a PMX molecule as shown in Table 2 or a sequence with at least 70%, 75%, 80%, 85%, 90% or 95% sequence identity thereto. In one embodiment, the canine antigen binding domain or antigen-binding fragment portion comprises or consists of the HCVR CDRs as shown for PMX160, PMX162, PMX169, PMX170, PMX171, PMX172, PMX186, PMX187, PMX190, PMX188 or PMX189.

In one embodiment, the antigen binding domain or antigen-binding portion comprises or consists of a heavy chain variable region and comprises the HCVR as set out for a PMX molecule as shown in Table 2 or a sequence with at least 70%, 75%, 80%, 85%, 90% or 95% sequence identity thereto. Thus, in one embodiment, the canine antigen binding domain or antigen-binding fragment portion comprises or consists of a HCVR having an amino acid sequence as set forth in Table 2 or a sequence with at least 70%, 75%, 80%, 85%, 90% or 95% sequence identity thereto.

In one embodiment, the canine antigen binding domain or antigen-binding fragment portion comprises or consists of the HCVR as shown for PMX160, PMX162, PMX169, PMX170, PMX171, PMX172, PMX186, PMX187, PMX190, PMX188 or PMX189.

In one embodiment, the antibody, antigen binding domain or antigen-binding portion thereof as described above comprises an Fc region, for example a canine Fc region, for example a canine IgGB Fc region.

In one embodiment, the canine antibody or antigen-binding portion described herein specifically binds to CD3 and comprises a VH CDR1 comprising the amino acid sequence of SEQ ID NO: 47; a VH CDR2 comprising the amino acid sequence of SEQ ID NO: 48; the VH CDR3 comprising the amino acid sequence of SEQ ID NO: 49; the VL CDR1 comprising the amino acid sequence of SEQ ID NO: 50; the VL CDR2 comprising the amino acid sequence of SEQ ID NO: 51; and the VL CDR3 comprising the amino acid sequence SEQ ID NO: 52. In another embodiment, the canine antibody or antigen-binding portion described herein specifically binds to CD3 and comprises a VH CDR1 comprising the amino acid sequence of SEQ ID NO: 87; a VH CDR2 comprising the amino acid sequence of SEQ ID NO: 88; the VH CDR3 comprising the amino acid sequence of SEQ ID NO: 89; the VL CDR1 comprising the amino acid sequence of SEQ ID NO: 90; the VL CDR2 comprising the amino acid sequence of SEQ ID NO: 91; and the VL CDR3 comprising the amino acid sequence SEQ ID NO: 92. In yet another embodiment, the canine antibody or antigen-binding portion described herein specifically binds to CD3 and comprises a VH CDR1 comprising the amino acid sequence of SEQ ID NO: 127; a VH CDR2 comprising the amino acid sequence of SEQ ID NO: 128; the VH CDR3 comprising the amino acid sequence of SEQ ID NO: 129; the VL CDR1 comprising the amino acid sequence of SEQ ID NO: 130; the VL CDR2 comprising the amino acid sequence of SEQ ID NO: 131; and the VL CDR3 comprising the amino acid sequence SEQ ID NO: 132. In one embodiment, the canine antibody or antigen-binding portion described herein specifically binds to CD3 and comprises a VH CDR1 comprising the amino acid sequence of SEQ ID NO: 167; a VH CDR2 comprising the amino acid sequence of SEQ ID NO: 168; the VH CDR3 comprising the amino acid sequence of SEQ ID NO: 169; the VL CDR1 comprising the amino acid sequence of SEQ ID NO: 170; the VL CDR2 comprising the amino acid sequence of SEQ ID NO: 171; and the VL CDR3 comprising the amino acid sequence SEQ ID NO: 172.

In one embodiment, the canine antibody or antigen-binding portion described herein specifically binds to CD3 and comprises a VH comprising the amino acid sequence of SEQ ID NO: 44; and a VL comprising the amino acid sequence of SEQ ID NO: 46. In another embodiment, the canine antibody or antigen-binding portion described herein specifically binds to CD3 and comprises a VH comprising the amino acid sequence of SEQ ID NO: 84; and a VL comprising the amino acid sequence of SEQ ID NO: 86. In yet another embodiment, the canine antibody or antigen-binding portion described herein specifically binds to CD3 and comprises a VH comprising the amino acid sequence of SEQ ID NO: 124; and a VL comprising the amino acid sequence of SEQ ID NO: 126. In one embodiment, the canine antibody or antigen-binding portion described herein specifically binds to CD3 and comprises a VH comprising the amino acid sequence of SEQ ID NO: 164; and a VL comprising the amino acid sequence of SEQ ID NO: 166.

The variable region sequences described herein, including but not limited to the amino acid and nucleotide sequences shown in Table 2 (and/or fragments thereof) may be used in combination with one or more amino acid sequences and/or nucleotide sequences encoding one or more constant chains (and/or a fragment thereof) of an antibody molecule. For instance, the variable region amino acid sequences shown in Table 2 may be joined to the constant regions of any antibody molecule of the same or a different species (e.g., human, goat, rat, sheep, chicken) of that from which the variable region amino acid sequence was derived. Preferably, the variable region amino acid sequences shown in Table 2 is joined to the constant regions of a canine antibody and may be the constant region from any of canine IgG A, B, C or D. In one embodiment, the constant region is canine IgG B constant region. Dog IGGB (SEQ ID NO: 28), dog IGK or dog IGLC5 constant regions may be used. Variants of the constant region which have altered effector regions may also be used, for example a variant of Dog IGGB (SEQ ID NO: 30). These variants may be formed by introducing mutations in canine IgG-B which abolishes the effector function. Canine IgG-B may be modified to reduce or abolish canine IgG-B effector function when compared to the same polypeptide comprising a wild-type IgG-B Fc domain. The regions targeted in the amino acid sequence of the Fc domain for modification may include the lower hinge, proline region and SHED region, where potential interactions with FcgammaR and C1q occur. Examples of such mutations are provided in WO 2023/012486 and are incorporated herein by reference.

Other such variants may comprise charge pair combinations in canine CH3 domains which may significantly enriches heavy chain heterodimersation over homodimer formation. This may minimise the formation of homodimer contaminants for the production of bispecific antibodies. These charge pair combinations may be in the canine IgG CH3 domain interface of the Fc region wherein said IgG is selected from IgG-A, B, C or D. A first canine IgG CH3 domain and a second canine IgG CH3 domain may both be engineered in a complementary manner so that each CH3 domain (or a polypeptide comprising it) substantially does not homodimerise with itself or homodimerises at a lower rate, but is forced to heterodimerise with the complementary engineered other CH3 domain. In other words, the first and second CH3 domain may heterodimerise and few homodimers between the two first or the two second CH3 domains are formed. Examples of such mutations are provided in WO 2021/214460 A1 and are incorporated herein by reference.

Variants may also comprise mutations in canine CH2 or CH3 IgG Fc domains which result in a differential affinity for a binding affinity reagent and/or improved stability. For example, the binding molecule may have a differential affinity for binding Protein A relative to the wild type IgG Fc domain. The differential affinity of the immunoglobulin heavy chains allows for optimised isolation of said binding proteins or antibodies. For example, suitable variant IgG Fc domains may comprise one or more amino acid substitution which increases affinity for binding protein A, or one or more amino acid substitution which decreases affinity for binding Protein A. Examples of such mutations are provided in GB2311984.5 and are incorporated herein by reference.

Thus, in one embodiment, the antigen binding domain, or antibody or antigen-binding portion thereof comprises mutant variants with deficient Fc binding arising from mutations in canine IgG-B. In another embodiment, the antibody or antigen-binding portion thereof comprises mutant variants with enriched heavy chain heterodimersation arising from mutations in canine CH3 domains of IgG-A, B, C or D. In another embodiment, the antigen binding domain, or antibody or antigen-binding portion thereof comprises mutant variants with mutations in canine CH2 or CH3 IgG Fc domains which result in a differential affinity for a binding affinity reagent and/or improved stability. The antigen binding domain, or antibody or antigen-binding portion thereof may have a single mutation which has a single mutant phenotype for example, decreased Fc effector function, or may have multiple mutations resulting in multiple phenotypes for example, decreased Fc effector function, enriched heavy chain heterodimersation and altered Protein A binding affinity.

Also within the scope of the invention are variants of the antibodies, antigen binding domain and antigen binding portions as described above.

A variant of an antibody, antigen binding domain or antigen binding portion thereof as described herein has at least 75%, 80%, 85%, 90%, 91%, 92%, 93%, 94%, 95%, 96%, 97%, 98% or 99% sequence identity to the non-variant molecule. In one embodiment, sequence identity is at least 95%. In one embodiment, the modification (i.e. difference in sequence) is a conservative sequence modification.

As used herein, the term “conservative sequence modifications” is intended to refer to amino acid modifications that do not significantly affect or alter the binding characteristics of the antibody containing the amino acid sequence. Such conservative modifications include amino acid substitutions, additions and deletions. Modifications can be introduced into an antibody or antigen binding portion thereof of the invention by standard techniques known in the art, such as site-directed mutagenesis and PCR-mediated mutagenesis.

Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within the CDR regions of an antibody of the invention can be replaced with other amino acid residues from the same side chain family and the altered antibody can be tested for retained function (i.e., CD3 binding) using the functional assays described herein.

Thus, these amino acid changes can typically be made without altering the biological activity, function, or other desired property of the polypeptide, such as its affinity or its specificity for antigen. In general, single amino acid substitutions in nonessential regions of a polypeptide do not substantially alter biological activity. Furthermore, substitutions of amino acids that are similar in structure or function are less likely to disrupt the polypeptides' biological activity. Abbreviations for the amino acid residues that comprise polypeptides and peptides described herein, and conservative substitutions for these amino acid residues are shown in Table 3 below.

TABLE 3
Amino Acid Residues and Examples of
Conservative Amino Acid Substitutions

| Original residue | Conservative |
|---|---|
| Three letter code, single letter code | substitution |
| Alanine, Ala, A | Gly, Ser |
| Arginine, Arg, R | Lys, His |
| Asparagine, Asn, N | Gln, His |
| Aspartic acid Asp, D | Glu, Asn |
| Cysteine, Cys, C | Ser, Ala |
| Glutamine, Gln, Q | Asn |
| Glutamic acid, Glu, E | Asp, Gln |
| Glycine, Gly, G | Ala |
| Histidine, His, H | Asn, Gln |
| Isoleucine, Ile, I | Leu, Val |
| Leucine, Leu, L | Ile, Val |
| Lysine, lys, K | Ar, His |
| Methionine, Met, M | Leu, Ile, Tyr |
| Phenylalanine, Phe, F | Tyr, Met, Leu |
| Proline, Pro, P | Ala |
| Serine, Ser, S | Thr |
| Threonine, Thr, T | Ser |
| Tryptophan, Trp, W | Tyr, Phe |
| Tyrosine, Tyr, Y | Try, Phe |
| Valine, Val, V | Ile, Leu |

In some embodiments, the invention provides an antibody, antigen binding domain or antigen binding portion thereof that is a variant of an antibody, antigen binding domain or antigen binding portion thereof compared to a sequence described herein, e.g. selected from the sequences shown in Table 3 that comprises one or more sequence modification and has improvements in one or more of a property such as binding affinity, specificity, thermostability, expression level, effector function, glycosylation, reduced immunogenicity, or solubility as compared to the unmodified antibody or fragment thereof.

Suitable methods for measuring properties which may suggest that the antigen binding domain or antibody can be successfully developed at scale include first purification using chromatography, such as affinity chromatography chromatography (Protein A: MabSelect Sure LX), anion exchange chromatography (Capto Q), cation exchange chromatography (Capto S) and buffer exchange (G-25 Fine), followed by assessment of whether the antibody remains intact (e.g. using SDS PAGE analysis to determine molecular weight, HPLC-SEC to calculate % of monomers, assess aggregation, and thermostability (Tm) studies.

A skilled person will know that there are different ways to identify, obtain and optimise the antigen binding molecules as described herein, including in vitro and in vivo expression libraries. This is further described in the Examples. Optimisation techniques known in the art, such as display (e.g., ribosome and/or phage display) and/or mutagenesis (e.g., error-prone mutagenesis) can be used. The invention therefore also comprises sequence optimised variants of the antibodies described herein.

In one embodiment, modifications can be made to decrease the immunogenicity of the antigen binding domain or antibody. For example, one approach is to revert one or more framework residues to the corresponding canine germline sequence. More specifically, an antibody that has undergone somatic mutation may contain framework residues that differ from the germline sequence from which the antibody is derived. Such residues can be identified by comparing the antibody framework sequences to the germline sequences from which the antibody is derived. In one embodiment, all framework sequences are germline sequence.

To return one or more of the amino acid residues in the framework region sequences to their germline configuration, the somatic mutations can be “backmutated” to the germline sequence by, for example, site-directed mutagenesis or PCR-mediated mutagenesis.

Another type of framework modification involves mutating one or more residues within the framework region, or even within one or more CDR regions, to remove T cell epitopes to thereby reduce the potential immunogenicity of the antigen binding domain or antibody.

In some embodiments, the antigen-binding proteins, fragments and derivatives thereof, and fusion proteins of the present disclosure undergo post-translational modifications, for example but not limited to, a glutamine can be cyclized or converted to pyroglutamic acid; additionally, or alternatively, amino acids can undergo deamidation, isomerization, glycation and/or oxidation. The polypeptides of the present disclosure can undergo additional post-translational modification, including glycosylation, for example N-linked or 0-linked glycosylation, at sites that are well-known in the art. Changes can be made in the amino acid sequence of a polypeptide to preclude or minimize such alterations, or to facilitate them in circumstances where such processing is beneficial. Polypeptides of the present disclosure include polypeptides that have been modified, for example, to: (1) reduce susceptibility to proteolysis, (2) reduce susceptibility to oxidation, (3) alter binding affinity for forming protein complexes, (4) alter binding affinities, and (4) confer or modify other physicochemical or functional properties.

Glycosylation can also be altered to, for example, increase the affinity of the antigen binding domain or antibody for antigen. Such carbohydrate modifications can be accomplished by, for example, altering one or more sites of glycosylation within the antigen binding domain or antibody sequence. For example, one or more amino acid substitutions can be made that result in elimination of one or more variable region framework glycosylation sites to thereby eliminate glycosylation at that site. Such glycosylation may increase the affinity of the antigen binding domain or antibody for the antigen.

In some applications, the antibody, antigen binding domain or antigen binding portion thereof may bind the desired target (canine CD3 and/or CD20) but has altered ability to bind Fc receptors as compared to standard binding agents. In one example, the binding agents are antigen binding domains or antibodies that have modified glycosylation patterns. IgG molecules, for example, typically contain N-linked oligosaccharides, for example fucose.

In one embodiment, the antibody, antigen binding domain or antigen-binding portion thereof as described herein is a-fucosylated. In cancer immunotherapy, antibodies may rely on the Fc-mediated immune effector function, antibody-dependent cellular cytotoxicity (ADCC), as the major mode of action to deplete tumour cells. It is well-known that this effector function is modulated by the N-linked glycosylation in the Fc region of the antibody. In particular, absence of core fucose on the Fc N-glycan has been shown to increase IgG1 Fc binding affinity to the FcγRIIIa present on immune effector cells such as natural killer cells and lead to enhanced ADCC activity. Therefore, a-fucosylated antigen binding domains or antibodies may have advantageous to improve therapeutic efficacy and absence/removal of the fucose enhances the ability of the antigen binding domain or antibody to interact with Fc receptors. Antigen binding domains or antibodies of this type may be referred to as “a-fucosylated”. Such antigen binding domains or antibodies may be produced using techniques described herein and/or that may be known in the art. In some embodiments, a nucleic acid sequence encoding an antigen binding domain or antibody may be expressed in a cell line that has modified glycosylation abilities (e.g., deleted, modified or lesser amount of fucosyl transferase) and fail to add the typical fucose moieties.

In one embodiment, the Fc portion of the antigen binding domain or antibody may be modified.

In one embodiment, the one or more substitution in the variant is in the CDR1, 2 and/or 3 region. For example, there may be 1, 2, 3, 4, 5 or more amino acid substitutions in the CDR1, 2 and/or 3 region. In another example, there may be 1 or 2 amino acid deletions.

In one embodiment, the one or more substitution is in the framework region. For example, there may be 1 to 20, e.g. 1 to 10, e.g. 1, 2, 3, 4, 5, 6, 7, 8, 9 or 10, amino acid substitutions in the HC variable region and/or LC variable region framework region.

The anti-CD3 antibodies, antigen binding domains or portions thereof of the invention preferably have KD, IC50 and/or EC50 values, e.g. a KD as further described herein and in the Examples. Suitably, the KD value is sufficient for the antigen binding domains or antibodies to have the desired biological effect. For example, the monovalent KD can be 100 nM-1000 nM or below 100 nM. KD, IC50 and/or EC50 values may be measured as known in the art, for example as described in the Examples.

The term “KD” refers to the “equilibrium dissociation constant” and refers to the value obtained in a titration measurement at equilibrium, or by dividing the dissociation rate constant (Koff) by the association rate constant (Kon). “KA” refers to the affinity constant. The association rate constant, the dissociation rate constant and the equilibrium dissociation constant are used to represent the binding affinity of an antigen binding domain or antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® SPR assay can be used.

The invention also relates to an isolated canine antibody, antigen binding domain or antigen-binding portion thereof that binds to canine CD3 competing with an antibody, antigen binding domain or antigen-binding portion thereof as described above. Antibodies, antigen binding domains, antibody fragments or antibody mimetics that bind at or near the same epitope or an overlapping epitope on canine CD3 as any of the CD3 antigen binding domains or antibodies of the invention have the ability to cross-compete for binding to CD3 with any of the antigen binding domains or antibodies of the invention. The antigen binding domains or antibodies of the invention can thus be used as a reference antigen binding domain or antibody to assess such cross-reactivity. Such cross-competing antigen binding domains or antibodies can be identified based on their ability to cross-compete with an antigen binding domain or antibody described herein in standard CD3 binding assays. For example, BIAcore® analysis, ELISA assays or flow cytometry may be used to demonstrate cross-competition with the antigen binding domains or antibodies.

### Immunoconjugates and Other Binding Agents

The invention relates to immunoconjugates and other binding agents comprising the antibody, antigen binding domain or antigen binding portion thereof according to the invention that binds CD3. For example, the antibody, antigen binding domain or antigen-binding portion thereof according to the invention may be conjugated to a therapeutic moiety or non-therapeutic moiety.

In one embodiment, the therapeutic moiety is a binding molecule that binds to a target antigen of interest, for example selected from an antibody, antigen binding domain or antibody fragment (e.g., a Fab, F(ab′)2, Fv, a single chain Fv fragment (scFv) or single domain antibody, for example a V_{H} or V_{HH} domain) or antibody mimetic protein.

In one embodiment, the proteins or polypeptides that comprise the antibody, antigen binding domain or antigen binding portion thereof that binds to CD20 as described herein and a second moiety are fusion proteins. In one embodiment, the proteins or polypeptides that comprise the antibody, antigen binding domain or antigen binding portion thereof that binds to CD3 as described herein and a second moiety are drug conjugates.

As used herein “conjugate” refers to a composition comprising the antibody or antigen binding domain that binds to CD3 as described herein that is bonded/conjugated to a drug.

Such conjugates include “drug conjugates” which comprise the antigen binding domain or antibody that binds to CD3 to which a drug is covalently bonded, and “non-covalent drug conjugates” which comprise the antigen binding domain or antibody that binds to CD3 to which a drug is noncovalently bonded.

As used herein, “drug conjugate” refers to a composition comprising the antigen binding domain or antibody to which a drug is covalently bonded. The drug can be covalently bonded to the antigen binding domain, or antibody or antibody fragment directly or indirectly through a suitable linker moiety. The drug can be bonded to the antigen binding domain or antibody at any suitable position, such as the amino-terminus, the carboxyl-terminus or through suitable amino acid side chains.

In one embodiment, the antibody is linked to the second moiety with a peptide linker or other suitable linker to connect the two moieties.

The term “peptide linker” refers to a peptide comprising one or more amino acids. A peptide linker comprises 1 to 50, for example 1 to 20 amino acids. Peptide linkers are known in the art and non-limiting examples are described herein. Suitable, non-immunogenic linker peptides are, for example, linkers that include G and/or S residues, (G4S)n, (SG4)n or G4(SG4)n peptide linkers, wherein “n” is generally a number between 1 and 10, e.g., 1, 2, 3, 4, 5, 6, 7, 8, 9 or 10.

The binding agent may be multispecific, for example bispecific.

In one embodiment, the binding molecule is bispecific. Thus, in one aspect, the invention relates to a bispecific molecule comprising an antibody, antigen binding domain or antigen binding portion thereof described herein linked to a second moiety having a different binding specificity than said antibody, antigen binding domain or antigen binding portion thereof. Thus, the second antibody, antigen binding domain or antigen binding portion thereof binds to a different target antigen, e.g. a target of interest. In one embodiment the target of interest may be a tumour antigen. In particular, the canine antibody, antigen binding domain or antigen-binding portion thereof that binds canine CD3 as described above may be used in a bispecific format to target another target, for example canine CD20. Such bispecific antigen binding domains or antibodies are further explained below.

The invention therefore also relates to the use of an antibody, antigen binding domain or antigen binding portion thereof that targets canine CD3 in a bispecific molecule, for example where the second antigen targeted is selected from the following canine antigens: CD20, CD19, CD20, BCMA, CD33, CD38, CEA, CLEC12A, DLL3, EGFRvIII, EpCAM, FcRH5, FLT3, GPC3, gpA33, GPRC5D, HER2, MUC16, P-cadherin, PSMA, SSTR2, CLDN18.

In one embodiment the binding molecule, e.g. the protein or construct is multispecific and comprises a further, i.e. third, fourth, fifth etc moiety.

The therapeutic moiety can also be selected from a half life extending moiety, cytotoxin, or radioisotope.

The non-therapeutic moiety can be selected from a label, liposome or nanoparticle. The label is detectable or functional. A label can be any molecule that produces or can be induced to produce a signal, including but not limited to fluorophores, fluorescers, radiolabels, enzymes, chemiluminescers, a nuclear magnetic resonance active label or photosensitizers. Thus, the binding may be detected and/or measured by detecting fluorescence or luminescence, radioactivity, enzyme activity or light absorbance.

According to the invention, an antibody, antigen binding domain or antigen binding portion that is linked to one moiety may further be linked to another moiety. For example, a may be linked to a therapeutic moiety and further linkage to a non-therapeutic moiety may be provided either via the antigen binding domain, or antibody or the moiety.

In one embodiment, the binding agent or the antibody, antigen binding domain or antigen binding portion thereof according to the invention may comprise a half life extending moiety. This may be selected from an antibody, antigen binding domain or antigen binding portion thereof that binds canine serum albumin. Alternatively, extended half life may be conferred through PEGylation.

The term “half-life” as used can generally refer to the time taken for the serum concentration of the amino acid sequence, compound or polypeptide to be reduced by 50%, in vivo, for example due to degradation of the sequence or compound and/or clearance or sequestration of the sequence or compound by natural mechanisms. The in vivo half-life of an amino acid sequence, compound or polypeptide of the invention can be determined in any manner known per se, such as by pharmacokinetic analysis. Suitable techniques will be clear to the person skilled in the art. The half-life can be expressed using parameters such as the tl/2-alpha, tl/2-beta and the area under the curve (AUC). Half-lives (t alpha and t beta) and AUC can be determined from a curve of serum concentration of conjugate or fusion against time. Thus, the term “half-life” as used herein in particular refers to the tl/2-beta or terminal half-life (in which the tl/2-alpha and/or the AUC or both may be kept out of considerations).

For example, in a first phase (the alpha phase) the drug composition (e. g., drug conjugate, noncovalent drug conjugate, drug fusion) is undergoing mainly distribution in the patient, with some elimination. A second phase (beta phase) is the terminal phase when the drug composition (e. g., drug conjugate, noncovalent drug conjugate, drug fusion) has been distributed and the serum concentration is decreasing as the drug composition is cleared from the patient. The t alpha half-life is the half-life of the first phase and the t beta half-life is the half-life of the second phase.

Bispecific Antibody or Antigen Binding Portions Thereof that Targets Canine CD3 and CD20

In another aspect, the invention relates to a bispecific canine antigen-binding molecule comprising a first antibody, antigen binding domain or antigen-binding portion thereof that specifically binds canine CD3, in particular CD3 εδ, and a second antibody, antigen binding domain or antigen-binding portion thereof that specifically binds canine CD20. The bispecific canine antigen binding molecule thus comprises one arm (or portion) that specifically binds canine CD3, and a second arm (or portion) that specifically binds canine CD20.

In one embodiment, the bispecific canine antibody, antigen binding domain or antigen-binding portion thereof has one or more of the following properties:
a) binds specifically to canine CD3, in particular CD3εδ;
b) binds specifically to canine CD20;
c) has a CD3 binding arm that binds canine CD3, in particular CD3εδ with a monovalent binding dissociation equilibrium constant (KD) of 100 nM-1000 nM;
d) binds to canine CD3, in particular CD3εδ in an agonistic fashion, activates the canine T cell receptor, and mediates target specific cell killing in a bispecific format, for example as shown in Example 4 in vitro and ex vivo;
e) triggers the T cell surface upregulation of markers such as CD25 and/or CD69 upon target mediated cell killing in a bispecific format as demonstrated, for example, in the Examples in vitro, ex vivo and in vivo; f) activates canine T cells with low, e.g. minimal, secretion of cytokines such as IFN-γ, e.g. in vitro as shown in the Examples and/or
g) mediates in vivo target specific cell killing with low/minimal cytokine release, for example, IFN-γ, IL-2, IL-6 and/or TNF-α release.

In one embodiment, the bispecific canine antigen binding molecule has a monovalent CD3 binding arm that binds canine CD3, in particular CD3εδ, with a binding dissociation equilibrium constant (KD) of about 100 nM to about 1000 nM.

Without wishing to be bound by theory, the inventors believe that an affinity for canine CD3 within this range provides sufficient binding affinity to elicit agonistic activity and mediate cell killing in the bispecific format, but also ensures low/minimal cytokine release. This is important as cytokine release is one of the major safety considerations for T cell engager bispecific antibodies. It is believed that higher affinity (i.e. lower than about 100 nM) leads to greater cytokine release which in turn has implications for safety.

In one embodiment, the first antibody, antigen binding domain or antigen-binding portion thereof that specifically binds canine CD3 has a sequence, e.g. a HCVR/LCVR CDR sequences, HCVR and/or LCVR, as described herein and shown in Table 2 which provides the SEQ ID NOs for CDR1, 2, 3, HCVR and LCVR polypeptides.

In one embodiment, the antigen binding domain or antigen-binding portion that specifically binds canine CD3 comprises the HCVR CDRs as set out for a PMX molecule as shown in Table 2 or a sequence with at least 70%, 75%, 80%, 85%, 90% or 95% sequence identity thereto. In one embodiment, the canine antigen binding domain or antigen-binding fragment portion that specifically binds canine CD3 comprises or consists of the HCVR CDRs as shown for PMX160, PMX162, PMX169, PMX170, PMX171, PMX172, PMX186, PMX187, PMX190, PMX188 or PMX189 as shown in Table 2.

In one embodiment, the antigen binding domain or antigen-binding portion that specifically binds canine CD3 comprises the HCVR sequence as set out for a PMX molecule as shown in Table 2 or a sequence with at least 70%, 75%, 80%, 85%, 90% or 95% sequence identity thereto. Thus, in one embodiment, the canine antigen-binding fragment portion comprises a HCVR having an amino acid sequence as set forth in Table 2 or a sequence with at least 70%, 75%, 80%, 85%, 90% or 95% sequence identity thereto.

In one embodiment, the canine antigen binding domain or antigen-binding fragment portion that specifically binds canine CD3 comprises or consists of the anti-CD3 HCVR as shown for PMX160, PMX162, PMX169, PMX170, PMX171, PMX172, PMX186, PMX187, PMX190, PMX188 or PMX189 as shown in Table 2.

In one embodiment, the antigen binding domain antigen-binding portion that specifically binds canine CD3 comprises the LCVR CDRs as set out for a PMX molecule as shown in Table 2 or a sequence with at least 70%, 75%, 80%, 85%, 90% or 95% sequence identity thereto. In one embodiment, the canine antigen binding domain or antigen-binding fragment portion thereof that specifically binds canine CD3 comprises or consists of the LCVR CDRs as shown for PMX272, PMX285, PMX286, PMX188 or PMX189 as shown in Table 2.

In one embodiment, the antigen binding domain or antigen-binding portion that specifically binds canine CD3 comprises the LCVR sequence as set out for a PMX molecule as shown in Table 2 or a sequence with at least 70%, 75%, 80%, 85%, 90% or 95% sequence identity thereto. In one embodiment, the canine antigen-binding fragment portion thereof that specifically binds canine CD3 comprises or consists of the LCVR as shown for PMX272, PMX285, PMX286, PMX188 or PMX189 as shown in Table 2.

In one embodiment, the second antibody, antigen binding domain or antigen-binding portion thereof that specifically binds canine CD20 has a sequence, e.g. a HC/LC CDR sequences, HCVR and/or LCVR, as described herein and shown in Table 4 which provides the SEQ ID NOs for CDR1, 2, 3, HCVR and LCVR polypeptides.

In one embodiment, the antigen binding domain or antigen-binding portion that specifically binds canine C20 comprises the HCVR CDRs as set out for a PMX molecule as shown in Table 4 or a sequence with at least 70%, 75%, 80%, 85%, 90% or 95% sequence identity thereto. In one embodiment, the canine antigen-binding fragment portion comprises or consists of the HC CDRs as shown for PMX227-271 as shown in Table 4. In one embodiment the canine antigen-binding fragment portion comprises VH CDR1, VH CDR2, and VH CDR3, wherein the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529.

In one embodiment, the antigen binding domain or antigen-binding portion that specifically binds canine CD20 comprises the HCVR sequence as set out for a PMX molecule as shown in Table 4 or a sequence with at least 70%, 75%, 80%, 85%, 90% or 95% sequence identity thereto. In one embodiment, the canine antigen-binding fragment portion comprises or consists of the HC CDRs as shown for PMX227-271 as shown in Table 4.

In one embodiment, the canine antigen binding domain antigen-binding fragment portion comprises the anti CD20 HCVR as shown for PMX230 as shown in Table 4 i.e. SEQ ID NO: 524.

In one embodiment, the antigen-binding portion that specifically binds canine CD20 comprises the LCVR sequence as set out for a PMX molecule as shown in Table 4 or a sequence with at least 70%, 75%, 80%, 85%, 90% or 95% sequence identity thereto. Thus, in one embodiment, the canine antigen-binding fragment portion comprises a LCVR having an amino acid sequence as set forth in Table 4 or a sequence with at least 70%, 75%, 80%, 85%, 90% or 95% sequence identity thereto.

Thus, according to the invention, any of the anti-CD3 HCVR of Table 2 or a sequence with at least 70%, 75%, 80%, 85%, 90% or 95% sequence identity thereto can be combined in a bispecific molecule with any of any of the anti-CD20 HCVR of Table 4 or a sequence with at least 70%, 75%, 80%, 85%, 90% or 95% sequence identity thereto. Furthermore, any of the anti-CD3 LCVR of Table 2 or a sequence with at least 70%, 75%, 80%, 85%, 90% or 95% sequence identity thereto can be combined in a bispecific molecule with any of any of the anti-CD20 LCVR of Table 4 or a sequence with at least 70%, 75%, 80%, 85%, 90% or 95% sequence identity thereto.

However, the inventors have shown that performance of the bispecific molecule can be improved if a common light chain is used. Therefore, in one embodiment, the first and second antibody, antigen binding domain or antigen-binding portion thereof of the bispecific molecules share a common light chain region, e.g. LCVR or full light chain. In one embodiment, the LCVR or full light chain is that of an antibody or antigen binding domain that binds canine CD3, for example having a SEQ ID NO. as described herein, e.g. in Table 2. In one embodiment, the LCVR has an amino acid sequence as set forth in Table 2 or a sequence with at least 70%, 75%, 80%, 85%, 90% or 95% sequence identity thereto. In one embodiment, the LCVR is that of PMX272, PMX188, PMX189, PMX285 or PMX286 as shown in Table 2.

In one embodiment, the bispecific canine antigen-binding molecule comprising a first antibody, antigen binding domain or antigen-binding portion thereof that specifically binds canine CD3, and a second antibody, antigen binding domain or antigen-binding portion thereof that specifically binds canine CD20, wherein the antigen-binding molecule is selected from PMX276, PMX277, PMX278, PMX279, PMX280, PMX281, PMX282, PMX283, PMX284, PMX287 or PMX288 as shown in Table 5 below. This table also shows the components of the CD3 and CD20 binding arm respectively. Tables 2 and 4 show the corresponding SEQ ID NOs. for CDR1, 2, 3, HCVR and LCVR polypeptides for the respective components of the CD3 and CD20 binding arm with reference to PMX numbers.

In one embodiment, the bispecific canine antigen-binding molecule comprises (a) a first antigen binding domain or antigen-binding portion thereof that specifically binds canine CD3, and comprises (i) a first heavy chain variable region (VH) comprising VH complementarity determining region (CDR)1, VH CDR2, and VH CDR3 and (ii) a first light chain variable region (VL) comprising VL CDR1, VL CDR2, and VL CDR3, wherein: the VH CDR1 comprises the amino acid sequence SEQ ID NO: 67; the VH CDR2 comprises the amino acid sequence SEQ ID NO: 68; the VH CDR3 comprises the amino acid sequence SEQ ID NO: 69; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds canine CD20, and comprises a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In another embodiment, the bispecific canine antigen-binding molecule comprises (a) a first antigen binding domain or antigen-binding portion thereof that specifically binds canine CD3, and comprises (i) a first heavy chain variable region (VH) comprising VH complementarity determining region (CDR)1, VH CDR2, and VH CDR3 and (ii) a first light chain variable region (VL) comprising VL CDR1, VL CDR2, and VL CDR3, wherein: the VH CDR1 comprises the amino acid sequence SEQ ID NO: 87; the VH CDR2 comprises the amino acid sequence SEQ ID NO: 88; the VH CDR3 comprises the amino acid sequence SEQ ID NO: 89; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds canine CD20, and comprises a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In yet another embodiment, the bispecific canine antigen-binding molecule comprises (a) a first antigen binding domain or antigen-binding portion thereof that specifically binds canine CD3, and comprises (i) a first heavy chain variable region (VH) comprising VH complementarity determining region (CDR)1, VH CDR2, and VH CDR3 and (ii) a first light chain variable region (VL) comprising VL CDR1, VL CDR2, and VL CDR3, wherein: the VH CDR1 comprises the amino acid sequence SEQ ID NO: 157; the VH CDR2 comprises the amino acid sequence SEQ ID NO: 158; the VH CDR3 comprises the amino acid sequence SEQ ID NO: 159; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds canine CD20, and comprises a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In one embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 167; the VH CDR2 comprises the amino acid sequence SEQ ID NO: 168; the VH CDR3 comprises the amino acid sequence SEQ ID NO: 169; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion that specifically binds canine CD20, and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In another embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 177; the VH CDR2 comprises the amino acid sequence SEQ ID NO: 178; the VH CDR3 comprises the amino acid sequence SEQ ID NO: 179; the VL CDR1 comprises the amino acid sequence SEQ ID NO: 460; and the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In yet another embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 187; the VH CDR2 comprises the amino acid sequence SEQ ID NO: 188; the VH CDR3 comprises the amino acid sequence SEQ ID NO: 189; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds to CD20 and comprises (i) a second V_{H} comprising V_{H} CDR1, V_{H} CDR2, and V_{H} CDR3 and (ii) a second V_{L} comprising V_{L} CDR1, V_{L} CDR2, and V_{L} CDR3, wherein: the V_{H} CDR1 comprises the amino acid sequence SEQ ID NO: 527; the V_{H} CDR2 comprises the amino acid sequence SEQ ID NO: 528; the V_{H} CDR3 comprises the amino acid sequence SEQ ID NO: 529; the V_{L} CDR1 comprises the amino acid sequence SEQ ID NO: 460; the V_{L} CDR2 comprises the amino acid sequence SEQ ID NO: 461; and the V_{L} CDR3 comprises the amino acid sequence SEQ ID NO: 462.

In one embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 327; the VH CDR2 comprises the amino acid sequence SEQ ID NO: 328; the VH CDR3 comprises the amino acid sequence SEQ ID NO: 329; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482; and (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482.

In another embodiment, the bispecific canine antigen-binding molecule comprises (a) a first antigen binding domain or antigen-binding portion thereof that specifically binds canine CD3, and comprises: (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 337; the VH CDR2 comprises the amino acid sequence SEQ ID NO: 338; the VH CDR3 comprises the amino acid sequence SEQ ID NO: 339; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492; and (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492.

In yet another embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 357; the VH CDR2 comprises the amino acid sequence SEQ ID NO: 358; the VH CDR3 comprises the amino acid sequence SEQ ID NO: 359; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462.

In one embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 347; the VH CDR2 comprises the amino acid sequence SEQ ID NO: 348; the VH CDR3 comprises the amino acid sequence SEQ ID NO: 349; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352; and (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352.

In another embodiment, the bispecific canine antigen-binding molecule comprises (a) a first antigen binding domain or antigen-binding portion thereof that specifically binds canine CD3, and comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 107; the VH CDR2 comprises the amino acid sequence SEQ ID NO: 108; the VH CDR3 comprises the amino acid sequence SEQ ID NO: 109; the VL CDR1 comprises the amino acid sequence SEQ ID NO: 110; and the VL CDR2 comprises the amino acid sequence SEQ ID NO: 111; the VL CDR3 comprises the amino acid sequence SEQ ID NO: 112; (b) a second antigen binding domain or antigen-binding portion thereof that specifically binds to CD20 and comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 110; the VL CDR2 comprises the amino acid sequence SEQ ID NO: 111; and the VL CDR3 comprises the amino acid sequence SEQ ID NO: 112.

In one embodiment, the bispecific canine antigen-binding molecule comprises a) a first VH that specifically binds canine CD3 comprising the amino acid sequence of SEQ ID NO: 64, b) a second VH that specifically binds CD20 comprising the amino acid sequence of SEQ ID NO: 524, and a first and second VL that binds CD3 and/or CD20 comprising the amino acid sequence of SEQ ID NO: 456. In another embodiment, the bispecific canine antigen-binding molecule comprises a) a first VH that specifically binds canine CD3 comprising the amino acid sequence of SEQ ID NO: 84, b) a second VH that specifically binds CD20 comprising the amino acid sequence of SEQ ID NO: 524, and a first and second VL that binds CD3 and/or CD20 comprising the amino acid sequence of SEQ ID NO: 456. In yet another embodiment, the bispecific canine antigen-binding molecule comprises a) a first VH that specifically binds canine CD3 comprising the amino acid sequence of SEQ ID NO: 154, b) a second VH that specifically binds CD20 comprising the amino acid sequence of SEQ ID NO: 524, and a first and second VL that binds CD3 and/or CD20 comprising the amino acid sequence of SEQ ID NO: 456. In one embodiment, the bispecific canine antigen-binding molecule comprises a) a first VH that specifically binds canine CD3 comprising the amino acid sequence of SEQ ID NO: 164, b) a second VH that specifically binds CD20 comprising the amino acid sequence of SEQ ID NO: 524, and a first and second VL that binds CD3 and/or CD20 comprising the amino acid sequence of SEQ ID NO: 456. In one embodiment, the bispecific canine antigen-binding molecule comprises a) a first VH that specifically binds canine CD3 comprising the amino acid sequence of SEQ ID NO: 174, b) a second VH that specifically binds CD20 comprising the amino acid sequence of SEQ ID NO: 524, and a first and second VL that binds CD3 and/or CD20 comprising the amino acid sequence of SEQ ID NO: 456. In another embodiment, the bispecific canine antigen-binding molecule comprises a) a first VH that specifically binds canine CD3 comprising the amino acid sequence of SEQ ID NO: 184, b) a second VH that specifically binds CD20 comprising the amino acid sequence of SEQ ID NO: 524, and a first and second VL that binds CD3 and/or CD20 comprising the amino acid sequence of SEQ ID NO: 456. In yet another embodiment, the bispecific canine antigen-binding molecule comprises a) a first VH that specifically binds canine CD3 comprising the amino acid sequence of SEQ ID NO: 324, b) a second VH that specifically binds CD20 comprising the amino acid sequence of SEQ ID NO: 524, and a first and second VL that binds CD3 and/or CD20 comprising the amino acid sequence of SEQ ID NO: 476. In one embodiment, the bispecific canine antigen-binding molecule comprises a) a first VH that specifically binds canine CD3 comprising the amino acid sequence of SEQ ID NO: 334, b) a second VH that specifically binds CD20 comprising the amino acid sequence of SEQ ID NO: 524, and a first and second VL that binds CD3 and/or CD20 comprising the amino acid sequence of SEQ ID NO: 486. In another embodiment, the bispecific canine antigen-binding molecule comprises a) a first VH that specifically binds canine CD3 comprising the amino acid sequence of SEQ ID NO: 74, b) a second VH that specifically binds CD20 comprising the amino acid sequence of SEQ ID NO: 524, and a first and second VL that binds CD3 and/or CD20 comprising the amino acid sequence of SEQ ID NO: 456. In yet another embodiment, the bispecific canine antigen-binding molecule comprises a) a first VH that specifically binds canine CD3 comprising the amino acid sequence of SEQ ID NO: 344, b) a second VH that specifically binds CD20 comprising the amino acid sequence of SEQ ID NO: 524, and a first and second VL that binds CD3 and/or CD20 comprising the amino acid sequence of SEQ ID NO: 346. In one embodiment, the bispecific canine antigen-binding molecule comprises a) a first VH that specifically binds canine CD3 comprising the amino acid sequence of SEQ ID NO: 104, b) a second VH that specifically binds CD20 comprising the amino acid sequence of SEQ ID NO: 524, and a first and second VL that binds CD3 and/or CD20 comprising the amino acid sequence of SEQ ID NO: 106.

Thus, in one embodiment, the bispecific molecule comprises
a) a HCVR that binds canine CD3 having a SEQ ID No. as shown for PMX160 in Table 2 and a HCVR that binds canine CD20 having a SEQ ID No. as shown for PMX230 in Table 4 and a common LCVR as shown for PMX272 in Table 2;
b) a HCVR that binds canine CD3 having a SEQ ID No. as shown for PMX162 in Table 2 and a HCVR that binds canine CD20 having a SEQ ID No. as shown for PMX230 in Table 4 and a common LCVR as shown for PMX272 in Table 2;
c) a HCVR that binds canine CD3 having a SEQ ID No. as shown for PMX169 in Table 2 and a HCVR that binds canine CD20 having a SEQ ID No. as shown for PMX230 in Table 4 and a common LCVR as shown for PMX272 in Table 2;
d) a HCVR that binds canine CD3 having a SEQ ID No. as shown for PMX170 in Table 2 and a HCVR that binds canine CD20 having a SEQ ID No. as shown for PMX230 in Table 4 and a common LCVR as shown for PMX272 in Table 2;
e) a HCVR that binds canine CD3 having a SEQ ID No. as shown for PMX171 in Table 2 and a HCVR that binds canine CD20 having a SEQ ID No. as shown for PMX230 in Table 4 and a common LCVR as shown for PMX272 in Table 2;
f) a HCVR that binds canine CD3 having a SEQ ID No. as shown for PMX172 in Table 2 and a HCVR that binds canine CD20 having a SEQ ID No. as shown for PMX230 in Table 4 and a common LCVR as shown for PMX272 in Table 2;
g) a HCVR that binds canine CD3 having a SEQ ID No. as shown for PMX186 in Table 2 and a HCVR that binds canine CD20 having a SEQ ID No. as shown for PMX230 in Table 4 and a common LCVR as shown for PMX285 in Table 2;
h) a HCVR that binds canine CD3 having a SEQ ID No. as shown for PMX187 in Table 2 and a HCVR that binds canine CD20 having a SEQ ID No. as shown for PMX230 in Table 4 and a common LCVR as shown for PMX286 in Table 2;
i) a HCVR that binds canine CD3 having a SEQ ID No. as shown for PMX190 in Table 2 and a HCVR that binds canine CD20 having a SEQ ID No. as shown for PMX230 in Table 4 and a common LCVR as shown for PMX272 in Table 2;
j) a HCVR that binds canine CD3 having a SEQ ID No. as shown for PMX188 in Table 2 and a HCVR that binds canine CD20 having a SEQ ID No. as shown for PMX230 in Table 4 and a common LCVR as shown for PMX188 in Table 2; or
k) a HCVR that binds canine CD3 having a SEQ ID No. as shown for PMX189 in Table 2 and a HCVR that binds canine CD20 having a SEQ ID No. as shown for PMX230 in Table 4 and a common LCVR as shown for PMX189 in Table 2.

TABLE 5
Exemplary bispecific molecules

| PMX number | CD3VH PMX | CD20VH PMX | VL |
|---|---|---|---|
| PMX276 | PMX160 | PMX230 | PMX272VL |
| PMX277 | PMX162 | PMX230 | PMX272VL |
| PMX278 | PMX169 | PMX230 | PMX272VL |
| PMX279 | PMX170 | PMX230 | PMX272VL |
| PMX280 | PMX171 | PMX230 | PMX272VL |
| PMX281 | PMX172 | PMX230 | PMX272VL |
| PMX282 | PMX186 | PMX230 | PMX285VL |
| PMX283 | PMX187 | PMX230 | PMX286VL |
| PMX284 | PMX190 | PMX230 | PMX272VL |
| PMX287 | PMX188 | PMX230 | PMX188VL |
| PMX288 | PMX189 | PMX230 | PMX189VL |

A bispecific antibody or antigen binding molecule according to the present invention is not limited to any particular bispecific format or method of producing it.

Examples of bispecific antibody or antigen binding molecules which may be used in the present invention comprise (i) a single antibody or antigen binding domain that has two arms comprising different antigen-binding regions; (ii) a single antibody, or antigen binding domain that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage; (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In one embodiment, the antibody, antigen binding domain or antigen-binding portion thereof comprises an Fc region, for example a canine Fc region, for example a canine IgGB Fc region. In one embodiment, the bispecific antibody or antigen binding molecule of the invention comprises a first Fc-region comprising a first CH3 region, and a second Fc-region comprising a second CH3 region. In one embodiment, the bispecific antibody as defined in any of the embodiments disclosed herein comprises first and second heavy chains, wherein each of said first and second heavy chain comprises at least a hinge region, a CH2 and CH3 region.

The bispecific antibody or antigen binding molecule according to the present invention may comprise modifications in the Fc region. Thus, In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain. For example, the invention includes bispecific antigen-binding molecules comprising one or more modifications in the Fc domain that results in a modified Fc domain having a modified binding interaction (e.g., enhanced or diminished) between Fc and the Fc receptor. In one embodiment, the bispecific antigen-binding molecule comprises a modification in a CH2 or a CH3 region.

The variable region sequences described herein, including but not limited to the amino acid and nucleotide sequences shown in Table 2 and Table 4 (and/or fragments thereof) may be used in combination with one or more amino acid sequences and/or nucleotide sequences encoding one or more constant chains (and/or a fragment thereof) of an antibody molecule. For instance, the variable region amino acid sequences shown in Table 2 or Table 4 may be joined to the constant regions of any antigen binding domain, or antibody molecule of the same or a different species (e.g., human, goat, rat, sheep, chicken) of that from which the variable region amino acid sequence was derived. Preferably, the variable region amino acid sequences shown in Table 2 or Table 4 is joined to the constant regions of a canine antigen binding domain, or canine antibody and may be the constant region from any of canine IgG A, B, C or D. In one embodiment, the constant region is canine IgG B constant region. Dog IGGB (SEQ ID NO: 28), dog IGK or dog IGLC5 constant regions may be used. Variants of the constant region which have altered effector regions may also be used, for example a variant of Dog IGGB (SEQ ID NO: 30). These variants may be formed by introducing mutations in canine IgG-B which abolishes the effector function. Canine IgG-B may be modified to reduce or abolish canine IgG-B effector function when compared to the same polypeptide comprising a wild-type IgG-B Fc domain. The regions targeted in the amino acid sequence of the Fc domain for modification may include the lower hinge, proline region and SHED region, where potential interactions with FcgammaR and C1q occur. Examples of such mutations are provided in WO 2023/012486 and are incorporated herein by reference.

Other such variants may comprise charge pair combinations in canine CH3 domains which may significantly enriches heavy chain heterodimersation over homodimer formation. This may minimise the formation of homodimer contaminants for the production of bispecific antibodies. These charge pair combinations may be in the canine IgG CH3 domain interface of the Fc region wherein said IgG is selected from IgG-A, B, C or D. A first canine IgG CH3 domain and a second canine IgG CH3 domain may both be engineered in a complementary manner so that each CH3 domain (or a polypeptide comprising it) substantially does not homodimerise with itself or homodimerises at a lower rate, but is forced to heterodimerise with the complementary engineered other CH3 domain. In other words, the first and second CH3 domain may heterodimerise and few homodimers between the two first or the two second CH3 domains are formed. Examples of such mutations are provided in WO 2021/214460 A1 and are incorporated herein by reference.

Variants may also comprise mutations in canine CH2 or CH3 IgG Fc domains which result in a differential affinity for a binding affinity reagent and/or improved stability. For example, the binding molecule may have a differential affinity for binding Protein A relative to the wild type IgG Fc domain. The differential affinity of the immunoglobulin heavy chains allows for optimised isolation of said binding proteins or antibodies. For example, suitable variant IgG Fc domains may comprise one or more amino acid substitution which increases affinity for binding protein A, or one or more amino acid substitution which decreases affinity for binding Protein A. Examples of such mutations are provided in GB2311984.5 and are incorporated herein by reference.

Thus, in one embodiment, the antibody, antigen binding domain or antigen-binding portion thereof comprises mutant variants with deficient Fc binding arising from mutations in canine IgG-B. In another embodiment, the antibody, antigen binding domain or antigen-binding portion thereof comprises mutant variants with enriched heavy chain heterodimersation arising from mutations in canine CH3 domains of IgG-A, B, C or D. In another embodiment, the antibody, antigen binding domain or antigen-binding portion thereof comprises mutant variants with mutations in canine CH2 or CH3 IgG Fc domains which result in a differential affinity for a binding affinity reagent and/or improved stability. The antibody, antigen binding domain or antigen-binding portion thereof may have a single mutation which has a single mutant phenotype for example, decreased Fc effector function, or may have multiple mutations resulting in multiple phenotypes for example, decreased Fc effector function, enriched heavy chain heterodimersation and altered Protein A binding affinity.

Canine Antibody or Antigen-Binding Portion Thereof that Binds Canine CD20

As explained above, the invention relates to a canine antibody, antigen binding domain or antigen-binding portion thereof that binds canine CD3 and canine CD20. Table 4 sets out examples of canine antibody, antigen binding domain or antigen-binding portion thereof that bind canine CD20 and which can be used in such a bispecific molecule.

In yet a further separate aspect, the invention also relates to a canine antibody, antigen binding domain or antigen-binding portion thereof that binds canine CD20. Such antibody, antigen binding domain or antigen-binding portion thereof can be used in monovalent format or in bispecific format, for example together with a CD3 antibody as described above.

In one embodiment, the antibody, antigen binding domain or antigen-binding portion thereof according to the invention that binds canine CD20 has one or more of the following properties:
a) binds specifically to canine CD20;
b) binds to canine CD20 with a KD as measured in the examples and for example as shown in the figures;
c) shows cell killing, such as CDC and/or ADCC, in canine lymphoma cell lines expressing CD20, as measured in the Examples;
d) promotes antibody dependent cellular phagocytosis (ADCP);
e) is capable of effectively depleting CD20 positive B cells in canine tissues;
f) is capable of binding to cells expressing canine CD20 and/or
g) is capable of depleting cells expressing canine CD20, suitably by direct cell killing via apoptosis.

In one embodiment, the antibody, antigen binding domain or antigen-binding portion thereof according to the invention has one or more of the properties above and optionally one or more of the following properties:
a) has CDC activity with an EC50 value of less than 20 nM and/or
b) has ADCC activity with an EC50 value of less than 0.3 nM.

In one embodiment, the canine antibody, antigen binding domain or antigen-binding fragment portion that binds canine CD20 comprises the complementarity determining regions (CDRs) of a heavy chain variable region (HCVR) having an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto. The complementarity determining regions (CDRs) refer to the three CDRs, i.e. CDR1, 2 and 3.

In one embodiment, the canine antibody, antigen binding domain or antigen-binding fragment portion thereof comprises: (a) the complementarity determining regions (CDRs) of a heavy chain variable region (HCVR) having an amino acid sequence as set forth in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto; and (b) the CDRs of a light chain variable region (LCVR) having an amino acid sequence as set forth in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto.

In other words, in one embodiment, the canine antibody, antigen binding domain or antigen-binding fragment portion thereof comprises: (a) the HC CDRs as set out for one of the PMX molecules in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto; and (b) the LC CDRs of a light chain variable region (LCVR) as set out for one of the PMX molecules in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto. In one embodiment, the canine antibody, antigen binding domain or antigen-binding fragment portion thereof comprises the HC CDRs and the LC CDRs of PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4.

In one embodiment, the invention relates to an isolated canine antibody, antigen binding domain or antigen-binding portion thereof which binds canine CD20 wherein said antibody, antigen binding domain or antigen-binding portion thereof comprises
a) a heavy chain (HC) CDR1 sequence comprising or consisting of a SEQ ID No. as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto,
b) a HC CDR2 sequence comprising or consisting of a SEQ ID No. as shown for the respective PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto,
c) a HC CDR3 sequence comprising or consisting of a SEQ ID No. as shown for the respective PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto,
d) a light chain (LC) CDR1 sequence comprising or consisting of a SEQ ID No. for the respective PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 60%, 70%, 80% or 90% sequence identity thereto,
e) a LC CDR2 sequence comprising or consisting of a SEQ ID No. for the respective PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% % sequence identity thereto and
f) a LC CDR3 sequence comprising or consisting of a SEQ ID No. for the respective PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto.

In one embodiment, the isolated canine antibody, antigen binding domain or antigen-binding portion thereof which binds canine CD20 wherein said antibody, antigen binding domain or antigen-binding portion thereof comprises
a) a HC CDR1 sequence comprising or consisting of a SEQ ID No. as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 molecule in Table 4,
b) a HC CDR2 sequence comprising or consisting of SEQ ID No. as shown for the respective PMX molecule in Table 4;
c) a HC CDR3 sequence comprising or consisting of SEQ ID No. as shown for the respective PMX molecule in Table 4,
d) a LC CDR1 sequence comprising or consisting of SEQ ID No. as shown for the respective PMX molecule in Table 4,
e) a LC CDR2 sequence comprising or consisting of SEQ ID No. as shown for the respective PMX molecule in Table 4 and
f) a LC CDR3 sequence comprising or consisting of SEQ ID No. as shown for the respective PMX molecule in Table 4;
   or an isolated canine antibody or antigen-binding portion thereof with a CDR as described above but which has one or more CDR which has 1, 2, 3, 4 or 5 amino acid substitutions in one or more LC or HC CDR compared to the reference CDR sequence.

In one embodiment, the canine antibody, antigen binding domain or antigen-binding fragment portion comprises: (a) a heavy chain variable region (HCVR) having an amino acid sequence as set forth for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto; and (b) a light chain variable region (LCVR) having an amino acid sequence as set forth for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or an amino acid sequence which has at least 70%, 75%, 80%, 85%, 90% or 95% sequence identity thereto. In one embodiment, the canine antibody, antigen binding domain or antigen-binding fragment portion thereof comprises a HC CDRs and a LC CDRs of PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4.

In one embodiment, the canine antibody, antigen binding domain or antigen-binding fragment portion thereof comprises the HCVR and the LCVR of PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4.

In one embodiment, the canine antibody, antigen binding domain or antigen-binding portion thereof comprises
a) a HCVR sequence comprising or consisting of a SEQ ID No. as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4 or a sequence with 1, 2, 3, 4, 5, 6, 7, 8 9, or 10 amino acid modifications (e.g. a deletion, addition or substitution) in the HCVR framework region compared to the reference HCVR and
b) a LCVR sequence comprising or consisting of SEQ ID No. as shown for the respective PMX molecule in Table 4 or a sequence with 1, 2, 3, 4, 5, 6, 7, 8 9, or 10 amino acid modifications (e.g. a deletion, addition or substitution) in the LCVR framework region compared to the reference LCVR.

In one embodiment, the antigen-binding portion thereof is a F(ab′)2, Fab, Fv, scFv, heavy chain, light chain, variable heavy (V_{H}) domain or variable light (V_{L}).

In one embodiment, the antigen binding domain or antigen-binding portion is a heavy chain and comprises the HC CDRs as set out for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4 or a sequence with at least 70%, 75%, 80%, 85%, 90% or 95% sequence identity thereto. In one embodiment, the canine antigen binding domain or antigen-binding fragment portion comprises or consists of the HC CDRs as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4.

In one embodiment, the antigen binding domain or antigen-binding portion is a heavy chain variable region and comprises the HCVR as set out for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4 or a sequence with at least 70%, 75%, 80%, 85%, 90% or 95% sequence identity thereto. Thus, in one embodiment, the canine antigen binding domain or antigen-binding fragment portion comprises a HCVR having an amino acid sequence for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as set forth in Table 4 or a sequence with at least 70%, 75%, 80%, 85%, 90% or 95% sequence identity thereto.

In one embodiment, the canine antigen binding domain or antigen-binding fragment portion comprises or consists of the HCVR as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 in Table 4.

In another embodiment, the invention relates to the use of these antibodies, antigen binding domains or antigen binding portions thereof in a bispecific antibody together with antibodies, antigen binding domains or antigen binding portions thereof that bind CD20.

In one embodiment, the antibody or antigen-binding portion thereof as described above comprises an Fc region, for example a canine Fc region, for example a canine IgGB Fc region.

In one embodiment, the canine antibody, antigen binding domain or antigen-binding portion thereof which binds canine CD20 comprises VH sequence as shown for PMX230 or a VHs with more than 80% identity.

In one embodiment, the canine antibody, antigen binding domain or antigen-binding portion thereof is an scFv, Fv, heavy chain or single domain antibody.

In one embodiment, the canine antibody, antigen binding domain or antigen-binding portion thereof is conjugated to a therapeutic moiety.

In one embodiment, the therapeutic moiety is a second or further antibody, antigen binding domain or antigen-binding portion thereof.

In one embodiment, the second antibody, antigen binding domain or antigen-binding portion thereof binds to a different target, for example a target that is not CD3 or CD20. In an embodiment the different target is a tumour antigen.

In one embodiment, the canine antibody, antigen binding domain or antigen-binding portion thereof is conjugated to a further moiety selected from a half life extending moiety, label, cytotoxin, liposome, nanoparticle or radioisotope.

The invention relates to immunoconjugates and other binding agents comprising the antibody, antigen binding domain or antigen binding portion thereof according to the invention that binds CD20. For example, the antibody, antigen binding domain or antigen-binding portion thereof according to the invention may be conjugated to a therapeutic moiety or non-therapeutic moiety.

### Pharmaceutical Compositions

In another aspect, there is provided a pharmaceutical composition comprising an antibody, antigen binding domain or fragment of the invention, i.e. a canine antibody, antigen binding domain or antigen-binding portion thereof that binds canine CD3, a canine antibody, antigen binding domain or antigen-binding portion thereof that binds both canine CD3 and CD20 or a canine antibody, antigen binding domain or antigen-binding portion thereof that binds canine CD20, and optionally a pharmaceutically acceptable carrier. The term pharmaceutical composition as used herein refers to a composition that is used to treat a companion animal, that is for veterinary use, i.e. a veterinary composition. In preferred embodiments, the animal that is treated is a dog.

The pharmaceutical composition may optionally comprise a pharmaceutically acceptable carrier. Antibodies, protein or construct or the pharmaceutical composition can be administered by any convenient route, including but not limited to oral, topical, parenteral, sublingual, rectal, vaginal, ocular, intranasal, pulmonary, intradermal, intravitreal, intramuscular, intraperitoneal, intravenous, subcutaneous, intracerebral, transdermal, transmucosal, by inhalation, or topical, particularly to the ears, nose, eyes, or skin or by inhalation.

Parenteral administration includes, for example, intravenous, intramuscular, intraarterial, intraperitoneal, intranasal, rectal, intravesical, intradermal, topical or subcutaneous administration. Preferably, the compositions are administered parenterally.

The pharmaceutically acceptable carrier or vehicle can be particulate, so that the compositions are, for example, in tablet or powder form. The term “carrier” refers to a diluent, adjuvant or excipient, with which a drug antibody conjugate of the present invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to an animal, the antigen binding domain, or antibody of the present invention or compositions and pharmaceutically acceptable carriers are sterile. Water is a preferred carrier when the drug antibody conjugates of the present invention are administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The pharmaceutical composition of the invention can be in the form of a liquid, e.g., a solution, emulsion or suspension. The liquid can be useful for delivery by injection, infusion (e.g., IV infusion) or subcutaneously. When intended for oral administration, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as carboxymethylcellulose, ethyl cellulose, microcrystalline cellulose, or gelatin; excipients such as starch, lactose or dextrins, disintegrating agents such as alginic acid, sodium alginate, corn starch and the like; lubricants such as magnesium stearate; glidants such as colloidal silicon dioxide; sweetening agents such as sucrose or saccharin; a flavoring agent such as peppermint, methyl salicylate or orange flavoring; and a coloring agent. When the composition is in the form of a capsule (e. g. a gelatin capsule), it can contain, in addition to materials of the above type, a liquid carrier such as polyethylene glycol, cyclodextrin or a fatty oil.

The composition can be in the form of a liquid, e. g. an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

Compositions can take the form of one or more dosage units. In specific embodiments, it can be desirable to administer the composition locally to the area in need of treatment, or by intravenous injection or infusion.

### Methods of Treating Disease

The invention further extends to methods for the treatment of a disease, administration of a pharmaceutical composition or formulation described herein or the antibody, antigen binding domain or antigen binding portion of the invention, i.e. a canine antibody, antigen binding domain or antigen-binding portion thereof that binds canine CD3, a canine antibody, antigen binding domain or antigen-binding portion thereof that binds both canine CD3 and CD20 or a canine antibody, antigen binding domain or antigen-binding portion thereof that binds canine CD20. Also envisaged is a pharmaceutical composition or formulation described herein or a binding molecule or fusion protein that comprises an antibody, antigen binding domain or antigen binding portion thereof as described herein, i.e. a canine antibody, antigen binding domain or antigen-binding portion thereof that binds canine CD3, a canine antibody, antigen binding domain or antigen-binding portion thereof that binds both canine CD3 and CD20 or a canine antibody, antigen binding domain or antigen-binding portion thereof that binds canine CD20, for use in the treatment of disease.

In particular, a canine antibody, antigen binding domain or antigen-binding portion thereof that binds canine CD3 may be used to treat a disease in a dog such as autoimmune disorders, for example, type 1 diabetes and graft-versus-host disease. In particular, a bispecific antibody that binds both canine CD3 and CD20 or a canine antibody, antigen binding domain or antigen-binding portion thereof that binds canine CD20 may be used for treating a condition mediated by B-cells.

In particular, the invention relates to a method of treating a condition mediated by B-cells in a canine subject in need thereof comprising administering an effective amount of the antibody, antigen binding domain or antigen-binding portion thereof as described herein that binds CD20.

An aspect of the invention is also an antibody, antigen binding domain or antigen-binding portion that binds CD20 thereof or the pharmaceutical composition as described herein for use in the treatment of a condition mediated by B-cells in a canine subject.

For example, the antibody, antigen binding domain or antigen-binding portion thereof may be used to deplete canine blood and/or tissues of B cell lymphoma cells. The condition mediated by B-cells is selected from a B cell lymphoma, (e.g., diffuse large cell B cell lymphoma, Hodgkin's and non-Hodgkin's lymphoma, follicular lymphoma, mucosa-associated lymphatic tissue lymphoma (MALT), small cell lymphocytic lymphoma, chronic lymphocytic leukemia, mantel cell lymphoma, Burkitt's lymphoma, mediastinal large B cell lymphoma, Waldenstrom macroglobulinemia, nodal marginal zone B cell lymphoma (NMZL), splenic marginal zone lymphoma (SMZL), intravascular large B-cell lymphoma, primary effusion lymphoma, lymphomatoid granulomatosis), leukemia or an immune mediated disease. The immune mediated disease may be an autoimmune disease. Examples may include, but are not limited to, autoimmune hemolytic anemia, immune-mediated thrombocytopenia, autoimmune blistering diseases, immune-mediated arthritis and atopic dermatitis, rheumatoid arthritis, systemic lupus erythematosus (SLE), Sjogren's syndrome, vasculitis, multiple sclerosis, Graves' disease, idiopathic thrombocytopenia, dermatomyositis, immune mediated thrombocytopenia, polymyocytosis, pemphigus, immune mediated hemolytic anemia and bullous pemphigoid.

The amount of the therapeutic that is effective/active in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances. Factors like age, body weight, sex, diet, time of administration, rate of excretion, condition of the host, drug combinations, reaction sensitivities and severity of the disease shall be taken into account.

Typically, the amount is at least about 0.01% of an antibody, antigen binding domain or fragment thereof of the present invention by weight of the composition. When intended for oral administration, this amount can be varied to range from about 0.1% to about 80% by weight of the composition. Preferred oral compositions can comprise from about 4% to about 50% of the antibody or fragment thereof of the present invention by weight of the composition.

Preferred compositions of the present invention are prepared so that a parenteral dosage unit contains from about 0.01% to about 2% by weight of the antibody, antigen binding domain or fragment thereof of the present invention.

For administration by injection, such as intravenous or sub-cutaneous injection, the composition can comprise from about typically about 0.01 mg/kg to about 250 mg/kg, for example 0.1 mg/kg to about 250 mg/kg of the subject's body weight, for example, between about 0.1 mg/kg and about 20 mg/kg of the animal's body weight, and more preferably about 1 mg/kg to about 10 mg/kg of the animal's body weight, although less than 0.1 mg/kg is also envisaged. In one embodiment, the composition is administered at a dose of about 0.5 to 30 mg/kg, e.g., about 5 to 25 mg/kg, about 10 to 20 mg/kg, about 0.5 to 5 mg/kg, about 0.5 to 2.5 mg/kg, about 0.5 to 2.0 mg/kg or about 2 or 3 mg/kg. In one embodiment, the composition is administered at a dose of 2 to 50 mg/ml. In one embodiment, the composition is administered at a dose of 0.5 mg/ml to 2.5 mg/ml, or 0.5 mg/ml to 5 mg/ml. The dosing schedule can vary from e.g., once a week to once every 2, 3, 4 weeks, or up to 8 weeks between doses. In one embodiment, the composition is administered at a dose of 0.5 mg/ml to 2.5 mg/ml every three to four weeks, e.g. 0.5 mg/ml or 2.5 mg/ml every three to four weeks.

Suitably the dose is chosen so as to give prolonged depletion of CD20 positive cells to allow a three to four week interval between doses. Multiple doses may be administered, suitably up to about 6 or more repeat doses.

In one embodiment, post-treatment, the subject has at least 7 days, or at least 14 days, or at least 21 days, or at least 28 days, or at least 40 days, or at least 50 days, or at least 60 days disease progression-free. In one embodiment, post-treatment, the subject has at least 7 days, or at least 14 days, or at least 21 days, or at least 28 days, or at least 40 days, or at least 50 days, or at least 60 days disease progression-free.

In one embodiment, the number of days of survival, the number of disease-free days, or the number of disease-progression free days is at least 2 months, or at least 3 months, or at least 4 months, e.g. at least 5 months, such as at least 6 months.

In one embodiment, the number of days of survival, the number of disease-free days, or the number of disease-progression free days is at least 9 months, 200 days, 300 days or 3 years or more. In one embodiment, it is least one, two, three or more years. The invention provides methods of treating or preventing CD3 and/or CD20-mediated diseases or disorders in a companion animal, e.g., a dog, comprising administering an effective amount of an antibody, antigen binding domain or fragment of the present invention to the animal in need thereof.

As used herein, “treat”, “treating” or “treatment” means inhibiting or relieving a disease or disorder. For example, treatment can include a postponement of development of the symptoms associated with a disease or disorder, and/or a reduction in the severity of such symptoms that will, or are expected, to develop with said disease. The terms include ameliorating existing symptoms, preventing additional symptoms, and ameliorating or preventing the underlying causes of such symptoms. Thus, the terms denote that a beneficial result is being conferred on at least some of the mammals, e.g., canine patients, being treated. Many medical treatments are effective for some, but not all, patients that undergo the treatment. In treatment of B cell lymphomas, for example, ameliorating symptoms can be assessed by measuring lymph nodes after treatment and observing a reduction in lymph node size as an indication of successful treatment.

The term “subject” or “patient” refers to a dog, which is the object of treatment, observation, or experiment. For the avoidance of doubt, the treatment of humans is excluded.

The molecules or pharmaceutical composition of the invention may be administered as the sole active ingredient or in combination with one or more other therapeutic agent, for example a cancer therapy. In some embodiments the cancer therapy is a radiation therapy. A therapeutic agent is a compound or molecule which is useful in the treatment of a disease. Examples of therapeutic agents include antibodies, antibody fragments, drugs, toxins, nucleases, hormones, immunomodulators, pro-apoptotic agents, anti-angiogenic agents, boron compounds, photoactive agents or dyes, radioisotopes, immunosuppressant or an immunological modulating agent, such as a cytokine or a chemokine. In one example, the molecules or pharmaceutical composition of the invention may be administered in combination with a multi-agent, CHOP (Cyclophosphamide, Hydroxydaunorubicin, Oncovin, and Prednisone)-based chemotherapy protocol incorporating several injectable and oral drugs (Lasparaginase, vincristine, Cytoxan, prednisone, and doxorubicin), given on a more-or-less weekly basis for a period of several months. Administration may be at the same time, prior or after administration of the compound of the invention.

Thus, the invention also relates to a combination therapy comprising an antibody, antigen binding domain or antigen-binding portion thereof that binds CD3 as described herein, a bispecific antibody as described herein, or a pharmaceutical composition as described herein and a further therapeutic moiety. The further therapeutic moiety may be an antibody, antigen binding domain or antigen-binding portion thereof that binds canine CD20. Thus, the invention relates to a combination therapy comprising the bispecific antibody as described herein and an antibody, antigen binding domain or antigen-binding portion thereof that binds canine CD20. Thus, the invention also relates to a combination therapy comprising an antibody, antigen binding domain or antigen-binding portion thereof that binds CD3 as described herein and an antibody, antigen binding domain or antigen-binding portion thereof that binds CD20, for example as described herein. The antibody, antigen binding domain or antigen-binding portion thereof, bispecific antibody or the pharmaceutical composition and the further therapeutic moiety are administered concurrently or sequentially.

The invention also relates to a method of inhibiting tumour growth or metastasis comprising contacting a tumour cell with an effective amount of the antibody, antigen binding domain or antigen-binding portion thereof or a pharmaceutical composition as described herein. The method can be in vitro, in vivo or ex vivo.

The invention also relates to a method of killing a tumour cell expressing CD20, comprising contacting the cell with an antibody or pharmaceutical composition as described herein, such that killing of the cell expressing CD20 occurs. The tumour cell is a canine tumour cell. The method can be in vitro, in vivo or ex vivo.

Methods for eliminating cells expressing canine CD20 using an antibody, antigen binding domain or pharmaceutical composition as described herein are also provided. The method can be in vitro, in vivo or ex vivo.

### Nucleic Acid Sequences, Vectors and Host Cells

The invention also relates to a nucleic acid sequence that encodes an amino acid sequence of a canine antibody or antigen binding portion thereof that binds CD3 as described herein, e.g. a HC variable region or LC variable region. Exemplary sequences are described in Table 2. In one embodiment, said nucleic acid is selected from a sequence as shown in Table 2 or a nucleic acid having at least 75%, 80% or 90% sequence homology thereto.

The invention also relates to a nucleic acid sequence that encodes an amino acid sequence of a canine antibody or antigen binding portion thereof that binds CD20 as described herein, e.g. a HC variable region or LC variable region of PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 as shown in Table 4.

The invention also relates to a nucleic acid sequence that encodes an amino acid sequence of a canine bispecific antibody or antigen binding portion thereof that binds CD3 and CD20 as described herein, e.g. a HC variable region or LC variable region. Exemplary sequences are described in Table 2. In one embodiment, the nucleic acid that encodes the CD3 binding portion is selected from a sequence as shown in Table 2 or a nucleic acid having at least 75%, 80% or 90% sequence homology thereto. In one embodiment, the nucleic acid that encodes the CD20 binding portion is selected from a sequence as shown in Table 4 or a nucleic acid having at least 75%, 80% or 90% sequence homology thereto.

In one embodiment, said nucleic acid sequence is linked with a linker to a second nucleic acid sequence. In one embodiment, said second nucleic acid encodes an additional therapeutic moiety. In one embodiment, said linker is a nucleic acid linker. An exemplary nucleic acid is shown below. However, a skilled person will understand that due to the degeneracy of the genetic code, other sequences are also envisaged and within the scope of the invention.

A nucleic acid according to the present invention may comprise DNA or RNA and may be wholly or partially synthetic or recombinantly produced. Reference to a nucleotide sequence as set out herein encompasses a DNA molecule with the specified sequence, and encompasses a RNA molecule with the specified sequence in which U is substituted for T, unless context requires otherwise.

Furthermore, the invention relates to a nucleic acid construct comprising at least one nucleic acid as defined above. The construct may be in the form of a plasmid, vector, transcription or expression cassette.

The invention also relates to a vector that comprises a nucleic acid encoding the CD3 or CD20 antibody, antigen binding domain or antigen binding portion thereof as described herein. The term “vector” refers to a nucleic acid molecule, preferably a DNA molecule derived, for example, from a plasmid, bacteriophage, or vims, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art. In an embodiment the vector is an adeno-associated virus (AAV) vector, such as those described in WO2021176362.

In some embodiments, the nucleic acid may also comprise a leader sequence. In another embodiment, it does not comprise a leader sequence. Any suitable leader sequences may be used including the native immunoglobulin germline leader sequence, such as SEQ ID NO: 943 (MESALSWVFLVTILKGVQG) for a heavy chain, SEQ ID NO: 944 (MAWTHLLLSLLALCTGSVA) for a light chain, or others, such as the Campath leader sequence (SEQ ID NO: 945 MGWSCIILFLVATATGVHS) (see U.S. Pat. No. 8,362,208 B2), may be chosen to enhance protein expression.

In some embodiments, the nucleic acid may also comprise a signal peptide, i.e. a short amino acid sequence (13-36 amino acids) on the N-terminus of a secretory protein (like an immunoglobulin) that mediates the translocation of a protein destined for secretion through the first membrane of the secretory pathway. This sequence is not present in the mature protein, being cleaved in a co-translational event, but mediates the secretion and correct expression of the protein. Suitable signal sequences can be used to optimize the expression of a recombinant protein.

The invention also relates to an isolated recombinant host cell comprising one or more nucleic acid construct as described above. Host cells useful in the present invention are prokaryotic, yeast, or higher eukaryotic cells and include but are not limited to microorganisms such as bacteria (e.g., E. coli, B. subtilis) transformed with recombinant bacteriophage DNA, plasmid DNA or cosmid DNA expression vectors containing antibody coding sequences; yeast (e.g., Saccharomyces, Pichia) transformed with recombinant yeast expression vectors containing antibody coding sequences; insect cell systems infected with recombinant virus expression vectors (e.g. Baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

Prokaryotes useful as host cells in the present invention include gram negative or gram-positive organisms such as E. coli, B. subtilis, Enterobacter, Erwinia, Klebsiella, Proteus, Salmonella, Serratia, and Shigella, as well as Bacilli, Pseudomonas, and Streptomyces. One cloning host is E. coli 294 (ATCC 31,446), although other strains such as E. coli B, E. coli X1776 (ATCC 31,537), and E. coli W3110 (ATCC 27,325) are suitable. In one embodiment, a method of making an anti-CD20 antibody as described herein is provided, wherein the method comprises culturing the host cell under conditions suitable for expression of the polynucleotide encoding the antibody and isolating the antibody.

Nucleic acids encoding antigen binding domains or antibodies can be used to administer an antigen binding domain or antibody to an individual in order to produce their encoded protein in vivo and mediate a therapeutic effect. Delivery of polynucleotides into a subject can be direct such that polynucleotides or expression vectors are administered to an individual e.g. through introduction of mRNA or DNA directly into cells e.g. muscle cells. Indirect introduction is also envisaged where polynucleotides are transformed into cells in vitro prior to administration. Viral vectors, such as defective or attenuated viruses, may also be used.

In one embodiment, a method of making an anti-CD3 antigen binding domain, or antibody as described herein is provided, wherein the method comprises culturing the host cell under conditions suitable for expression of the polynucleotide encoding the antibody and isolating the antibody.

The invention also relates to a heterologous assay or expression system comprising a canine CD3 and a cell line derived from a different species, e.g. a human cell line such as HEK.

The assay comprises contacting a canine CD3 with a cell line derived from a different species, e.g. a cell line from a different mammal, e.g. a rodent cell line or a human cell line such as HEK. For example, the cell line is transfected with canine CD3 such that it expresses canine CD3 in a stable or transient manner.

### Kit

In another aspect, the invention provides a kit for the treatment or prevention of a disease for example as listed herein or an immune response and/or for detecting CD3, CD20 and/or CD20 and CD3 for diagnosis, prognosis or monitoring disease comprising an antigen binding domain or antibody of the invention and optionally instructions for use. Such a kit may contain other components, packaging, instructions, or material to aid in the detection of CD20, CD3 and/or CD20 and CD3 protein. The kit may include a labelled antigen binding domain or antibody that binds to CD20 or a binding molecule comprising an antibody that binds to CD20, CD3 and/or CD20 and CD3 and one or more compounds for detecting the label.

### Methods of Making the Antibodies

An antibody described herein can be obtained from a transgenic mammal, for example a rodent, that expresses canine antibodies upon stimulation with an CD3 antigen or a CD20 antigen. Such rodents are described in WO20018/189520 and WO2020/074874.

Thus, an antibody or fragment described herein can be obtained from a mammal, for example a rodent, for example a transgenic animal, that expresses antibodies upon stimulation with a canine CD3 antigen or a CD20 antigen. The transgenic rodent, for example a mouse, may have a reduced capacity to express endogenous antibody genes. Thus, in one embodiment, the rodent has a reduced capacity to express endogenous light and/or heavy chain antibody genes. The rodent, for example a mouse, may therefore comprise modifications to disrupt expression of endogenous kappa and lambda light and/or heavy chain antibody genes so that no functional mouse light and/or heavy chains are produced, for example as further explained below. Such transgenic rodents are described in the art and this is further explained in the Examples below.

Also within the scope of the invention is a method for producing canine antibodies capable of binding CD3 said method comprising
a) immunising a transgenic rodent, e.g. a mouse, with an CD3 antigen wherein said rodent expresses a nucleic acid construct comprising unrearranged canine V, D and J genes,
b) isolating canine antibodies.

Also within the scope of the invention is a method for producing antibodies capable of binding canine CD3 said method comprising
a) immunising a transgenic rodent, e.g. a mouse, with an CD3 antigen wherein said rodent expresses a nucleic acid construct comprising unrearranged canine V, D and J genes,
b) generating a library of sequences comprising heavy chain and light chain sequences from said rodent, e.g. a mouse and
c) isolating antibodies comprising heavy chain and light chain sequences from said libraries.

Further steps may include identifying an antibody that binds to CD3, for example by using functional assays as shown in the Examples.

Methods for preparing or generating the polypeptides, nucleic acids, host cells, products and compositions described herein using in vitro expression libraries can comprise the steps of:
a) providing a set, collection or library of nucleic acid sequences encoding amino acid sequences; and
b) screening said set, collection or library for amino acid sequences that can bind to/have affinity for CD3 and
c) isolating the amino acid sequence(s) that can bind to/have affinity for CD3.

In the above method, the set, collection or library of amino acid sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art (see for example Phage Display of Peptides and Proteins: A Laboratory Manual, Academic Press; 1st edition (Oct. 28, 1996) Brian K. Kay, Jill Winter, John McCafferty). Libraries, for example phage libraries, are generated by isolating a cell or tissue expressing an antigen-specific antibody or fragment thereof, cloning the sequence encoding the antibody or fragment thereof mRNA derived from the isolated cell or tissue and displaying the encoded protein using a library. The sequences can be expressed in bacterial, yeast or other expression systems.

Another aspect also relates to an isolated antibody obtained or obtainable by a method described above.

### Other Methods and Uses

In another aspect, the antibody, antigen binding domain or antigen-binding portion thereof as described herein is used for non-therapeutic purposes, such as diagnostic tests and assays. The invention thus also relates to a method for detecting a canine cell expressing canine CD3 or detecting a canine CD3 protein in a biological sample from a canine subject, comprising contacting a biological sample with the antibody or antigen-binding portion thereof of the invention wherein said antibody or antigen-binding portion thereof is linked to a detectable label. The invention thus also relates to a method for detecting a canine cell expressing canine CD20 or detecting a canine CD20 protein in a biological sample from a canine subject, comprising contacting a biological sample with the antibody, antigen binding domain or antigen-binding portion of the invention wherein said antibody, antigen binding domain or antigen-binding portion thereof is linked to a detectable label. The biological sample may be a biopsy, tissue, blood, serum, plasma, or lymphatic fluid sample.

In certain embodiments, the method may include comparing the amount of binding in the test biological sample to the amount of binding in a control biological sample, wherein increased binding to the test biological sample relative to the control biological sample may indicate the presence of one or more lymphoma cells in the test biological sample. In some embodiments, the biological sample is canine blood or a needle aspirate. These methods are also provided in an in vivo and/or in vitro format.

Modifications of antibodies for diagnostic purposes are well known in the art. For example, antibodies may be modified with a ligand group such as biotin, or a detectable marker group such as a fluorescent group, a radioisotope, or an enzyme. Compounds of the invention can be used for diagnostic purposes and e.g. labelled using conventional techniques. Suitable detectable labels include but are not limited to fluorophores, chromophores, radioactive atoms, electron-dense reagents, enzymes, and ligands having specific binding partners.

In another aspect, the antibody, antigen binding domain or antigen-binding portion thereof of the invention that binds CD20 is used in the isolation and/or identification of cells expressing canine CD20 or cells that contain a cell surface protein that reacts with these binding agents (e.g., B cells, B lymphoma cells, canine CD20).

In another aspect, the antibody, antigen binding domain or antigen-binding portion thereof of the invention that binds CD3 is used in the isolation and/or identification of cells expressing canine CD3 or cells that contain a cell surface protein that reacts with these binding agents (e.g., B cells, B lymphoma cells, canine CD3).

The antibody, antigen binding domain or antigen-binding portion thereof as described herein can also be used in an assay to determine the level of CD20 expression/CD3 expression respectively. The level of expression may then be correlated with base (e.g., control) levels to determine whether a particular disease is present within the patient, the patient's prognosis, or whether a particular treatment regimen is effective.

The invention thus relates to the following clauses.

1. A canine antibody or antigen-binding portion thereof that binds canine CD3.

2. The canine antibody or antigen-binding portion thereof according to clause 1 that binds canine CD3 with a monovalent binding dissociation equilibrium constant (KD) of 100 nM-1000 nM as measured using surface plasmon resonance.

3. The canine antibody or antigen-binding portion thereof according to clause 1 that binds canine CD3 with a monovalent binding dissociation equilibrium constant (KD) lower than 100 nM as measured using surface plasmon resonance.

4. The canine antibody or antigen-binding portion thereof according to a preceding clause that binds to canine CD3, in particular CD3εδ in an agonistic fashion and activates the canine T cell receptor.

5. The canine antibody or antigen-binding fragment portion according to a preceding clause, wherein the antibody or antigen-binding fragment comprises the complementarity determining regions (CDRs) of a heavy chain variable region (HCVR) having an amino acid sequence as set forth in Table 2.

6. The canine antibody or antigen-binding fragment portion according to a preceding clause, wherein the antibody or antigen-binding fragment comprises: (a) the complementarity determining regions (CDRs) of a heavy chain variable region (HCVR) having an amino acid sequence as set forth in Table 2; and (b) the CDRs of a light chain variable region (LCVR) having an amino acid sequence as set forth in Table 2.

7. The canine antibody or antigen-binding fragment portion according to a preceding clause, wherein the antibody or antigen-binding fragment comprises a heavy chain variable region (HCVR) having an amino acid sequence as set forth in Table 2.

8. The canine antibody or antigen-binding fragment portion according to a preceding clause, wherein the antibody or antigen-binding fragment comprises: (a) a heavy chain variable region (HCVR) having an amino acid sequence as set forth in Table 2; and (b) a light chain variable region (LCVR) having an amino acid sequence as set forth in Table 2.

9. The canine antibody or antigen-binding fragment portion according to a preceding clause, wherein the antibody or antigen-binding fragment comprises the CDRs of a HCVR or the HCVR having an amino acid sequence as set forth in Table 2 for PMX157, PMX158, PMX160, PMX190, PMX162, PMX163, PMX189, PMX165, PMX167, PMX168, PMX169, PMX170, PMX171, PMX172, PMX173, PMX174, PMX175, PMX176, PMX177, PMX178, PMX179, PMX180, PMX181, PMX182, PMX183, PMX184, PMX185, PMX186, PMX187, PMX188, PMX190, PMX192, PMX193, PMX194, PMX195, PMX196, PMX197, PMX198, PMX200, PMX272, PMX273, PMX285 or PMX28.

10. The canine antibody or antigen-binding portion thereof according to a preceding clause wherein said antigen-binding portion thereof is an scFv, Fv, heavy chain or single domain antibody.

11. The canine antibody or antigen-binding portion thereof according to a preceding clause wherein the anti-CD3 antibody comprises one or more heavy chain constant domains.

12. A canine antibody or antigen-binding portion thereof that binds to canine CD3 competing with an antibody or antigen-binding portion thereof according to a preceding clause.

13. The canine antibody or antigen-binding portion thereof according to a preceding clause wherein said antibody or antigen-binding portion thereof is conjugated to a therapeutic moiety.

14. The canine antibody or antigen-binding portion thereof according to clause 13 wherein said therapeutic moiety is a second antibody or antigen-binding portion thereof.

15. The canine antibody or antigen-binding portion thereof according to clause 14 wherein said second antibody or antigen-binding portion thereof binds to a different target.

16. The canine antibody or antigen-binding portion thereof according to a preceding clause wherein said antibody or antigen-binding portion thereof is conjugated to a further moiety selected from a half life extending moiety, label, cytotoxin, liposome, nanoparticle or radioisotope.

17. A bispecific antibody comprising a first antigen-binding domain that binds canine CD3 and a second antigen-binding domain that binds a second target antigen, wherein the first antigen-binding domain comprises an antibody or antigen-binding fragment of any one of clauses 1 to 16.

18. A pharmaceutical composition comprising an antibody or antigen-binding portion thereof according to any of clauses 1 to 16 or a bispecific antibody according to clause 17.

19. The antibody or antigen-binding portion thereof according to any of clauses 1 to 16, the bispecific antibody according to clause 17 or the pharmaceutical composition according to clause 18 for use in the treatment of disease.

20. A method of treating a disease in a canine subject in need thereof comprising administering an effective amount of the antibody or antigen-binding portion thereof of any of clauses 1 to 16, bispecific antibody according to clause 17 or a pharmaceutical composition according to clause 18.

21. The canine antibody or antigen-binding portion thereof, bispecific antibody or the pharmaceutical composition according to clause 19 or the method of clause 20 wherein the disease is cancer, a disease mediated by B-cells, for example a B cell lymphoma, leukemia or an immune mediated disease.

22. The canine antibody or antigen-binding portion thereof, bispecific antibody or the pharmaceutical composition according to clause 19 or 21 or the method of clause 20 or 21 further comprising separately administering another therapeutic agent to the subject.

23. The canine antibody or antigen-binding portion thereof, bispecific antibody or the pharmaceutical composition according to clause 22 or the method of clause 22 wherein the therapeutic agent is a cytotoxic agent or a radiotoxic agent, an immunosuppressant or an immunological modulating agent, such as a cytokine or a chemokine.

24. A method for increasing an immune response in a subject, the method comprising administering to the subject a canine antibody or antigen-binding portion of any of clauses 1 to 16, a bispecific antibody according to clause 17 or a pharmaceutical composition according to clause 18.

25. A kit comprising a canine antibody or antigen-binding portion thereof according to any of clauses 1 to 16, a bispecific antibody according to clause 17 or a pharmaceutical composition according to clause 18.

26. The kit according to clause 25 further comprising a reagent for the detection of a canine antibody or antigen-binding portion thereof.

27. A nucleic acid sequence that encodes an antibody or antibody antigen-binding portion thereof according to any of clauses 1 to 16.

28. The nucleic acid sequence according to clause 27 comprising a sequence selected from a SEQ ID as shown in Table 2.

29. A vector comprising a nucleic acid sequence according to any of clauses 27 to 28.

30. A host cell comprising the nucleic acid sequence according to any of clauses 27 to 28 or a vector of clause 29.

31. A method for making a canine antibody that binds CD3 comprising culturing the isolated host cell of clause 30 and recovering said antibody.

32. A method for making a canine antibody that binds CD3 comprising the steps of
a) immunising a transgenic mouse that expresses a nucleic acid construct comprising canine heavy chain V genes and canine light chain V genes with CD3 antigen,
b) generating a library of antibodies from said mouse and
c) isolating an antibody from said library.

33. A method for detecting a CD3 protein or an extracellular domain of a CD3 protein in a biological sample from a canine subject, comprising contacting a biological sample with the antibody or antigen-binding portion thereof of any of clauses 1 to 16 wherein said antibody or antigen-binding portion thereof is linked to a detectable label.

34. A combination therapy comprising an antibody or antigen-binding portion thereof of any of clauses 1 to 16, a bispecific antibody of any of clauses 17 or a pharmaceutical composition of any of clauses 18 and a further therapeutic moiety.

35. The combination therapy according to clause 34 wherein the antibody or antigen-binding portion thereof, bispecific antibody or the pharmaceutical composition and the further therapeutic moiety are administered concurrently or sequentially.

36. A bispecific canine antigen-binding molecule comprising a first antibody or antigen-binding portion thereof that specifically binds canine CD3, and a second antibody or antigen-binding portion thereof that specifically binds canine CD20.

37. The bispecific canine antigen-binding molecule according to clause 36 that binds human CD3 with a monovalent binding dissociation equilibrium constant (KD) of 100 nM-1000 nM as measured using surface plasmon resonance.

38. The bispecific canine antigen-binding molecule according to any of clauses 36 to 37 which provides target specific cell killing.

39. The bispecific canine antigen-binding molecule according to any of clauses 36 to 38 which triggers the T cell surface upregulation of CD25 and/or CD69 upon target mediated cell killing.

40. The bispecific canine antigen-binding molecule according to any of clauses 36 to 39 which activates canine T cells with low INF-γ secretion in vitro and induces T-cell mediated cytotoxicity of human B-cells.

41. The bispecific canine antigen-binding molecule according to any of clauses 36 to 40 which induces T-cell mediated cytotoxicity of human B-cells.

42. The bispecific canine antigen-binding molecule according to any of clauses 36 to 41, wherein the first antibody or antigen-binding portion thereof that specifically binds canine CD3 comprises the heavy chain complementarity determining regions (HCDR1, HCDR2 and HCDR3) from a heavy chain variable region (HCVR) comprising an amino acid sequence selected from the SEQ ID NO. as shown in Table 2 and the light chain complementarity determining regions (LCDR1, LCDR2 and LCDR3) from a light chain variable region (LCVR) comprising an amino acid sequence selected from the group consisting of SEQ ID NO. as shown in Table 2.

43. The bispecific canine antigen-binding molecule according to any of clauses 36 to 42, wherein the second antibody or antigen-binding portion thereof that specifically binds canine CD20 comprises the heavy chain complementarity determining regions (HCDR1, HCDR2 and HCDR3) from a heavy chain variable region (HCVR) comprising a SEQ ID NO. and the light chain complementarity determining regions (LCDR1, LCDR2 and LCDR3) from a light chain variable region (LCVR) comprising an amino acid sequence selected a SEQ ID NO. as shown in Table 2.

44. The bispecific canine antigen-binding molecule according to any of clauses 36 to 43 wherein the second antibody or antigen-binding portion thereof that specifically binds canine comprises PMX272, PMX285, PMX286, PMX188 or PMX189 as shown in Table 2.

45. The bispecific canine antigen-binding molecule according to any of clauses 36 to 44 wherein the antigen-binding molecule is selected from PMX276, PMX277, PMX278, PMX279, PMX280, PMX281, PMX282, PMX283, PMX284, PMX287 or PMX288.

46. The bispecific canine antigen-binding molecule according to any of clauses 36 to 43 wherein said antigen-binding portion thereof is an scFv, Fv, heavy chain or single domain antibody.

47. The bispecific canine antigen-binding molecule according to any of clauses 36 to 45 wherein the anti-CD3 antibody comprises one or more heavy chain constant domains.

48. The bispecific canine antigen-binding molecule according to any of clauses 36 to 47 wherein said antibody or antigen-binding portion thereof is conjugated to a therapeutic moiety.

49. The bispecific canine antigen-binding molecule according to clause 48 wherein said therapeutic moiety is a second antibody or antigen-binding portion thereof.

50. The bispecific canine antigen-binding molecule according to clause 49 wherein said second antibody or antigen-binding portion thereof binds to a different target.

51. The bispecific canine antigen-binding molecule according to any of clauses 36 to 50 wherein said bispecific antigen-binding molecule is conjugated to a further moiety selected from a half life extending moiety, label, cytotoxin, liposome, nanoparticle or radioisotope.

52. A pharmaceutical composition comprising a bispecific antigen-binding molecule according to any of clauses 36 to 51.

53. The bispecific canine antigen-binding molecule according to any of clauses 36 to 51, or the pharmaceutical composition according to clause 52 for use in the treatment of disease.

54. A method of treating cancer or a condition mediated by B-cells in a canine subject in need thereof comprising administering an effective amount of a bispecific canine antigen-binding molecule according to any of clauses 36 to 51, or a pharmaceutical composition according to clause 52.

55. The bispecific canine antigen-binding molecule or the pharmaceutical composition according to clause 53 or the method of clause 54 wherein the disease is cancer, a disease mediated by B-cells, for example a B cell lymphoma, leukemia or an immune mediated disease.

56. The bispecific canine antigen-binding molecule according to any of clauses 53 or 55 or the pharmaceutical composition according to clause 53 or 55 or the method of clause 53 or 55 further comprising separately administering another therapeutic agent to the subject.

57. The bispecific canine antigen-binding molecule according to clause 56, the pharmaceutical composition according to clause 56 or the method of clause 56 wherein the therapeutic agent is a cytotoxic agent or a radiotoxic agent, an immunosuppressant or an immunological modulating agent, such as a cytokine or a chemokine.

58. A method for increasing an immune response in a subject, the method comprising administering to the subject a bispecific canine antigen-binding molecule according to any of clauses 36 to 51 or a pharmaceutical composition of clause 52.

59. A kit comprising a bispecific canine antigen-binding molecule according to any of clauses 36 to 51, bispecific antibody or a pharmaceutical composition according to clause 52.

60. The kit according to clause 59 further comprising a reagent for the detection of the antibody or antigen-binding portion thereof.

61. A nucleic acid sequence that encodes a bispecific canine antigen-binding molecule according to any of clauses 36 to 51.

62. The nucleic acid sequence according to clause 61 comprising a sequence selected from a SEQ ID as shown in Table 2 and/or Table 4.

63. A vector comprising a nucleic acid sequence according to any of clauses 61 to 62.

64. A host cell comprising the nucleic acid sequence according to any of clauses 59 to 60 or a vector of clause 61.

65. A method for making a bispecific antigen-binding molecule comprising culturing the isolated host cell of clause 64 and recovering said antibody.

66. A method for detecting a CD3 protein and a CD20 protein in a biological sample from a canine subject, comprising contacting a biological sample with the bispecific antigen-binding molecule according to any of clauses 36 to 51 wherein said antibody or antigen-binding portion thereof is linked to a detectable label.

67. An canine antibody or antigen-binding portion thereof which binds canine CD20 wherein said antibody comprises In one embodiment, the canine antibody or antigen-binding fragment portion that binds canine CD20 comprises the complementarity determining regions (CDRs) of a heavy chain variable region (HCVR) having an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto.

68. The canine antibody or antigen-binding portion thereof according to clause 67 wherein said antibody or antigen-binding portion thereof comprises a HC variable region sequence comprising an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 75%, 80%, 85% or 90% sequence identity thereto and a LC variable region sequence comprising an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto

69. The canine antibody or antigen-binding portion thereof according to clause 67 or 689 wherein said antigen-binding portion thereof is an scFv, Fv, heavy chain or single domain antibody.

70. The canine antibody or antigen-binding portion thereof according to any of clauses 67 to 69 wherein said antibody or antigen-binding portion thereof is conjugated to a therapeutic moiety.

71. The canine antibody or antigen-binding portion thereof according to clause 70 wherein said therapeutic moiety is a second antibody or antigen-binding portion thereof.

72. The canine antibody or antigen-binding portion thereof according to clause 71 wherein said second antibody or antigen-binding portion thereof binds to a different target.

73. The canine antibody or antigen-binding portion thereof according to a any of clauses 67 to 72 wherein said antibody or antigen-binding portion thereof is conjugated to a further moiety selected from a half life extending moiety, label, cytotoxin, liposome, nanoparticle or radioisotope.

74. A pharmaceutical composition comprising an antibody or antigen-binding portion thereof according to any of clauses 67 to 73.

75. The canine antibody or antigen-binding portion thereof according to any of clauses 65 to 71 or the pharmaceutical composition according to clause 75 for use in the treatment of disease.

76. A method of treating a condition mediated by B-cells in a canine subject in need thereof comprising administering an effective amount of the antibody or antigen-binding portion thereof of any of clauses 67 to

73 or a pharmaceutical composition according to clause 74.

77. The canine antibody or antigen-binding portion thereof or the pharmaceutical composition according to clause 75 or the method of clause 76 wherein the disease is a disease mediated by B-cells, for example a B cell lymphoma, leukemia or an immune mediated disease.

78. The canine antibody or antigen-binding portion thereof or the pharmaceutical composition according to clause 75 or 77 or the method of clause 76 or 77 further comprising separately administering another therapeutic agent to the subject.

79. The canine antibody or antigen-binding portion thereof or the pharmaceutical composition according to clause 78 or the method of clause 78 wherein the therapeutic agent is a cytotoxic agent or a radiotoxic agent, an immunosuppressant or an immunological modulating agent, such as a cytokine or a chemokine.

80. A nucleic acid sequence that encodes an antibody or antibody antigen-binding portion thereof according to any of clauses 67 to 72.

81. The nucleic acid sequence according to clause 80 comprising a sequence selected from SEQ ID NOs. as shown in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269.

82. A vector comprising a nucleic acid sequence according to any of clauses 80 to 81.

83. A host cell comprising the nucleic acid sequence according to any of clauses 80 to 81 or a vector of clause 82.

84. A kit comprising an antibody or antigen-binding portion thereof according to any of clauses 67 to 71 or a pharmaceutical composition according to clause 74.

85. The kit according to clause 84 further comprising a reagent for the detection of the antibody or antigen-binding portion thereof.

86. A method for making a canine antibody that binds CD20 according to any of clauses 67 to 73 comprising culturing the isolated host cell of clause 81 and recovering said antibody.

87. A method for making a canine antibody that binds CD20 according to any of clauses 67 to 73 comprising the steps of
a) immunising a transgenic mouse that expresses a nucleic acid construct comprising canine heavy chain V genes and canine light chain V genes with CD20 antigen,
b) generating a library of antibodies from said mouse and
c) isolating an antibody from said library.

88. A method for detecting a CD20 protein or an extracellular domain of a CD20 protein in a biological sample from a canine subject, comprising contacting a biological sample with the antibody or antigen-binding portion thereof of any of clauses 67 to 73 wherein said antibody or antigen-binding portion thereof is linked to a detectable label.

89. The method of clause 88, wherein the biological sample is a biopsy, tissue, blood, serum, plasma, or lymphatic fluid sample.

90. A method of inhibiting tumor growth or metastasis comprising contacting a tumor cell with an effective amount of the antibody or antigen-binding portion thereof according to any of clauses 67 to 73 or pharmaceutical composition according to clause 74.

91. A method of killing a tumor cell expressing CD20, comprising contacting the cell with the antibody of any one of clauses 67 to 73 or pharmaceutical composition according to clause 74, such that killing of the cell expressing CD20 occurs.

92. The method of clause 91 wherein the tumor cell is a canine tumor cell.

The invention thus also relates to the following embodiments.

1. A bispecific canine antigen-binding molecule comprising a first antigen binding domain or antigen binding portion thereof that specifically binds canine CD3, and a second antigen binding domain that specifically binds canine CD20.

2. The bispecific canine antigen-binding molecule according to embodiment 1 that binds canine CD3 with a monovalent binding dissociation equilibrium constant (KD) of 100 nM-1000 nM as measured using surface plasmon resonance.

3. The bispecific canine antigen-binding molecule according to embodiment 1 or 2, which provides target specific cell killing.

4. The bispecific canine antigen-binding molecule according to any one of the preceding embodiments, which triggers the T cell surface upregulation of CD25 and/or CD69 upon target mediated cell killing.

5. The bispecific canine antigen-binding molecule according to any one of the preceding embodiments, which activates canine T cells with low IFN-γ secretion in vitro and induces T-cell mediated cytotoxicity of human B-cells.

6. The bispecific canine antigen-binding molecule according to any one of the preceding embodiments, which induces T-cell mediated cytotoxicity of human B-cells.

7. The bispecific canine antigen-binding molecule according to any one of the preceding embodiments, wherein the first antigen binding domain or antigen binding portion thereof that specifically binds canine CD3 comprises the heavy chain complementarity determining regions (HCDR1, HCDR2 and HCDR3) from a heavy chain variable region (HCVR) comprising an amino acid sequence selected from the SEQ ID NO. as shown in Table 2 and the light chain complementarity determining regions (LCDR1, LCDR2 and LCDR3) from a light chain variable region (LCVR) comprising an amino acid sequence selected from the group consisting of SEQ ID NO. as shown in Table 2.

8. The bispecific canine antigen-binding molecule according to any one of the preceding embodiments, wherein the second antigen binding domain or antigen binding portion thereof that specifically binds canine CD20 comprises the heavy chain complementarity determining regions (HCDR1, HCDR2 and HCDR3) from a heavy chain variable region (HCVR) comprising an amino acid sequence selected from the SEQ ID NO. as shown in Table 4 and the light chain complementarity determining regions (LCDR1, LCDR2 and LCDR3) from a light chain variable region (LCVR) comprising an amino acid sequence selected from a SEQ ID NO. as shown in Table 2.

9. The bispecific canine antigen-binding molecule according to any one of the preceding embodiments, wherein the first and/or second antigen binding domain or antigen binding portion thereof comprises a light chain variable region (VL) as shown in PMX272, PMX285, PMX286, PMX188 or PMX189 as shown in Table 2.

10. The bispecific canine antigen-binding molecule according to any one of the preceding embodiments, wherein the first and/or second antigen binding domain or antigen-binding portion thereof comprises a light chain variable region (VL) comprising VL CDR1, VL CDR2, and VL CDR3, wherein:
a) the VL CDR1 comprises the amino acid sequence SEQ ID NO: 460; the VL CDR2 comprises the amino acid sequence SEQ ID NO: 461; the VL CDR3 comprises the amino acid sequence SEQ ID NO: 462;
b) the VL CDR1 comprises the amino acid sequence SEQ ID NO: 480; the VL CDR2 comprises the amino acid sequence SEQ ID NO: 481; the VL CDR3 comprises the amino acid sequence SEQ ID NO: 482;
c) the VL CDR1 comprises the amino acid sequence SEQ ID NO: 490; the VL CDR2 comprises the amino acid sequence SEQ ID NO: 491; the VL CDR3 comprises the amino acid sequence SEQ ID NO: 492;
d) the VL CDR1 comprises the amino acid sequence SEQ ID NO: 350; the VL CDR2 comprises the amino acid sequence SEQ ID NO: 351; the VL CDR3 comprises the amino acid sequence SEQ ID NO: 352; or
e) the VL CDR1 comprises the amino acid sequence SEQ ID NO: 110; the VL CDR2 comprises the amino acid sequence SEQ ID NO: 111; the VL CDR3 comprises the amino acid sequence SEQ ID NO: 112.

11. The bispecific canine antigen-binding molecule according to any one of the preceding embodiments, wherein the antigen-binding molecule is selected from PMX276, PMX277, PMX278, PMX279, PMX280, PMX281, PMX282, PMX283, PMX284, PMX287 or PMX288.

12. The bispecific canine antigen-binding molecule according to any one of the preceding embodiments, wherein the molecule comprises:
(a) the first antigen binding domain or antigen-binding portion thereof and the second antigen binding domain or antigen-binding portion thereof wherein:
the first antigen binding domain or antigen-binding portion thereof comprises (i) a first heavy chain variable region (VH) comprising VH complementarity determining region (CDR)1, VH CDR2, and VH CDR3 and (ii) a first light chain variable region (VL) comprising VL CDR1, VL CDR2, and VL CDR3, wherein: the VH CDR1 comprises the amino acid sequence SEQ ID NO: 67; the VH CDR2 comprises the amino acid sequence SEQ ID NO: 68; the VH CDR3 comprises the amino acid sequence SEQ ID NO: 69; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(b) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 87; the VH CDR2 comprises the amino acid sequence SEQ ID NO: 88; the VH CDR3 comprises the amino acid sequence SEQ ID NO: 89; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(c) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 157; the VH CDR2 comprises the amino acid sequence SEQ ID NO: 158; the VH CDR3 comprises the amino acid sequence SEQ ID NO: 159 the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(d) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 167; the VH CDR2 comprises the amino acid sequence SEQ ID NO: 168; the VH CDR3 comprises the amino acid sequence SEQ ID NO: 169; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(e) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 177; the VH CDR2 comprises the amino acid sequence SEQ ID NO: 178; the VH CDR3 comprises the amino acid sequence SEQ ID NO: 179; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(f) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 187; the VH CDR2 comprises the amino acid sequence SEQ ID NO: 188; the VH CDR3 comprises the amino acid sequence SEQ ID NO: 189; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(g) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 327; the VH CDR2 comprises the amino acid sequence SEQ ID NO: 328; the VH CDR3 comprises the amino acid sequence SEQ ID NO: 329; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482;
(h) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 337; the VH CDR2 comprises the amino acid sequence SEQ ID NO: 338; the VH CDR3 comprises the amino acid sequence SEQ ID NO: 339; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492;
(i) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 357; the VH CDR2 comprises the amino acid sequence SEQ ID NO: 358; the VH CDR3 comprises the amino acid sequence SEQ ID NO: 359; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; and the VL CDR2 comprises the amino acid sequence SEQ ID NO: 461; the VL CDR3 comprises the amino acid sequence SEQ ID NO: 462;
(j) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 347; the VH CDR2 comprises the amino acid sequence SEQ ID NO: 348; the VH CDR3 comprises the amino acid sequence SEQ ID NO: 349; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352; or
(k) the first antigen binding domain or antigen-binding portion thereof and the second antigen binding domain or antigen-binding portion thereof wherein:
the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 107; the VH CDR2 comprises the amino acid sequence SEQ ID NO: 108; the VH CDR3 comprises the amino acid sequence SEQ ID NO: 109; the VL CDR1 comprises the amino acid sequence SEQ ID NO: 110; the VL CDR2 comprises the amino acid sequence SEQ ID NO: 111; and the VL CDR3 comprises the amino acid sequence SEQ ID NO: 112;
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 110; the VL CDR2 comprises the amino acid sequence SEQ ID NO: 111; and the VL CDR3 comprises the amino acid sequence SEQ ID NO: 112.

13. The bispecific canine antigen-binding molecule according to any one of the preceding embodiments, wherein the molecule comprises:
(a) a first VH comprising the amino acid sequence of SEQ ID NO: 64; a second VH comprising the amino acid sequence of SEQ ID NO: 524, and a first and second VL comprising the amino acid sequence of SEQ ID NO: 456;
(b) a first VH comprising the amino acid sequence of SEQ ID NO: 84; a second VH comprising the amino acid sequence of SEQ ID NO: 524, and a first and second VL comprising the amino acid sequence of SEQ ID NO: 456;
(c) a first VH comprising the amino acid sequence of SEQ ID NO: 154; a second VH comprising the amino acid sequence of SEQ ID NO: 524, and a first and second VL comprising the amino acid sequence of SEQ ID NO: 456;
(d) a first VH comprising the amino acid sequence of SEQ ID NO: 164; a second VH comprising the amino acid sequence of SEQ ID NO: 524, and a first and second VL comprising the amino acid sequence of SEQ ID NO: 456;
(e) a first VH comprising the amino acid sequence of SEQ ID NO: 174; a second VH comprising the amino acid sequence of SEQ ID NO: 524, and a first and second VL comprising the amino acid sequence of SEQ ID NO: 456;
(f) a first VH comprising the amino acid sequence of SEQ ID NO: 184; a second VH comprising the amino acid sequence of SEQ ID NO: 524, and a first and second VL comprising the amino acid sequence of SEQ ID NO: 456;
(g) a first VH comprising the amino acid sequence of SEQ ID NO: 324; a second VH comprising the amino acid sequence of SEQ ID NO: 524, and a first and second VL comprising the amino acid sequence of SEQ ID NO: 476;
(h) a first VH comprising the amino acid sequence of SEQ ID NO: 334; a second VH comprising the amino acid sequence of SEQ ID NO: 524, and a first and second VL comprising the amino acid sequence of SEQ ID NO: 486;
(i) a first VH comprising the amino acid sequence of SEQ ID NO: 74; a second VH comprising the amino acid sequence of SEQ ID NO: 524, and a first and second VL comprising the amino acid sequence of SEQ ID NO: 456;
(j) a first VH comprising the amino acid sequence of SEQ ID NO: 344; a second VH comprising the amino acid sequence of SEQ ID NO: 524, and a first and second VL comprising the amino acid sequence of SEQ ID NO: 346; or
(k) a first VH comprising the amino acid sequence of SEQ ID NO: 104; a second VH comprising the amino acid sequence of SEQ ID NO: 524, and a first and second VL comprising the amino acid sequence of SEQ ID NO: 106.

14. The bispecific canine antigen-binding molecule according to any one of the preceding embodiments, wherein said antigen-binding domain or antigen binding portion thereof is an scFv, Fv, heavy chain or single domain antibody.

15. The bispecific canine antigen-binding molecule according to any one of the preceding embodiments, wherein the anti-CD3 antigen-binding domain comprises one or more heavy chain constant domains.

16. The bispecific canine antigen-binding molecule according to any one of the preceding embodiments, wherein said antigen-binding domain or antigen binding portion thereof is conjugated to a therapeutic moiety.

17. The bispecific canine antigen-binding molecule according to embodiment 16, wherein said therapeutic moiety is a second antigen-binding domain.

18. The bispecific canine antigen-binding molecule according to embodiment 17 wherein said second antigen-binding domain binds to a tumour antigen.

19. The bispecific canine antigen-binding molecule according to any one of the preceding embodiments wherein said bispecific antigen-binding molecule is conjugated to a further moiety selected from a half life extending moiety, label, cytotoxin, liposome, nanoparticle or radioisotope.

20. A pharmaceutical composition comprising a bispecific antigen-binding molecule according to any one of the preceding embodiments.

21. The bispecific canine antigen-binding molecule according to any one of embodiments 1 to 19, or the pharmaceutical composition according to embodiment 20 for use in the treatment of disease.

22. A method of treating cancer or a condition mediated by B-cells in a canine subject in need thereof comprising administering an effective amount of the bispecific canine antigen-binding molecule according to any one of embodiments 1 to 19, or a pharmaceutical composition according to embodiment 20.

23. The bispecific canine antigen-binding molecule or the pharmaceutical composition according to embodiment 21 or the method of embodiment 22 wherein the disease is cancer, a disease mediated by B-cells, for example a B cell lymphoma, leukemia or an immune mediated disease.

24. The bispecific canine antigen-binding molecule according to any one of embodiments 21 or 23, or the pharmaceutical composition according to embodiment 21 or 23, or the method of embodiment 22 or 23 further comprising separately administering another therapeutic agent to the subject.

25. The bispecific canine antigen-binding molecule according to embodiment 24, the pharmaceutical composition according to embodiment 24 or the method of embodiment 24, wherein the therapeutic agent is a cytotoxic agent, a radiotoxic agent, an immunosuppressant, an immunological modulating agent, such as a cytokine or a chemokine, or an antibody or antigen-binding portion thereof that binds canine CD20.

26. A method for increasing an immune response in a subject, the method comprising administering to the subject a bispecific canine antigen-binding molecule according to any one of embodiments 1 to 19 or a pharmaceutical composition of embodiments 20.

27. A kit comprising a bispecific canine antigen-binding molecule according to any of embodiments 1 to 19, bispecific antibody or a pharmaceutical composition according to embodiment 20.

28. The kit according to embodiment 27 further comprising a reagent for the detection of the antibody or antigen-binding portion thereof.

29. A nucleic acid sequence that encodes a bispecific canine antigen-binding molecule according to any of embodiments 1 to 19.

30. The nucleic acid sequence according to embodiment 29 comprising a nucleic acid sequence selected from a SEQ ID as shown in Table 2 and/or Table 4.

31. A vector comprising a nucleic acid sequence according to embodiment 29 to 30.

32. A host cell comprising the nucleic acid sequence according to embodiment 29 to 30, or a vector of embodiment 30.

33. A method for making a bispecific antigen-binding molecule comprising culturing the isolated host cell of embodiment 32 and recovering said antibody.

34. A method for detecting a CD3 protein and a CD20 protein in a biological sample from a canine subject, comprising contacting a biological sample with the bispecific antigen-binding molecule according to any of embodiments 1 to 19 wherein said antigen-binding molecule is linked to a detectable label.

35. A combination therapy comprising the bispecific canine antigen-binding molecule according to any of embodiments 1 to 19 and an antibody or antigen-binding portion thereof that binds canine CD20.

36. A canine antibody or antigen-binding portion thereof which binds canine CD20 wherein said antibody comprises the complementarity determining regions (CDRs) of a heavy chain variable region (HCVR) having an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto.

37. The canine antibody or antigen-binding portion thereof according to embodiment 36 wherein said antibody or antigen-binding portion thereof comprises a HC variable region sequence comprising an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 75%, 80%, 85% or 90% sequence identity thereto and a LC variable region sequence comprising an amino acid sequence as set forth in Table 4 as shown for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269 or a sequence with at least 70%, 75%, 80%, 85%, 90% or 95% sequence identity thereto.

38. The canine antibody or antigen-binding portion thereof according to embodiment 36 or 37 wherein said antigen-binding portion thereof is an scFv, Fv, heavy chain or single domain antibody.

39. The canine antibody or antigen-binding portion thereof according to any one of embodiments 36 to 38 wherein said antibody or antigen-binding portion thereof is conjugated to a therapeutic moiety.

40. The canine antibody or antigen-binding portion thereof according to embodiment 39 wherein said therapeutic moiety is a second antibody or antigen-binding portion thereof.

41. The canine antibody or antigen-binding portion thereof according to embodiment 40 wherein said second antibody or antigen-binding portion thereof binds to a tumour antigen.

42. The canine antibody or antigen-binding portion thereof according to any one of embodiments 36 to 41 wherein said antibody or antigen-binding portion thereof is conjugated to a further moiety selected from a half life extending moiety, label, cytotoxin, liposome, nanoparticle or radioisotope.

43. A pharmaceutical composition comprising an antibody or antigen-binding portion thereof according to any one of embodiments 36 to 42.

44. The canine antibody or antigen-binding portion thereof according to any one of embodiments 36 to 42 or the pharmaceutical composition according to embodiment 43 for use in the treatment of disease.

45. A method of treating a condition mediated by B-cells in a canine subject in need thereof comprising administering an effective amount of the antibody or antigen-binding portion thereof of any one of embodiments 36 to 42 or a pharmaceutical composition according to embodiment 43.

46. The canine antibody or antigen-binding portion thereof or the pharmaceutical composition according to embodiment 44 or the method of embodiment 45 wherein the disease is a disease mediated by B-cells, for example a B cell lymphoma, leukemia or an immune mediated disease.

47. The canine antibody or antigen-binding portion thereof or the pharmaceutical composition according to embodiment 44 or 46 or the method of embodiment 45 or 46 further comprising separately administering another therapeutic agent to the subject.

48. The canine antibody or antigen-binding portion thereof or the pharmaceutical composition according to embodiment 47 or the method of embodiment 47 wherein the therapeutic agent is a cytotoxic agent or a radiotoxic agent, an immunosuppressant or an immunological modulating agent, such as a cytokine or a chemokine.

49. A nucleic acid sequence that encodes an antibody or antibody antigen-binding portion thereof according to any of embodiments 36 to 42.

50. The nucleic acid sequence according to embodiment 49 comprising a sequence selected from SEQ ID NOs: as shown in Table 4 for PMX232, PMX233, PMX234, PMX235, PMX237, PMX241, PMX243, PMX244, PMX245, PMX247, PMX248, PMX249, PMX250, PMX251, PMX252, PMX253, PMX254, PMX255, PMX256, PMX257, PMX258, PMX259, PMX262, PMX263, PMX264, PMX265, PMX266, PMX267, PMX268 or PMX269.

51. A vector comprising a nucleic acid sequence according to embodiment 49 or 50.

52. A host cell comprising the nucleic acid sequence according to any one of embodiments 49 to 50 or a vector of embodiment 49.

53. A kit comprising an antibody or antigen-binding portion thereof according to any one of embodiments 36 to 42 or a pharmaceutical composition according to embodiment 43.

54. The kit according to embodiment 53 further comprising a reagent for the detection of the antibody or antigen-binding portion thereof.

55. A method for making a canine antibody that binds CD20 according to any one of embodiments 36 to 42 comprising culturing the isolated host cell of embodiment 52 and recovering said antibody.

56. A method for making a canine antibody that binds CD20 according to any one of embodiments 36 to 42 comprising the steps of
a) immunising a transgenic mouse that expresses a nucleic acid construct comprising canine heavy chain V genes and canine light chain V genes with CD20 antigen,
b) generating a library of antibodies from said mouse and
c) isolating an antibody from said library.

57. A method for detecting a CD20 protein or an extracellular domain of a CD20 protein in a biological sample from a canine subject, comprising contacting a biological sample with the antibody or antigen-binding portion thereof of any one of embodiments 36 to 42 wherein said antibody or antigen-binding portion thereof is linked to a detectable label.

58. The method of embodiment 57, wherein the biological sample is a biopsy, tissue, blood, serum, plasma, or lymphatic fluid sample.

59. A method of inhibiting tumour growth or metastasis comprising contacting a tumour cell with an effective amount of the antibody or antigen-binding portion thereof according to any of embodiments 36 to 42 or pharmaceutical composition according to embodiment 43.

60. A method of killing a tumour cell expressing CD20, comprising contacting the cell with the antibody of any one of embodiments 36 to 42 or pharmaceutical composition according to embodiment 43, such that killing of the cell expressing CD20 occurs.

61. The method of embodiment 60 wherein the tumour cell is a canine tumour cell.

62. A canine antibody or antigen-binding portion thereof that binds canine CD3.

63. The canine antibody or antigen-binding portion thereof according to embodiment 62 that binds canine CD3 with a monovalent binding dissociation equilibrium constant (KD) of 100 nM-1000 nM as measured using surface plasmon resonance.

64. The canine antibody or antigen-binding portion thereof according to embodiment 62 or 63 that binds canine CD3 with a monovalent binding dissociation equilibrium constant (KD) lower than 100 nM as measured using surface plasmon resonance.

65. The canine antibody or antigen-binding portion thereof according to any one of embodiments 62 to 63 that binds to canine CD3, in particular CD3εδ in an agonistic fashion and activates the canine T cell receptor.

66. The canine antibody or antigen-binding portion according to any one of embodiments 62 to 65, wherein the antibody or antigen-binding fragment comprises the complementarity determining regions (CDRs) of a heavy chain variable region (HCVR) having an amino acid sequence as set forth in Table 2.

67. The canine antibody or antigen-binding portion according to any one of embodiments 62 to 66, wherein the antibody or antigen-binding fragment comprises: (a) the complementarity determining regions (CDRs) of a heavy chain variable region (HCVR) having an amino acid sequence as set forth in Table 2; and (b) the CDRs of a light chain variable region (LCVR) having an amino acid sequence as set forth in Table 2.

68. The canine antibody or antigen-binding portion according to any one of embodiments 62 to 67, wherein the antibody or antigen-binding fragment comprises a heavy chain variable region (HCVR) having an amino acid sequence as set forth in Table 2.

69. The canine antibody or antigen-binding portion according to any one of embodiments 62 to 68, wherein the antibody or antigen-binding portion comprises: (a) a heavy chain variable region (HCVR) having an amino acid sequence as set forth in Table 2; and (b) a light chain variable region (LCVR) having an amino acid sequence as set forth in Table 2.

70. The canine antibody or antigen-binding portion according to any of embodiments 62 to 69, wherein the antibody or antigen-binding portion comprises the CDRs of a HCVR or the HCVR having an amino acid sequence as set forth in Table 2 for PMX157, PMX158, PMX160, PMX190, PMX162, PMX163, PMX189, PMX165, PMX167, PMX168, PMX169, PMX170, PMX171, PMX172, PMX173, PMX174, PMX175, PMX176, PMX177, PMX178, PMX179, PMX180, PMX181, PMX182, PMX183, PMX184, PMX185, PMX186, PMX187, PMX188, PMX190, PMX192, PMX193, PMX194, PMX195, PMX196, PMX197, PMX198, PMX200, PMX272, PMX273, PMX285 or PMX286.

71. The canine antibody or antigen-binding portion thereof according to any one of embodiments 62 to 70, wherein the antibody or antigen-binding portion comprises (i) a heavy chain variable region (V_{H}) comprising V_{H} complementarity determining region (CDR)1, V_{H} CDR2, and V_{H} CDR3; and (ii) a light chain variable region (V_{L}) comprising V_{L} CDR1, V_{L} CDR2, and V_{L} CDR3, wherein:
(a) the V_{H} CDR1 comprises the amino acid sequence SEQ ID NO: 47; the V_{H} CDR2 comprises the amino acid sequence SEQ ID NO: 48; the V_{H} CDR3 comprises the amino acid sequence SEQ ID NO: 49; the V_{L} CDR1 comprises the amino acid sequence SEQ ID NO: 50; the V_{L} CDR2 comprises the amino acid sequence SEQ ID NO: 51; the V_{L} CDR3 comprises the amino acid sequence SEQ ID NO: 52;
(b) the V_{H} CDR1 comprises the amino acid sequence SEQ ID NO: 87; the V_{H} CDR2 comprises the amino acid sequence SEQ ID NO: 88; the V_{H} CDR3 comprises the amino acid sequence SEQ ID NO: 89; the V_{L} CDR1 comprises the amino acid sequence SEQ ID NO: 90; the V_{L} CDR2 comprises the amino acid sequence SEQ ID NO: 91; the V_{L} CDR3 comprises the amino acid sequence SEQ ID NO: 92;
(c) the V_{H} CDR1 comprises the amino acid sequence SEQ ID NO: 127; the V_{H} CDR2 comprises the amino acid sequence SEQ ID NO: 128; the V_{H} CDR3 comprises the amino acid sequence SEQ ID NO: 129; the V_{L} CDR1 comprises the amino acid sequence SEQ ID NO: 130; the V_{L} CDR2 comprises the amino acid sequence SEQ ID NO: 131; the V_{L} CDR3 comprises the amino acid sequence SEQ ID NO: 132; or
(d) the V_{H} CDR1 comprises the amino acid sequence SEQ ID NO: 167; the V_{H} CDR2 comprises the amino acid sequence SEQ ID NO: 168; the V_{H} CDR3 comprises the amino acid sequence SEQ ID NO: 169; the V_{L} CDR1 comprises the amino acid sequence SEQ ID NO: 170; the V_{L} CDR2 comprises the amino acid sequence SEQ ID NO: 171; the V_{L} CDR3 comprises the amino acid sequence SEQ ID NO: 172.

72. The canine antibody or antigen-binding portion thereof according to any one of embodiments 62 to 71, wherein the molecule comprises:
(a) a V_{H} comprising the amino acid sequence of SEQ ID NO: 44; a V_{L} comprising the amino acid sequence of SEQ ID NO: 46;
(b) a V_{H} comprising the amino acid sequence of SEQ ID NO: 84; a V_{L} comprising the amino acid sequence of SEQ ID NO: 86;
(c) a V_{H} comprising the amino acid sequence of SEQ ID NO: 124; a V_{L} comprising the amino acid sequence of SEQ ID NO: 126; or
(d) a V_{H} comprising the amino acid sequence of SEQ ID NO: 164; a V_{L} comprising the amino acid sequence of SEQ ID NO: 166.

73. The canine antibody or antigen-binding portion thereof according to any one of embodiments 62 to 72, wherein said antigen-binding portion thereof is an scFv, Fv, heavy chain or single domain antibody.

74. The canine antibody or antigen-binding portion thereof according to any one of embodiments 62 to 73, wherein the anti-CD3 antibody comprises one or more heavy chain constant domains.

75. A canine antibody or antigen-binding portion thereof that binds to canine CD3 competing with an antibody or antigen-binding portion thereof according to any one of embodiments 62 to 75.

76. The canine antibody or antigen-binding portion thereof according to any one of embodiments 62 to 75 wherein said antibody or antigen-binding portion thereof is conjugated to a therapeutic moiety.

77. The canine antibody or antigen-binding portion thereof according to embodiment 76 wherein said therapeutic moiety is a second antibody or antigen-binding portion thereof.

78. The canine antibody or antigen-binding portion thereof according to embodiment 76 wherein said second antibody or antigen-binding portion thereof binds to a tumour antigen.

79. The canine antibody or antigen-binding portion thereof according to any one of embodiments 62 to 78, wherein said antibody or antigen-binding portion thereof is conjugated to a further moiety selected from a half life extending moiety, label, cytotoxin, liposome, nanoparticle or radioisotope.

80. A bispecific antibody comprising a first antigen-binding domain that binds canine CD3 and a second antigen-binding domain that binds a second target antigen, wherein the first antigen-binding domain comprises an antibody or antigen-binding fragment of any one of embodiments 62 to 79.

81. A pharmaceutical composition comprising an antibody or antigen-binding portion thereof according to any one of embodiments 62 to 79 or a bispecific antibody according to embodiment 80.

82. The antibody or antigen-binding portion thereof according to any one of embodiments 62 to 79, the bispecific antibody according to embodiment 80 or the pharmaceutical composition according to embodiment

81 for use in the treatment of disease.

83. A method of treating a disease in a canine subject in need thereof comprising administering an effective amount of the antibody or antigen-binding portion thereof of any one of embodiments 62 to 79, bispecific antibody according to embodiment 80 or a pharmaceutical composition according to embodiment 81.

84. The canine antibody or antigen-binding portion thereof, bispecific antibody or the pharmaceutical composition according to embodiment 82 or the method of embodiment 83 wherein the disease is cancer, a disease mediated by B-cells, for example a B cell lymphoma, leukemia or an immune mediated disease.

85. The canine antibody or antigen-binding portion thereof, bispecific antibody or the pharmaceutical composition according to embodiment 82 or 84 or the method of embodiment 83 or 84 further comprising separately administering another therapeutic agent to the subject.

86. The canine antibody or antigen-binding portion thereof, bispecific antibody or the pharmaceutical composition according to embodiment 85 or the method of embodiment 85 wherein the therapeutic agent is a cytotoxic agent or a radiotoxic agent, an immunosuppressant or an immunological modulating agent, such as a cytokine or a chemokine.

87. A method for increasing an immune response in a subject, the method comprising administering to the subject a canine antibody or antigen-binding portion thereof of any one of embodiments 62 to 79, a bispecific antibody according to embodiment 80 or a pharmaceutical composition according to embodiment 81.

88. A kit comprising a canine antibody or antigen-binding portion thereof according to any one of embodiments 62 to 80, a bispecific antibody according to embodiment 80 or a pharmaceutical composition according to embodiment 81.

89. The kit according to embodiment 84 further comprising a reagent for the detection of a canine antibody or antigen-binding portion thereof.

90. A nucleic acid sequence that encodes an antibody or antibody antigen-binding portion thereof according to any of embodiments 62 to 80.

91. The nucleic acid sequence according to embodiment 90 comprising a sequence selected from a SEQ ID as shown in Table 2.

92. A vector comprising a nucleic acid sequence according to any of embodiments 90 to 91.

93. A host cell comprising the nucleic acid sequence according to any of embodiments 90 to 91 or a vector of embodiment 92.

94. A method for making a canine antibody that binds CD3 comprising culturing the isolated host cell of embodiment 93 and recovering said antibody.

95. A method for making a canine antibody that binds CD3 comprising the steps of
a) immunising a transgenic mouse that expresses a nucleic acid construct comprising canine heavy chain V genes and canine light chain V genes with CD3 antigen,
b) generating a library of antibodies from said mouse and
c) isolating an antibody from said library.

96. A method for detecting a CD3 protein or an extracellular domain of a CD3 protein in a biological sample from a canine subject, comprising contacting a biological sample with the antibody or antigen-binding portion thereof of any one of embodiments 62 to 80 wherein said antibody or antigen-binding portion thereof is linked to a detectable label.

97. A combination therapy comprising an antibody or antigen-binding portion thereof of any one of embodiments 62 to 80, a bispecific antibody of embodiment 80 or a pharmaceutical composition of embodiment 80 and a further therapeutic moiety.

98. The combination therapy according to embodiment 97 wherein the antibody or antigen-binding portion thereof, bispecific antibody or the pharmaceutical composition and the further therapeutic moiety are administered concurrently or sequentially.

Unless otherwise defined herein, scientific and technical terms used in connection with the present disclosure shall have the meanings that are commonly understood by those of ordinary skill in the art. While the foregoing disclosure provides a general description of the subject matter encompassed within the scope of the present disclosure, including methods, as well as the best mode thereof, of making and using this disclosure, the following Examples are provided to further enable those skilled in the art to practice this disclosure. However, those skilled in the art will appreciate that the specifics of these Examples should not be read as limiting on the invention, the scope of which should be apprehended from the claims and equivalents thereof appended to this disclosure. Various further aspects and embodiments of the present disclosure will be apparent to those skilled in the art in view of the present disclosure.

All documents mentioned in this specification are incorporated herein by reference in their entirety, including references to gene accession numbers, scientific publications and references to patent publications.

“and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein. Unless context dictates otherwise, the descriptions and definitions of the features set out above are not limited to any particular aspect or embodiment of the invention and apply equally to all aspects and embodiments which are described.

The invention is further illustrated in the following non-limiting examples.

### EXAMPLES

### Example 1: Ky9™ Mice Immunisation

### Antigen Preparation

### 1. Generation of Canine CD3εδ and CD3εγ Expressing Mouse Embryonic Fibroblasts (MEFs)

The coding sequence (CDS) DNA and amino acid sequences of canine CD3ε, CD3δ and CD3γ are listed in Table 1 (SEQ ID NOs: 1 to 6). MEFs were grown on 90 mm round tissue culture plates as monolayers in DMEM-high glucose (Life Technologies) supplemented with 10% FBS, 1 mM Sodium pyruvate (Sigma-Aldrich), 0.5 mM p-mercaptoethanol (Gibco) and 1% MEM non-essential amino acids (Sigma-Aldrich) at 37° C., with 5% CO₂. To generate CD3εδ and CD3εγ expressing MEFs, wild type MEFs were stably transfected, using Lipofectamine LTX with PLUS™ reagent (ThermoFisher Scientific) according to the manufacturer's recommended instructions, with two mammalian expression vectors, one encoding for canine CD3ε extra cellular (ECD) and transmembrane (TM) domains, the other encoding for CD3δ or CD3γ ECD and TM domains. Both constructs contain PiggyBac terminal inverted repeats to mediate transposition when co-transfected with PiggyBac transposase. CD3ε expressing vector contains puromycin resistant cassette, while CD3δ and CD3γ expressing vectors carry hygromycin resistant cassette. After dual selection with puromycin and hygromycin, cells were stained using anti-canine CD3 antibody (clone CA17.2A12, Biorad) to confirm surface expression and high expressing cells were further enriched by FACs sorting using BD FACS Aria. Sorted cells were expanded and frozen to create master and working cell banks which were used for mice immunisation, serum titre and candidates screening.

### 2. Generation of Canine CD20 Expressing MEFs

### Cloning Canine CD20

A search of the CAMFAM_3.1 boxer reference genome was conducted using the UCSC Genome Browser. The genomic sequence for CD20 (MS4A1) was downloaded along with mRNA sequence AB210085.1. Using this sequence data, primers were designed to enable amplification of CD20 from cDNA with added sequence to allow for seamless cloning. This allowed for confirmation of the CD20 sequence in dog blood and seamless cloning into a piggyBac cloning vector.

Isolation of Canine CD20 mRNA and Generation of cDNA

Beagle whole blood was supplied by Envigo RMS (Alconbury, Huntingdon, UK) and PBMCs were isolated using a Ficoll gradient. Briefly, 10 ml whole blood was diluted with 25 ml phosphate buffered saline (PBS) and layered onto 15 ml Ficoll Paque Plus (Sigma Aldrich) before centrifuging at 800 rcf for 10 min, room temp, with slow acceleration and no brake. The interphase disk was collected into PBS. Total RNA was isolated from PBMCs with the QIAGEN RNeasy Mini Kit (Qiagen, Hilden, DE) and standard procedures, with an on-column DNAse digestion. cDNA generation was undertaken using the SuperScript™ IV First-Strand Synthesis System following standard procedures and anchored oligo dT primers (ThermoFisher, Massachusetts, US).

To generate canine CD20 expressing MEFs, cells were co-transfected with a vector containing the canine CD20 DNA coding sequence (Table 1, SEQ ID NO: 22), flanked by PiggyBac terminal inverted repeats, and PiggyBac transposase containing vector using Lipofectamine LTX with PLUS^{T}M reagent (ThermoFisher Scientific) according to the manufacturer's recommended instructions. Stably transfected cells were selected 48 hrs later using puromycin.

### Ky9™ Mice Immunisation

Ky9™ mice, substantially as described in WO2018/189520 and WO2020/074874, have been genetically engineered to carry canine immunoglobulin heavy (IGH) chain and light chain (IGL) variable (V) region genes, IGH D region genes and IGH and IGL J region genes 5′ to the mouse constant regions. Ky9™ mice therefore produce chimeric antibodies with canine heavy and light variable regions with mouse constant regions. Information concerning, or the nucleic acid comprising, the variable region of such chimeric antibody chains may be used to generate fully canine antibodies, for therapeutic use in dogs for example. The rodent containing the canine DNA may also serve as an animal model for understanding of disease and testing of medicines.

Ky9™ mice were immunised with MEFs expressing either canine CD3εδ, or canine CD20. MEFs were thawed a week before immunisation and split twice to reach the required number. On the day of injection, MEFs were trypsinised, washed twice with PBS, and counted before resuspending in injection solution. For prime immunisation, each mouse was injected intraperitoneally with 200 μl of resuspended MEFs in PBS mixed with adjuvant. For boost immunisation, the same protocol was used in the absence of adjuvant.

### Serum Titre Determination

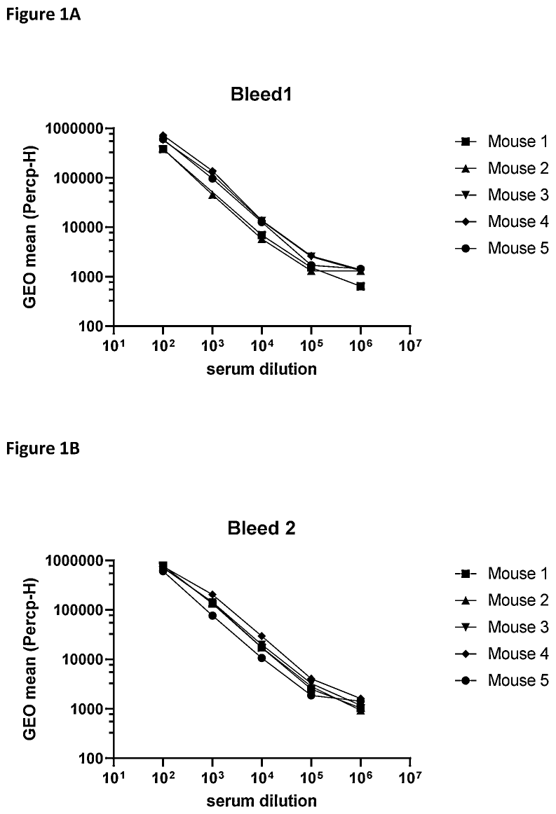

Ky9™ mice were bled 10 days prior to prime immunisation and 10 days after each subsequent boost immunisation. Serum and red blood cells were separated using microvette 200 Z-gel tubes (Starstedt AG & Co. KG, Germany) and titres of the canine CD3εδ-specific antibody response was evaluated using a BD Accuri C6 Flow Cytometer (Becton Dickinson, NJ, USA) or Beckman Coulter's CytoFLEX S. Post-immunisation serum was serially diluted in FACS buffer (PBS+3% FBS) and added to either 10⁵ wild type MEF or HEK cells or 10⁵ of the same cells stably-expressing canine CD3εδ or canine CD20 MEF or HEK cells. Mouse antibodies were detected with 1/200 dilution of BB700 conjugated 2° monoclonal antibodies against isotypes IgG1, IgG2a, IgG2b (BD OptiBuild™, Becton Dickinson), and binding on these cells was compared to pre-immunisation serum. FIG. 1 exemplifies the results obtained in a cohort of Ky9™ mice immunised with MEFs expressing canine CD3 εδ after boost immunisations.

### Example 2: Isolation of Antibody Producing Cells

Following immunisation of Ky9™ mice with MEFs expressing either canine CD3εδ, or canine CD20 antibody producing cells were isolated as described below.

### Tissue Isolation

Spleens, lymph nodes and bone marrow were harvested from immunised mice. Splenocytes were prepared by cutting the spleen into pieces and forcing these through a 45 μm cell strainer (Falcon) while rinsing with RPMI-1640 (Lonza, Basel, CH)+10% FBS on ice. A similar process was used for lymphocytes isolation from the lymph nodes. Bone marrow was collected from femur and tibia by flushing the marrow with RPMI-1640 using a 21-gauge needle, through a 45 μm cell strainer pre-wetted with RPMI-1640. All cell types were pelleted at 300 g for 5 min, before either being directly used for flow sorting or resuspended in FBS+10% dimethyl sulfoxide (DMSO) and being frozen at −150° C.

### Cell Sorting

Antigen-specific cells can be captured by fluorescent labelled virus-like particles (VLP) or antigen protein probes. VLPs are generated from HEK cells stably transfected with canine CD3εδ (Table 1, SEQ ID NO: 1 and SEQ ID NO:2) or canine CD20 (Table 1, SEQ ID NO: 22, and SEQ ID NO: 24), and subsequently transiently transfected with the retrovirus gag protein, and fluorescently labelled MA (gag matrix fragment p15-GFP fusion protein); the gag expression enables VLP budding from cells, and MA labels the VLPs for fluorescence detection. Briefly, human embryonic kidney (HEK) 293 cells were grown on 90 mm round tissue culture plates as monolayers in DMEM/F12 (Life Technologies) supplemented with 10% fetal bovine serum (FBS; Sigma Aldrich) at 37° C., with 5% CO₂. To generate HEK cells expressing canine CD3εδ, HEK293 cells were transfected either with two mammalian expression vectors, one encoding for canine CD3ε extra cellular (ECD) and transmembrane (TM) domains, the other encoding for CD3δ or CD3γ ECD and TM domains, together with a vector encoding for PiggyBac transposase using polyethyleneimine (PEI MAX: 40 kDa, Polysciences Inc., Eppelheim, Germany). 30 μl of PEI MAX (1 mg ml⁻¹), 5 μg cDNA and 1 ml DMEM/F12 were incubated for 10 min at room temperature, and was added dropwise to a 90 mm plate of 70-80% confluent HEK293 cells, and incubated for 2 days before use. To generate HEK cells expressing canine CD20, HEK293 cells were transfected with a vector containing the canine CD20 cDNA sequence (Table 1, SEQ ID NOs: 22 and 24), flanked by PiggyBac terminal inverted repeats, and a vector encoding for PiggyBac transposase using polyethyleneimine (PEI MAX: 40 kDa, Polysciences Inc., Eppelheim, Germany) as described before. Stably transfected cells were selected using suitable antibiotics 48 hours post transfection for 7-10 days.

Antigen-specific B cells can also be captured by labelled antigen protein probes. To produce heterodimeric canine CD3εδ-Fc fusion proteins (Table 1, SEQ ID NOs: 7-10), vectors encoding canine CD3ε-hIgG4PE-knob and canine CD3δ-hIgG4PE-hole were co-transfected in CHO cells. The human Fc contain the Knob-in-Hole (KiH) mutations to favour heterodimerization (Merchant A. M et al Nat Biotechnol. 1998 July; 16(7):677-81). Fc tagged probes were purified from culture supernatant using Mab select protein A resin (Cytiva) or by AKTA using Mab Select SuRe columns (Cytiva). Conjugation of CD3εδ-Fc fusion protein to Alexa Fluor 647 was carried out using the Alexa Fluor 647 Antibody labelling kit (Molecular Probes—Invitrogen) following the manufacturer's protocol. The degree of labelling was determined using a NanoDrop spectrophotometer. Different dilutions of both probes were tested on splenocytes of mice immunised with CD3εδ-expressing MEFs or with an unrelated immunogen in conjunction with CD3εδ overexpressing HEK 293 cell derived Gag-GFP VLP to identify and sort antigen specific B cells by flow cytometry. The optimal dilution displaying minimal background staining on un-relevant material was then used for antigen-specific B cells identification and sorting by BD FACSAria Fusion cell sorter (BD Biosciences).

Sorted B cells were prepared using the 10XGenomics Chromium Single Cell Immune Profiling system and the V(D)J Kit (10XGenomics) according to manufacturer's instructions. Nucleotide sequences of expressed antibodies were determined by Illumina MiSeq sequencing with 600 cycles (2×300 cycles) or by Ilumina iSeq, Miseq, MiniSeq, Nextseq, Hiseq 4000 or Novaseq sequencing with 2×150 cycles. The sequences are analysed using custom tools based on the pRESTO/Change-O (Yale University)/IgBlast (NCBI, USA) software to identify paired VH and VL sequences and a clonal lineage information is also constructed based on the identities of the heavy chain V, D, J and light chain V, J genes.

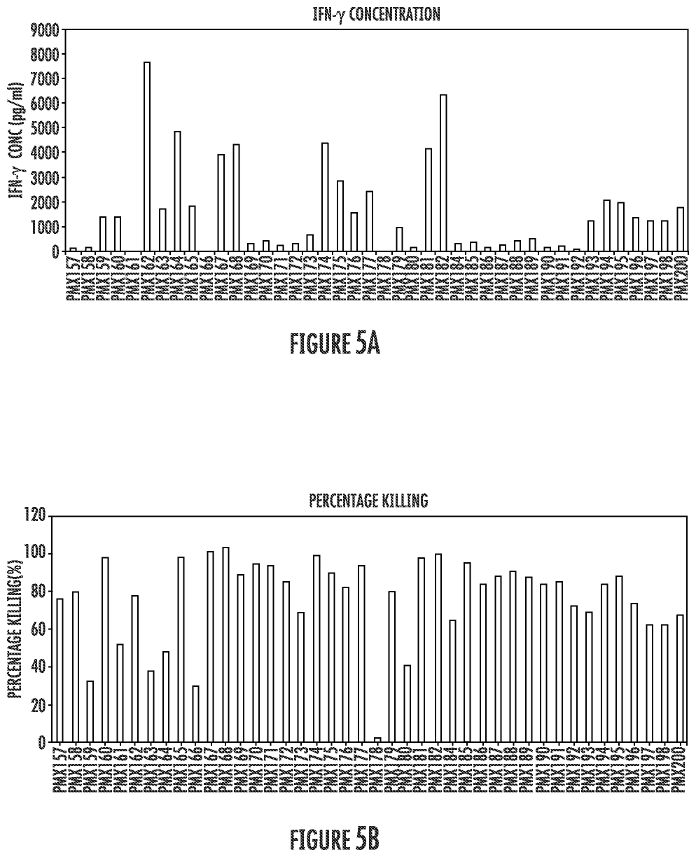

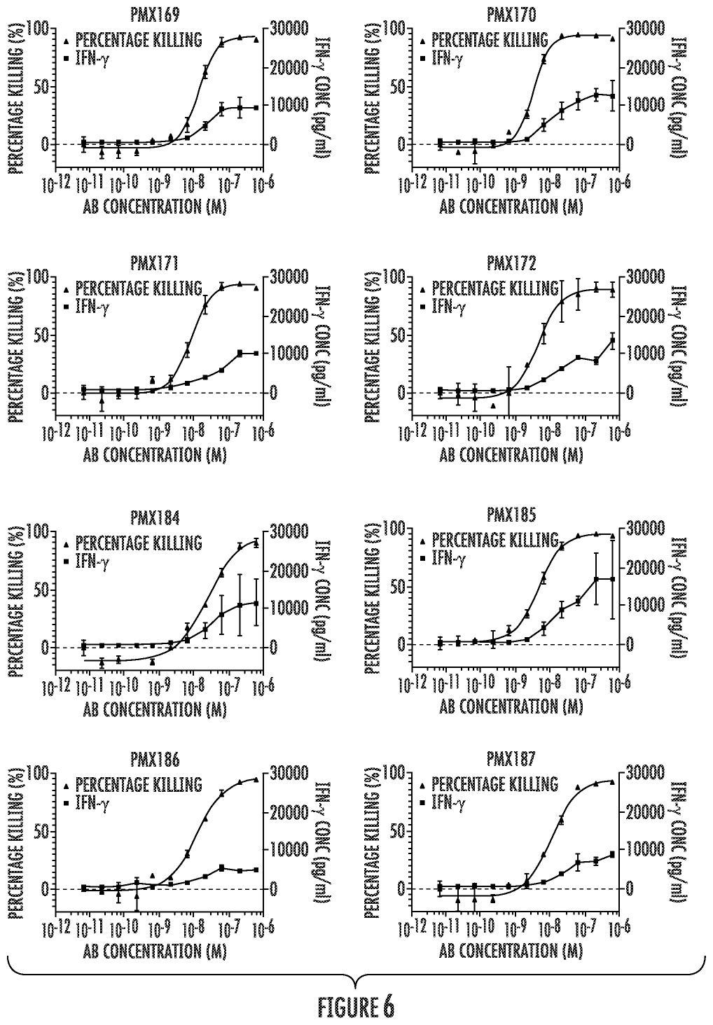

An example of the analysis of antibody sequences of sorted antigen-specific single B cells is shown in FIG. 5 of WO2015/040401 and shows antibody sequences that are arranged by heavy-chain V-gene family usage, and clustered to generate the displayed phylogenetic trees. From phylogenetic trees such as these, candidate clones are selected. The nucleic acid and amino acid sequences, VH and VL and their corresponding CDRs for anti-CD3 and anti-CD20 candidates are provided in the Sequences Table 2 and Table 4 respectively.

### Example 3: Candidate Antibody Format for Expression

### Anti-Canine CD3 Candidate Bispecific Format

### Generation of CD3 Arm

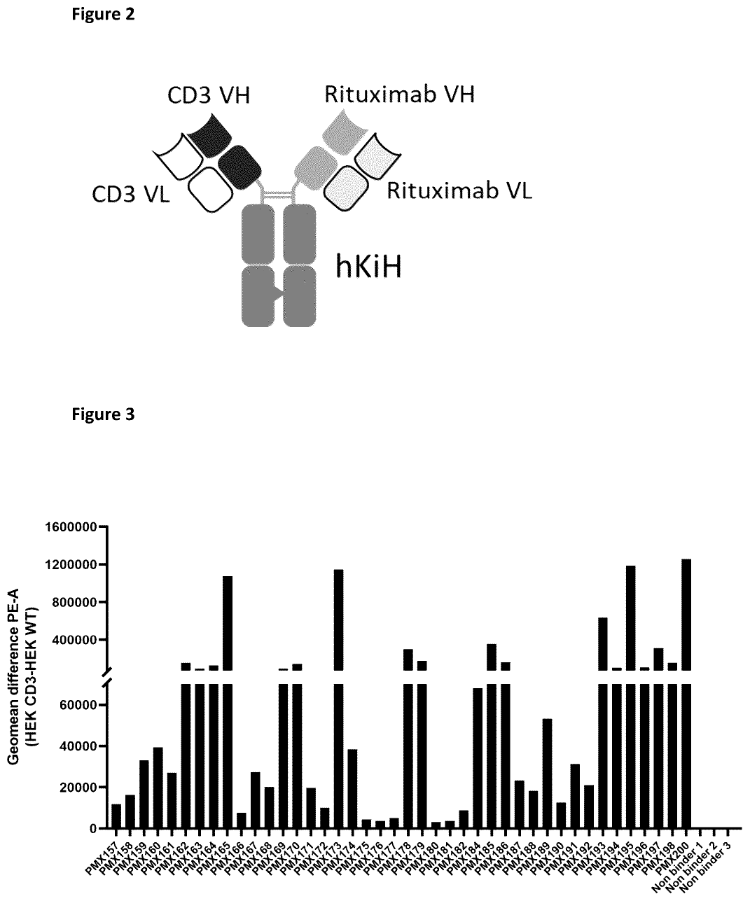

To screen for agonistic anti-CD3 candidates, we opted for a bispecific killing format with one canine CD3 arm and one Rituximab arm (FIG. 2). To achieve this, VH DNA sequences of the selected anti-canine CD3 candidates were seamlessly cloned into expression vectors upstream of the human IgG4PE constant region (CH1-hinge-CH2-CH3) with the hole and protein A mutations using Gibson assembly (Table 1, SEQ ID NOs: 11 and and 12). V_{L} DNA sequences were cloned into expression vectors upstream constant regions of human kappa light chain (Table 1, SEQ ID NOs: 13 and 14).

### Generation of CD20 Arm (Targeted Against Human CD20)

Rituximab V_{H} DNA sequence (Table 1, SEQ ID NOs: 17 and 18) was cloned in expression vector containing human IgG4PE constant regions (CH1-hinge-CH2-CH3) with the knob mutations (Table 1, SEQ ID NOs: 15 and 16), while Rituximab VL DNA sequence (Table 1, SEQ ID NOs: 19 and 20) was cloned in expression vector containing constant regions of human kappa light chain (Table 1, SEQ IS NOs: 13 and 14).

The four expression vectors encoding the CD3 arm heavy chain and light chains and the Rituximab arm heavy and light chains respectively were co-transfected in 1:1:1:1 ratio into a suitable mammalian cell line such as CHO cells for production as described below.

### Anti-Canine CD3 Candidate Monospecific Format

Agonistic anti-CD3 VH and VL pairs identified by the capacity of bispecific killing (Example 4) were seamlessly cloned into expression vectors upstream of canine IgG-B effector function deficient Fc (Table 1, SEQ ID NOs: 29 and 30). VL DNA sequences were cloned into expression vectors upstream of canine Lambda C5 constant region (Table 1, SEQ ID NOs:13 and 14).

### Anti-Canine CD20 Candidate Format

To screen for anti-CD20 candidates, DNA encoding VH of the anti-canine CD20 candidates were cloned into expression vectors containing the wild type canine IgG-B constant region sequence (Table 1, SEQ ID NOs: 27 and 28), while V_{L} DNA sequences were cloned upstream of canine lambda C5 constant region (Table 1, SEQ ID NOs: 31 and SEQ ID NO:32). The expression vectors encoding both the anti-CD20 heavy and light chains were co-transfected into a suitable mammalian cell line such as CHO cells for production as described below.

### Antibody Production

For overgrowth production, 6×10⁶ selected CHO cells were seeded in 3 ml culture media and incubated at 32° C., 5% CO₂ with shaking at 200 rpm. 4% HyClone Cell Boost 7a supplement+0.4% HyClone Cell Boost 7b supplement+1% glucose was added to the media on days 1, 4, 7 and 10. Culture supernatants were collected on day 12 and the antibody concentration was determined using surface plasmon resonance (Biacore 8K, Cytiva Life Sciences) for protein A binding.

### Example 4: Screening Cascade to Identify Agonistic Bispecific CD3 Candidates

Candidate anti-CD3 sequences, in the bispecific format described in Example 3, were screened for canine CD3εδ and CD3εγ binding and target-specific cell killing in order to identify suitable agonistic anti-CD3 candidates.

Binding Screen Using Cells with Surface CD3 Expression

CHO cell supernatant containing bispecific antibodies were diluted to 1, 5 or 10 μg/ml in FACS buffer (PBS containing 3% FBS) and screened for their ability to bind cell surface canine CD3εδ heterodimer. In brief, HEK293 or MEF cells expressing canine CD3εδ or CD3εγ at the cell surface are incubated with 100 μl of FACS buffer containing the candidate antibody for 30′ on ice. As a control, the parental cell line (HEK293 or MEF cells not expressing canine CD3) was also stained with the same antibody solution. After staining, cells were washed with 150 μl FACS buffer followed by centrifugation at 300 g for 3 min. Supernatant was removed, and the cell pellet was resuspended in FACS buffer containing a 1:1000 dilution of a fluorescently labelled secondary antibody recognising the human Fc region of the test antibody for 30 min in the dark, followed by washing with 150 μl FACS buffer and centrifugation at 300×g for 3 mins. Cells were resuspended in FACS buffer and flow cytometry performed using either a Cytoflex (Beckton Dickinson) or an Accuri (Beckman Coulter) cytometer, followed by data analysis using FlowJo (FIG. 3).

### Secondary SPR Based Binding Screen Using CD3εδ-Fc Fusion Protein

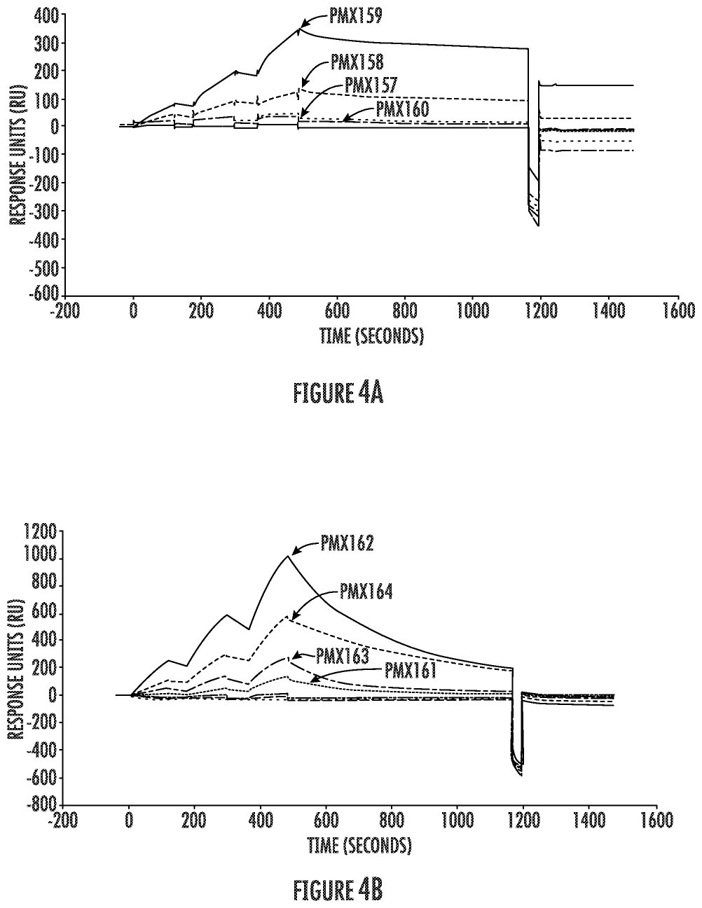

Candidates CD3 bispecific antibodies were also screened for their ability to bind canine CD3εδ-Fc using surface plasmon resonance (SPR) using a Biacore 8K (Cytiva). Canine CD3εδ-Fc was covalently bound by amine coupling to the surface of a CM5 chip (Cytiva). CHO cell supernatant was diluted to 66, 33 and 16.5 nM in HBS-EP+ buffer and run on the chip single cycle kinetic protocol. Data was analysed using the dedicated software (Biacore Insight Evaluation Software). Examples of the resulting sensograms are presented in FIGS. 4A-4B.

### In Vitro Cell Based Functional Assay to Identify Agnostic CD3 Bispecific Candidates

Candidate CD3 bispecific antibodies as generated in Example 3 were tested in an in vitro T cell mediated cell killing assay, using a canine MDCK cell lines expressing CD20 as target cells and peripheral blood mononuclear cells (PBMCs) as effector cells in order to assess the capacity of T cell activation and target mediated lysis.

To generate the target cells, canine cell line MDCK II (ATCC) was stably transfected with a piggyBac based expression construct encoding either the human CD20 (Table 1, SEQ ID NO: 21, and 23) or dog CD20 (Table 1, SEQ ID NOs: 22 and 24) alongside a construct expressing GFP (Table 1, SEQ ID NO: 25) and the piggyBac transposase vector. Human CD20+GFP transfected cells were selected for puromycin and blasticidin resistance and hCD20^{high}GFP (top 5%) cells were FACS sorted by staining for hCD20 expression using anti-human CD20 antibody (clone: 2H7, BioLegend) and GFP expression using the FITC channel.

Canine CD20+GFP transfected cells were selected for puromycin and blasticidin resistance and dCD20^{high}GFP (top 5%) cells were FACS sorted by staining for dCD20 expression using anti-dog CD20 antibody (Invivogen) and GFP expression using the FITC channel.

To assess bispecific killing, canine peripheral blood mononuclear cells (PBMCs, Envigo) were used as a source of effector cells. PBMCs were isolated from freshly drawn whole blood, with sodium heparin anticoagulant, using Ficoll-Paque plus (Cytiva, GE17-1440-02) density gradient centrifugation. In brief, canine blood was diluted 1:1 with phosphate buffer saline (PBS) and carefully layered on top of Ficoll-paque plus, centrifugated at 800×g for 20 minutes with slow acceleration and no break. The top layer and interphase disk were diluted with PBS and centrifuged at 420×g for 10 minutes to collect the PBMCs in the pellet, which was washed a second time in PBS to remove all remnants of Ficoll. After a second centrifugation, PBMCs were resuspended in media (PBMC media=RPMI+10% heat inactivated foetal bovine serum+1% penicillin-streptomycin+1% non-essential amino acids+1% L-glutamine+1% sodium pyruvate+1% HEPES) before use in the bispecific killing assay.

To set up the bispecific killing assay, 10,000 MDCK II cells expressing hCD20+GFP or dCD20+GFP were co-cultured with PBMCs at an effector: target ratio of 20:1 in black walled 96 well plates. The cell antibody mix was incubated for 48 h at 37° C., 5% CO₂, in a 1:1 mix of MDCK II media (DMEM+1% L-glutamine+10% fetal bovine serum) and PBMC media. Initial screening was carried out using an antibody concentration of 1 ug/ml. Following the 48 hour incubation, 40 ul supernatant was then collected from each well for cytokine quantification. IFNγ was measured by ELISA (MABtech) according to the manufacturer's instructions, with supernatant diluted 1:30 in diluent buffer.

Bispecific killing was assessed using GFP signal, which is proportional to the number of live cells and was used as an endpoint measure of surviving cells. GFP signal was measured with CLARIOstar (BMG Labtech). Data was analysed using MARS (BMG Labtech) and percentage of killing in the presence of antibodies was calculated using Microsoft Excel. Background signal was obtained from a media only control and subtracted from the signal obtained from each test sample. Max signal (0% killing) was obtained from a sample of cells treated identically but where antibodies were omitted. Graphs were plotted in Graph Prism.

The results for candidate screening for both bispecific killing capacity and IFNγ release is summarised in FIG. 5. It is worth noting that bispecific killing potency in vitro does not correlate with IFNγ release. For example, PMX165 and PMX182 has similar in vitro killing potency, and yet PMX165 mediated killing releases a third of the IFNγ compared to PMX182. This phenomenon has been previously observed in human anti-CD3 bispecific antibodies (Trinklein et al. 2019 mAbs 11(4):639-652). Our data supports the hypothesis that T cell's capacity to kill target cells can be decoupled from cytokine release. Cytokine release seems to correlate with TCR activation, the stronger the agonistic TCR binding, the stronger the cytokine release (Staflin et al (2020) JCI insight. 5(7):e133757). Cytokine release is one of the major safety considerations for T cell engager bispecific antibodies. Candidates capable of killing and mediate with low IFNγ release was used as the top criteria for candidate shortlisting.

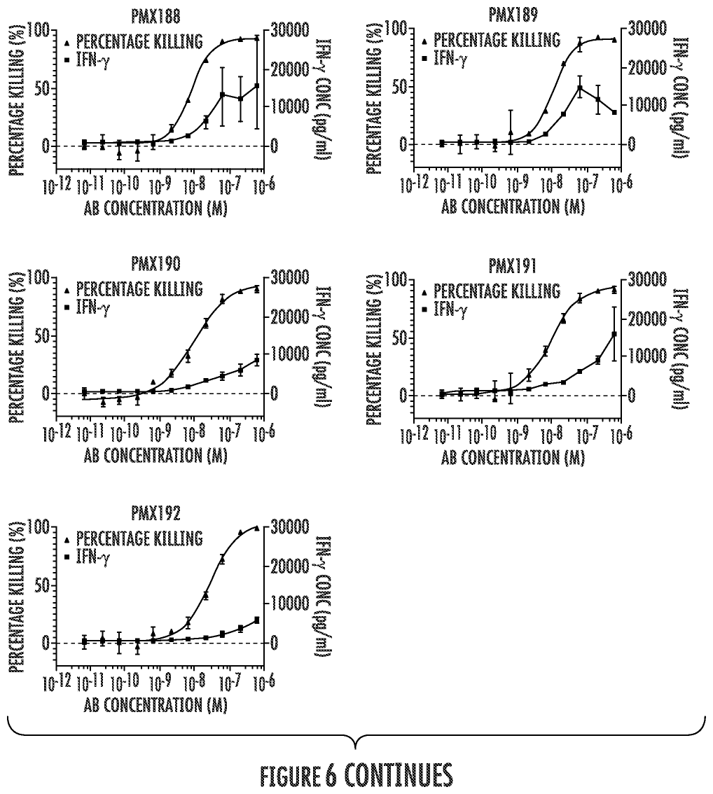

Based on this criteria, a shortlist of anti-CD3 candidates was selected for full characterisation. A dose titration of shortlisted candidates ranging from 10 ug/ml to 0.0001 ug/ml was conducted to further characterise the candidates killing potency and IFNγ cytokine release and the results of which is shown in FIG. 6, with the calculated EC50 of killing potency for each dose response curve found in Table 6.

TABLE 6
Related to FIG. 5

| Sample | EC50 (M) |
|---|---|
| PMX169 | 1.40E−08 |
| PMX170 | 3.32E−09 |
| PMX171 | 8.52E−09 |
| PMX172 | 4.66E−09 |
| PMX184 | 2.55E−08 |
| PMX185 | 4.79E−09 |
| PMX186 | 1.29E−08 |
| PMX187 | 1.14E−08 |
| PMX188 | 8.23E−09 |
| PMX189 | 1.10E−08 |
| PMX190 | 1.00E−08 |
| PMX191 | 9.44E−09 |
| PMX192 | 3.21E−08 |

### Example 5: CD3 Affinity Determination for Shortlisted CD3 Candidates in Bispecific Format

It is known in humans that although high affinity CD3 agonism correlates with bispecific killing potency in vitro, in vivo killing potency is neither correlative to in vitro killing potency nor dependent on CD3 affinity. On the other hand, the severity of the cytokine release is positively correlating with CD3 affinity both in vitro and in vivo (Staflin et al (2020) JCI insight. 5(7):e133757. Haber et al. (2021) Scientific Reports 11:14397.). Therefore, CD3 affinity is used as a selection criterion for identifying potentially safer CD3 candidate arms.

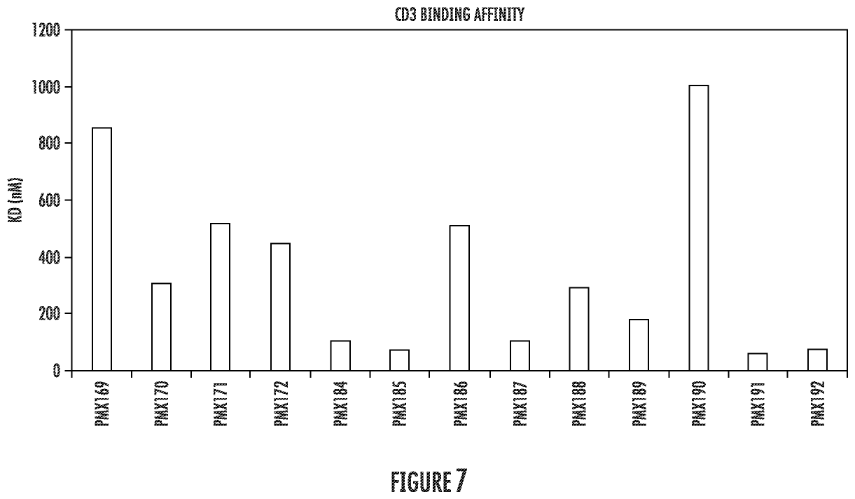

Binding affinity of shortlisted candidates, described in Example 3, were assessed using Biacore 8K (Cytiva). Briefly, a CM5 Sensor Chip (Cytiva) was docked onto a Biacore 8K, equilibrated for 30′ at RT and then Running Buffer (10 mM HEPES pH6 150 mM NaCl 3 mM EDTA and 0.005% Tween20) was applied to the SPR chip surface. Canine CD3εδ-Fc was diluted into 10 mM acetate buffer pH4.5 and immobilised using standard amine coupling reaction. Antibodies dilutions were prepared by diluting the shortlisted candidates from 600 nM to 7.4 nM (5 concentrations with 1:3 dilutions) in running buffer and kinetics was assessed using multi-cycle kinetics method (120 sec association-300 sec dissociation). Kinetics and/or Affinity quantification was performed using Biacore Insight following standard analyses methods. Affinity to CD3 of the shortlisted candidates is shown in FIG. 7 and associated Table 7.

TABLE 7
Related to FIG. 7

| Sample | KD (nM) |
|---|---|
| PMX169 | 854.4506 |
| PMX170 | 304.4051 |
| PMX171 | 517.2645 |
| PMX172 | 447.0086 |
| PMX184 | 99.80019 |
| PMX185 | 72.10497 |
| PMX186 | 512.6375 |
| PMX187 | 106.1404 |
| PMX188 | 291.8425 |
| PMX189 | 177.398 |
| PMX190 | 999.022 |
| PMX191 | 62.1122 |
| PMX192 | 74.37936 |

### Example 6: Usage of Monospecific CD3 Candidates for Ex Vivo T Cell Activation

Another application of agonistic anti-CD3 antibodies is for the ex vivo activation and expansion of canine T cells. This can be achieved by using the monospecific anti-CD3 antibodies alone or in combination with anti-CD28 antibodies and/or other T cell stimulatory factors (such as IL-2 for instance).

Although we have conducted a screen to identify agnostic anti-CD3 arms, producing CD3 candidates in monospecific format allows the identification of CD3 variable region sequences which enables the ex vivo activation of canine T cells as described in Example 4 anti-CD3 monospecific format. Monospecific CD3 antibodies were produced in CHO cells as described in Example 3 and purified from the CHO supernatant using protein A resin (MabSelect, Cytiva). Purified antibodies were quality controlled by HPLC-size exclusion chromatography (H-SEC) and were all above 99% monomeric.

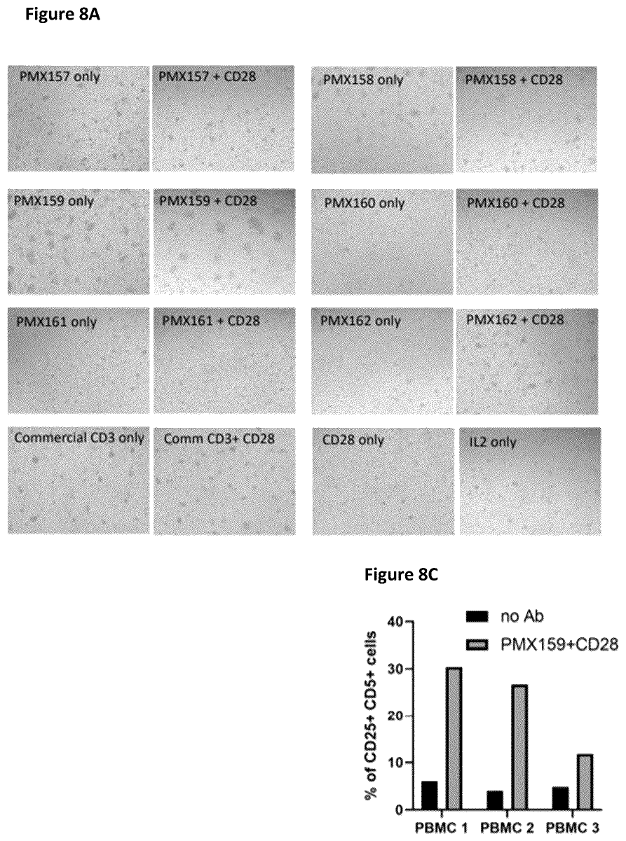

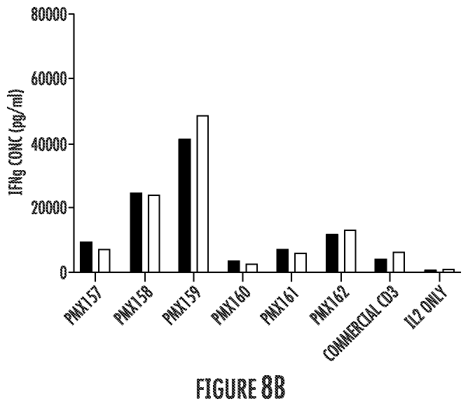

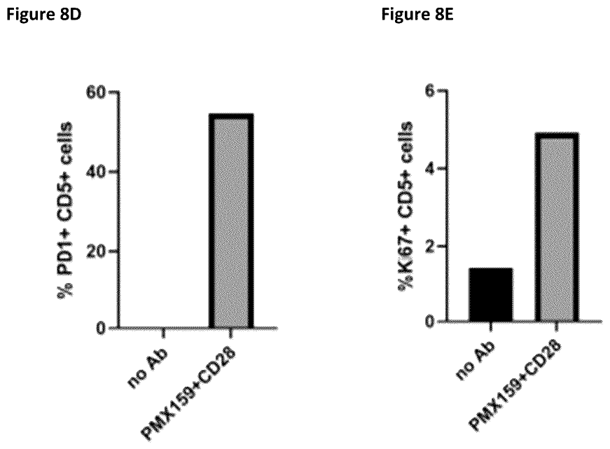

To evaluate the ability of these monospecific CD3 candidates to activate canine T cells, thawed frozen or fresh canine PBMC, isolated as previously described, were cultured in PBMC medium supplemented with canine IL-2 at 50 ng/ml in cell culture plated precoated for 2 to 3 hours at 37° C. or overnight at 4° C. with our candidate monospecific CD3 antibody at 10 μg/ml in PBS alone or in combination with anti-canine CD28 antibody (clone 1C6, Fisher scientific) at 5 μg/ml in PBS. Seventy-two hours after, cells were imaged to determine the presence of T cell clusters (FIG. 8A). Concentration of IFNγ in supernatant was determined at 72 and 96 hours using canine IFNγ ELISA (Mabtech) (FIG. 8B). Cell surface upregulation of CD25 and PD-1 could also be detected as exemplified by using PMX159 together with anti-canine CD28 (FIGS. 8C-8D). Furthermore, canine T cell proliferation could also be detected by the increase in Ki67 staining (FIG. 8E). Since the anti-CD3 candidates were pre-selected based on TCR agonism in bispecific killing, most of fully canine anti-CD3 mAbs tested were able to activate canine T cell ex vivo equally good or better than the commercially available anti-canine CD3 antibody (clone CA17.2A12, Biorad) as demonstrated in FIG. 8B.

Agonistic anti-human CD3 antibody OKT3 has been shown to act as a T cell immunosuppressant. It was approved in 1985 for the use of managing rejection of organ transplants. Although highly effective in immunomodulation, the severe side effect of cytokine release syndrome due to the strong initial T cell activation, led to its withdrawal. Second generation design with the use of effector function deficient OKT3 circumvents the high cytokine release problem and has led to the FDA approval of Teplizumab in 2022 for treatment of patients recently diagnosed with T1 D. These pioneering human studies have paved the way for anti-canine CD3 therapy to realise its therapeutic potential Our discovery and demonstration of agonistic anti-CD3 coupled with canine effector function deficient Fc allows the therapeutic investigation of this class of candidates in canine autoimmune related conditions such as canine T1 D.

### Example 7: Binding Assays of Monospecific Anti-Canine CD20 Candidate Antibodies

To identify anti-CD20 variable region sequences which can be used as monospecific anti-CD20 and is compatible for CD3/CD20 bispecific formatting, monospecific anti-CD20 antibodies as described in Example 3 were screened using a cell-based CD20 binding assay, where CD20 is displayed on the cell surface as its natural confirmation.

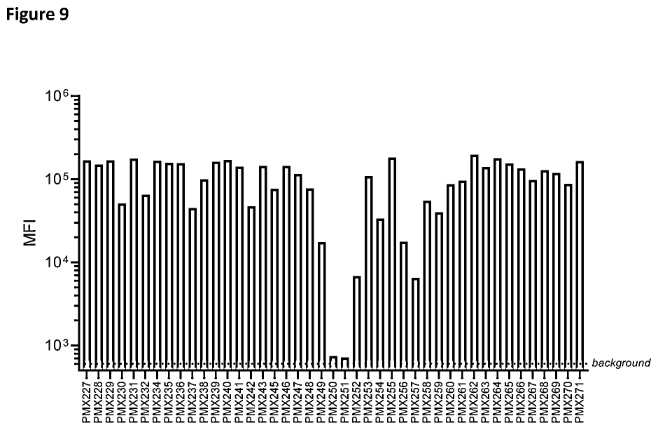

A CD20 binding screen was performed on candidate proteins produced from the canine CD20 immunisation as described in Examples 2 and 3. CHO cells supernatants containing candidate antibodies were diluted to 10 μg/ml in FACS buffer (PBS containing 3% FBS) and screened for their ability to bind canine CD20 expressed on cell surface. In brief, 1-2×10⁵ canine CD20-expressing HEK cells were incubated with candidate mAbs for one hour at 4° C., at a fixed concentration of 10 μg/ml followed by incubation with 5 μg/ml of FITC-conjugated anti-canine IgG secondary antibody (Bethyl Laboratories) for 1 hour at 4° C. Cells incubated with anti-canine IgG FITC secondary antibody and without primary anti-canine CD20 antibody, or with isotype control primary antibody, were used as negative controls. The data were acquired on either a Beckman Coulter CytoFLEX or a BD Accuri C6 Plus flow cytometer and analysed using FlowJo software. Results of binding assays of PMX227, PMX228, PMX229, PMX230, PMX231, PMX232, PMX234, PMX235, PMX235, PMX237, PMX238, PMX239, PMX240, PMX241, PMX242, PMX243, PMX244, PMX245, PMX246, PMX247, PMX248, PMX249, PMX250, PMX251, PMX252, PMX253, PMX254, PMX255, PMX256, PMX257, PMX258, PMX259, PMX260, PMX261, PMX262, PMX263, PMX264, PMX265, PMX266, PMX267, PMX268, PMX269, PMX270, PMX271 are shown in FIG. 9. All candidates shown, with exception of PMX250 and PMX251, demonstrated strong binding capacity to CD20 expressing cells.

### Example 8: Killing Capacity of Monospecific Anti-Canine CD20 Binders

To demonstrate the cytotoxic killing capability, CD20 binders in the monospecific format with the canine wild type IgG-B Fc (effector function proficient), as described in Example 3 were further examined for their functional capacity to mediate cytotoxicity as described below, complement dependent cytotoxicity and antibody dependent cellular cytotoxicity Both mechanism of killing replies on the recruitment of native immune effectors, complement and NK cells respectively via the effector function proficient canine Fc.

### Complement Dependent Cytotoxicity (CDC) Activity

CLBL-1 canine lymphoma tumour cell line (University of Veterinary Medicine Vienna) that natively expresses canine CD20 (Table 1, SEQ ID NOs: 22 and 24) was used as the target cell line for a CDC assay. 10,000 CLBL-1 cells per well of 96-well plate (white with clear bottom) were incubated with canine complement preserved serum (BioIVT) at a final dilution of 1:4 and 1 μg/ml of anti-canine CD20 antibody, for 2 hours at 37° C., 5% CO₂. The assay was set up using media (RPMI+1% L-glutamine+20% fetal bovine serum) made using heat inactivated serum so that canine complement preserved serum would be the only source of complement.

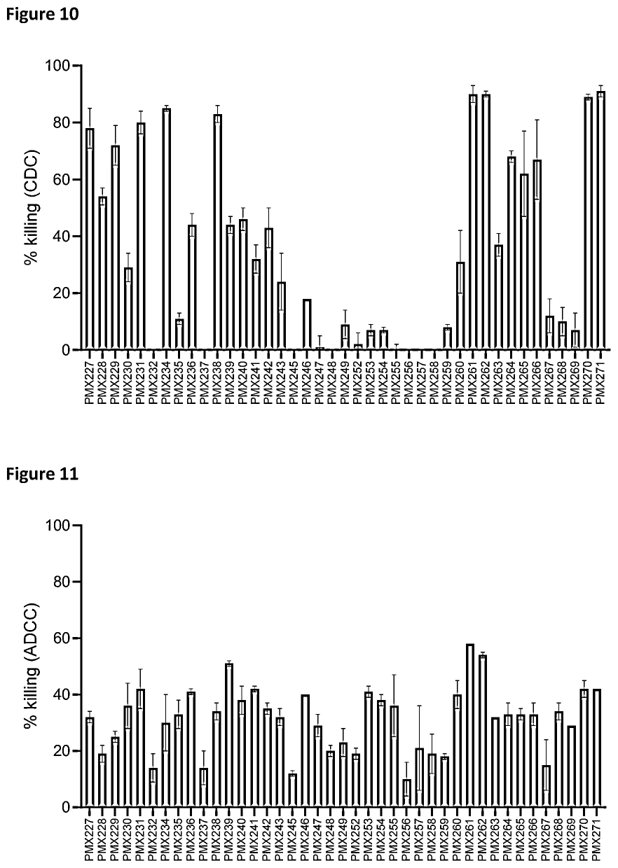

Live cells were then quantified using CellTitre-Glo@ Luminescent Cell Viability Assay (Promega) following the assay protocol. This assay uses the ATP content of live cells as an indication of cell viability. Luminescence was measured on a CLARIOstar (BMG Labtech). Data were analyzed using MARS software (BMG Labtech) and the number of live cells remaining was used to calculate the percentage of killing in the presence of antibodies using Microsoft Excel, using wells without antibody as baseline. Graphs were plotted in GraphPad Prism. Results of CDC assay using monospecific anti-CD20 antibodies PMX227, PMX228, PMX229, PMX230, PMX231, PMX232, PMX234, PMX235, PMX235, PMX237, PMX238, PMX239, PMX240, PMX241, PMX242, PMX243, PMX244, PMX245, PMX246, PMX247, PMX248, PMX249, PMX252, PMX253, PMX254, PMX255, PMX256, PMX257, PMX258, PMX259, PMX260, PMX261, PMX262, PMX263, PMX264, PMX265, PMX266, PMX267, PMX268, PMX269, PMX270, PMX271 are presented in FIG. 10.

### Antibody Dependent Cellular Cytotoxicity (ADCC) Activity

Canine cell line, such as MDCK II (ATCC), was stably transfected with a construct encoding for the canine CD20 (Table 1, SEQ ID NOs: 22 and 24) protein and a construct expressing GFP (SEQ ID NO: 25). Either MDCK II cell line expressing the fluorescent protein, but not antigen, or an isotype control antibody were used as negative control for the experiment.

Canine peripheral blood mononuclear cells (PBMCs, Envigo) were used as a source of effector cells. PBMCs were isolated from freshly drawn whole blood, with sodium heparin anticoagulant, using Ficoll-Paque plus (Cytiva, GE17-1440-02) density gradient centrifugation by following the recommended protocol. PBMCs were resuspended in media (PBMC media=RPMI+10% heat inactivated foetal bovine serum+1% penicillin-streptomycin+1% non-essential amino acids+1% L-glutamine+1% sodium pyruvate+2% HEPES) supplemented with 50 ng/ml of recombinant canine IL-2 (R&D systems) and incubated for 24 hours at 37° C. before being used in ADCC assay.

To assess ADCC activity, 10,000 MDCK II cells were co-cultured with PBMCs at an effector: target ratio of 35:1 and 0.01 μg/ml of anti-CD20 antibodies, for 24 h at 37° C., in a 1:1 mix of MDCK II media (DMEM+1% L-glutamine+10% fetal bovine serum) and PBMC media. Rituximab-cIGGB was used as the negative (isotype) control antibody.

GFP signal, which is proportional to the number of live cells per well, was used as a measure for surviving cells at the end of the 24 h incubation. GFP signal was measured on a CLARIOstar (BMG Labtech).

Data was analysed using MARS software (BMG Labtech) and percentage of killing in the presence of antibodies was calculated using Microsoft Excel, using wells without antibody as baseline. Graphs were plotted with GraphPad Prism.

FIG. 11 shows ADCC activity was observed in the presence of monospecific anti-CD20 antibodies PMX227, PMX228, PMX229, PMX230, PMX231, PMX232, PMX234, PMX235, PMX235, PMX237, PMX238, PMX239, PMX240, PMX241, PMX242, PMX243, PMX244, PMX245, PMX246, PMX247, PMX248, PMX249, PMX252, PMX253, PMX254, PMX255, PMX256, PMX257, PMX258, PMX259, PMX260, PMX261, PMX262, PMX263, PMX264, PMX265, PMX266, PMX267, PMX268, PMX269, PMX270, PMX271.

All strong CD20 binders can mediate ADCC at different levels, in contrast to CDC effect shown in FIG. 10. This data suggests that CDC killing mechanism has a more stringent conformational requirement to ADCC killing. Candidates which possess both ADCC and CDC are ideal candidates as monospecific anti-CD20 therapeutics.

### Example 9: Canine CD3 and CD20 Variable Region Bispecific Antibody Assembly in a Common Light Chain Format with Human KiH Heterodimerisation

We noticed that the canine V_{H} and V_{L} genes used in some of the CD3 candidate sequences are identical pairs to some of the CD20 candidates. Given the V_{L} somatic hypermutation rate in both CD3 and CD20 candidates is lower than V_{H}, we hypothesized that direct assembly of canine CD20 and CD3 VHs with either CD20 or CD3 VLs may be possible to give rise to functional canine CD3/CD20 bispecific antibodies.

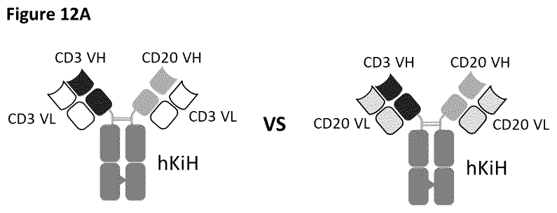

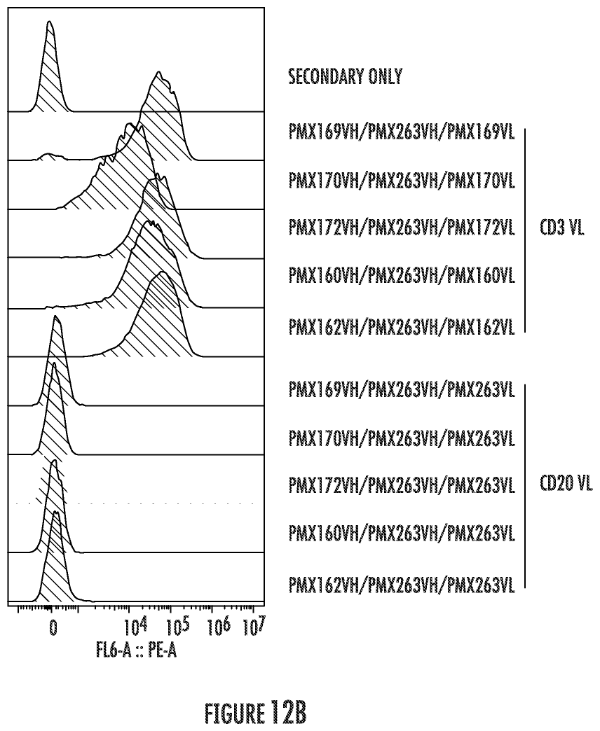

To test this approach, we selected a few CD3 candidates and assembled the heavy chain of these CD3 candidates with a single CD20 candidate heavy chain PMX263 V_{H}, which was selected based on the highest similarity of the CD20 light chain sequences to the light chain of the CD3 candidates. The heavy chain heterodimers were assembled using human KiH Fc as described earlier. Either CD20 light chain or CD3 light chain were assembled with the heavy chain heterodimer (FIG. 12A). Three chain bispecific molecules were produced in CHO cells and the post protein A purification yield was similar between constructs either assembled with CD3 V_{L} and CD20 V_{L}. Cell-based CD3 binding assay as described in Example 4 was conducted to validate the CD3 binding capacity using 10 ug/ml of protein A purified material. Interestingly, all bispecific molecules which contain the CD20 V_{L} resulted in the complete loss of CD3 binding capacity in contrast to the molecules which pair with the CD3 VLs (FIG. 12B).

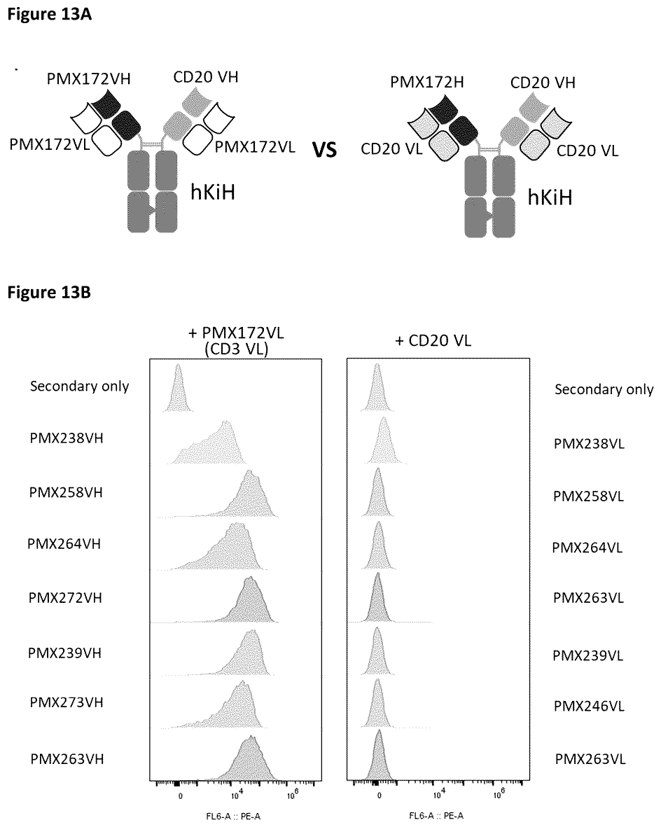

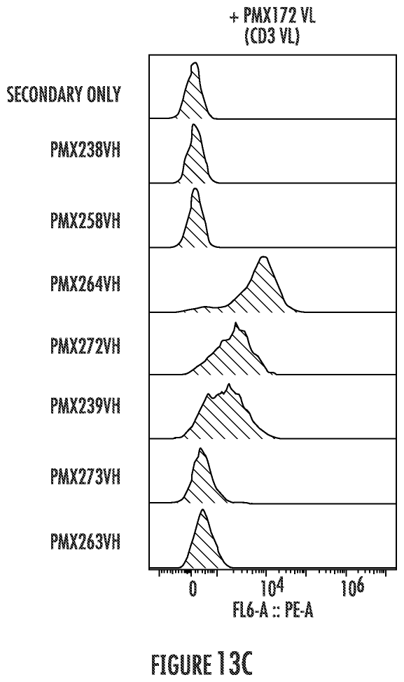

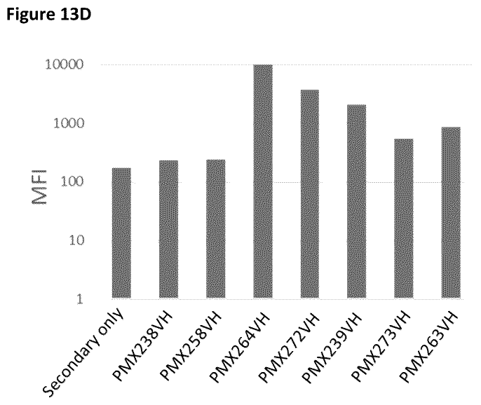

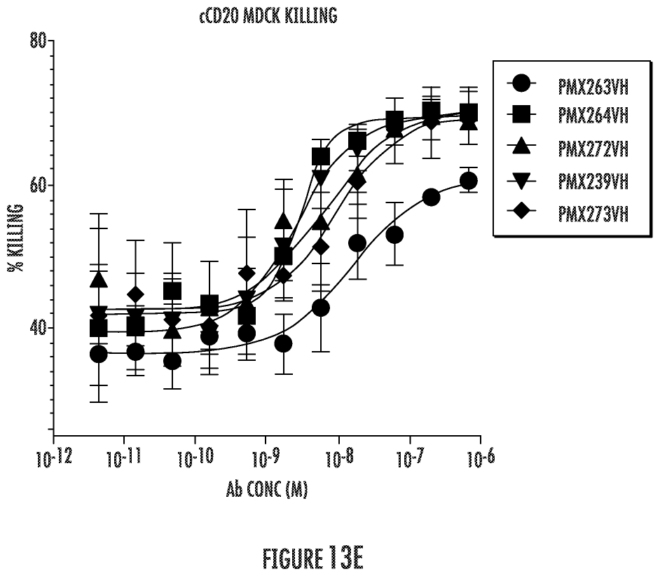

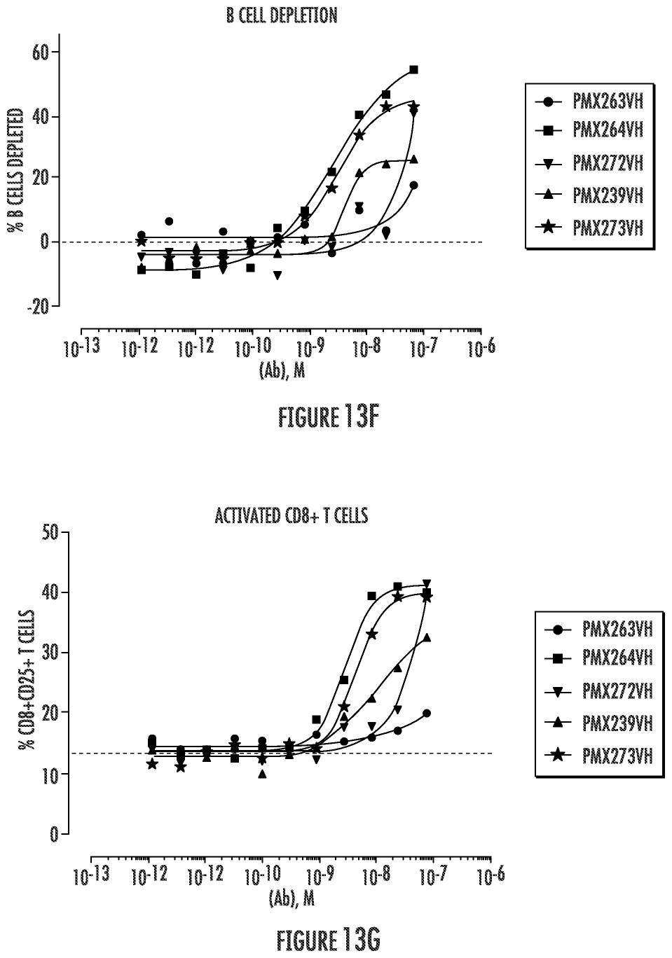

It is plausible that the selected CD20 candidate is not amenable for assembly in such a format. We further explored the compatibility of CD3 V_{L} with different CD20 heavy chains. The heavy chain of a single CD3 candidate, PMX172 V_{H}, was used to assemble with a selection of different CD20 candidates using human KiH Fc for heterodimerisation. The selection of CD20 heavy chain were based on the CD20 V_{L} similarity to anti-CD3 PMX172 V_{L}. Either CD20 light chain or CD3 light chain were assembled with the heavy chain heterodimer (FIG. 13A). The post protein A purification yield was similar between molecules assembled with CD3 V_{L} and CD20 V_{L}. Similarly, as before, none of the molecules assembled with CD20 V_{L} could bind to CD3 (FIG. 13B).

To confirm the CD20 binding capacity, molecules with CD3 V_{L} were tested for CD20 binding using canine MDCK cells overexpressing canine CD20 as described in Example 7. Out of seven CD20 candidates tested, five are able to bind to CD20 albeit with different binding strengths (FIGS. 13C-13D). Taken together, this suggests that CD3 binding is more stringent on the V_{L} sequence specificity than CD20 binding. Since five of these three chain bispecific assemblies are able to bind to both CD3 and CD20, a bispecific killing assay was conducted using canine PBMC mixed with canine CD20+GFP expressing MDCK cells described as before. All five molecules were able to induce killing, the best of which has a EC50 2.59 nM (FIG. 13E and associated Table 8).

TABLE 8
Related to FIG. 13E

| | EC50 (nM) |
|---|---|
| PMX172VH/PMX263VH/PMX172VL | 15.73 |
| PMX172VH/PMX264VH/PMX172VL | 2.88 |
| PMX172VH/PMX272VH/PMX172VL | 5.87 |
| PMX172VH/PMX239VH/PMX172VL | 2.59 |
| PMX172VH/PMX273VH/PMX172VL | 9.63 |

Endogenous PBMC B cell depletion was carried out to further validate the bispecific killing capability using endogenous target to effector ratio. To this end, canine PBMCs were isolated from freshly drawn whole blood, with sodium heparin anticoagulant, using Ficoll-Paque plus (Cytiva, GE17-1440-02) density gradient centrifugation as described previously. 100,000 isolated PBMC was treated with bispecific antibodies with intended concentration diluted in PBMC media for 48 hours at 37° C., 5% CO₂. Cells were harvested and stained with anti-canine CD8 FITC (Biorad, 1:40), anti-canine CD21 Alexa647 (Biorad, 1:40), anti-canine CD25 PE (Thermo Fisher, 1:40) together with anti-canine Fc receptor binding inhibitor (Invitrogen, 1:25). Prior to FACs analysis, cells were also stained with DAPI (1 ug/ml) for live/dead cell discrimination. Endogenous B cell depletion was observed (FIG. 13F) and the potency of B cell killing correlates with the proportion of activated CD8 T cells measured by CD25 activation (FIG. 13G).

### Example 10: Screening for Further Canine CD20 Heavy Chains which can be Assembled with CD3 VH to Mediate Bispecific Killing

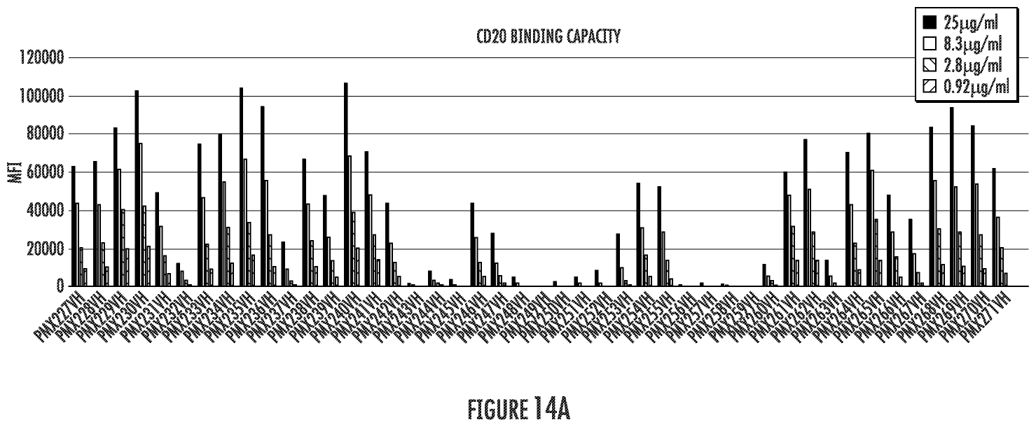

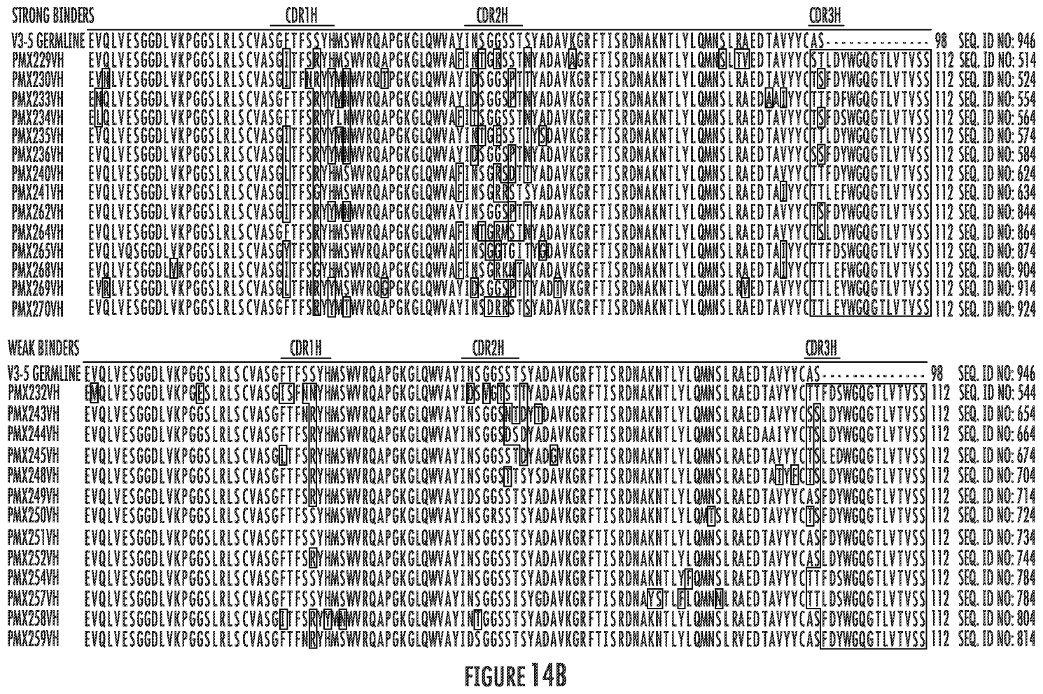

Since in vitro assembly is possible as demonstrated in the previous Example, we conducted a screen by assembling anti-CD3 PMX172 VH together with the VHs from all our CD20 binders identified previously in Example 7 using human KiH Fc to mediate the heavy chain heterodimerisation. PMX172 VL was used as the common light chain. To test binding capacity, a cell based canine CD20 binding assay was conducted using four concentrations of the three chain bispecific candidates as shown in FIG. 14A. Several new CD20 candidates were identified which bind to CD20 similarly to PMX264VH in bispecific format assembled with PMX172VH. Based on the binding strength, multiple sequence alignment was conducted for the anti-CD20 VH sequences in strong binder and weak binder groups. Higher somatic hypermutation in the anti-CD20 VH sequence is associated with strong CD20 binding especially around the CDR1-L and CDR2-L regions compared to the germline sequence FIG. 14B.

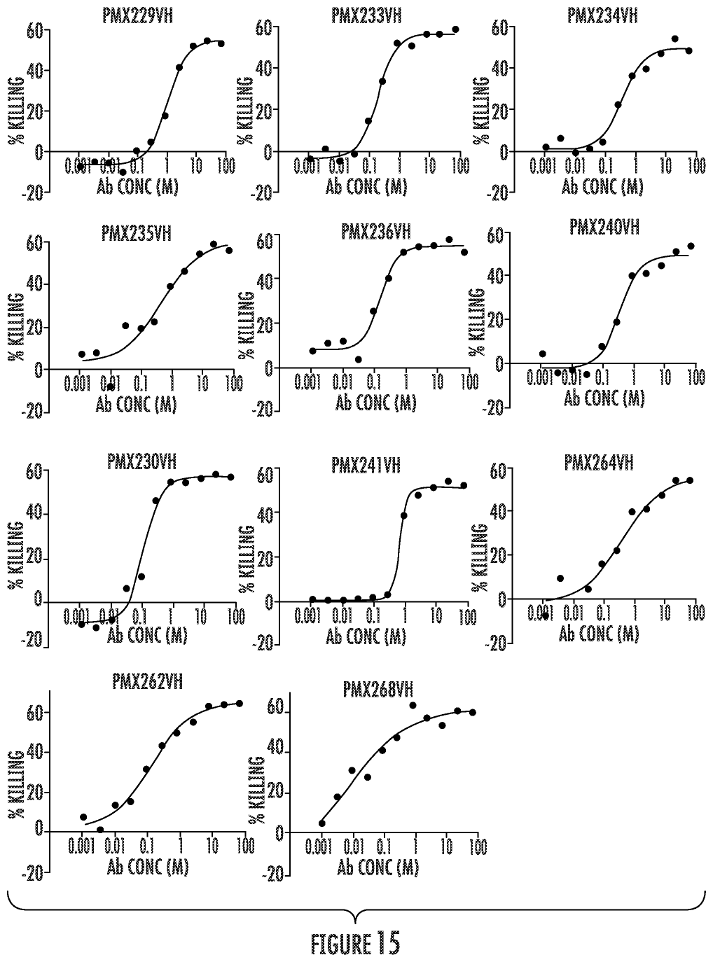

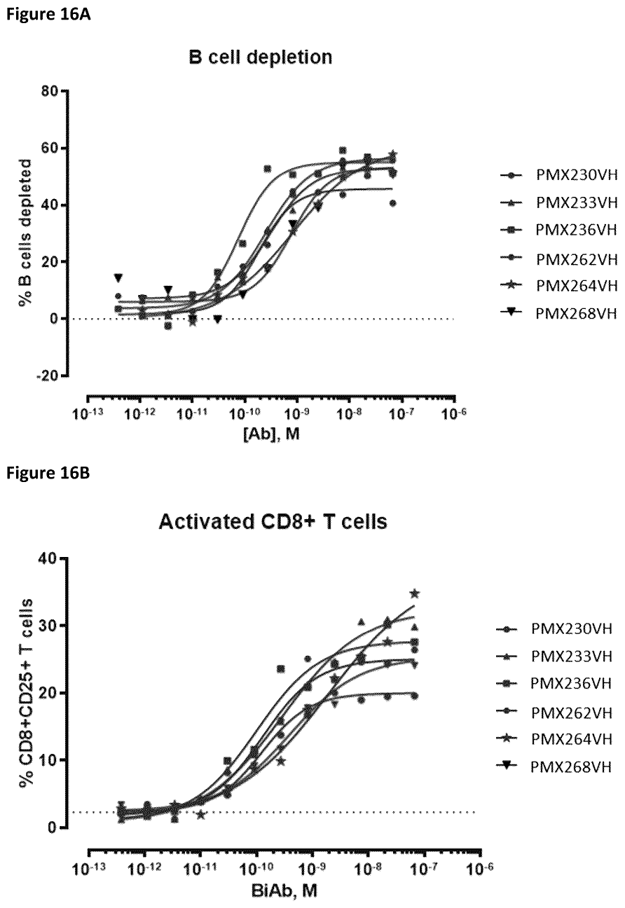

These candidates not only bind to CD20, they were also able to mediate killing using the canine CD20+GFP MDCK cell lines incubated with freshly isolated PBMC (FIG. 15). A subset of potent bispecific killers was further examined in endogenous B cell depletion in PBMC, and killing potency is associated with a proportion of activated CD8+CD25+ T cells (FIGS. 16A-16B). EC50 values associated with FIG. 15 and FIG. 16A are shown in Table 9 and Table 10 respectively.

TABLE 9
Related to FIG. 15

| Sample | EC50 (nM) |
|---|---|
| PMX172VH/PMX229VH/PMX172VL | 1.017 |
| PMX172VH/PMX233VH/PMX172VL | 0.188 |
| PMX172VH/PMX234VH/PMX172VL | 0.4122 |
| PMX172VH/PMX235VH/PMX172VL | 0.445 |
| PMX172VH/PMX236VH/PMX172VL | 0.153 |
| PMX172VH/PMX240VH/PMX172VL | 0.341 |
| PMX172VH/PMX230VH/PMX172VL | 0.121 |
| PMX172VH/PMX214VH/PMX172VL | 0.616 |
| PMX172VH/PMX264VH/PMX172VL | 0.361 |
| PMX172VH/PMX262VH/PMX172VL | 0.129 |
| PMX172VH/PMX268VH/PMX172VL | unstable |

TABLE 10
Related to FIG. 16A

| Sample | EC50 (nM) |
|---|---|
| PMX172VH/PMX230VH/PMX172VL | 0.183 |
| PMX172VH/PMX233VH/PMX172VL | 0.307 |
| PMX172VH/PMX236VH/PMX172VL | 0.075 |
| PMX172VH/PMX262VH/PMX172VL | 0.247 |
| PMX172VH/PMX264VH/PMX172VL | 0.828 |
| PMX172VH/PMX268VH/PMX172VL | 0.764 |

### Example 11: Fully Canine CD20/CD3 Bispecific Antibody Assembly and Characterisation

Previously, we have identified charge pair combinations in canine CH3 domain which significantly enriches heavy chain heterodimersation over homodimer formation (WO 2021/214460 A1). In addition, we have also identified mutations in canine IgG-B which abolish the effector function (SEQ ID NOs.: 29 and SEQ ID NO: 30). We combined the effector function deficiency mutations together with a selected combination of charge pair mutations and used the combination as the Fc region for the fully canine bispecific antibody candidates. For the variable regions, we used some of the shortlisted CD3 candidates described in Examples 4-5 and elected PMX230 V_{H} for the CD20 arm (Example 10) to evaluate the fully canine CD3/CD20 bispecific antibody functionality. The common light chain for each candidate is the cognate CD3 light chain identified from the CD3 screening campaign.

To generate these candidates, CHO cell supernatant harvested from either transient or stable transfection of bispecific mAb clones, have been cultured as described in Example 3, were filtered using 0.22 um filters after being incubated for 10 minutes with Sartoclear Dynamics® Lab V (SDLV-0500-20C-E). Cleared supernatant was loaded into Mabselect sure LX prepacked 20 mL column (17547402), pre-equilibrated with PBS. After loading, the column was washed with 40 mL of PBS (2CV), then was washed with high salt PBS (500 mM NaCl added to 1×PBS) to remove any impurities. Bound proteins was then washed with 40 mL of 50 mM NaOAc (or MES) 200 mM NaCl pH5.5 (2CV) before eluting the bound fraction using gradient (0-100% in 2CV) of 50 mM NaOAc (or MES) 200 mM NaCl pH3. Separate fractions from washes and elution were subjected to cation ion exchange and intact mass spectrometry analysis. Heterodimer enriched fractions were pooled together, concentrated and buffer exchanged to 50 mM NaOAc (or MES) 20 mM NaCl pH5.5 for subsequent cation exchange purification.

Buffer exchanged protein fractions were concentrated to 5 mL and were loaded onto 50 mM NaOAc (or MES) 20 mM NaCl pH5.5 pre-equilibrated Capto S column as a second step purification. A salt gradient elution (from 20 mM to 500 mM) in 20CV have been applied. Heterodimeric fractions were pooled and protein concentration was assessed using NanoDrop™ One (Thermo Scientific™).

Despite the VH and VL combinations were tested previously as described in Example 10, all molecules were tested for CD20 binding and CD3 affinity as described previously in Example 7 and Example 5 respectively since the constant regions have been swapped from human KiH Fc to canine charge paired based Fc. All bispecific molecules were binding to CD20 and CD3 as expected.

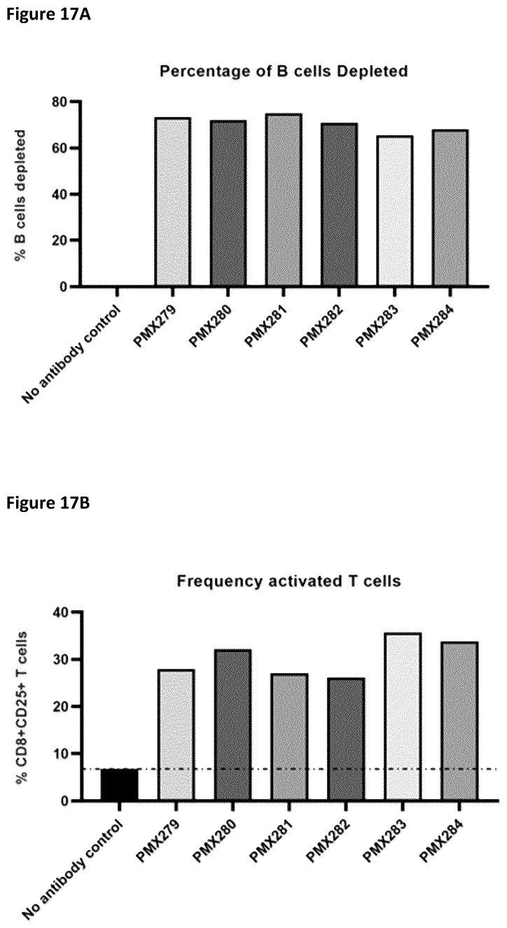

Endogenous PBMC B cell depletion was carried out as described previously in Example 9 to further validate the fully canine 3-chain assemblies killing capacity with the endogenous B:T cell ratio. In this instance, a single concentration of 1.25 ug/ml of antibody was used. Endogenous B cell depletion of over 60% was observed with all of the tested molecules, with an approximately three-fold enrichment in the proportion of activated CD8 T cells, as measured by CD25 activation (FIGS. 17A-17B).

Complementary to purified PBMC based B cell depletion, we have also developed an ex-vivo whole blood B cell depletion assay. In this assay, fresh canine whole blood (Envigo) was diluted 1:2 in PBMC media (PBMC media=RPMI+10% heat inactivated foetal bovine serum+1% penicillin-streptomycin+1% non-essential amino acids+1% L-glutamine+1% sodium pyruvate+1% HEPES) and a single dose of 1.25 ug/ml of the fully canine 3-chain CD3/CD20 bispecific candidate antibodies was incubated for 48 hours at 37° C. Post incubation, the cells were pelleted and resuspended in FACs buffer (3% FCS in PBS) containing the same cell staining mix as used in the naïve PBMC B cell killing assay (Example 9) and incubated at room temperature for 30 minutes. Red blood cells (RBC) were lysed using Biolegend Lyse/Fix solution as per the manufacturer's instruction. Following incubation and RBC lysis, cells were pelleted and washed with FACs buffer, and resuspended in FACs buffer containing DAPI (1 ug/ml) for discrimination of nucleated cells from other blood components.

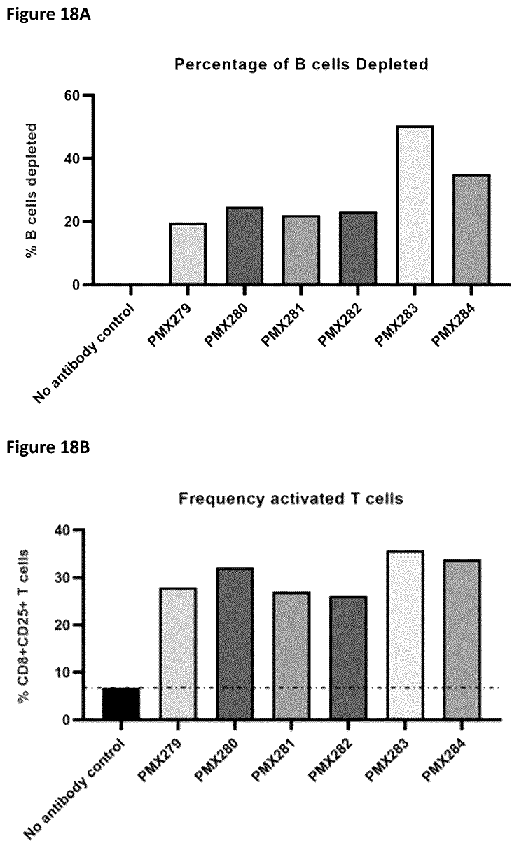

Similar to that observed in the PBMC B cell depletion, B cells were depleted by over 20% with an approx. three-fold enrichment in the proportion of activated CD8+ T cells, as measured by CD25 activation (FIGS. 18A-18B).

### Example 12: Canine CD3 and CD20 Double Knock-In Mouse Models for In Vivo Examination of Fully Canine CD3/CD20 Bispecific Antibodies

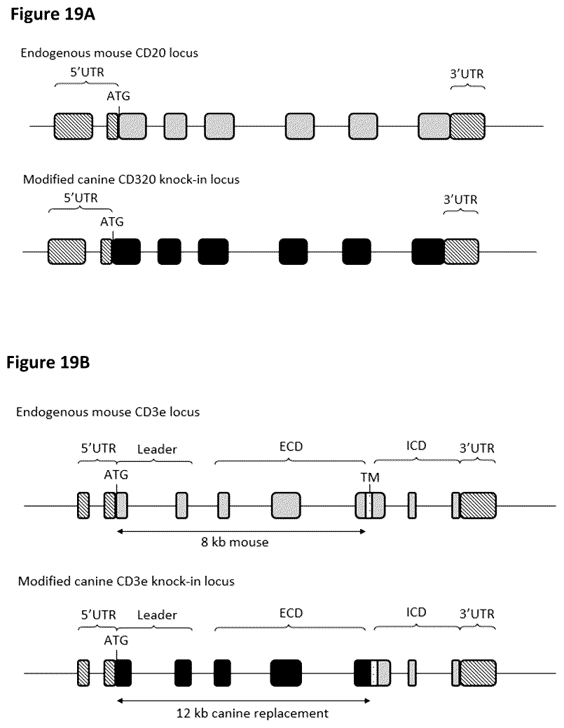

To evaluate the in vivo efficacy of fully canine CD3/CD20 bispecific antibodies, canine CD20 and canine CD3ε double knock-in mice were generated. For canine CD20 knock in, the design is to replace the genomic region encoding all coding exons of mouse CD20 with the canine corresponding genomic region (FIG. 19A). For canine CD3 knock in, the design is to replace the mouse genomic region encoding the leader and extracellular domain with canine corresponding genomic region (FIG. 19B). Both knock in alleles were introduced into mouse ES cells by homologous recombination driven gene targeting. Targeted mouse ES cells were microinjected into host E3.5-E4.5 blastocysts. Injected blastocysts were subsequently transferred to pseudo pregnant females. Chimeric off-springs produced were first identified by coat colour transmission and further confirmed by genomic PCR. Chimeric males were further bred to achieve germ line transmission, which was confirmed by genomic PCR. Germline transmitted males were used as founder mice to establish knock in allele colonies. Canine CD3 and canine CD20 founder mice were independently established and then subsequently bred together to generate study cohorts of double knock-in mice.

Using the double knock-in cohort mice, pre-bleeds were performed 7 days prior to agent administration. Peripheral T and B cell normality was assessed by flow cytometric analysis of common B and T cell markers including mouse CD8, CD4, CD90.1, CD19. Canine CD3 and CD20 surface expression were also assessed to confirm the CD20 and CD3 dual knock-in. Fully canine CD3/CD20 bispecific antibodies were then administered by intravenous injection at given tested doses, with blood being drawn at designated time periods until the terminal bleed and spleen collection. The collected blood draws were centrifuged, and serum isolated for cytokine quantification. Cytokines were measured using commercial ELISA IFNg, IL6 and TNFa kits (Biolegend). The remaining blood cells post centrifugation was stained with (anti-mouse CD19 FITC, anti-mouse CD8 APC-Cy7, anti-mouse CD4 APC, anti-mouse CD69 Pacific blue, and anti-mouse CD25 PE for 30 min prior to treatment with red blood cell lysis and fix buffer. Cytometric analysis was performed using the same panel as per the pre-bleeds to assess B cell depletion and T cell activation and proliferation. Deep tissue penetration and B cell depletion mediated by the bispecific candidates was also assessed using the terminal spleen harvest, with lymphocytes isolated and flow cytometric analysis performed to assess differing B and T cell populations present.

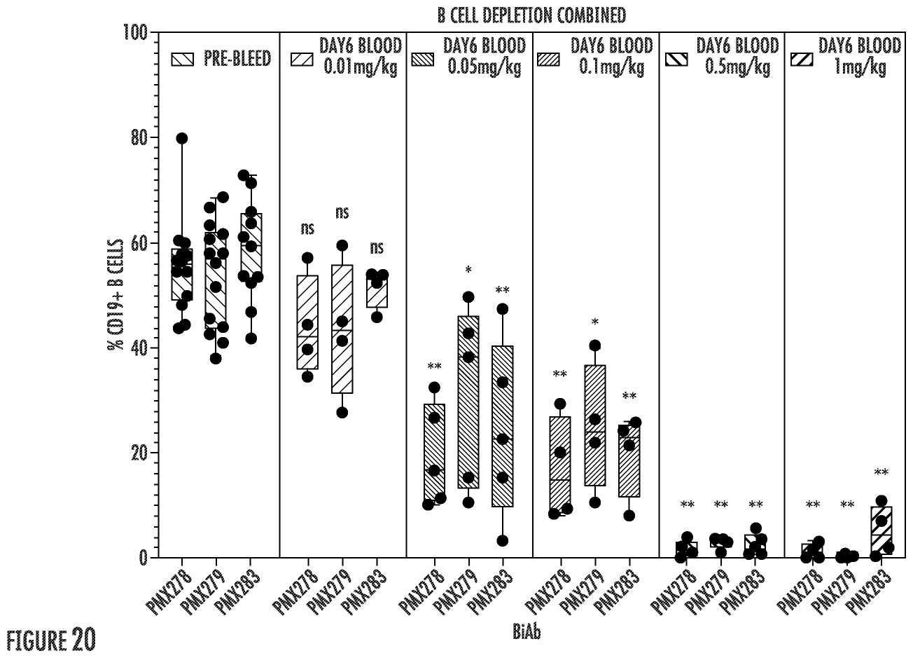

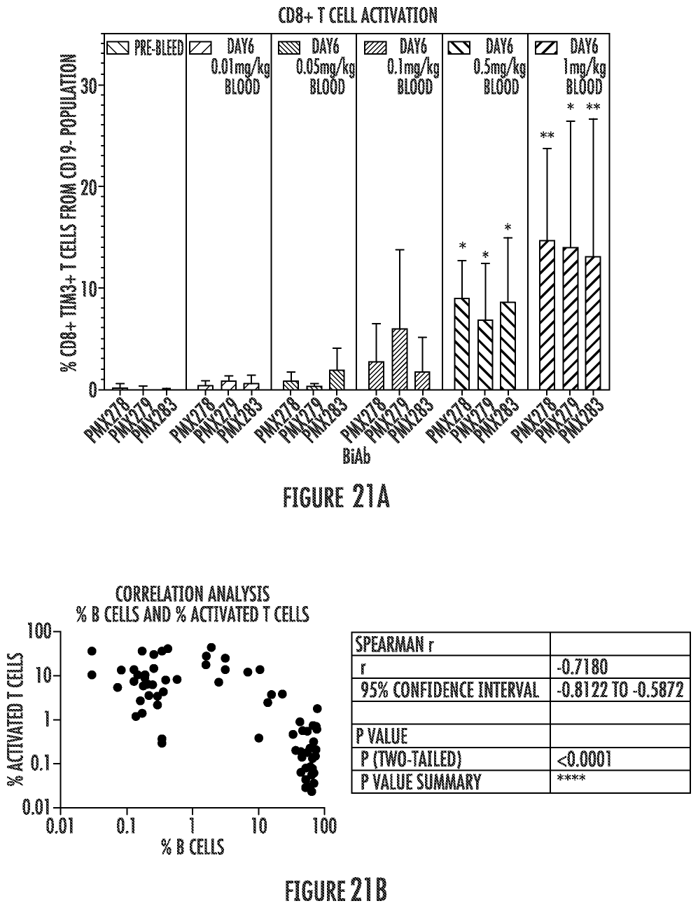

The dose effect on peripheral B cell depletion were examined using the canine dual CD3 and CD20 KI mice. As shown in FIG. 20, administration of fully canine CD3/CD20 bispecific antibody candidates PMX278, PMX279 and PMX283 at dose levels of 0.5 and 1 mg/kg resulted in depletion of circulating peripheral B cells to near undetectable levels by day six post dose, as measured by the percentage of CD19 positive lymphocytes. Dose levels of 0.1, 0.05 and 0.01 mg/kg resulted in only partial B cell depletion or no depletion. Higher doses (0.5 mg and 1.0 mg/kg) also resulted in a greater degree of CD8+ T cell activation in peripheral blood, as measured by an increase in the percentage of PD1 and TIM3 double positive cells (FIG. 21A). Irrespective to the candidate antibody treatment and doses, we observe a strong negative correlation (Spearman r=−0.718, p<0.0001) between the percentage of circulating B cells and the percentage of activated T cells present (FIG. 21B).

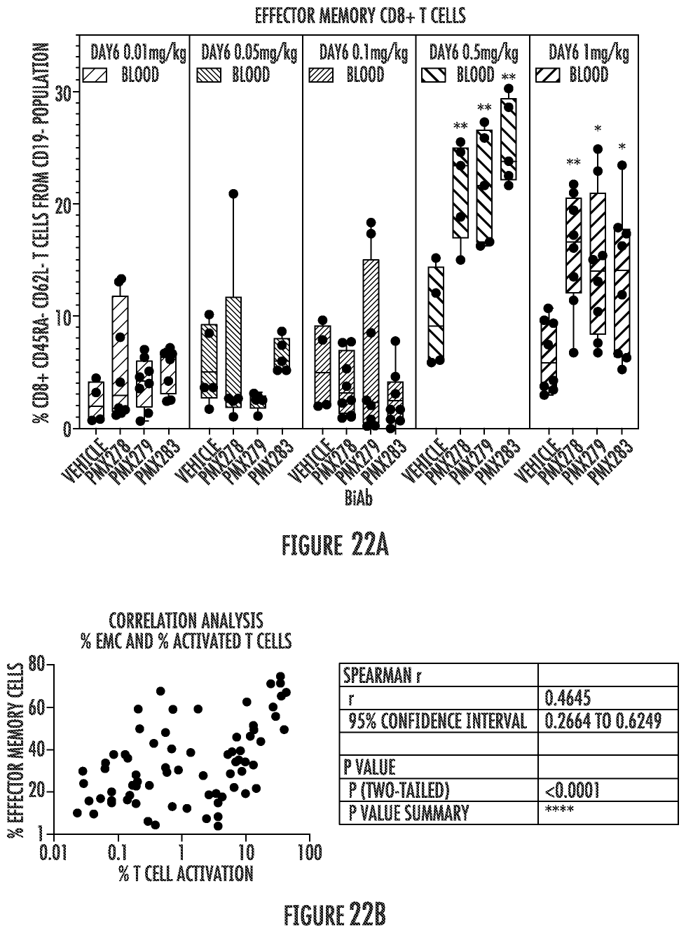

Upon T cell activation during infection, effector memory differentiation is an important aspect in forming longer lasting memory cell function, which can rapidly acquire cytotoxic function. To assess the effector memory differentiation upon CD3/CD20 bispecific antibody treatment, the proportion of effector memory CD8+ T cells were also examined by CD45RA and CD62L staining. In general, an increase in effector memory CD45RA and CD62L double negative cells were observed in 0.5 and 1.0 mg/kg doses. Irrespective of the candidates and doses, the percentage of activated CD8+ T cells in peripheral blood was moderately positively correlated (Spearman r=−0.4645, p<0.0001) with the percentage of effector memory cells present, as measured by an increase in the percentage of CD45RA and CD62L double negative CD8+ T cells compared to a vehicle control FIGS. 22A-22B. Taken together, this evidence suggests that our fully canine bispecific CD3/CD20 antibodies elicit dose dependent T cell activation and memory differentiation which in turn results in target cell killing dose dependency.

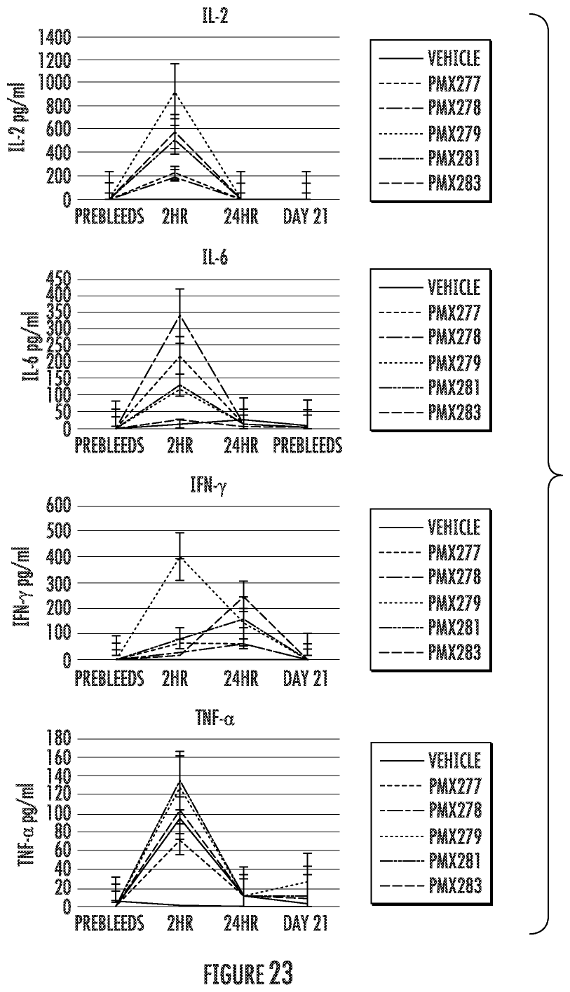

Cytokine release is a complementary measure for T cell activation to cell surface-based marker activation. Sustained T cell activation could result in severe cytokine release, which is a safety concern in general for T cell engager based bispecific antibodies. To evaluate cytokine levels in our candidate bispecific antibody treatment, plasma samples were also harvested and analysed for IL-2, IL-6, IFN-γ and TNF-α using a pre-defined LEGENDplex™ Mouse Th Cytokine bead based multiplex immunoassay from Biolegend as per the manufacturer's instructions. Administration of 0.5 mg/kg of all candidates induced a transient increase in IL-2, IL-6, and TNF-α plasma cytokine levels at two-hour post dose and these cytokines return to near pre-dose level at 24 hours (FIG. 23). For IFN-γ, apart from PMX279, administration of PMX278, PMX281 and PMX283 induced a peak IFN-γ concentration around 24 hours rather than 2 hours post dosing. All cytokine levels returned to pre-dose levels by 21 days post dosing. (Sun et al. (2015) Sci Trans Med 7(287)287ra70).

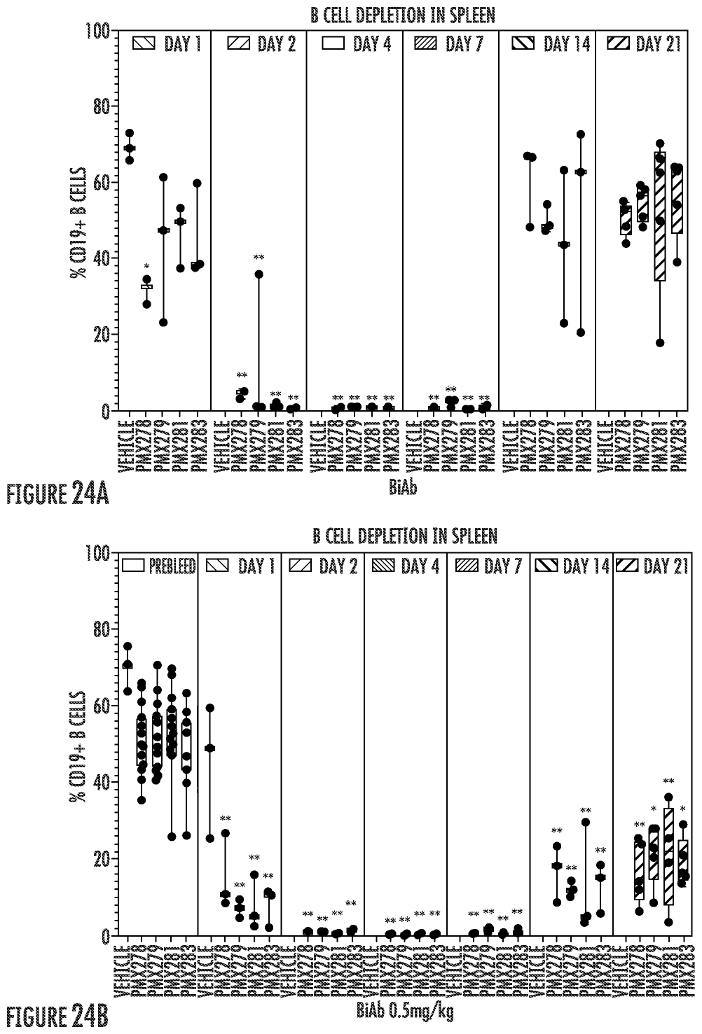

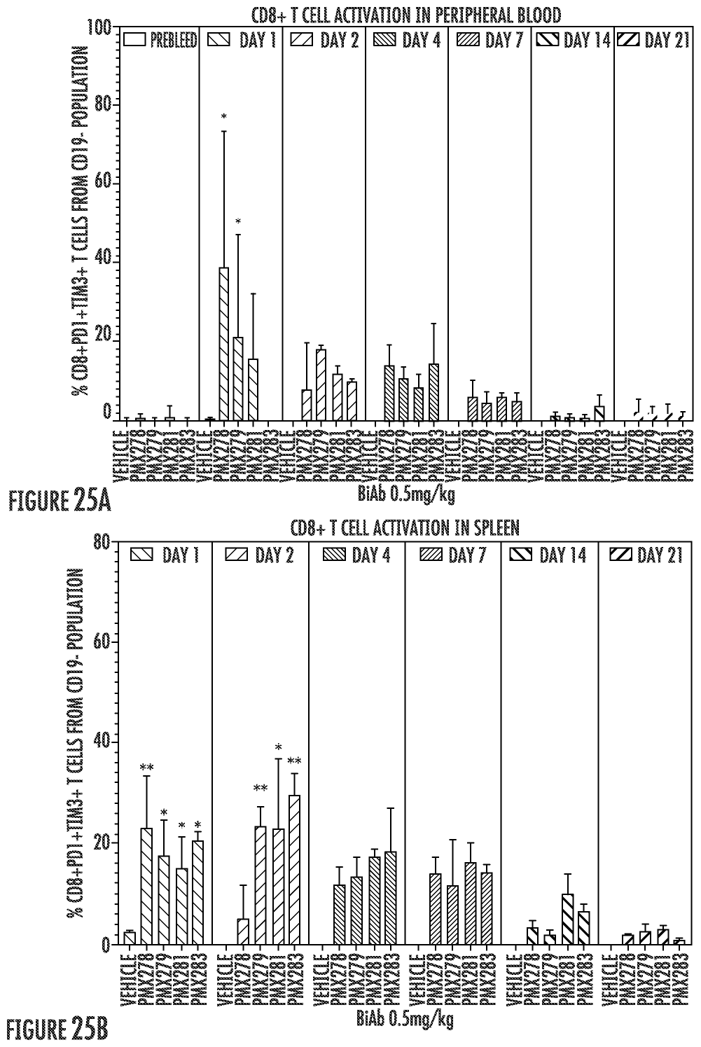

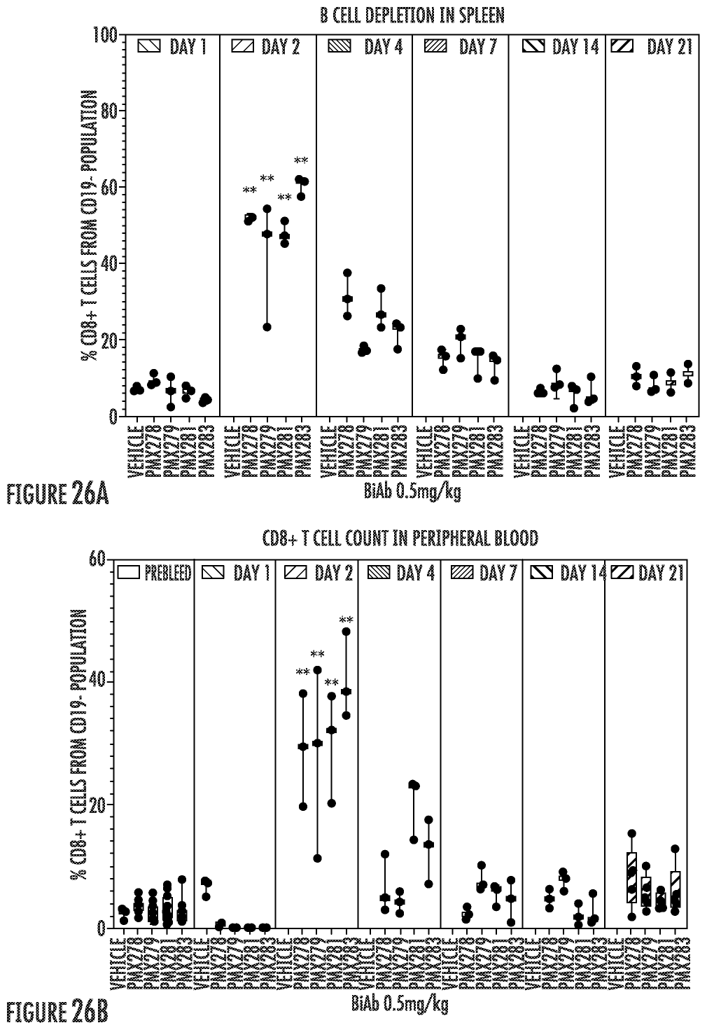

To assess the fully canine bispecific antibody's ability for deep tissue killing, spleens were harvested at different timepoints over 21 days post dosing alongside peripheral blood following administration with 0.5 mg/kg BiAb. Spleens were homogenised, and the frequency of B & T cell was assessed by flow cytometry. B-cell depletion was significantly reduced by day 2 and was maintained until day 7 post dosing. B-cell levels recovered back to pre-dose levels by day 14 (FIG. 24A). Full depletion was observed within 48-hours. Some recovery of B cell depletion in peripheral blood was observed 14 days post doing, with partial depletion being maintained up to 21 days (FIG. 24B). In both peripheral blood and spleen, T cells displayed an activated phenotype, measured by PD1, TIM3 double positive staining, on day 1 which steadily declined back to baseline levels by day 14 (FIGS. 25A-25B). CD8+ T cell counts in both peripheral blood and spleen increased by day 2, and the timing and size of the increase were suggestive of a rapid effector expansion. On day 4 when splenic and peripheral B cells were cleared, CD8+ T cell counts started to decrease significantly following the period of expansion (FIGS. 26A-26B). This rapid expansion and contraction of differentiated effector cells is similar to natural T cell behaviour upon viral insult (Wherry & Ahmed (2004) J Virol. 78(11); e5535-5545).

### Example 13: Anti-Tumour Activity of Canine CD3 and CD20 Bispecifics in a Caninized Syngeneic Mouse Tumour Model

The in vivo anti-tumour efficacy of the fully canine CD3/CD20 bispecific antibodies was assessed using a caninized syngeneic mouse tumour model. This tumour model was generated by subcutaneous inoculation of murine B cell lymphoma A20 cell line engineered to over express canine CD20 into canine CD20 and canine CD3ε double knock-in mice. Since the A20 tumour cell line is derived from BALB/C mice, canine CD20 and canine CD3ε double knock-in mice were bred to BALB/C background. In this model 1×10⁶ canine CD20 overexpressing A20 cells were resuspended in Matrigel before injecting subcutaneously into the double knock-in mice. Mice were then monitored for tumour development. Once masses were observed, during critical periods of tumour development tumour growth was monitored by palpation/calliper measurement and every 2-3 days thereafter. Once animals reach the general humane end point, when tumour were 1.5 cm in any direction, tumour tissue was harvested and placed in harvest media (RPMI+15% FBS).

Tumour tissue was dissociated by mincing the tissue into small pieces using a scalpel blade before transferring to a 15 mL round bottom tube containing tumour digestion medium (500 μL Collagenase/Hyaluronidase, 750 μL DNase I solution (1 mg/mL) in 3.75 mL RPMI 1640 medium). This was incubated at 37° C. for 25 minutes before being passed through a strainer. Strained cells were then washed and resuspended in ammonium chloride solution (0.8% NH4Cl, 0.1 mM EDTA in water, buffered with KHCO3 to achieve a final pH of 7.2-7.6) and incubated for 5 minutes to remove any contaminating RBCs. Following this, cells were washed and resuspended in FACS buffer ready for antibody staining.

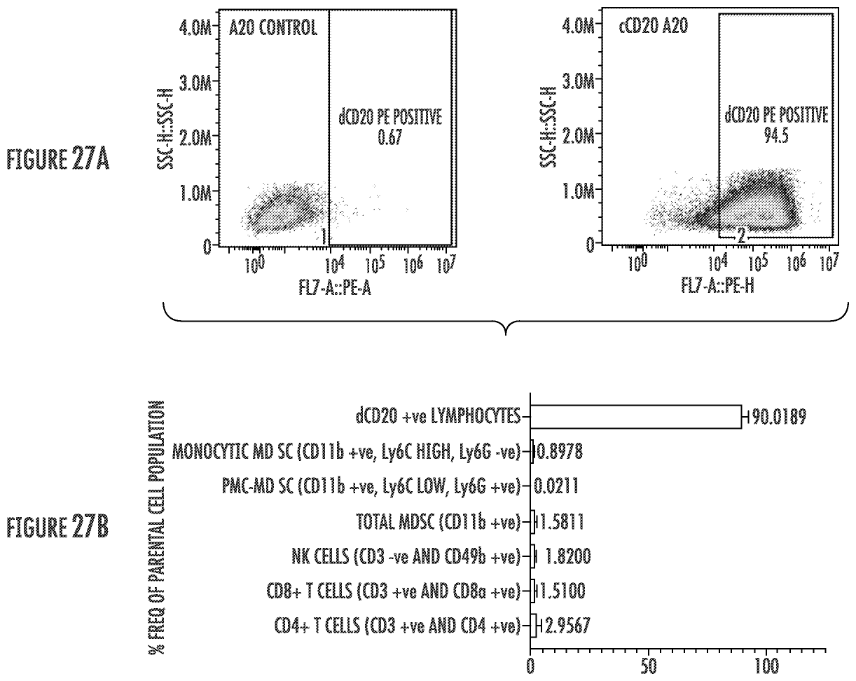

To confirm the tumours expressed sufficient target canine CD20 levels, harvested tumour cells were stained with anti-canine CD20 PE (InvivoGen) for 30 minutes and then assessed by flow cytometric analysis. Live lymphocytes were gated and the percentage of PE cCD20 positive cells within this gate were assessed. As shown in FIG. 27A, tumours formed following injection of A20 control cells had <1% positive staining for canine CD20 in comparison to those tumours generated from A20 canine CD20 overexpressing cells which showed ˜95% positive staining. Immunophenotyping of canine lymph nodes from dogs with B cell lymphoma have shown that around 92% of residing cells are positive for B cell markers, being very poorly T cell-infiltrated which is suggestive of a “cold tumour” phenotype (Thalheim, L et al. (2013) 27:1509-1516). To determine the tumour microenvironment of the generated A20 canine CD20 expressing tumours, tumour cells were assessed by flow cytometry using staining panels consisting of effector T cells (CD3+ve, CD4+ve), cytotoxic T cells (CD3+ve, CD8a+ve), regulatory T cells (CD3+ve, FOXP3+ve) NK cell markers (CD3−ve, CD49b+ve), PMC-MDSC markers (CD11 b+ve, Ly6C low, Ly6G+ve), and monocytic MDSC markers (CD11 b}+ve, Ly6C high, Ly6G−ve). As shown in FIG. 27B, 90% of the cell population were positive for the canine B cell marker CD20, with very few infiltrating T cells, NK cells or MDSCs much like that observed in cold tumours highlighting the suitability of the model.

Having confirmed that A20 canine CD20 expressing tumours can be established and provide a valid model for studying the in vivo activity of bispecific molecules, experiments will be carried out to determine whether these tumours can be cleared following dosing. Mice will be randomized into treatment and control and treatment groups. Tumours will be induced as described previously and candidate therapeutics will be administered following tumour induction by parenteral routes.

To better understand the mechanism of antitumor activity, T and B cell dynamics in both peripheral blood and tumour masses will be investigated. Upon administration of each does, time- and dose-dependent depletion of circulating and tumour bearing B cells will be detected using flow cytometry over a 21-day period. Similarly infiltrating T cells will be characterised for clonality, differentiation and activation status and compared to those T cells in peripheral blood throughout the time course. Tumour size will be monitored over the 21 days and compared to the vehicle control. Tumour tissue harvested throughout the time course will be sectioned into 3 vials, one containing formalin for immunohistochemistry, one containing RNA-later for RNA-sequencing and one containing harvest media for flow cytometric analysis and cytokine analysis.

Tumour tissue collected in harvest media will be separated in two, one being dissociated as described above using collagenase for flow cytometric analysis and the other being snap frozen in liquid nitrogen for cytokine analysis. Cell for flow cytometry will be stained as described previously. Tumour lysate will be prepared to measure cytokine levels within the tumours by pulverizing the snap frozen tumour piece into a fine powder using a cold mortar and pestle. Tumour powder will be placed into cell lysis buffer (Cell Signaling Technology) and homogenized using Lysing Matrix D (MPBio). This will then be centrifuged, and the supernatant collected and measured for protein content using the Pierce BCA protein assay kit (Thermo Fisher). The totai protein content will then be normalized between samples by dilution. Cytokine analysis of the tumour lysate will be performed using the LEGENDplex™ Pre-defined Mouse Cytokine Release Syndrome Panel (Biolegend) as per the manufacturer's instructions.

Immunohistochemistry will be performed on 4-um thick formalin-fixed, paraffin-embedded tumour tissue sections mounted on glass slides. Staining with primary antibodies against B and T cell markers will be performed to assess levels of B cell depletion and T cell tumour infiltration in the presence of CD3/CD20 bispecific antibody.

Tumour sample stored in RNAlater at −20° C. will be homogenized before RNA is extracted using standard methods. Single cell RNA-sequencing will then be performed to compare the differential gene expression patters, with a particular emphasis on the T cell phenotype.

We hypothesise that the fully canine CD3/CD20 bispecific antibodies will efficiently reduce tumour burden compared to a non-binding or vehicle control. We predict to see a differences in the tumour-infiltrating lymphocytes (TILs) population dynamics in tumours treated with the fully canine CD3/CD20 bispecific antibodies, which will be indicative of efficient redirection of T cell and subsequent lysis of target tumour cells.

### Example 14: Pharmacokinetic and Pharmacodynamic Characteristics of Canine CD3 and CD20 Bispecific Abs in Dogs

Building on our finding from the syngeneic mouse studies, an in vivo in dog dose escalation study was designed to evaluate pharmacokinetic parameters, efficacy, and safety of three canine CD3×CD20 bispecific antibody candidates, PMX278, PMX279 and PMX283. Owing to the increasing frequency and severity of cytokine release syndrome (CRS) observed with high dose levels of human CD3×CD20 bispecifics in human clinical trials, step-up dosing was utilised. Such a dosing regime has been shown to minimise CRS by priming the body's immune system; thereby regulating the balance between T cell activation and expansion, and cytokine-mediated efficacy and toxicity (Ball (2023) Mabs. 15(1): 2181016). With this in mind, dogs were treated with a first dose of 0.01 mg/kg, second dose of 0.05 mg/kg, and third dose 0.5 mg/kg, by intravenous infusion. Five dogs were assigned to each treatment group. Dogs were dosed at 0.01 mg/kg on day 0 with step-up doses being administered on D8 and D15. Although CRS in canine cancer patients has not been as comprehensively described as that in humans, similar clinical and serological changes have been reported in dogs treated with autologous CAR-T therapies for relapsed B cell lymphoma (Atherton (2022) Front Vet Sci. 9:824982). Such clinical signs include lethargy, pyrexia and moderate tachycardia.

Following each dosing event dogs vital signs including temperature, heart rate, blood pressure and mucous membrane colour were therefore assessed at T2h, T8h, T24h, T72h, T120h and T144h post injection in order to monitor for signs of CRS. Blood sampling was also conducted throughout at target time points for haematology and biochemistry analysis to assess whole organ effects.

The pharmacokinetic profile of each bispecific antibody was evaluated by analysing serum samples collected at pre dose and up to T672h after D15 administration. The total concentration of bispecific antibody was determined using ELISA techniques. Briefly, canine CD20 fusion protein was captured onto Nunc 96-well micro-well plates overnight at 4° C., before blocking with 5% BSA in PBS. Following 2 hours of blocking, samples and standards were added and incubated for 1 hour at room temperature. Plates were washed 3× with wash buffer (PBS, 0.2% Tween 20). Biotin labelled anti-canine Fc secondary antibody (Sigma Aldrich) was added and incubated for 1 hour at room temperature. Plates were then washed 3× with wash buffer and 1000× Streptavidin HRP (BioLegend) added and incubated for 30 minutes at room temperature. TMB substrate (Thermo Fisher) was used for development as per the manufacturer's recommendation.

Like in humans, canine CRS serological profiles share many similarities in terms of elevated cytokine levels, with most notably rapid elevations in IL-6 and IFN-γ (Atherton (2022) Front Vet Sci. 9:824982). In this study, serum samples were therefore analysed to measure cytokine levels (including IFN-γ, IL-2, IL-6, IL-10, TNF-α) using the Milliplex® Canine Cytokine Magnetic Bead Immunoassay (Millipore) following each dosing event. In addition, fine needle aspirates of peripheral lymph nodes were collected pre-dose, and at 24 h and 144 h post DO, D8 and D15 administrations in order to assess tissue penetration of the bispecific antibodies. Samples were disposed as smear on histology slides before staining with B cell markers as per standard immunocytochemistry protocols.

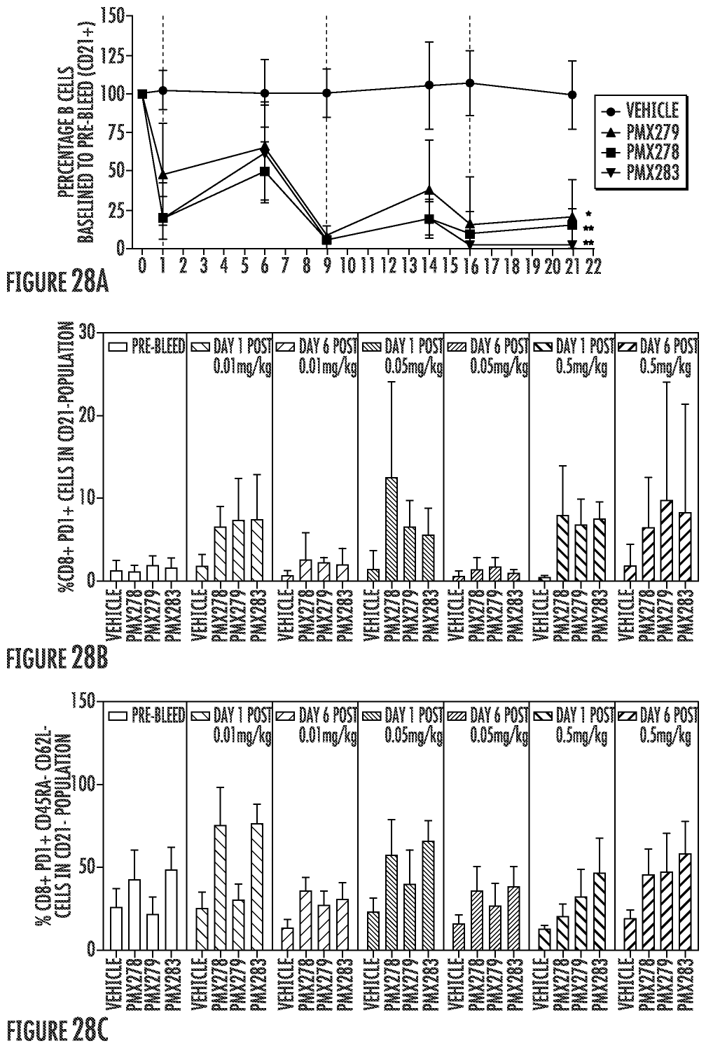

The pharmacodynamic effects were investigated using flow cytometry. Peripheral blood draws were drawn on K3-EDTA tubes, from jugular vein with single use needles and syringes at the following sampling time points: Pre-dose then at T24h and T144h after DO administration, at T24h and T144h after D8 administration, and at T24h, T144h, T360h, T336h, T504h and T696h, T672h after D15 administration. Peripheral T and B cell normality was assessed by flow cytometric analysis of common B and T cell markers including dog CD8, CD4, and CD21. As shown in FIG. 28A 24 hours following administration of 0.01 mg/kg of all three candidate antibodies resulted in over 50% depletion in B cells compared to the pre-bleed control. Subsequent dosing at 0.05 and 0.5 mg/kg resulted in B cell clearance using candidate PMX283, with this B cell depletion being maintained up to day 6 post the final dose escalation. Like that in Example 12, CD8+ T cell activation was investigated using an anti-canine PD-1 antibody. FIG. 28B shows the percentage of CD8+ T cells also expressing PD1, with a trend of increased T cell activation being observed after each dosing event compared to the vehicle control. To assess the effector memory differentiation upon CD3/CD20 bispecific antibody treatment, the proportion of effector memory CD8+ T cells were also examined by CD45RA and CD62L staining using antibodies shown to be cross reactive with dog. FIG. 28C shows the percentage of CD8+ T cells which were negative for CD45RA and CD62L, which is indicative of effector memory T cells (EMC) with a trend of increased EMC cells being observed after each dosing event compared to the vehicle control.

A similar study will be performed at higher doses to determine the maximum tolerated dose.

### Example 15: In Vivo Efficacy of Canine Anti-CD20 Monoclonal Antibody in Combination with Anti-CD20×CD3 Bispecific Antibody Against CD20 B Cells

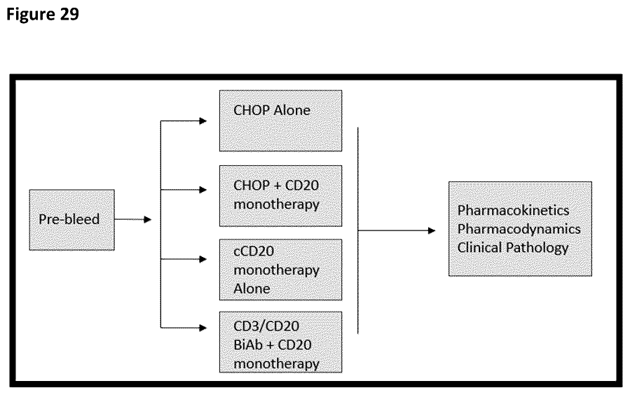

In this Example, the anti-tumour effect of anti-dog CD20 monoclonal antibody in combination with anti-dog CD20×CD3 bispecific antibody will be examined using a sequential dosing regimen to assess the safety, maximum tolerate dose (MTD) and CD3×CD20 activity following prior treatment with a CD20 monoclonal antibody. Sample grouping will be devised as described in FIG. 29. Sampling will be conducted as per Example 14 measuring pharmacokinetics, pharmacodynamics and clinical pathology.

A combination approach may be expected to see improved results compared to a monotherapy alone.

### Example 16: Binding Screen of Monospecific Anti-CD3 Candidates

Anti-CD3 candidates with agonistic activity (see Example 4), were selected to be tested for immunosuppression properties. To this end the antibodies were reformatted in a monospecific format as described previously with canine IgG-B effector function deficient Fc (SEQ ID NO: 30).

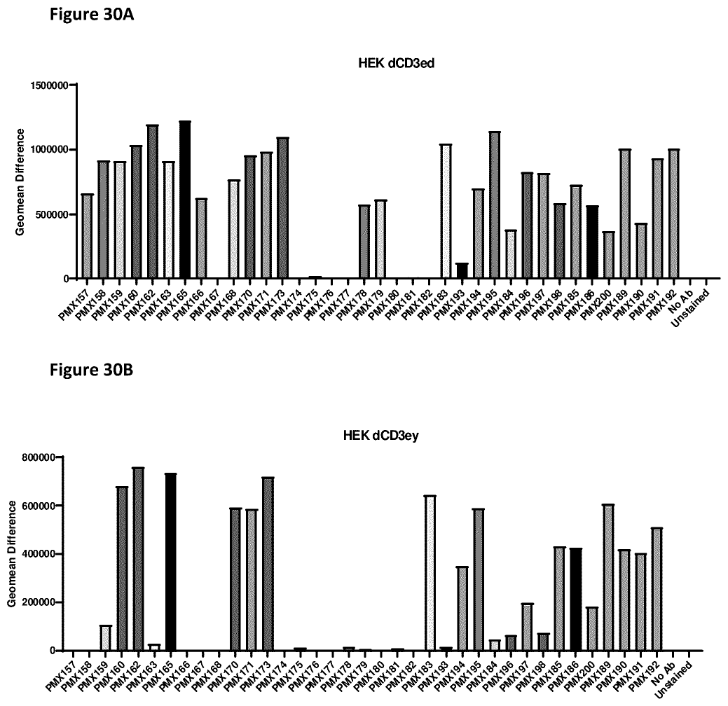

Monospecific anti-CD3 candidates were first confirmed to maintain their ability to bind canine CD3εγ and CD3εδ expressed on the cell surface of HEK cells (see Example 3 for method). The results confirmed binding strength from Example 3 (FIGS. 30A-30B).

### Example 17: In Vitro Cell Based Functional Assay to Identify Immunosuppressive CD3 Candidates

T cell anergy is a long-term state of hypo/non-responsiveness. It is induced by the stimulation of T cells via TCR in the absence of co-stimulatory signals eg. CD28. Inducing T cell anergy leads to a state of immunosuppression which can be used to treat autoimmune diseases, such as type I diabetes. Measures of T cell activation are known in the art and include surface markers, proliferation of T cells and cytokine release. These are complementary measures, and all were assessed.

The ability of CD3 monospecific candidate antibodies to induce T cell anergy, was tested in an in vitro T cell mediated agonist assay using canine peripheral blood mononuclear cells (PBMCs, Envigo).

PBMCs were isolated from freshly drawn whole blood, with sodium heparin anticoagulant, using Ficoll-Paque plus (Cytiva, GE17-1440-02) density gradient centrifugation. In brief, canine blood was diluted 1:1 with phosphate buffer saline (PBS) and carefully layered on top of Ficoll-paque plus, centrifugated at 800×g for 20 minutes with slow acceleration and no break. The top layer and interphase disk were diluted with PBS and centrifuged at 400×g for 10 minutes to collect the PBMCs in the pellet, which was washed a second time in PBS to remove all remnants of Ficoll. After a second centrifugation, PBMCs were resuspended in media (PBMC media=RPMI+10% heat inactivated foetal bovine serum+1% penicillin-streptomycin+1% non-essential amino acids+1% L-glutamine+1% sodium pyruvate+1% HEPES). PBMCs were either used fresh or frozen for storage and then thawed when required for use in the agonist assay.

To assess the agonistic activity of monospecific anti-CD3 mAbs, candidate antibodies were pre-coated to a flat-bottomed cell culture treated plate at 10 μg/ml in 40 ul of PBS and incubated for 2-3 hrs at 37° C. The plates were then washed with 150 ul of PBS to remove unbound antibody and PBMCs were seeded at a density of 1.5×10⁵-2.5×10⁵ per well in PBMC medium. Alternatively, candidate antibodies were not pre-coated to the assay plate but instead diluted in PBMC media and added in solution at the time the PBMCs were seeded to a final concentration of 10 μg/ml. The antibodies and cell mix were co-cultured at 37° C., 5% CO₂, for four to six days. Following incubation period the cells were resuspended and transferred into a v-bottomed plate where the cells were spun down at 400 g for 4 mins. The supernatant was collected for IFNγ quantification by ELISA (canine IFNγ basic ELISA kit, MABtech) according to the manufacturer's instructions, with supernatant diluted 1:5 in diluent buffer. Cells were stained, fixed and permeabilized using eBioscience FOXP3/Transcription Factor Staining Buffer Set as per the manufacturer's instructions. Alternatively, cells were not fixed and permeabilized and only surface stained. The T cell surface markers CD5, CD4 and CD8 were used to assess changes in the different T cell populations (T helper cells and T cytotoxic cell). CD25, the alpha-chain of IL-2 receptor is commonly used as a marker for T cell activation, and it was used as such in this assay. Ki67 was used as a marker for T cell proliferation. Alternative methods for assessing T cell proliferation can be used and include Cell Tracer™ CFSE proliferation kit (Invitrogen) tracking multiple generations of proliferation through dye dilution via flow cytometry.

An agonist was defined by the ability to increase the expression of the activation marker CD25 on the T cell population, increase proliferation of the T cell population determined using either Ki67 or Cell Tracer™ CFSE proliferation kit and increase the production of IFNγ compared to unstimulated PBMCs (Table 11). All candidates that were still able to bind in monospecific format were agonists to varying degrees.

In order to assess the ability of candidate anti-CD3 antibodies to induce T cell anergy, canine PBMC were incubated with anti-CD3 antibodies for 4 to 6 days as described above. At the end of the incubation period, cells were further stimulated with either concanavalin A (ConA) or phytohemagglutinin (PHA) and left in culture for a further 3-4 days. If the CD3 mAbs were able to induce T cell anergy in the first 4-6 days of culture then the T cells would be unresponsive to the further stimulation by ConA or PHA and the level of CD25, proliferation and IFNγ release would remain unchanged or decrease (Table 11). The candidates that show characteristics for induction of anergy include; PMX158, PMX162, PMX163, PMX164, PMX165, PMX166, PMX170, PMX171, PMX175, PMX194, PMX199.

TABLE 11
In vitro cell based functional assay to identify immunosuppressive CD3 candidates

| | IFNy Concentration pg/ml | | | CD25 Activation | | | | Ki67 Proliferation | | | |
|---|---|---|---|---|---|---|---|---|---|---|---|
| PMX | Day 6 | Day 9 | Day 9 | Day 6 | Day 6 | Day 9 | Day 9 | Day 6 | Day 6 | Day 9 | Day 9 |
| numbers | IS | PB | IS | PB | IS | PB | IS | PB | IS | PB | IS |
| PMX157 | 44.37 | 3138.84 | 209.4 | 11.6 | 9.65 | 8.72 | 9.96 | 0.4 | 1.24 | 0.34 | 0.85 |
| PMX158 | 48.165 | 2438.085 | 88.295 | 9.65 | 13.1 | 12.3 | 5.45 | 0.54 | 2.63 | 0 | 0.45 |
| PMX159 | 137.115 | 3049.46 | 261.555 | 11.5 | 11.3 | 13.1 | 9.13 | 0.37 | 1.69 | 0 | 1.28 |
| PMX160 | 36.45 | 1461.36 | 113.875 | 9.42 | 10.6 | 5.98 | 7.14 | 0.12 | 0.84 | 0.33 | 1.87 |
| PMX162 | 61.125 | 329.94 | 89.225 | 8.64 | 16 | 10.4 | 8.75 | 0.36 | 1.73 | 0.43 | 0.51 |
| PMX163 | 56.2 | 3769.595 | 145.165 | 18.8 | 13.3 | 14.6 | 10.1 | 0.3 | 4.91 | 0.75 | 0.71 |
| PMX165 | 74.595 | 1368.67 | 107.76 | 13.4 | 12.1 | 23.6 | 10.4 | 0.26 | 1.67 | 0 | 1.3 |
| PMX166 | 67.625 | 2781.335 | 112.285 | 14.5 | 11.4 | 22 | 9.15 | 0.95 | 2.77 | 1.28 | 0.92 |
| PMX167 | 63.545 | 1293.825 | 801.815 | 4.93 | 7.09 | 12.8 | 11.6 | 0 | 1.55 | 0.25 | 2.66 |
| PMX168 | 61.125 | 2454.935 | 300.935 | 13.4 | 7.17 | 6.52 | 8.87 | 0.15 | 1.57 | 0.36 | 1.53 |
| PMX170 | 71.3 | 282.29 | 114.02 | 11.8 | 9.44 | 11.3 | 7.39 | 0.27 | 1.99 | 0.47 | 0.79 |
| PMX171 | 105.995 | 277.335 | 145.845 | 13.7 | 11.9 | 6.22 | 10.4 | 0.57 | 0.98 | 0.52 | 1.91 |
| PMX173 | 56.735 | 1107.59 | 124.865 | 13.3 | 8.5 | 11.5 | 9.37 | 0.57 | 1.01 | 0.34 | 1.38 |
| PMX174 | 17.385 | 590.58 | 627.25 | 8.61 | 7.37 | 8.48 | 8.21 | 0.089 | 0.95 | 0 | 1.03 |
| PMX175 | 50.95 | 705.88 | 232.525 | 8.76 | 14.5 | 9.46 | 10 | 0.59 | 2.03 | 0.95 | 1 |
| PMX176 | 30.34 | 594.245 | 278.28 | 9.61 | 10.3 | 10.1 | 12.3 | 1.45 | 0.93 | 0 | 2.2 |
| PMX177 | 28.81 | 2206.83 | 762.63 | 12.6 | 8.02 | 18.4 | 13.7 | 1.37 | 1.41 | 0.47 | 3.19 |
| PMX178 | 24.735 | 2107.31 | 222.745 | 20.5 | 9.79 | 9.23 | 8.41 | 1.13 | 0.84 | 0.16 | 1.83 |
| PMX179 | 43.41 | 2752.975 | 162 | 22 | 8.73 | 22.9 | 10.9 | 0.48 | 1.27 | 0.31 | 2.06 |
| PMX180 | 55.485 | 772.83 | 317.565 | 8.6 | 10.7 | 17 | 13.9 | 0.21 | 1.2 | 0.58 | 1.77 |
| PMX181 | 33.125 | 1688.59 | 118.48 | 5.68 | 10.6 | 23.9 | 9.09 | 0.55 | 2.15 | 0.7 | 2.69 |
| PMX182 | 50.21 | 1470.52 | 1343.11 | 5.89 | 7.88 | 21.4 | 14.6 | 0.7 | 2.01 | 1.55 | 1.72 |
| PMX183 | 63.715 | 144.895 | 152.84 | 13.6 | 13.3 | 13.3 | 10.1 | 0.36 | 1.61 | 0.48 | 1.99 |
| PMX193 | 52.595 | 400.6 | 543.91 | 11.1 | 8.3 | 16.1 | 12.6 | 0.11 | 0.64 | 0.18 | 1.53 |
| PMX194 | 132.835 | 568.4 | 169.995 | 17.3 | 13 | 17.1 | 7.96 | 0.21 | 1.46 | 1.11 | 1.49 |
| PMX195 | 63.89 | 626.67 | 208.03 | 16.8 | 10.4 | 14.7 | 7.58 | 0.43 | 2.94 | 0.67 | 2.33 |
| PMX184 | 74.76 | 3286.04 | 321.85 | 28.8 | 10.4 | 28.2 | 12.3 | 1.47 | 2.42 | 0.75 | 1.64 |
| PMX196 | 34.8 | 2081.225 | 159.62 | 23 | 10.4 | 17.1 | 6.6 | 1.41 | 0.92 | 0 | 0.94 |
| PMX197 | 57.975 | 2341.04 | 306.53 | 23.3 | 10.5 | 14 | 9.24 | 0.67 | 1.43 | 0 | 3.21 |
| PMX198 | 82.8 | 2785.105 | 190.815 | 26.8 | 11.3 | 25.5 | 10.4 | 0.61 | 0.87 | 0 | 4.37 |
| PMX185 | 86.735 | 165.94 | 216.585 | 11.9 | 14.7 | 13.9 | 10.3 | 0.85 | 0.93 | 0.39 | 2.65 |
| PMX186 | 27.695 | 1218.565 | 216.585 | 20 | 10.5 | 17.6 | 10.9 | 0.2 | 2.63 | 0.74 | 1.09 |
| PMX200 | 59.555 | 822.035 | 363.47 | 19.5 | 8.83 | 15 | 13.1 | 2.92 | 0.76 | 0.78 | 1.29 |
| PMX190 | 54.405 | 699.18 | 384.455 | 14.8 | 11.3 | 11.8 | 13.5 | 0.98 | 0.73 | 2.73 | 0.9 |
| PMX191 | 31.85 | 744.48 | 369.635 | 10.5 | 8.97 | 14.9 | 14.2 | 0.35 | 0.66 | 0.6 | 2.89 |
| PMX192 | 75.9 | 757.725 | 209.895 | 12.7 | 10.4 | 11.3 | 10.3 | 0.66 | 1.29 | 1.56 | 3.65 |
| No Ab | 77.845 | 1219.87 | 1210.175 | 5.66 | 7.42 | 17.3 | 10.3 | 0.47 | 0.85 | 2.63 | 0.94 |

IFNγ concentration, CD25 activation and Ki67 were assessed at 6 days for level of agonistic activation of T cells and then again at day 9 post re-stimulation with Con A or PHA to assess anergy induction and immunosuppression. Two methods were tested using these candidate antibodies, plate bound (PB) and in solution (IS). For these experiments the in-solution data is especially relevant to future in vivo investigations as antibodies will be administered in liquid solution.

### Example 18: Canine CD3 Knock-In Mouse Models for In Vivo Examination of Fully Canine Immunosuppressive Antibodies

To evaluate the in vivo efficacy of fully canine CD3 monospecific immunosuppressive antibodies canine CD3e knock-in mice were generated as previously described in Example 12.

Canine CD3 knock-in mice will be used to determine the level of T cell activation, anergy induction and thus immunosuppression induced by monospecific immunosuppressive CD3 candidate antibodies. CD3 antibodies will be administered at a set dose and using peripheral blood to assess peripheral T cell changes using cytometric analysis. Common mouse T cell markers include but not limited to CD4, CD8, CD90.1.

Serum can be isolated for cytokine quantification, using commercially available ELISA IFNy, IL6, TNFa kits (Biolegend). This experiment will enable the evaluation of safety of these molecules along with in vivo characterisation of anergy induction.

To assess the ability of candidate anti-CD3 antibodies to induce T cell anergy in vivo, PBMC can be isolated from peripheral blood of canine CD3 knock-in mice after administration of anti-canine CD3 antibodies and cultured in presence of ConA or PHA. If the CD3 mAbs are able to induce T cell anergy in vivo then the T cells will be unresponsive to the further stimulation by ConA or PHA and the level of T cell activation markers, proliferation and IFNγ release will remain unchanged or decreased compared to that in PBMC isolated from untreated mice.

The ability of anti-canine CD3 antibodies to induce immunosuppression in vivo can also be assessed by quantifying their ability to reduce an immune response in canine CD3 knock-in mice. Briefly, after administration of anti-canine CD3 antibodies canine CD3 knock-in mice can be immunised with well-known T cell antigens, like Keyhole Limpet Hemocyanin (KLH). T cell response and T-cell dependent antibody response against KLH can be assessed by well-known techniques. Those techniques may include but are not limited to anti-KLH antibodies serum titre quantification and IFNγ ELISpot to determine the number of KLH-reactive T cells following immunisation.

### Example 19: In Vivo Dog Antigen Recall Examination of Fully Canine Immunosuppressive Antibodies

After evaluating the safety and efficacy of candidate anti-CD3 antibodies in transgenic mice, the best candidates will be evaluated in dogs. This will start at a low dose and may involve a dose escalation to evaluate the pharmacokinetic parameters, efficacy, and safety of the candidate CD3 immunosuppressive antibodies. Pharmacokinetic profile of each antibody will be evaluated by analysing serum samples collected pre dose and at various time points post administration throughout the study using ELISA techniques. For safety the serum samples collected will also be used to determine cytokine concentrations using ELISA techniques.

Antigen recall will be used to assess the level of immune T cell activation and then immunosuppression by candidate CD3 antibodies. PBMCs will be isolated from peripheral blood samples collected at various timepoints and these will be assessed in an ELISpot.

Peripheral blood will also be assessed for T cell activation markers.

TABLE 1
Sequences used in Examples

| SEQ | | |
|---|---|---|
| ID | | |
| NO: | Description | Sequence |
| 1 | Canine | ATGCAGTCGAGGAACCTCTGGAGAATTCTG |
| | CD3ϵ CDS | GGACTCTGTCTCTTATCAGTTGGTGCTTGG |
| | DNA | GGGCAGGACGAGGATTTCAAAGCTTCTGAT |
| | sequence | GACTTGACAAGTATATCTCCAGAGAAACGG |
| | | TTTAAGGTCTCCATCTCTGGAACCGAGGTA |
| | | GTGGTGACATGCCCTGATGTTTTTGGATAT |
| | | GATAATATAAAATGGGAAAAAAATGATAAC |
| | | CTTGTGGAAGGTGCTAGTAACAGAGAGCTA |
| | | TCTCAGAAGGAGTTTTCAGAAGTGGACGAC |
| | | AGTGGTTATTATGCCTGCTATGCAGATTCC |
| | | ATAAAGGAGAAGAGCTATCTCTACCTGAGA |
| | | GCAAGAGTGTGTGCAAACTGCATAGAGGTG |
| | | AATCTGATGGCAGTGGTCACAATCATTGTA |
| | | GCTGACATCTGCCTTACTCTGGGGTTGCTG |
| | | CTGATGGTGTATTACTGGAGCAAGACTAGA |
| | | AAGGCCAATGCCAAGCCTGTGATGAGAGGA |
| | | ACAGGTGCCGGCAGCAGGCCCAGGGGACAA |
| | | AACAAGGAGAAGCCACCACCTGTTCCCAAT |
| | | CCAGACTACGAGCCCATCCGGAAAGGCCAG |
| | | CAGGACCTGTATTCTGGCCTGAATCAGAGA |
| | | GGCATCTGA |
| 2 | Canine | ATGGAACACTGCAGGTTTCTGGCTGGCCTG |
| | CD3δ CDS | ATCCTGGCTGTCCTCCTCTCCCGAGTGAGC |
| | DNA | CCCTTCAAGGTATCTGTGGAGGAACTTGAG |
| | sequence | GACAGAGTGTTCCTGAGTTGCAATACCAGT |
| | | GTCCTAAGGATAGAGGGAACTATGGGAATA |
| | | CAACTCCCACACAGTAGAACTCTGGACTTG |
| | | GGGAAACGCATCCTGGACCCACGAGGAATA |
| | | TATCGGTGCAATGCGACAGAAGAACAATCC |
| | | GACAAAGCACCTTATATGCAAGTATATTAT |
| | | CGAATGTGTCAGAACTGTGTGGAGCTGAAC |
| | | TCAGCTACCCTGGCTGGCATTGTCATCGCT |
| | | GACATAATCGCCACTCTGCTCCTTGCTTTG |
| | | GGAGTCTATTGCTTTGCTGGACACGAGACT |
| | | GGAAGGTCCTCTAAGTCTGCTGACACTCAA |
| | | ATTCTGTTGGAAAATGACCAGCTCTATCAG |
| | | CCCCTTCGAGATCGGAATGATGCTCAGTAT |
| | | AGTCATCTTGGTGAAAACTGGCCTCGGAAG |
| | | AAGTGA |
| 3 | Canine | ATGGAGCTGGGGAAGCATCTGGCTGGCCTC |
| | CD3γ CDS | ATTTTGGCTGTCACTCTTCTTCAAGGTGCT |
| | DNA | ACAGCCCAGAAGGAACAAGGATTTCCCATG |
| | sequence | ATAAAAGTAGATGGTAATCGAGAAGATGGT |
| | | TCAGTACTTCTGATTTGTGACTCACAAAGC |
| | | AAAGATATCAAATGGTTTGAAGACGGGAAG |
| | | GAAAGAACTTTACCAAAGAAAGATAAAAAG |
| | | ACGTTGGATCTGGGAAGTAGTATGAAGGAC |
| | | CCTCAAGGGATATATCAGTGTCAAGTAGCA |
| | | AAGAACATTTCAAAGCCACTCCAAGTATAT |
| | | TACAGAATGTGTCAGAACTGCATTGAGCTG |
| | | AATGCAGGCACTATAATCGGCTTTGTCTTC |
| | | GCTGAAATCATCAGCATTTTCTTCCTTGCT |
| | | GTTGGGGTTTACTTCATTGCTGGACAGGAT |
| | | GGAGTTCGCCAGTCAAGAGCCTCAGACAAG |
| | | CAGACTCTGTTGTCCAATGACCAGCTTTAC |
| | | CAGCCCCTCAGGGATCGAGAAAATGACCAA |
| | | TATGGCCACCTTCAAGGAAAGCGACTGAGG |
| | | AAAAATTGA |
| 4 | Canine | MQSRNLWRILGLCLLSVGAWGQDEDFKASD |
| | CD3ϵ amino | DLTSISPEKRFKVSISGTEVVVTCPDVFGY |
| | acid | DNIKWEKNDNLVEGASNRELSQKEFSEVDD |
| | sequence | SGYYACYADSIKEKSYLYLRARVCANCIEV |
| | | NLMAVVTIIVADICLTLGLLLMVYYWSKTR |
| | | KANAKPVMRGTGAGSRPRGQNKEKPPPVPN |
| | | PDYEPIRKGQQDLYSGLNQRGI |
| 5 | Canine | MEHCRFLAGLILAVLLSRVSPFKVSVEELE |
| | CD3δ amino | DRVFLSCNTSVLRIEGTMGIQLPHSRTLDL |
| | acid | GKRILDPRGIYRCNATEEQSDKAPYMQVYY |
| | sequence | RMCQNCVELNSATLAGIVIADIIATLLLAL |
| | | GVYCFAGHETGRSSKSADTQILLENDQLYQ |
| | | PLRDRNDAQYSHLGENWPRKK |
| 6 | Canine | MELGKHLAGLILAVTLLQGATAQKEQGFPM |
| | CD3γ | IKVDGNREDGSVLLICDSQSKDIKWFEDGK |
| | amino acid | ERTLPKKDKKTLDLGSSMKDPQGIYQCQVA |
| | sequence | KNISKPLQVYYRMCQNCIELNAGTIIGFVF |
| | | AEIISIFFLAVGVYFIAGQDGVRQSRASDK |
| | | QTLLSNDQLYQPLRDRE |
| 7 | Canine | ATGCAGTCGAGGAACCTCTGGAGAATTCTG |
| | CD3ϵ- | GGACTCTGTCTCTTATCAGTTGGTGCTTGG |
| | hIgG4-knob | GGGCAGGACGAGGATTTCAAAGCTTCTGAT |
| | DNA | GACTTGACAAGTATATCTCCAGAGAAACGG |
| | sequence | TTTAAGGTCTCCATCTCTGGAACCGAGGTA |
| | | GTGGTGACATGCCCTGATGTTTTTGGATAT |
| | | GATAATATAAAATGGGAAAAAAATGATAAC |
| | | CTTGTGGAAGGTGCTAGTAACAGAGAGCTA |
| | | TCTCAGAAGGAGTTTTCAGAAGTGGACGAC |
| | | AGTGGTTATTATGCCTGCTATGCAGATTCC |
| | | ATAAAGGAGAAGAGCTATCTCTACCTGAGA |
| | | GCAAGAGTGTGTGCAAACTGCATAGAGGTG |
| | | AATGAGAGCAAGTACGGCCCTCCCTGCCCT |
| | | CCTTGTCCTGCCCCCGAGTTCGAAGGCGGA |
| | | CCCAGCGTGTTCCTGTTCCCTCCTAAGCCC |
| | | AAGGACACCCTCATGATCAGCCGGACACCC |
| | | GAGGTGACCTGCGTGGTGGTGGATGTGAGC |
| | | CAGGAGGACCCTGAGGTCCAGTTCAACTGG |
| | | TATGTGGATGGCGTGGAGGTGCACAACGCC |
| | | AAGACAAAGCCCCGGGAAGAGCAGTTCAAC |
| | | TCCACCTACAGGGTGGTCAGCGTGCTGACC |
| | | GTGCTGCATCAGGACTGGCTGAACGGCAAG |
| | | GAGTACAAGTGCAAGGTCAGCAATAAGGGA |
| | | CTGCCCAGCAGCATCGAGAAGACCATCTCC |
| | | AAGGCTAAAGGCCAGCCCCGGGAACCTCAG |
| | | GTGTGCACCCTGCCTCCCAGCCAGGAGGAG |
| | | ATGACCAAGAACCAGGTGAGCCTGTGGTGC |
| | | CTGGTGAAGGGATTCTACCCTTCCGACATC |
| | | GCCGTGGAGTGGGAGTCCAACGGCCAGCCC |
| | | GAGAACAATTATAAGACCACCCCTCCCGTC |
| | | CTCGACAGCGACGGATCCTTCTTTCTGTAC |
| | | TCCAGGCTGACCGTGGATAAGTCCAGGTGG |
| | | CAGGAAGGCAACGTGTTCAGCTGCTCCGTG |
| | | ATGCACGAGGCCCTGCACAATCACTACACC |
| | | CAGAAGTCCCTGAGCCTGTCCCTGGGAAAG |
| | | TGA |
| 8 | Canine | ATGGAACACTGCAGGTTTCTGGCTGGCCTG |
| | CD3δ- | ATCCTGGCTGTCCTCCTCTCCCGAGTGAGC |
| | hIgG4-hole | CCCTTCAAGGTATCTGTGGAGGAACTTGAG |
| | DNA | GACAGAGTGTTCCTGAGTTGCAATACCAGT |
| | sequence | GTCCTAAGGATAGAGGGAACTATGGGAATA |
| | | CAACTCCCACACAGTAGAACTCTGGACTTG |
| | | GGGAAACGCATCCTGGACCCACGAGGAATA |
| | | TATCGGTGCAATGCGACAGAAGAACAATCC |
| | | GACAAAGCACCTTATATGCAAGTATATTAT |
| | | CGAATGTGTCAGAACTGTGTGGAGCTGAAC |
| | | TCAGCTACCCTGGCTGAGAGCAAGTACGGC |
| | | CCTCCCTGCCCTCCTTGTCCTGCCCCCGAG |
| | | TTCGAAGGCGGACCCAGCGTGTTCCTGTTC |
| | | CCTCCTAAGCCCAAGGACACCCTCATGATC |
| | | AGCCGGACACCCGAGGTGACCTGCGTGGTG |
| | | GTGGATGTGAGCCAGGAGGACCCTGAGGTC |
| | | CAGTTCAACTGGTATGTGGATGGCGTGGAG |
| | | GTGCACAACGCCAAGACAAAGCCCCGGGAA |
| | | GAGCAGTTCAACTCCACCTACAGGGTGGTC |
| | | AGCGTGCTGACCGTGCTGCATCAGGACTGG |
| | | CTGAACGGCAAGGAGTACAAGTGCAAGGTC |
| | | AGCAATAAGGGACTGCCCAGCAGCATCGAG |
| | | AAGACCATCTCCAAGGCTAAAGGCCAGCCC |
| | | CGGGAACCTCAGGTGTACACCCTGCCTCCC |
| | | AGCCAGTGCGAGATGACCAAGAACCAGGTG |
| | | AGCCTGAGCTGCGCCGTGAAGGGATTCTAC |
| | | CCTTCCGACATCGCCGTGGAGTGGGAGTCC |
| | | AACGGCCAGCCCGAGAACAATTATAAGACC |
| | | ACCCCTCCCGTCCTCGACAGCGACGGATCC |
| | | TTCTTTCTGGTGTCCAGGCTGACCGTGGAT |
| | | AAGTCCAGGTGGCAGGAAGGCAACGTGTTC |
| | | AGCTGCTCCGTGATGCACGAGGCCCTGCAC |
| | | AATAGGTTCACCCAGAAGTCCCTGAGCCTG |
| | | TCCCTGGGAAAGTGA |
| 9 | Canine | MQSRNLWRILGLCLLSVGAWGQDEDFKASD |
| | CD3ϵ- | DLTSISPEKRFKVSISGTEVVVTCPDVFGY |
| | hIgG4-knob | DNIKWEKNDNLVEGASNRELSQKEFSEVDD |
| | amino acid | SGYYACYADSIKEKSYLYLRARVCANCIEV |
| | sequence | NESKYGPPCPPCPAPEFEGGPSVFLFPPKP |
| | | KDTLMISRTPEVTCVVVDVSQEDPEVQFNW |
| | | YVDGVEVHNAKTKPREEQFNSTYRVVSVLT |
| | | VLHQDWLNGKEYKCKVSNKGLPSSIEKTIS |
| | | KAKGQPREPQVCTLPPSQEEMTKNQVSLWC |
| | | LVKGFYPSDIAVEWESNGQPENNYKTTPPV |
| | | LDSDGSFFLYSRLTVDKSRWQEGNVFSCSV |
| | | MHEALHNHYTQKSLSLSLGK |
| 10 | Canine | MEHCRFLAGLILAVLLSRVSPFKVSVEELE |
| | CD3δ- | DRVFLSCNTSVLRIEGTMGIQLPHSRTLDL |
| | hIgG4-hole | GKRILDPRGIYRCNATEEQSDKAPYMQVYY |
| | amino acid | RMCQNCVELNSATLAESKYGPPCPPCPAPE |
| | sequence | FEGGPSVFLFPPKPKDTLMISRTPEVTCVV |
| | | VDVSQEDPEVQFNWYVDGVEVHNAKTKPRE |
| | | EQFNSTYRVVSVLTVLHQDWLNGKEYKCKV |
| | | SNKGLPSSIEKTISKAKGQPREPQVYTLPP |
| | | SQCEMTKNQVSLSCAVKGFYPSDIAVEWES |
| | | NGQPENNYKTTPPVLDSDGSFFLVSRLTVD |
| | | KSRWQEGNVFSCSVMHEALHNRFTQKSLSL |
| | | SLGK |
| 11 | Human IgG4 | GCCAGCACCAAGGGCCCTTCCGTGTTCCCC |
| | hole Protein | CTGGCCCCTTGCAGCAGGAGCACCTCCGAA |
| | A mutant | TCCACAGCTGCCCTGGGCTGTCTGGTGAAG |
| | DNA | GACTACTTTCCCGAGCCCGTGACCGTGAGC |
| | sequence | TGGAACAGCGGCGCTCTGACATCCGGCGTC |
| | | CACACCTTTCCTGCCGTCCTGCAGTCCTCC |
| | | GGCCTCTACTCCCTGTCCTCCGTGGTGACC |
| | | GTGCCTAGCTCCTCCCTCGGCACCAAGACC |
| | | TACACCTGTAACGTGGACCACAAACCCTCC |
| | | AACACCAAGGTGGACAAACGGGTCGAGAGC |
| | | AAGTACGGCCCTCCCTGCCCTCCTTGTCCT |
| | | GCCCCCGAGTTCGAAGGCGGACCCAGCGTG |
| | | TTCCTGTTCCCTCCTAAGCCCAAGGACACC |
| | | CTCATGATCAGCCGGACACCCGAGGTGACC |
| | | TGCGTGGTGGTGGATGTGAGCCAGGAGGAC |
| | | CCTGAGGTCCAGTTCAACTGGTATGTGGAT |
| | | GGCGTGGAGGTGCACAACGCCAAGACAAAG |
| | | CCCCGGGAAGAGCAGTTCAACTCCACCTAC |
| | | AGGGTGGTCAGCGTGCTGACCGTGCTGCAT |
| | | CAGGACTGGCTGAACGGCAAGGAGTACAAG |
| | | TGCAAGGTCAGCAATAAGGGACTGCCCAGC |
| | | AGCATCGAGAAGACCATCTCCAAGGCTAAA |
| | | GGCCAGCCCCGGGAACCTCAGGTGTACACC |
| | | CTGCCTCCCAGCCAGTGCGAGATGACCAAG |
| | | AACCAGGTGAGCCTGAGCTGCGCCGTGAAG |
| | | GGATTCTACCCTTCCGACATCGCCGTGGAG |
| | | TGGGAGTCCAACGGCCAGCCCGAGAACAAT |
| | | TATAAGACCACCCCTCCCGTCCTCGACAGC |
| | | GACGGATCCTTCTTTCTGGTGTCCAGGCTG |
| | | ACCGTGGATAAGTCCAGGTGGCAGGAAGGC |
| | | AACGTGTTCAGCTGCTCCGTGATGCACGAG |
| | | GCCCTGCACAATAGGTTCACCCAGAAGTCC |
| | | CTGAGCCTGTCCCTGGGAAAGTGA |
| 12 | Human IgG4 | ASTKGPSVFPLAPCSRSTSESTAALGCLVK |
| | hole Protein | DYFPEPVTVSWNSGALTSGVHTFPAVLQSS |
| | A mutant | GLYSLSSVVTVPSSSLGTKTYTCNVDHKPS |
| | amino acid | NTKVDKRVESKYGPPCPPCPAPEFEGGPSV |
| | sequence | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVYTLPPSQCEMTK |
| | | NQVSLSCAVKGFYPSDIAVEWESNGQPENN |
| | | YKTTPPVLDSDGSFFLVSRLTVDKSRWQEG |
| | | NVFSCSVMHEALHNRFTQKSLSLSLGK |
| 13 | Human | CGTACGGTGGCCGCTCCCTCCGTGTTCATC |
| | kappa light | TTCCCACCTTCCGACGAGCAGCTGAAGTCC |
| | chain | GGCACCGCTTCTGTCGTGTGCCTGCTGAAC |
| | constant | AACTTCTACCCCCGCGAGGCCAAGGTGCAG |
| | DNA | TGGAAGGTGGACAACGCCCTGCAGTCCGGC |
| | sequence | AACTCCCAGGAATCCGTGACCGAGCAGGAC |
| | | TCCAAGGACAGCACCTACTCCCTGTCCTCC |
| | | ACCCTGACCCTGTCCAAGGCCGACTACGAG |
| | | AAGCACAAGGTGTACGCCTGCGAAGTGACC |
| | | CACCAGGGCCTGTCTAGCCCCGTGACCAAG |
| | | TCTTTCAACCGGGGCGAGTGTTGA |
| 14 | Human | RTVAAPSVFIFPPSDEQLKSGTASVVCLLN |
| | kappa amino | NFYPREAKVQWKVDNALQSGNSQESVTEQD |
| | acid chain | SKDSTYSLSSTLTLSKADYEKHKVYACEVT |
| | constant | HQGLSSPVTKSFNRGEC |
| | sequence | |
| 15 | Human IgG4 | GCCAGCACCAAGGGCCCTTCCGTGTTCCCC |
| | knob DNA | CTGGCCCCTTGCAGCAGGAGCACCTCCGAA |
| | sequence | TCCACAGCTGCCCTGGGCTGTCTGGTGAAG |
| | | GACTACTTTCCCGAGCCCGTGACCGTGAGC |
| | | TGGAACAGCGGCGCTCTGACATCCGGCGTC |
| | | CACACCTTTCCTGCCGTCCTGCAGTCCTCC |
| | | GGCCTCTACTCCCTGTCCTCCGTGGTGACC |
| | | GTGCCTAGCTCCTCCCTCGGCACCAAGACC |
| | | TACACCTGTAACGTGGACCACAAACCCTCC |
| | | AACACCAAGGTGGACAAACGGGTCGAGAGC |
| | | AAGTACGGCCCTCCCTGCCCTCCTTGTCCT |
| | | GCCCCCGAGTTCGAAGGCGGACCCAGCGTG |
| | | TTCCTGTTCCCTCCTAAGCCCAAGGACACC |
| | | CTCATGATCAGCCGGACACCCGAGGTGACC |
| | | TGCGTGGTGGTGGATGTGAGCCAGGAGGAC |
| | | CCTGAGGTCCAGTTCAACTGGTATGTGGAT |
| | | GGCGTGGAGGTGCACAACGCCAAGACAAAG |
| | | CCCCGGGAAGAGCAGTTCAACTCCACCTAC |
| | | AGGGTGGTCAGCGTGCTGACCGTGCTGCAT |
| | | CAGGACTGGCTGAACGGCAAGGAGTACAAG |
| | | TGCAAGGTCAGCAATAAGGGACTGCCCAGC |
| | | AGCATCGAGAAGACCATCTCCAAGGCTAAA |
| | | GGCCAGCCCCGGGAACCTCAGGTGTGCACC |
| | | CTGCCTCCCAGCCAGGAGGAGATGACCAAG |
| | | AACCAGGTGAGCCTGTGGTGCCTGGTGAAG |
| | | GGATTCTACCCTTCCGACATCGCCGTGGAG |
| | | TGGGAGTCCAACGGCCAGCCCGAGAACAAT |
| | | TATAAGACCACCCCTCCCGTCCTCGACAGC |
| | | GACGGATCCTTCTTTCTGTACTCCAGGCTG |
| | | ACCGTGGATAAGTCCAGGTGGCAGGAAGGC |
| | | AACGTGTTCAGCTGCTCCGTGATGCACGAG |
| | | GCCCTGCACAATCACTACACCCAGAAGTCC |
| | | CTGAGCCTGTCCCTGGGAAAGTGA |
| 16 | Human IgG4 | ASTKGPSVFPLAPCSRSTSESTAALGCLVK |
| | knob amino | DYFPEPVTVSWNSGALTSGVHTFPAVLQSS |
| | acid | GLYSLSSVVTVPSSSLGTKTYTCNVDHKPS |
| | sequence | NTKVDKRVESKYGPPCPPCPAPEFEGGPSV |
| | | FLFPPKPKDTLMISRTPEVTCVVVDVSQED |
| | | PEVQFNWYVDGVEVHNAKTKPREEQFNSTY |
| | | RVVSVLTVLHQDWLNGKEYKCKVSNKGLPS |
| | | SIEKTISKAKGQPREPQVCTLPPSQEEMTK |
| | | NQVSLWCLVKGFYPSDIAVEWESNGQPENN |
| | | YKTTPPVLDSDGSFFLYSRLTVDKSRWQEG |
| | | NVFSCSVMHEALHNHYTQKSLSLSLGK |
| 17 | Rituximab | CAGGTTCAGCTGCAACAGCCTGGCGCCGAG |
| | variable | CTTGTGAAACCTGGCGCCTCTGTGAAGATG |
| | heavy chain | AGCTGCAAGGCCAGCGGCTACACCTTCACC |
| | DNA | AGCTACAACATGCACTGGGTCAAGCAGACC |
| | sequence | CCTGGCAGAGGCCTGGAATGGATCGGAGCC |
| | | ATCTATCCCGGCAACGGCGACACCTCCTAC |
| | | AACCAGAAGTTCAAGGGCAAAGCCACACTG |
| | | ACCGCCGACAAGAGCAGCAGCACAGCCTAC |
| | | ATGCAGCTGAGCAGCCTGACCAGCGAAGAT |
| | | AGCGCCGTGTACTACTGCGCCAGAAGCACC |
| | | TATTACGGCGGCGACTGGTACTTCAACGTG |
| | | TGGGGAGCTGGCACCACCGTGACAGTTTCT |
| | | GCT |
| 18 | Rituximab | QVQLQQPGAELVKPGASVKMSCKASGYTFT |
| | variable | SYNMHWVKQTPGRGLEWIGAIYPGNGDTSY |
| | heavy chain | NQKFKGKATLTADKSSSTAYMQLSSLTSED |
| | amino acid | SAVYYCARSTYYGGDWYFNVWGAGTTVTVS |
| | sequence | A |
| 19 | Rituximab | CAGATTGTGCTGTCTCAGAGCCCCGCCATC |
| | variable | CTGAGTGCTTCTCCAGGCGAGAAAGTGACC |
| | light chain | ATGACCTGCAGAGCCAGCAGCAGCGTGTCC |
| | DNA | TACATCCACTGGTTCCAGCAGAAGCCTGGC |
| | sequence | AGCAGCCCTAAGCCTTGGATCTACGCCACA |
| | | AGCAATCTGGCCAGCGGCGTGCCAGTCAGA |
| | | TTTTCTGGCTCTGGCAGCGGCACCAGCTAC |
| | | AGCCTGACAATCTCTAGAGTGGAAGCCGAG |
| | | GACGCCGCCACCTACTATTGTCAGCAGTGG |
| | | ACCAGCAATCCTCCTACCTTTGGCGGAGGC |
| | | ACCAAGCTGGAAATCAAAC |
| 20 | Rituximab | QIVLSQSPAILSASPGEKVTMTCRASSSVS |
| | variable | YIHWFQQKPGSSPKPWIYATSNLASGVPVR |
| | light chain | FSGSGSGTSYSLTISRVEAEDAATYYCQQW |
| | amino acid | TSNPPTFGGGTKLEIK |
| | sequence | |
| 21 | Human | ATGACAACACCCAGAAATTCAGTAAATGGG |
| | CD20 DNA | ACTTTCCCGGCAGAGCCAATGAAAGGCCCT |
| | coding | ATTGCTATGCAATCTGGTCCAAAACCACTC |
| | sequence | TTCAGGAGGATGTCTTCACTGGTGGGCCCC |
| | | ACGCAAAGCTTCTTCATGAGGGAATCTAAG |
| | | ACTTTGGGGGCTGTCCAGATTATGAATGGG |
| | | CTCTTCCACATTGCCCTGGGGGGTCTTCTG |
| | | ATGATCCCAGCAGGGATCTATGCACCCATC |
| | | TGTGTGACTGTGTGGTACCCTCTCTGGGGA |
| | | GGCATTATGTATATTATTTCCGGATCACTC |
| | | CTGGCAGCAACGGAGAAAAACTCCAGGAAG |
| | | TGTTTGGTCAAAGGAAAAATGATAATGAAT |
| | | TCATTGAGCCTCTTTGCTGCCATTTCTGGA |
| | | ATGATTCTTTCAATCATGGACATACTTAAT |
| | | ATTAAAATTTCCCATTTTTTAAAAATGGAG |
| | | AGTCTGAATTTTATTAGAGCTCACACACCA |
| | | TATATTAACATATACAACTGTGAACCAGCT |
| | | AATCCCTCTGAGAAAAACTCCCCATCTACC |
| | | CAATACTGTTACAGCATACAATCTCTGTTC |
| | | TTGGGCATTTTGTCAGTGATGCTGATCTTT |
| | | GCCTTCTTCCAGGAACTTGTAATAGCTGGC |
| | | ATCGTTGAGAATGAATGGAAAAGAACGTGC |
| | | TCCAGACCCAAATCTAACATAGTTCTCCTG |
| | | TCAGCAGAAGAAAAAAAAGAACAGACTATT |
| | | GAAATAAAAGAAGAAGTGGTTGGGCTAACT |
| | | GAAACATCTTCCCAACCAAAGAATGAAGAA |
| | | GACATTGAAATTATTCCAATCCAAGAAGAG |
| | | GAAGAAGAAGAAACAGAGACGAACTTTCCA |
| | | GAACCTCCCCAAGATCAGGAATCCTCACCA |
| | | ATAGAAAATGACAGCTCTCCTTAA |
| 22 | Canine | ATGACAACACCCAGAAATTCAATGAGTGGA |
| | CD20 DNA | ACTCTCCCGGTAGATCCTATGAAAAGCCCT |
| | coding | ACTGCCATGTATCCTGTTCAAAAAATAATT |
| | sequence | CCCAAAAGGATGCCTTCAGTGGTGGGCCCT |
| | | ACACAAAACTTCTTCATGAGGGAATCTAAG |
| | | ACACTGGGGGCTGTCCAGATTATGAATGGG |
| | | CTCTTCCACATTGCCCTAGGCAGCCTCCTG |
| | | ATGATTCACACGGATGTCTATGCGCCCATC |
| | | TGTATAACTATGTGGTACCCTCTCTGGGGA |
| | | GGCATTATGTTCATCATTTCTGGATCACTC |
| | | CTGGCAGCAGCGGACAAAAACCCCAGGAAG |
| | | AGTTTGGTCAAAGGAAAAATGATAATGAAC |
| | | TCATTGAGCCTCTTTGCTGCTATTTCTGGA |
| | | ATAATTTTTTTGATCATGGACATATTTAAT |
| | | ATTACCATTTCCCATTTTTTTAAAATGGAG |
| | | AATTTGAATCTTATTAAAGCTCCCATGCCA |
| | | TATGTTGACATACACAACTGTGACCCAGCT |
| | | AACCCCTCTGAGAAAAACTCTTTATCTATA |
| | | CAATATTGTGGCAGCATACGATCTGTTTTC |
| | | TTGGGCGTTTTTGCTGTGATGGTGATCTTT |
| | | ACCTTTTTCCAGAAACTTGTGACAGCTGGC |
| | | ATTGTTGAGAATGAATGGAAAAAACTGTGC |
| | | TCTAAACCTAAATCTGATGTAGTTGTTCTG |
| | | TTAGCTGCTGAAGAAAAAAAAGAACAGCCG |
| | | ATTGAAACAACAGAAGAAATGGTTGAGCTG |
| | | ACTGAAATAGCTTCCCAACCAAAGAAAGAA |
| | | GAAGACATTGAAATTATTCCAGTCCAAGAA |
| | | GAAGAAGAGGAACTGGAAATAAACTTTGCA |
| | | GAACCTCCCCAGGAGCAGGAATCTTCACCA |
| | | ATAGAAAACGACAGCATCCCTTAA |
| 23 | Human | MTTPRNSVNGTFPAEPMKGPIAMQSGPKPL |
| | CD20 amino | FRRMSSLVGPTQSFFMRESKTLGAVQIMNG |
| | acid | LFHIALGGLLMIPAGIYAPICVTVWYPLWG |
| | sequence | GIMYIISGSLLAATEKNSRKCLVKGKMIMN |
| | | SLSLFAAISGMILSIMDILNIKISHFLKME |
| | | SLNFIRAHTPYINIYNCEPANPSEKNSPST |
| | | QYCYSIQSLFLGILSVMLIFAFFQELVIAG |
| | | IVENEWKRTCSRPKSNIVLLSAEEKKEQTI |
| | | EIKEEVVGLTETSSQPKNEEDIEIIPIQEE |
| | | EEEETETNFPEPPQDQESSPIENDSSP |
| 24 | Canine | MTTPRNSMSGTLPVDPMKSPTAMYPVQKII |
| | CD20 amino | PKRMPSVVGPTQNFFMRESKTLGAVQIMNG |
| | acid | LFHIALGSLLMIHTDVYAPICITMWYPLWG |
| | sequence | GIMFIISGSLLAAADKNPRKSLVKGKMIMN |
| | | SLSLFAAISGIIFLIMDIFNITISHFFKME |
| | | NLNLIKAPMPYVDIHNCDPANPSEKNSLSI |
| | | QYCGSIRSVFLGVFAVMVIFTFFQKLVTAG |
| | | IVENEWKKLCSKPKSDVVVLLAAEEKKEQP |
| | | IETTEEMVELTEIASQPKKEEDIEIIPVQE |
| | | EEEELEINFAEPPQEQESSPIENDSIP |
| 25 | Green | ATGGTGAGCAAGGGCGAGGAGCTGTTCACC |
| | Fluorescent | GGGGTGGTGCCCATCCTGGTCGAGCTGGAC |
| | Protein | GGCGACGTAAACGGCCACAAGTTCAGCGTG |
| | DNA | TCCGGCGAGGGCGAGGGCGATGCCACCTAC |
| | sequence | GGCAAGCTGACCCTGAAGTTCATCTGCACC |
| | | ACCGGCAAGCTGCCCGTGCCCTGGCCCACC |
| | | CTCGTGACCACCCTGACCTACGGCGTGCAG |
| | | TGCTTCAGCCGCTACCCCGACCACATGAAG |
| | | CAGCACGACTTCTTCAAGTCCGCCATGCCC |
| | | GAAGGCTACGTCCAGGAGCGCACCATCTTC |
| | | TTCAAGGACGACGGCAACTACAAGACCCGC |
| | | GCCGAGGTGAAGTTCGAGGGCGACACCCTG |
| | | GTGAACCGCATCGAGCTGAAGGGCATCGAC |
| | | TTCAAGGAGGACGGCAACATCCTGGGGCAC |
| | | AAGCTGGAGTACAACTACAACAGCCACAAC |
| | | GTCTATATCATGGCCGACAAGCAGAAGAAC |
| | | GGCATCAAGGTGAACTTCAAGATCCGCCAC |
| | | AACATCGAGGACGGCAGCGTGCAGCTCGCC |
| | | GACCACTACCAGCAGAACACCCCCATCGGC |
| | | GACGGCCCCGTGCTGCTGCCCGACAACCAC |
| | | TACCTGAGCACCCAGTCCGCCCTGAGCAAA |
| | | GACCCCAACGAGAAGCGCGATCACATGGTC |
| | | CTGCTGGAGTTCGTGACCGCCGCCGGGATC |
| | | ACTCTCGGCATGGACGAGCTGTACAAGTAA |
| 26 | Canine | MTTPRNSMSGTLPVDPMKSPTAMYPVQKII |
| | CD20 amino | PKRMPSVVGPTQNFFMRESKTLGAVQIMNG |
| | acid | LFHIALGSLLMIHTDVYAPICITMWYPLWG |
| | sequence | GIMFIISGSLLAAADKNPRKSLVKGKMIMN |
| | | SLSLFAAISGIIFLIMDIFNITISHFFKME |
| | | NLNLIKAPMPYVDIHNCDPANPSEKNSLSI |
| | | QYCGSIRSVFLGVFAVMVIFTFFQKLVTAG |
| | | IVENEWKKLCSKPKSDVVVLLAAEEKKEQP |
| | | IETTEEMVELTEIASQPKKEEDIEIIPVQE |
| | | EEEELEINFAEPPQEQESSPIENDSIP |
| 27 | Genomic | TGCAGCCGCAGAAACACGAGGCCCTGGGGC |
| | sequence of | GGCAGAGAGCTACGCCGGGCGGCTGGTCTG |
| | canine IgGB | CAGACTTCCTGGAGGGCCACCCCAAGCCCG |
| | Coding | GGCACCTGATAACCTGCCCGGACCCTCCCA |
| | regions are | GGAGCGAGGCCCTGGTGGTCACACAGCCTC |
| | indicated | CTCTCTTGCAGCCTCCCCCACGGCCCCATC |
| | by an | GGTGTTCCCACTGGCCCCCAGCTGCGGGAC |
| | underscore. | CACATCTGGCGCCACCGTGGCCCTGGCCTG |
| | | CCTGGTGTTAGGCTACTTCCCTGAGCCGGT |
| | | GACCGTGTCCTGGAACTCCGGCGCCCTGAC |
| | | CAGCGGTGTGCACACCTTCCCATCCGTCCT |
| | | GCAGGCCTCGGGGCTCTACTCTCTCAGCAG |
| | | CATGGTGACAGTGCCCTCCAGCAGGTGGCT |
| | | CAGTGACACCTTCACCTGCAACGTGGCCCA |
| | | CCGGCCCAGCAGCACCAAAGTGGACAAGAC |
| | | CGGTAAGAGGGTGTCCACTGGGAGACAGGC |
| | | CAGGTCAGCCGGTCCACCTGGACCCCGGGG |
| | | CACAGGAGAGGCACCTCTGTCCCCCAAGCT |
| | | CGGGGAGGAGGGCCTCTGGGCCTGGCTGCC |
| | | TGGTCCAGTGTGAGCACAGGCTGCACAGCC |
| | | CCACCATACTTCATCTCCACTGGCAGGCAC |
| | | CAGTCTGGGACGCCCCCCTGACCTGCCCTG |
| | | ACCTAGCCCACCCCAAGAGCTGTCCCCACC |
| | | CCTGGCCCCCACTAACCTCCAGTCTGGTCT |
| | | CTCTGCAGTACCCAAAACAGCGTCTACAAT |
| | | AGAGTCCAAAACCGGGGAAGGTCCCAAATG |
| | | CCCAGGTGAGTCACCAGGGCACCACCTTGC |
| | | ACACCAAGGCAATGGCCCGCATCCAGGGAC |
| | | TGCCTGGTGGGGGGAATGTTCAAGAGGATC |
| | | CCCCCCAAATGCTGACCCCTGCATTGTGTC |
| | | TCCCACACCAGTTCCTGAGATTCCTGGAGC |
| | | ACCGTCCGTCTTCATCTTCCCCCCAAAACC |
| | | CAAGGACACCCTCTCGATTTCCCGGACGCC |
| | | CGAGGTCACGTGCTTGGTGGTGGACTTGGG |
| | | CCCAGATGACTCCAATGTCCAGATCACATG |
| | | GTTTGTGGATAACACCGAGATGCACACAGC |
| | | CAAGACGAGGCCGCGTGAGGAGCAGTTCAA |
| | | CAGCACCTACCGTGTGGTCAGTGTCCTCCC |
| | | CATCCTACACCAGGACTGGCTCAAGGGGAA |
| | | GGAGTTCAAGTGCAAGGTCAACAGCAAATC |
| | | CCTCCCCTCTGCCATGGAGAGGACCATCTC |
| | | CAAGGCCAAAGGTGGGCAACAGGACAGATG |
| | | GGGCACAGGGAGGTCGAGTGGGGCCTGGTG |
| | | GACCCAGGCCAGCCCTCCACTCTGGGAGTG |
| | | ACCATCTGTGCTGACCTCTGACCCCACAGG |
| | | ACAGCCCCACGAGCCCCAGGTGTACGTCCT |
| | | GCCTCCAACCCAGGAGGAGCTCAGCGAGAA |
| | | CAAAGTCAGTGTGACCTGCCTGATCAAAGG |
| | | CTTCCACCCGCCTGACATTGCCGTCGAGTG |
| | | GGAGATCACCGGACAGCCGGAGCCAGAGAA |
| | | CAACTACCAGACGACCCCGCCCCAGCTGGA |
| | | CAGCGACGGGACCTACTTCCTGTACAGCAG |
| | | GCTCTCGGTGGACAGGTCCCACTGGCAGAG |
| | | GGGAAACACCTACACCTGCTCGGTGTCACA |
| | | CGAAGCTCTGCACAGCCACCACACACAGAA |
| | | ATCCCTCACCCAATCTCCGGGTAAATGAGC |
| | | AGCGCGCCCAGCCCCCCAGGAGGCCCCCGC |
| | | GGGCTCTGAGCGCCCACCCCTGTGTACATC |
| | | CCCCCCCCCCGGGCAGGCACCCTGCATGAA |
| | | ATAAAGCACCCAGCACTGCCCTGGGACCTG |
| | | TGACACTGTCGTGCGTCTTTCCAAGGCAGA |
| | | GCTCAGGACACCCGGGCCTCTGGGAGGTGC |
| | | GGGCAGGCCAGGCTCTGAGGCTCAGGGGTC |
| | | ACCAT |
| 28 | Canine | ASTTAPSVFPLAPSCGSTSGSTVALACLVS |
| | IgGB amino | GYFPEPVTVSWNSGSLTSGVHTFPSVLQSS |
| | acid | GLYSLSSMVTVPSSRWPSETFTCNVAHPAS |
| | sequence | KTKVDKPVPKRENGRVPRPPDCPKCPAPEM |
| | | LGGPSVFIFPPKPKDTLLIARTPEVTCVVV |
| | | DLDPEDPEVQISWFVDGKQMQTAKTQPREE |
| | | QFNGTYRVVSVLPIGHQDWLKGKQFTCKVN |
| | | NKALPSPIERTISKARGQAHQPSVYVLPPS |
| | | REELSKNTVSLTCLIKDFFPPDIDVEWQSN |
| | | GQQEPESKYRTTPPQLDEDGSYFLYSKLSV |
| | | DKSRWQRGDTFICAVMHEALHNHYTQKSLS |
| | | HSPGK |
| 29 | Effector | GCCTCCACCACGGCCCCCTCGGTTTTCCCA |
| | function | CTGGCCCCCAGCTGCGGGTCCACTTCCGGC |
| | deficient | TCCACGGTGGCCCTGGCCTGCCTGGTGTCA |
| | canine IgGB | GGCTACTTCCCCGAGCCTGTAACTGTGTCC |
| | DNA | TGGAATTCCGGCTCCTTGACCAGCGGTGTG |
| | sequence | CACACCTTCCCGTCCGTCCTGCAGTCCTCA |
| | | GGGCTCTACTCCCTCAGCAGCATGGTGACA |
| | | GTGCCCTCCAGCAGGTGGCCCAGCGAGACC |
| | | TTCACCTGCAACGTGGCCCACCCGGCCAGC |
| | | AAAACTAAAGTAGACAAGCCAGGTGAGACG |
| | | TCGGACCTCAGAGAGGGGTCCACTCGGGGA |
| | | CAAGCCAGATCAGCTGTCCCTCCCAGACCC |
| | | ACGTCACCGGGGAGTCACCTCAGTGTCCCC |
| | | GTCCTCAAGGCCTCCCCTTTCTGGGAAGGT |
| | | GTCCTCTGGGCGTGGCTGCCTGGTCCAGAT |
| | | GACCACAGGCTGCATTCCCCACTGTATTCC |
| | | CAGGACCCATTGGGTGCCACTGCTCGAGGG |
| | | TCCCCTAAGTTCACCCCAAGACCTGTGCCC |
| | | ACACCAGGCCCCCAGTAACCCCCAGTCTGC |
| | | TCTCTCTGCAGTGCCCAAAAGAGAAAATGG |
| | | AAGAGTTCCTCGCCCACCTGATTGTCCCAA |
| | | ATGCCCAGGTGAGTCAGCAGGGCCCTGCTC |
| | | TGCATCCCAAGCCGATGGTGCACACCCAGG |
| | | CACAGCCTGATGGGCTAATGGGTGTTGGAG |
| | | AAGTCCACCGAAGTGCTACCTCATCCTTGT |
| | | GTCTTCCATTTTAGCCCCTGAAAGCCTGGG |
| | | AGGGCCTTCGGTCTTCATCTTTCCCCCGAA |
| | | ACCCAAGGACACCCTCTTGATTGCCCGAAC |
| | | ACCTGAGGTCACATGTGTGGTGGTGGATCT |
| | | GGGCCGCGAAGACCCTGAGGTGCAGATCAG |
| | | CTGGTTCGTGGACGGTAAGCAGATGCAAAC |
| | | AGCCAAGACTCAGCCTCGTGAGGAGCAGTT |
| | | CAATGGCACCTACCGTGTGGTCAGTGTCCT |
| | | CCCCATTGGGCACCAGGACTGGCTCAAGGG |
| | | GAAGCAGTTCACGTGCAAAGTCAACCACAT |
| | | TGGCCTCCCATCCCCGATCGAGAGGACCAT |
| | | CTCCAAGGCCAGAGGTAGGCAGCAGGGCAT |
| | | AGGGGCTGCAGGGAGGGAGAGTTGCCCTGT |
| | | AAATTGATACCAGTCCTCCACCCTGATAGT |
| | | GACCATCTGTGCTGATCCTTTACCCCATAG |
| | | GGCAGGCCCATCAACCCAGTGTGTATGTCC |
| | | TGCCGCCATCCCGGGAGGAGTTGAGCAAGA |
| | | ACACAGTCAGCTTGACATGCCTGATCAAAG |
| | | ACTTCTTCCCACCTGACATTGATGTGGAGT |
| | | GGCAGAGCAATGGACAGCAGGAGCCTGAGA |
| | | GCAAGTACCGCACGACCCCGCCCCAGCTGG |
| | | ACGAGGACGGGTCCTACTTCCTGTACAGCA |
| | | AGCTCTCTGTGGACAAGAGCCGCTGGCAGC |
| | | GGGGAGACACCTTCATATGTGCGGTGATGC |
| | | ATGAAGCTCTACACAACCACTACACACAGA |
| | | AATCCCTCTCCCATTCTCCGGGTAAATGA |
| 30 | Effector | ASTTAPSVFPLAPSCGSTSGSTVALACLVS |
| | function | GYFPEPVTVSWNSGSLTSGVHTFPSVLQSS |
| | deficient | GLYSLSSMVTVPSSRWPSETFTCNVAHPAS |
| | canine IgGB | KTKVDKPVPKRENGRVPRPPDCPKCPAPES |
| | amino acid | LGGPSVFIFPPKPKDTLLIARTPEVTCVVV |
| | sequence | DLGREDPEVQISWFVDGKQMQTAKTQPREE |
| | | QFNGTYRVVSVLPIGHQDWLKGKQFTCKVN |
| | | HIGLPSPIERTISKARGQAHQPSVYVLPPS |
| | | REELSKNTVSLTCLIKDFFPPDIDVEWQSN |
| | | GQQEPESKYRTTPPQLDEDGSYFLYSKLSV |
| | | DKSRWQRGDTFICAVMHEALHNHYTQKSLS |
| | | HSPGK |
| 31 | Canine | GGTCAGCCCAAGGCCTCCCCTTCGGTCACA |
| | lambda L5 | CTCTTCCCGCCCTCCTCTGAGGAGCTTGGC |
| | DNA | GCCAACAAGGCCACCCTGGTGTGCCTCATC |
| | sequence | AGCGACTTCTACCCCAGCGGCGTGACAGTG |
| | | GCCTGGAAGGCAGACGGCAGCCCCATCACC |
| | | CAGGGTGTGGAGACCACCAAGCCCTCCAAG |
| | | CAGAGCAACAACAAGTACGCGGCCAGCAGC |
| | | TACCTGAGCCTGACGCCTGACAAGTGGAAA |
| | | TCTCACAGCAGCTTCAGCTGCCTGGTCACG |
| | | CACGAGGGGAGCACCGTGGAGAAGAAGGTG |
| | | GCCCCCGCAGAGTGCTCTTAG |
| 32 | Canine | GQPKASPSVTLFPPSSEELGANKATLVCLI |
| | lambda L5 | SDFYPSGVTVAWKADGSPITQGVETTKPSK |
| | amino acid | QSNNKYAASSYLSLTPDKWKSHSSFSCLVT |
| | sequence | HEGSTVEKKVAPAECS |

TABLE 2
Sequences of antibodies/portions thereof
that bind canine CD3

| SEQ | | |
|---|---|---|
| ID | | |
| NO: | Description | Sequence |
| 33 | PMX157 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGCAGTTACTTCTGGAACTGGATCCGCCAG |
| | | CGCCCTGGGAGGGGACTGGAATGGATGGGG |
| | | TACTGGACAGGTAGCACAATCTACAGCCCG |
| | | GCATTCCAGGGACGCATCTCCATCACTCCT |
| | | GACACGGCCAAGAACCAGTTCTCCCTGCAG |
| | | CTGAGCTCCATGACCACCGAGGACACGGCC |
| | | GTGTATTACTGTTCAAGAGAGGGAGGTAGC |
| | | ACCTTGGGGGCTTTTGCTTACTGGGGCCAG |
| | | GGCACCCTGGTCACTGTCTCCTCAG |
| 34 | PMX157 | ELTLQESGPGLVKPSQTLSLTCVVSGGSVT |
| | VH_AA | SSYFWNWIRQRPGRGLEWMGYWTGSTIYSP |
| | | AFQGRISITPDTAKNQFSLQLSSMTTEDTA |
| | | VYYCSREGGSTLGAFAYWGQGTLVTVSS |
| 35 | PMX157 | TCCTATGTGCTGACACAGTTGCCATCCAAA |
| | VL_NT | AATGTGACCCTGAAGCAGCCGGCCCACATC |
| | | ACCTGTGGGGGAGACAAAATTGGAAGTAAA |
| | | AGTGTTCACTGGTACCAGCAGAAGCTGGGC |
| | | CAGGCCCCTGTATTGATTATCTATTATGAT |
| | | AGCAGCAGGCCGACAGGGATCCCTGAGCGA |
| | | TTCTCCGGCGCCAATTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 36 | PMX157 | SYVLTQLPSKNVTLKQPAHITCGGDKIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 37 | PMX157 | GGSVTSS |
| | CDR1H | |
| 38 | PMX157 | GYWTGS |
| | CDR2H | |
| 39 | PMX157 | CSREGGSTLGAFAYW |
| | CDR3H | |
| 40 | PMX157 | GGDKIGSKSVH |
| | CDR1L | |
| 41 | PMX157 | YDSSRPT |
| | CDR2L | |
| 42 | PMX157 | CQVWDSSAKASVF |
| | CDR3L | |
| 43 | PMX158 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAGTGGATGGGG |
| | | TACTGGACAGGTACCACAAACTACAATCCG |
| | | GCATTTCAGGGACGCATCTCCATCACTGCT |
| | | GACACGGCCAAGAACCAGTGCTCCCTGCAG |
| | | CTGAGCTCCATGACCACCGAGGACACGGCC |
| | | GTGTATTACTGTGCAAGGGAGGGGGGAAAG |
| | | CTACTGGGAGCTTTTGGTCACTGGGGCCAG |
| | | GGCACCCTGGTCACTGTCTCCTCAG |
| 44 | PMX158 | ELTLQESGPGLVKPSQTLSLTCVVSGGSVT |
| | VH_AA | RSYYWNWIRQRPGRGLEWMGYWTGTTNYNP |
| | | AFQGRISITADTAKNQCSLQLSSMTTEDTA |
| | | VYYCAREGGKLLGAFGHWGQGTLVTVSS |
| 45 | PMX158 | TCCTATGTGCTGACACAGCTGCCATCCAAA |
| | VL_NT | AATGTGACCCTGAAGCAGCCGGCCCACATC |
| | | ACCTGTGGGGGAGACAAGATTGGAAGTAAA |
| | | AGTGTTCACTGGTACCAGCAGAAGCTGGGC |
| | | CAGGCCCCTGTACTGATTATCTATTATGAT |
| | | AGCAGCAGGTTGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 46 | PMX158 | SYVLTQLPSKNVTLKQPAHITCGGDKIGSK |
| | VL_AA | SVHWYQQKLGQAPVLIIYYDSSRLTGIPER |
| | | FSGANSGNTATLTISGALAEDEADYYCQVW |
| | | DSSAKASVFGGGTHLTVL |
| 47 | PMX158 | GGSVTRS |
| | CDR1H | |
| 48 | PMX158 | GYWTGT |
| | CDR2H | |
| 49 | PMX158 | CAREGGKLLGAFGHW |
| | CDR3H | |
| 50 | PMX158 | GGDKIGSKSVH |
| | CDR1L | |
| 51 | PMX158 | YDSSRLT |
| | CDR2L | |
| 52 | PMX158 | CQVWDSSAKASVF |
| | CDR3L | |
| 53 | PMX159 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAATGGATGGGG |
| | | TACTGGACAGGTAGCACAATCTACAACCCG |
| | | ACATTCCAGGGACGCATCTCCATCACTGCT |
| | | GACACTGCCAAGAGCCAGTTCTCCCTACAA |
| | | CTGAGGTCCATGACCACCGAGGACACGGCC |
| | | ATGTATTTCTGTGCAAGAGAAGGCGGGAAG |
| | | TCGCTGGGAGCTTTTGCTTACTGGGGCCAG |
| | | GGCACCCTGGTCACTGTCTCCTCAG |
| 54 | PMX159 | ELTLQESGPGLVKPSQTLSLTCVVSGGSVT |
| | VH_AA | RSYYWNWIRQRPGRGLEWMGYWTGSTIYNP |
| | | TFQGRISITADTAKSQFSLQLRSMTTEDTA |
| | | MYFCAREGGKSLGAFAYWGQGTLVTVSS |
| 55 | PMX159 | TCCTATGTGCTGACACAGCTGCCATCCAAA |
| | VL_NT | AATGTGACCCTGAAGCAGCCGGCCCACATC |
| | | ACCTGTGGGGGAGACAAGATTGGAAGTAAA |
| | | AATGTTCACTGGTACCAGCAGAAGCTGGGC |
| | | CAGGCCCCTGTAATGATTATCTATTATGAT |
| | | AGTAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTGCTGTGTTCGGC |
| | | GGAGGCACCCACCTGACCGTCCTCG |
| 56 | PMX159 | SYVLTQLPSKNVTLKQPAHITCGGDKIGSK |
| | VL_AA | NVHWYQQKLGQAPVMIIYYDSSRPTGIPER |
| | | FSGANSGNTATLTISGALAEDEADYYCQVW |
| | | DSSAKAAVFGGGTHLTVL |
| 57 | PMX159 | GGSVTRS |
| | CDR1H | |
| 58 | PMX159 | WTGSTI |
| | CDR2H | |
| 59 | PMX159 | CAREGGKSLGAFAYW |
| | CDR3H | |
| 60 | PMX159 | GGDKIGSKNVH |
| | CDR1L | |
| 61 | PMX159 | YDSSRPT |
| | CDR2L | |
| 62 | PMX159 | CQVWDSSAKAAVF |
| | CDR3L | |
| 63 | PMX160 | GAGGTGCAGATGGTGGAGTCTGGGGGAGAC |
| | VH_NT | CTGGTGAAGCCTGGGGGGTCCCTGAGATTC |
| | | TCTTGTGTGGCCTCTGGATTCACCTTCAGT |
| | | AGCTACCACATGAGCTGGGTCCGCCAGGCT |
| | | CCAGGGAAGGGGCTTCAGTGGGTCGCATAT |
| | | ATTAATAGTGGTGGAAGTAGAACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAAGAC |
| | | ACGGCCGTGTATTATTGTGCGAGTGGGAGA |
| | | AGCCCCCCCCCCCTAGGGAGTTTTGACTAT |
| | | GGTTTGGACTACTGGGGCCATGGCACCTCA |
| | | CTCTTCGTGTCCTCAG |
| 64 | PMX160 | EVQMVESGGDLVKPGGSLRFSCVASGFTFS |
| | VH_AA | SYHMSWVRQAPGKGLQWVAYINSGGSRTSY |
| | | ADAVKGRFTISRDNAKNTLYLQMNSLRAED |
| | | TAVYYCASGRSPPPLGSFDYGLDYWGHGT |
| | | SLFVSS |
| 65 | PMX160 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTATTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 66 | PMX160 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 67 | PMX160 | GFTFSSY |
| | CDR1H | |
| 68 | PMX160 | NSGGSR |
| | CDR2H | |
| 69 | PMX160 | CASGRSPPPLGSFDYGLDYW |
| | CDR3H | |
| 70 | PMX160 | GGDNIGSKSVH |
| | CDR1L | |
| 71 | PMX160 | YDSSRPT |
| | CDR2L | |
| 72 | PMX160 | CQVWDSSAKAWVF |
| | CDR3L | |
| 73 | PMX190 | GAGGTTCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCAAATGAGCTGGGTCCGCCAGGCT |
| | | CCAGGGAAGGGGCTTCAGTGGGTCGCATAC |
| | | ATTAGCAGTGGTGGCGGTAGGACAACCTAT |
| | | GCAGACGCTGTGAGGGGCCGATTAACCATC |
| | | TCCAGAGACAACGCCAAGAACACCCTGTAT |
| | | CTTCAGATGAACAGCCTGAGAGCCGAGGAC |
| | | GCGGCCGTGTATTACTGTGCGCGTGAGGGG |
| | | GGGGGGGAGATATATGGATACAAAGACTAT |
| | | GGTATGGACTACTGGGGCCATGGCACCTCA |
| | | CTCTTCGTGTCCTCAG |
| 74 | PMX190 | EVQLVESGGDLVKPGGSLRLSCVASGFTFS |
| | VH_AA | SYQMSWVRQAPGKGLQWVAYISSGGGRTTY |
| | | ADAVRGRLTISRDNAKNTLYLQMNSLRAED |
| | | AAVYYCAREGGGEIYGYKDYGMDYWGHGTS |
| | | LFVSS |
| 75 | PMX190 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTTTTCGGT |
| | | GAAGGGACCCAGCTGACCGTCCTCG |
| 76 | PMX190 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 77 | PMX190 | GFTFSSY |
| | CDR1H | |
| 78 | PMX190 | SSGGGR |
| | CDR2H | |
| 79 | PMX190 | CAREGGGEIYGYKDYGMDYW |
| | CDR3H | |
| 80 | PMX190 | GGDNIGSKSVH |
| | CDR1L | |
| 81 | PMX190 | YDSSRPT |
| | CDR2L | |
| 82 | PMX190 | CQVWDSSAKAWVF |
| | CDR3L | |
| 83 | PMX162 | GAGATGCAATTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | ACTTACCACATGAGTTGGGTCCGCCAGGCT |
| | | CCAGGGAAGGGGCTTCAGTGGGTCGCATAC |
| | | AGCAATAGTGGTGGAACTAGGACATATTAT |
| | | GCAGACGCTGTGAAGGGCCGATTCACCATC |
| | | TCCAGAGACAACGTCAAGAACACGCTGTAT |
| | | CTTCAGATGAACAGCCTGAGAGCCGAGGAC |
| | | ACGGCCGTGTACTACTGTGCGAGTGAGGGG |
| | | AGTGGGGGGGGGAGATATGGATTTAAAGAC |
| | | TATGGTATGGACTACTGGGGCCATGGCACC |
| | | TCACTCTTCGTGTCCACAG |
| 84 | PMX162 | EMQLVESGGDLVKPGGSLRLSCVASGFTFS |
| | VH_AA | TYHMSWVRQAPGKGLQWVAYSNSGGTRTYY |
| | | ADAVKGRFTISRDNVKNTLYLQMNSLRAED |
| | | TAVYYCASEGSGGGRYGFKDYGMDYWGHG |
| | | TSLFVST |
| 85 | PMX162 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 86 | PMX162 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 87 | PMX162 | GFTFSTY |
| | CDR1H | |
| 88 | PMX162 | NSGGTR |
| | CDR2H | |
| 89 | PMX162 | CASEGSGGGRYGFKDYGMDYW |
| | CDR3H | |
| 90 | PMX162 | GGDNIGSKSVH |
| | CDR1L | |
| 91 | PMX162 | YDSSRPT |
| | CDR2L | |
| 92 | PMX162 | CQVWDSSAKAWVF |
| | CDR3L | |
| 93 | PMX163 | GAACTCGCACTGCAGGAGTCAGGGCCAGGA |
| | VH_NT | CTGGTGAGGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAATGGATGGGT |
| | | TACTGGACAGGTACCACAATCTACAACCCG |
| | | GCATTCCAGGGACGCATCTCTATCACAGTT |
| | | GACACGGCCAAGAATCAGTTCTCCCTGCAT |
| | | CTGATCTCCATGACCACCGAGGACACGGCC |
| | | GTGTATTATTGTGGAAGAGAGGGGGGTTCT |
| | | TCCCTAGGTGCTATTGCTTACTGGGGCCAG |
| | | GGCACCCGGGTCACTGTCTCCTCAG |
| 94 | PMX163 | ELALQESGPGLVRPSQTLSLTCVVSGGSVT |
| | VH_AA | RSYYWNWIRQRPGRGLEWMGYWTGTTIYNP |
| | | AFQGRISITVDTAKNQFSLHLISMTTEDTA |
| | | VYYCGREGGSSLGAIAYWGQGTRVTVSS |
| 95 | PMX163 | TCCTATGTGTTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTG |
| | | ACCATCAGCGGGGCCCTGGCCGAGGACGAG |
| | | GCTGACTATTACTGCCAGGTGTGGGACAGC |
| | | AGTGCTAAGGCTGCTGTGTTCGGCGGAGGC |
| | | ACCCACCTGACCGTCCTCG |
| 96 | PMX163 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAAVFGGGTHLTVL |
| 97 | PMX163 | GGSVTRS |
| | CDR1H | |
| 98 | PMX163 | GYWTGT |
| | CDR2H | |
| 99 | PMX163 | CGREGGSSLGAIAYW |
| | CDR3H | |
| 100 | PMX163 | GGDNIGSKSVH |
| | CDR1L | |
| 101 | PMX163 | YDSSRPT |
| | CDR2L | |
| 102 | PMX163 | CQVWDSSAKAAVF |
| | CDR3L | |
| 103 | PMX189 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AACAATTACTACTGGAACTGGATCCGCCAG |
| | | CGCCCTGGGAGGGGACTGGAATGGATGGGT |
| | | TACTGGACAGGTAGCACAAAGTATAACCCG |
| | | GCATTCCAGGGACGCATCTCCATCACTGCT |
| | | GACACGGCCAACAACCAGTTCTCTCTGCAC |
| | | CTGAACTCCATGAGTTCCGAGGACACGGCC |
| | | GTTTATTACTGTGCAAGAGATCGAGATCGG |
| | | CGAGTCCCACATGCTTATGGTTACTGGGGC |
| | | CAGGGCACCCTGGTCACTGTCTCCTCAG |
| 104 | PMX189 | ELTLQESGPGLVKPSQTLSLTCVVSGGSVT |
| | VH_AA | NNYYWNWIRQRPGRGLEWMGYWTGSTKYNP |
| | | AFQGRISITADTANNQFSLHLNSMSSEDTA |
| | | VYYCARDRDRRVPHAYGYWGQGTLVTVSS |
| 105 | PMX189 | TCCTATGTGCTGACACAGCTGCCATCCAAA |
| | VL_NT | AATGTGACCCTTAAACAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTATTGCCAGGTGTGG |
| | | GACAGCAGTGCTAAGGCTAGTGTGTTCGGC |
| | | GGAGGCACCCATCTGACCGTCCTCG |
| 106 | PMX189 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 107 | PMX189 | GGSVTNN |
| | CDR1H | |
| 108 | PMX189 | GYWTGS |
| | CDR2H | |
| 109 | PMX189 | CARDRDRRVPHAYGYW |
| | CDR3H | |
| 110 | PMX189 | GGDNIGSKSVH |
| | CDR1L | |
| 111 | PMX189 | YDSSRPT |
| | CDR2L | |
| 112 | PMX189 | CQVWDSSAKASVF |
| | CDR3L | |
| 113 | PMX165 | GAGGTGCAGCTGGTGGAGTCTGGGGGAGAC |
| | VH_NT | CTGGTGAAGCCGGGGGGGTCCCTGAGACTT |
| | | TCCTGTGTGGCCTCTGGATTCAACTTCAGT |
| | | GCCTCCCACATGAGCTGGGTCCGCCAGGCT |
| | | CCAGGGAAGGGGCTTCAGTGGGTCGCATAC |
| | | ATTACCAATGCTGGAGGTAGAATAAGTTAT |
| | | GCCGACGCTGTGAAGGGCCGATTCACCATC |
| | | TCCAGAGACAACGCCAAGAACACGCTATAT |
| | | CTTCAGATGAGCAGCCTGAGAGTCGAAGAC |
| | | ACGGCCGTGTATTATTGTGCGACTGAAAGG |
| | | CCCGGACGGCGGATGGGTCTAAAGGACTAT |
| | | GGTATGGACTACTGGGGCCATGGCACCTCA |
| | | CTCTTCGTGTCCTCAG |
| 114 | PMX165 | EVQLVESGGDLVKPGGSLRLSCVASGFNFS |
| | VH_AA | ASHMSWVRQAPGKGLQWVAYITNAGGRISY |
| | | ADAVKGRFTISRDNAKNTLYLQMSSLRVED |
| | | TAVYYCATERPGRRMGLKDYGMDYWGHGTS |
| | | LFVSS |
| 115 | PMX165 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 116 | PMX165 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 117 | PMX165 | GFNFSAS |
| | CDR1H | |
| 118 | PMX165 | TNAGGR |
| | CDR2H | |
| 119 | PMX165 | CATERPGRRMGLKDYGMDYW |
| | CDR3H | |
| 120 | PMX165 | GGDNIGSKSVH |
| | CDR1L | |
| 121 | PMX165 | YDSSRPT |
| | CDR2L | |
| 122 | PMX165 | CQVWDSSAKASVF |
| | CDR3L | |
| 123 | PMX166 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGCAGTTACTTCTGGAACTGGATCCGCCAG |
| | | CGCCCTGGGAGGGGACTGGAATGGATGGGG |
| | | TACTGGACAGGTAGCACAATCTACAGCCCG |
| | | GCATTCCAGGGACGCATCTCCATCACTCCT |
| | | GACACGGCCAAGAACCAGTTCTCCCTGCAG |
| | | CTGAGCTCCATGACCACCGAGGACACGGCC |
| | | GTGTATTACTGTTCAAGAGAGGGAGGTAGC |
| | | ACCTTGGGGGCTTTTGCTTACTGGGGCCAG |
| | | GGCACCCTGGTCACTGTCTCCTCAG |
| 124 | PMX166 | ELTLQESGPGLVKPSQTLSLTCVVSGGSVT |
| | VH_AA | SSYFWNWIRQRPGRGLEWMGYWTGSTIYSP |
| | | AFQGRISITPDTAKNQFSLQLSSMTTEDTA |
| | | VYYCSREGGSTLGAFAYWGQGTLVTVSS |
| 125 | PMX166 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 126 | PMX166 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 127 | PMX166 | GGSVTSS |
| | CDR1H | |
| 128 | PMX166 | GYWTGS |
| | CDR2H | |
| 129 | PMX166 | CSREGGSTLGAFAYW |
| | CDR3H | |
| 130 | PMX166 | GGDNIGSKSVH |
| | CDR1L | |
| 131 | PMX166 | YDSSRPT |
| | CDR2L | |
| 132 | PMX166 | CQVWDSSAKASVF |
| | CDR3L | |
| 133 | PMX167 | GAGGTGAAGTTGGTGGAGTCTGGGGGAGAC |
| | VH_NT | CTGGTGAAGCCTGGGGGGTCCCTGAGACTT |
| | | TCCTGTATGGCCTCTGGATTCACCCTCAAT |
| | | AATTACTACATGAGTTGGGTCCGCCAGGCT |
| | | CCAGGGAAGGGGCTTCAGTGGGTCGCATAC |
| | | ATTAAAAGTGGTGGAAGTGACACAAGTTAT |
| | | GTAGACGCTGTGAAGGGCCGATTCACCATC |
| | | TCCAGAGACAATGCCAAGAACACGCTGCAT |
| | | CTTCAGATGAACAGCCTGAGAGTCGAGGA |
| | | CACGGCCGTATATTATTGTGCGACTGACGG |
| | | ATATATATGGGCCTGGGGCCAGGGAACCCT |
| | | GGTCACCGTCTCCTCAG |
| 134 | PMX167 | EVKLVESGGDLVKPGGSLRLSCMASGFTLN |
| | VH_AA | NYYMSWVRQAPGKGLQWVAYIKSGGSDTSY |
| | | VDAVKGRFTISRDNAKNTLHLQMNSLRVED |
| | | TAVYYCATDGYIWAWGQGTLVTVSS |
| 135 | PMX167 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AACAGCAGGCCGACAGGGATCCCTGAGCGA |
| | | TTCTCCGGCGCCAAGTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTTCTAAGGCTTACGTGTTCGGC |
| | | TCAGGAACCCAACTGACCGTCCTCG |
| 136 | PMX167 | SYVLTQLPSKNVTLKQPAHITCGGDNIGSK |
| | VL_AA | SVHWYQQKLGQAPVLIIYYDNSRPTGIPER |
| | | FSGAKSGNTATLTISGALAEDEADYYCQVW |
| | | DSSSKAYVFGSGTQLTVL |
| 137 | PMX167 | GFTLNNY |
| | CDR1H | |
| 138 | PMX167 | KSGGSD |
| | CDR2H | |
| 139 | PMX167 | CATDGYIWAW |
| | CDR3H | |
| 140 | PMX167 | GGDNIGSKSVH |
| | CDR1L | |
| 141 | PMX167 | YDNSRPT |
| | CDR2L | |
| 142 | PMX167 | CQVWDSSSKAYVF |
| | CDR3L | |
| 143 | PMX168 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAATGGATGGGG |
| | | TACTGGACAGGTACCACAAACTACAATCCG |
| | | GCATTTCAGGGACGCATCTCCATCACTGCT |
| | | GACACGGCCAAGAACCAGTGCTCCCTGCAG |
| | | CTGAGCTCCATGACCACCGAGGACACGGCC |
| | | GTTTATTATTGTGCAAGGGAGGGGGGAAAG |
| | | CTACTGGGAGCTTTTGGTCACTGGGGCCAG |
| | | GGCACCCTGGTCACTGTCTCCTCAG |
| 144 | PMX168 | ELTLQESGPGLVKPSQTLSLTCVVSGGSVT |
| | VH_AA | RSYYWNWIRQRPGRGLEWMGYWTGTTNYNP |
| | | AFQGRISITADTAKNQCSLQLSSMTTEDTA |
| | | VYYCAREGGKLLGAFGHWGQGTLVTVSS |
| 145 | PMX168 | TCCTATGTGCTGACACAGCTGCCATCCAAA |
| | VL_NT | AATGTGACCCTGAAGCAGCCGGCCCACATC |
| | | ACCTGTGGGGGAGACAAGATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 146 | PMX168 | SYVLTQLPSKNVTLKQPAHITCGGDKIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 147 | PMX168 | GGSVTRS |
| | CDR1H | |
| 148 | PMX168 | GYWTGT |
| | CDR2H | |
| 149 | PMX168 | CAREGGKLLGAFGHW |
| | CDR3H | |
| 150 | PMX168 | GGDKIGSKSVH |
| | CDR1L | |
| 151 | PMX168 | YDSSRPT |
| | CDR2L | |
| 152 | PMX168 | CQVWDSSAKASVF |
| | CDR3L | |
| 153 | PMX169 | GAGGTGCAGGTGGTGGAGTCTGGGGGAGAC |
| | VH_NT | CTGGTGAAGCCTGGGGGGTCCCTGAGACTT |
| | | TCCTGTGTGACCTCTGGATTCGCCTTCAAT |
| | | AACTACCACATGAGCTGGGTCCGCCAGGCT |
| | | CCAGGGAAGGGGCTTCAGTGGGTCGCATAC |
| | | ATTAACAGTGGTGGAAGTAGAATAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTTTATTACTGTGCGAGTGAGGGG |
| | | GGGGGGGAGATATATGGATTCAAAGACTAT |
| | | GGTATGGACTACTGGGGCCATGGCACCTCA |
| | | CTCTTCGTGTCCTCAG |
| 154 | PMX169 | EVQVVESGGDLVKPGGSLRLSCVTSGFAFN |
| | VH_AA | NYHMSWVRQAPGKGLQWVAYINSGGSRISY |
| | | ADAVKGRFTISRDNAKNTLYLQMNSLRAED |
| | | TAVYYCASEGGGEIYGFKDYGMDYWGHGTS |
| | | LFVSS |
| 155 | PMX169 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 156 | PMX169 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 157 | PMX169 | GFAFNNY |
| | CDR1H | |
| 158 | PMX169 | NSGGSR |
| | CDR2H | |
| 159 | PMX169 | CASEGGGEIYGFKDYGMDYW |
| | CDR3H | |
| 160 | PMX169 | GGDNIGSKSVH |
| | CDR1L | |
| 161 | PMX169 | YDSSRPT |
| | CDR2L | |
| 162 | PMX169 | CQVWDSSAKAWVF |
| | CDR3L | |
| 163 | PMX170 | GAGGTGCAGTTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGTTACCACATGAGCTGGGTCCGCCAGGCT |
| | | CCAGGGAAGGGGCTTCAGTGGGTCGCATAC |
| | | AGTAACAGTGGTGGAAGTAGGACAAGTTAT |
| | | GCAGACGCTGTGAAGGGCCGATTCACCATC |
| | | TCCAGAGACAACGCCAAGAACACGCTGTAT |
| | | CTTCAGATGAGCAGCCTGAGAGCCGAGGAC |
| | | ACGGCCGTATACTACTGTGCGAGTGAGGGG |
| | | GGGGGGGGGAGATATGGATTCAAAGACTAT |
| | | GGTATGGACTACTGGGGCCATGGCACCTCA |
| | | CTCTTCGTGTCCTCAG |
| 164 | PMX170 | EVQLVESGGDLVKPGGSLRLSCVASGFTFS |
| | VH_AA | SYHMSWVRQAPGKGLQWVAYSNSGGSRTSY |
| | | ADAVKGRFTISRDNAKNTLYLQMSSLRAED |
| | | TAVYYCASEGGGGRYGFKDYGMDYWGHGT |
| | | SLFVSS |
| 165 | PMX170 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 166 | PMX170 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 167 | PMX170 | GFTFSSY |
| | CDR1H | |
| 168 | PMX170 | NSGGSR |
| | CDR2H | |
| 169 | PMX170 | CASEGGGGRYGFKDYGMDYW |
| | CDR3H | |
| 170 | PMX170 | GGDNIGSKSVH |
| | CDR1L | |
| 171 | PMX170 | YDSSRPT |
| | CDR2L | |
| 172 | PMX170 | CQVWDSSAKAWVF |
| | CDR3L | |
| 173 | PMX171 | GAGGTGCAGCTGGTGGAGTCTGGGGGAGAC |
| | VH_NT | CTGGTGAAGCCTGGGGGGTCCCTGAGACTT |
| | | TCCTGTGAGGCCTCTGGATTCACCTTCAGT |
| | | AGCTACCATATGAGTTGGGTCCGCCAGGCT |
| | | CCAGGGAAGGGGCTTCAGTGGGTCGCATAC |
| | | ATTAACAATGGTGGAAGTGGTACAAGCTAT |
| | | GCAGACGCTGTGAAGGGCCGATTCACCATC |
| | | TCCAGAGACAACGCCAAGAACACGCTCTAT |
| | | CTTCAGATGAACAGCCTGCGAGCCGAGGAC |
| | | ACGGCCGTTTATTTCTGTGCGAGTGAGGGG |
| | | GGGGGGAAGAGATATGGATACAAAGACTAT |
| | | GGTATGGACTACTGGGGCCATGGCACCTCA |
| | | CTCTTCGTGTCCTCAG |
| 174 | PMX171 | EVQLVESGGDLVKPGGSLRLSCEASGFTFS |
| | VH_AA | SYHMSWVRQAPGKGLQWVAYINNGGSGTSY |
| | | ADAVKGRFTISRDNAKNTLYLQMNSLRAED |
| | | TAVYFCASEGGGKRYGYKDYGMDYWGHGTS |
| | | LFVSS |
| 175 | PMX171 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 176 | PMX171 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 177 | PMX171 | GFTFSSY |
| | CDR1H | |
| 178 | PMX171 | NNGGSG |
| | CDR2H | |
| 179 | PMX171 | CASEGGGKRYGYKDYGMDYW |
| | CDR3H | |
| 180 | PMX171 | GGDNIGSKSVH |
| | CDR1L | |
| 181 | PMX171 | YDSSRPT |
| | CDR2L | |
| 182 | PMX171 | CQVWDSSAKAWVF |
| | CDR3L | |
| 183 | PMX172 | GAGGTGCAGTTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGTTACCACATGAGCTGGGTCCGCCAGGCT |
| | | CCAGGGAAGGGGCTTCAGTGGGTCGCATAC |
| | | AGTAACAGTGGTGGAAGTAGGACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTACTACTGTGCGAGTGAGGGG |
| | | GGGGGGGGGAGATATGGATACAAAGACTAT |
| | | GGTATGGACTACTGGGGCCATGGCACCTCA |
| | | CTCTTCGTGTCCTCAG |
| 184 | PMX172 | EVQLVESGGDLVKPGGSLRLSCVASGFTFS |
| | VH_AA | SYHMSWVRQAPGKGLQWVAYSNSGGSRTSY |
| | | ADAVKGRFTISRDNAKNTLYLQMNSLRAED |
| | | TAVYYCASEGGGGRYGYKDYGMDYWGHGT |
| | | SLFVSS |
| 185 | PMX172 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 186 | PMX172 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 187 | PMX172 | GFTFSSY |
| | CDR1H | |
| 188 | PMX172 | NSGGSR |
| | CDR2H | |
| 189 | PMX172 | CASEGGGGRYGYKDYGMDYW |
| | CDR3H | |
| 190 | PMX172 | GGDNIGSKSVH |
| | CDR1L | |
| 191 | PMX172 | YDSSRPT |
| | CDR2L | |
| 192 | PMX172 | CQVWDSSAKAWVF |
| | CDR3L | |
| 193 | PMX173 | GAGGTGCAATTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | ACTTACCACATGAGTTGGGTCCGCCAGGCT |
| | | CCAGGGAAGGGGCTTCAGTGGGTCGCCTAC |
| | | AGCCATAGTGGTGGAACTAGGACATATTAT |
| | | GCAGACGCTGTGAAGGGCCGATTCACCATC |
| | | TCCAGAGACAACGGCAAGAACACGCTGTAT |
| | | CTTCAGATGAACAGCCTGAGAGCCGAGGAC |
| | | ACGGCCGTGTACTACTGTGCGAGTGAGGGG |
| | | AGTGGGGGGGGGAGATATGGATTTAAAGAC |
| | | TATGGTATGGACTACTGGGGCCATGGCACC |
| | | TCACTCTTCGTGTCCACAG |
| 194 | PMX173 | EVQLVESGGDLVKPGGSLRLSCVASGFTFS |
| | VH_AA | TYHMSWVRQAPGKGLQWVAYSHSGGTRTYY |
| | | ADAVKGRFTISRDNGKNTLYLQMNSLRAED |
| | | TAVYYCASEGSGGGRYGFKDYGMDYWGHGT |
| | | SLFVST |
| 195 | PMX173 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 196 | PMX173 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 197 | PMX173 | GFTFSTY |
| | CDR1H | |
| 198 | PMX173 | HSGGTR |
| | CDR2H | |
| 199 | PMX173 | CASEGSGGGRYGFKDYGMDYW |
| | CDR3H | |
| 200 | PMX173 | GGDNIGSKSVH |
| | CDR1L | |
| 201 | PMX173 | YDSSRPT |
| | CDR2L | |
| 202 | PMX173 | CQVWDSSAKAWVF |
| | CDR3L | |
| 203 | PMX174 | GAGGTGCAGCTGGTGGAGTCTGGGGGAGAC |
| | VH_NT | CTGGTGAAGGCTGGGGTGTCCCTGAGACTT |
| | | TCCTGTGTGGCCTCTGGATTCACCTTCAGT |
| | | AACTATCACTTGAACTGGGTCCGCCAGGCT |
| | | CCAGGGAAGGGGCCTCAGTGGGTCGCATCC |
| | | ATTAACAGTGGTGGAAGTAGCACATACTAT |
| | | GCAGACGCTGTGAAGGGCCGATTCACCATC |
| | | TCCAGAGACAACGCCAAGAACACGCTTTAT |
| | | CTTCAGATGAACAGCCTGAGAGCCGAGGAC |
| | | ACGGCCGTGTATTACTGTGCGAGTGACCAC |
| | | GGTAGCTACCGGGGTATGGACTATTGGGGC |
| | | CATGGCACCTCACTCTTCGTGTCCTCAG |
| 204 | PMX174 | EVQLVESGGDLVKAGVSLRLSCVASGFTFS |
| | VH_AA | NYHLNWVRQAPGKGPQWVASINSGGSSTYY |
| | | ADAVKGRFTISRDNAKNTLYLQMNSLRAED |
| | | TAVYYCASDHGSYRGMDYWGHGTSLFVSS |
| 205 | PMX174 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAATTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTGTGTTCGGCGGA |
| | | GGCACCCATCTGACCGTCCTCG |
| 206 | PMX174 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAVFGGGTHLTVL |
| 207 | PMX174 | GFTFSNY |
| | CDR1H | |
| 208 | PMX174 | NSGGSS |
| | CDR2H | |
| 209 | PMX174 | CASDHGSYRGMDYW |
| | CDR3H | |
| 210 | PMX174 | GGDNIGSKSVH |
| | CDR1L | |
| 211 | PMX174 | YDSSRPT |
| | CDR2L | |
| 212 | PMX174 | CQVWDSSAKAVF |
| | CDR3L | |
| 213 | PMX175 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGTACTTACTACTGGAACTGGATCCGCCAG |
| | | CGCCCTGGGAGGGGACTGGAATGGATGGGG |
| | | TACTGGACAGGTACCACAAACTACAACCCG |
| | | GCATTCCAGGGACGCATCTCCATCACTGCT |
| | | GACACGGCCAAGAACCAGTTCTCCCTGCAG |
| | | CTGAGTTCCATGACCACCGAGGACACGGCC |
| | | GTATATTACTGTGCAAGAGAAGATTTAGTA |
| | | ACAGTTGGTTCCGGTGCTTTTAATTACTGG |
| | | GGCCAGGGCACCCTGGTCACTGTCTCCTCA |
| | | G |
| 214 | PMX175 | ELTLQESGPGLVKPSQTLSLTCVVSGGSVT |
| | VH_AA | STYYWNWIRQRPGRGLEWMGYWTGTTNYNP |
| | | AFQGRISITADTAKNQFSLQLSSMTTEDTA |
| | | VYYCAREDLVTVGSGAFNYWGQGTLVTVSS |
| 215 | PMX175 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTACTTGGGTATTCGGTGAA |
| | | GGGACCCAGCTGACCGTCCTCG |
| 216 | PMX175 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSATWVFGEGTQLTVL |
| 217 | PMX175 | GGSVTST |
| | CDR1H | |
| 218 | PMX175 | GYWTGT |
| | CDR2H | |
| 219 | PMX175 | CAREDLVTVGSGAFNYW |
| | CDR3H | |
| 220 | PMX175 | GGDNIGSKSVH |
| | CDR1L | |
| 221 | PMX175 | YDSSRPT |
| | CDR2L | |
| 222 | PMX175 | CQVWDSSATWVF |
| | CDR3L | |
| 223 | PMX176 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGGTTCACCTTCAGT |
| | | AGCTACCACATGAGCTGGGTCCGCCAGGCT |
| | | CCAGGGAAGGGGCTTCAGTGGGTCGCATAC |
| | | ATTAACAGTGCTGGAAGTACCACAAGCTAT |
| | | ACAGACGCTGTGCAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTGAGTGG |
| | | ATATTTATGCCCTGGGGCCAGGGATCCCTG |
| | | GTCACCGTCTCCTCAG |
| 224 | PMX176 | EVQLVESGGDLVKPGGSLRLSCVASGFTFS |
| | VH_AA | SYHMSWVRQAPGKGLQWVAYINSAGSTTSY |
| | | TDAVQGRFTISRDNAKNTLYLQMNSLRAE |
| | | DTAVYYCASEWIFMPWGQGSLVTVSS |
| 225 | PMX176 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAG |
| | | CAGAAGCTGGGCCAGGCCCCTGTACTGATT |
| | | ATCTATTATGATAGCAGCAGGCCGACAGGG |
| | | ATCCCTGAGCGATTCTCCGGCGCCAACTCG |
| | | GGGAACACGGCCACCCTGACCATCAGCGGG |
| | | GCCCTGGCCGAGGACGAGGCTGACTATTAC |
| | | TGCCAGGTGTGGGACAGCAGTGCTAAGGCT |
| | | AGGGTATTCGGTGAAGGGACCCAGCTGACC |
| | | GTCCTCG |
| 226 | PMX176 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKARVFGEGTQLTVL |
| 227 | PMX176 | GFTFSSY |
| | CDR1H | |
| 228 | PMX176 | NSAGST |
| | CDR2H | |
| 229 | PMX176 | CASEWIFMPW |
| | CDR3H | |
| 230 | PMX176 | GGDNIGSKSVH |
| | CDR1L | |
| 231 | PMX176 | YDSSRPT |
| | CDR2L | |
| 232 | PMX176 | CQVWDSSAKARVF |
| | CDR3L | |
| 233 | PMX177 | GAAGTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGCAGTTTTTACTGGAACTGGATCCGCCAG |
| | | CGCCCTGGGAGGGGACTGGAATGGATGGGA |
| | | TACTGGACAGGTAGCACAAATTACAACCCG |
| | | GCATTCCAGGGACGCATCTCCATCACTGCT |
| | | GACACGGCCAAGAACCAGTTCTCCCTGCAC |
| | | CTGGGCTCCATGACCACCGATGACACGGCC |
| | | ATGTATTACTGTGCAAGAGAGGAGGGTAGT |
| | | TGGGGGAGGGTCTATTTTGACTACTGGGGC |
| | | CAGGGAACCCTGGTCACCGTCTCCTCAG |
| 234 | PMX177 | EVTLQESGPGLVKPSQTLSLTCVVSGGSVT |
| | VH_AA | SSFYWNWIRQRPGRGLEWMGYWTGSTNYNP |
| | | AFQGRISITADTAKNQFSLHLGSMTTDDTA |
| | | MYYCAREEGSWGRVYFDYWGQGTLVTVSS |
| 235 | PMX177 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTACTAAGACTTGGGTATTCGGT |
| | | GAAGGGACCCAGCTGACCGTCCTCG |
| 236 | PMX177 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSTKTWVFGEGTQLTVL |
| 237 | PMX177 | GGSVTSS |
| | CDR1H | |
| 238 | PMX177 | WTGSTN |
| | CDR2H | |
| 239 | PMX177 | CAREEGSWGRVYFDYW |
| | CDR3H | |
| 240 | PMX177 | GGDNIGSKSVH |
| | CDR1L | |
| 241 | PMX177 | YDSSRPT |
| | CDR2L | |
| 242 | PMX177 | CQVWDSSTKTWVF |
| | CDR3L | |
| 243 | PMX178 | GAACTCGCACTGCAGGAGTCAGGGCCAGGA |
| | VH_NT | CTGGTGAGGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAATGGATGGGT |
| | | TACTGGACAGGTACCACAATCTACAACCCG |
| | | GCATTCCAGGGACGCATCTCCATCACAGTT |
| | | GACACGGCCAAGAATCAGTTCTCCCTGCAT |
| | | CTGATCTCCATGACCACCGAGGACACGGCC |
| | | GTGTATTATTGTGGAAGAGAGGGGGGTTCT |
| | | TCCCTAGGTGCTATTGCTTACTGGGGCCAG |
| | | GGCACCCGGGTCACTGTCTCCTCAG |
| 244 | PMX178 | ELALQESGPGLVRPSQTLSLTCVVSGGSVT |
| | VH_AA | RSYYWNWIRQRPGRGLEWMGYWTGTTIYNP |
| | | AFQGRISITVDTAKNQFSLHLISMTTEDTA |
| | | VYYCGREGGSSLGAIAYWGQGTRVTVSS |
| 245 | PMX178 | TCCTATGTGTTGACACAGCTGCCATCCAAA |
| | VL_NT | AATGTGACCCTGAAGCAGCCGGCCCACATC |
| | | ACCTGTGGGGGAGACAACATTGGAAGAAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTGCTGTGTTCGGC |
| | | GGAGGCACCCACCTGACCGTCCTCG |
| 246 | PMX178 | SYVLTQLPSKNVTLKQPAHITCGGDNIGRK |
| | VL_AA | SVHWYQQKLGQAPVLIIYYDSSRPTGIPER |
| | | FSGANSGNTATLTISGALAEDEADYYCQVW |
| | | DSSAKAAVFGGGTHLTVL |
| 247 | PMX178 | GGSVTRS |
| | CDR1H | |
| 248 | PMX178 | GYWTGT |
| | CDR2H | |
| 249 | PMX178 | CGREGGSSLGAIAYW |
| | CDR3H | |
| 250 | PMX178 | GGDNIGRKSVH |
| | CDR1L | |
| 251 | PMX178 | YDSSRPT |
| | CDR2L | |
| 252 | PMX178 | CQVWDSSAKAAVF |
| | CDR3L | |
| 253 | PMX179 | GAACTCGCACTGCAGGAGTCAGGGCCAGGA |
| | VH_NT | CTGGTGAGGCCCTCACAGACCCTCTCTCTC |
| | | ACCTGTGTTGTGTCCGGAGGCTCCGTCACC |
| | | AGAAGTTACTACTGGAACTGGATCCGCCAG |
| | | CGCCCTGGGAGGGGACTGGAATGGATGGGT |
| | | TACTGGACAGGTACCACAATCTACAACCCG |
| | | GCATTCCAGGGACGCATCTCCATCACAGTT |
| | | GACACGGCCAAGAATCAGTTCTCCCTGCAT |
| | | CTGATCTCCATGACCACCGAGGACACGGCC |
| | | GTGTATTATTGTGGAAGAGAGGGGGGTTCT |
| | | TCCCTAGGTGCTATTGCTTACTGGGGCCAG |
| | | GGCACCCGGGTCACTGTCTCCTCAG |
| 254 | PMX179 | ELALQESGPGLVRPSQTLSLTCVVSGGSVT |
| | VH_AA | RSYYWNWIRQRPGRGLEWMGYWTGTTIYNP |
| | | AFQGRISITVDTAKNQFSLHLISMTTEDTA |
| | | VYYCGREGGSSLGAIAYWGQGTRVTVSS |
| 255 | PMX179 | TCCTATGTGTTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTGCTGTGTTCGGC |
| | | GGAGGCACCCACCTGACCGTCCTCG |
| 256 | PMX179 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAAVFGGGTHLTVL |
| 257 | PMX179 | GGSVTRS |
| | CDR1H | |
| 258 | PMX179 | GYWTGT |
| | CDR2H | |
| 259 | PMX179 | CGREGGSSLGAIAYW |
| | CDR3H | |
| 260 | PMX179 | GGDNIGSKSVH |
| | CDR1L | |
| 261 | PMX179 | YDSSRPT |
| | CDR2L | |
| 262 | PMX179 | CQVWDSSAKAAVF |
| | CDR3L | |
| 263 | PMX180 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGCAATTACTACTGGAACTGGATCCGCCAG |
| | | CGCCCTGGGAGGGGACTGGAATGGATGGGG |
| | | TACTGGACAGGTAGCACAAACTACAACCCG |
| | | GCATTCCAGGGACGCATCTCCATCACTGCT |
| | | GACACGGCCAAGAAGCAGTTCTCCCTGCAG |
| | | CTGAGGTCCATGACCATTGAAGACACGGCC |
| | | GTGTATTACTGTGCAAGAAGAGGAATACCT |
| | | ATATTTTTTGACTACTGGGGCCAGGGAACC |
| | | CTGGTCACCGTCTCCTCAG |
| 264 | PMX180 | ELTLQESGPGLVKPSQTLSLTCVVSGGSVT |
| | VH_AA | SNYYWNWIRQRPGRGLEWMGYWTGSTNYNP |
| | | AFQGRISITADTAKKQFSLQLRSMTIEDTA |
| | | VYYCARRGIPIFFDYWGQGTLVTVSS |
| 265 | PMX180 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAGTGTGTTCGGCGGAGGC |
| | | ACCCATCTGACCGTCCTCG |
| 266 | PMX180 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSASVFGGGTHLTVL |
| 267 | PMX180 | GGSVTSN |
| | CDR1H | |
| 268 | PMX180 | GYWTGS |
| | CDR2H | |
| 269 | PMX180 | CARRGIPIFFDYW |
| | CDR3H | |
| 270 | PMX180 | GGDNIGSKSVH |
| | CDR1L | |
| 271 | PMX180 | YDSSRPT |
| | CDR2L | |
| 272 | PMX180 | CQVWDSSASVF |
| | CDR3L | |
| 273 | PMX181 | GAACTCACACTGCAGGAGTCAGGGCCAGGA |
| | VH_NT | CTGGTGAAGCCCTCACAGACCCTCTCTCTC |
| | | ACCTGTATTGTGTCCGGAGGCTCCGTCACC |
| | | AACAATTACTACTGGACCTGGATCCGCCAG |
| | | CGCCCTGGGAGGGGACTGGAATGGATGGGA |
| | | TACTGGACAGGTTTCACAAACTACAATCCG |
| | | GCATTCCAGGGACGCATCTCCATCATTGCT |
| | | AACACGGCCAAGAAACAATTGTCCCTGCAG |
| | | CTGAGCTCCTTGACCACCGAAGACACGGCC |
| | | GTATATTACTGTGCAAGAAGGGCGATACCT |
| | | ACCTATTTTGACTACTGGGGCCAGGGAACC |
| | | CTAGTCACCGTCTCCTCCG |
| 274 | PMX181 | ELTLQESGPGLVKPSQTLSLTCIVSGGSVT |
| | VH_AA | NNYYWTWIRQRPGRGLEWMGYWTGFTNYNP |
| | | AFQGRISIIANTAKKQLSLQLSSLTTEDTA |
| | | VYYCARRAIPTYFDYWGQGTLVTVSS |
| 275 | PMX181 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAGA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAGTGTGTTCGGCGGAGGC |
| | | ACCCATCTGACCGTCCTCG |
| 276 | PMX181 | SYVLTQLPSKNVTLKQPAHITCGGDNIGSR |
| | VL_AA | SVHWYQQKLGQAPVLIIYYDSSRPTGIPER |
| | | FSGANSGNTATLTISGALAEDEADYYCQVW |
| | | DSSASVFGGGTHLTVL |
| 277 | PMX181 | GGSVTNNYYW |
| | CDR1H | |
| 278 | PMX181 | FHKLQSGI |
| | CDR2H | |
| 279 | PMX181 | CARRAIPTYFDYW |
| | CDR3H | |
| 280 | PMX181 | GGDNIGSRSVH |
| | CDR1L | |
| 281 | PMX181 | YDSSRPT |
| | CDR2L | |
| 282 | PMX181 | CQVWDSSASVF |
| | CDR3L | |
| 283 | PMX182 | GAGGTGCAGCTGGTGGAGTCTGGGGGAGAC |
| | VH_NT | CTGGTGAAGCCTGGGGGGTCCCTGAGACTT |
| | | TCCTGTGTGACATCTGGATTCACCTTCAGT |
| | | AACTATCACATGAATTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCATGTATTACTGTGCGAGTGACTGG |
| | | GACTACTGTGCTGATGATACCTGTGAGGGT |
| | | ATGGGCTACTGGGGCCATGGCACCTCACTC |
| | | TTCGTGTCCTCAG |
| 284 | PMX182 | EVQLVESGGDLVKPGGSLRLSCVTSGFTFS |
| | VH_AA | NYHMNWVRQAPGKGLQWVAYINSGGSSTSY |
| | | ADAVKGRFTISRDNAKNTLYLQMNSLRAED |
| | | TAMYYCASDWDYCADDTCEGMGYWGHGTSL |
| | | FVSS |
| 285 | PMX182 | TCCTATGTGCTGACACAGCTGCCATCCAAA |
| | VL_NT | AATGTGACCCTGAAACAGCCGGCCCACATC |
| | | ACCTGTGGGGGAGACAATATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 286 | PMX182 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 287 | PMX182 | GFTFSNY |
| | CDR1H | |
| 288 | PMX182 | NSGGSS |
| | CDR2H | |
| 289 | PMX182 | CASDWDYCADDTCEGMGYW |
| | CDR3H | |
| 290 | PMX182 | GGDNIGSKSVH |
| | CDR1L | |
| 291 | PMX182 | YDSSRPT |
| | CDR2L | |
| 292 | PMX182 | CQVWDSSAKAWVF |
| | CDR.3L | |
| 293 | PMX183 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AACAGTTACTACTGGAACTGGATCCGCCAG |
| | | CGCCCTGGGCGGGAACTGGAATGGATGGGG |
| | | TACTGGACAGGTGGCACAAAGTACAACCCG |
| | | GCATTCCAGGGACGCATCTCCATCAGTGCT |
| | | GACACGGCCAAGAATCAGTTCTCCCTGCAG |
| | | CTGACCTCCATGACCACCGAGGACACGGCC |
| | | GTGTATTACTGTGCAAGAGATCGAGATCGG |
| | | AGAGTCCCACATGCTTATGGTTACTGGGGC |
| | | CAGGGCACCCTGGTCACTGTCTCCTCAG |
| 294 | PMX183 | ELTLQESGPGLVKPSQTLSLTCVVSGGSVT |
| | VH_AA | NSYYWNWIRQRPGRELEWMGYWTGGTKYNP |
| | | AFQGRISISADTAKNQFSLQLTSMTTEDTA |
| | | VYYCARDRDRRVPHAYGYWGQGTLVTVSS |
| 295 | PMX183 | TCCTATGTACTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 296 | PMX183 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKASVFGGGTHLTVL |
| 297 | PMX183 | GGSVT |
| | CDR1H | |
| 298 | PMX183 | GYWTGG |
| | CDR2H | |
| 299 | PMX183 | CARDRDRRVPHAYGYW |
| | CDR3H | |
| 300 | PMX183 | GGDNIGSKSVH |
| | CDR1L | |
| 301 | PMX183 | YDSSRPT |
| | CDR2L | |
| 302 | PMX183 | CQVWDSSAKASVF |
| | CDR3L | |
| 303 | PMX184 | GAACTCACACTGCAGGAGTCAGGGCCAGGA |
| | VH_NT | CTGGTGAAGCCCTCACAGACCCTCTCTCTC |
| | | ACCTGTGGTGTGTCCGGAGGCTCCGTCACC |
| | | AGAAGTCACTACTGGAATTGGGTCCGCCAG |
| | | CGCCCTGGGAGGGGACTGGAATGGATGGGA |
| | | TACTGGACAGGTAGCACAAACTACAACCCG |
| | | GCATTCCAGGGACGCATCTCCATCACTGCT |
| | | GACACGGCCAAGAACCAGTTCTCCCTGCAG |
| | | CTGAGGTCCATGACCACCGAGGACACGGCC |
| | | GTATATTTCTGTGCAAGAGAGACTACAATA |
| | | CCTACTTTTTTTTTTGACTACTGGGGCCAG |
| | | GGAACCCTGGTCACCGTCTCCTCAG |
| 304 | PMX184 | ELTLQESGPGLVKPSQTLSLTCGVSGGSVT |
| | VH_AA | RSHYWNWVRQRPGRGLEWMGYWTGSTNYNP |
| | | AFQGRISITADTAKNQFSLQLRSMTTEDTA |
| | | VYFCARETTIPTFFFDYWGQGTLVTVSS |
| 305 | PMX184 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCGGTGCTAAGGCTTGGGTTTTCGGT |
| | | GAAGGGACCCAGCTGACCGTCCTCG |
| 306 | PMX184 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSGAKAWVFGEGTQLTVL |
| 307 | PMX184 | GGSVTRS |
| | CDR1H | |
| 308 | PMX184 | GYWTGS |
| | CDR2H | |
| 309 | PMX184 | CARETTIPTFFFDYW |
| | CDR3H | |
| 310 | PMX184 | GGDNIGSKSVH |
| | CDR1L | |
| 311 | PMX184 | YDSSRPT |
| | CDR2L | |
| 312 | PMX184 | CQVWDSGAKAWVF |
| | CDR3L | |
| 313 | PMX185 | GAGGTGCAGCTGGTGGAGTCTGGGGGAGAC |
| | VH_NT | CTGGTGAAGCCTGGGGGGTCCCTGAGACTG |
| | | TCCTGTGTGGCCTCTGGATTCACCTTCAGT |
| | | AGCTACCACATGAGTTGGGTCCGTCAGGCT |
| | | CCAGGGAAGGGGCTTCAATGGGTCGCATCC |
| | | ATTAACAGTGGTGGAAGTAGAACAAGCTAT |
| | | GTAGACGCTGTGAAGGGCCGATTCACCATC |
| | | TCCAGAGACAACGCCAAGAAAACGCTGTAT |
| | | CTTCAGATGAATAGCCTGAGAGCCGAGGAC |
| | | ACGGCCGTTTATTACTGTGCGAGTGGGCGA |
| | | ACCGGGCCCAAGTTGGGACTAGGAGACTAT |
| | | GGTATGGACTCCTGGGGCCATGGCACCTCA |
| | | CTCTTCGTGTCCTCAG |
| 314 | PMX185 | EVQLVESGGDLVKPGGSLRLSCVASGFTFS |
| | VH_AA | SYHMSWVRQAPGKGLQWVASINSGGSRTSY |
| | | VDAVKGRFTISRDNAKKTLYLQMNSLRAED |
| | | TAVYYCASGRTGPKLGLGDYGMDSWGHGTS |
| | | LFVSS |
| 315 | PMX185 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTAATGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 316 | PMX185 | SYVLTQLPSKNVTLKQPAHITCGGDNIGSK |
| | VL_AA | SVHWYQQKLGQAPVMIIYYDSSRPTGIPER |
| | | FSGANSGNTATLTISGALAEDEADYYCQVW |
| | | DSSAKAWVFGEGTQLTVL |
| 317 | PMX185 | GFTFSSY |
| | CDR1H | |
| 318 | PMX185 | NSGGSR |
| | CDR2H | |
| 319 | PMX185 | CASGRTGPKLGLGDYGMDSW |
| | CDR3H | |
| 320 | PMX185 | GGDNIGSKSVH |
| | CDR1L | |
| 321 | PMX185 | YDSSRPT |
| | CDR2L | |
| 322 | PMX185 | CQVWDSSAKAWVF |
| | CDR3L | |
| 323 | PMX186 | GAGGTGAAGCTGGTGGAGTCTGGGGGAGAC |
| | VH_NT | CTGGTGAAGCCTGGGGGGTCCCTGAGACTT |
| | | TCCTGTGTGGCCTCTGGAGTCACCTTCAGT |
| | | AGGTATTACATGAGCTGGGTCCGCCAGGCT |
| | | CCAGGGAAGGGGCTTCAGTGGGTCGCATAC |
| | | ATTAACAGTGGTGGAAGTACCACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTGAGGGG |
| | | GTGGGGGAGATATATGGATACAAAGACTTT |
| | | GGTATGGACTACTGGGGCCATGGCACCTCA |
| | | CTCTTCGTGTCCTCAG |
| 324 | PMX186 | EVKLVESGGDLVKPGGSLRLSCVASGVTFS |
| | VH_AA | RYYMSWVRQAPGKGLQWVAYINSGGSTTSY |
| | | ADAVKGRFTISRDNAKNTLYLQMNSLRAED |
| | | TAVYYCASEGVGEIYGYKDFGMDYWGHGT |
| | | SLFVSS |
| 325 | PMX186 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGAAGGCCGACAGGGATCCCAGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 326 | PMX186 | SYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSAKAWVFGEGTQLTVL |
| 327 | PMX186 | GVTFSRY |
| | CDR1H | |
| 328 | PMX186 | NSGGST |
| | CDR2H | |
| 329 | PMX186 | CASEGVGEIYGYKDFGMDYW |
| | CDR3H | |
| 330 | PMX186 | GGDNIGSKSVH |
| | CDR1L | |
| 331 | PMX186 | YDSRRPT |
| | CDR2L | |
| 332 | PMX186 | CQVWDSSAKAWVF |
| | CDR3L | |
| 333 | PMX187 | GAGGTGCAGCTGGTGGAGTCTGGGGGAGAC |
| | VH_NT | CTGGTGAAGCCTGGGGGGTCCCTGAGACTG |
| | | TCCTGTGTGGCCTCTGGATTCACCTTCAGT |
| | | AGCTACCACATGAGTTGGGTCCGTCAGGCT |
| | | CCAGGGAAGGGGCTTCAGTGGGTCGCATCC |
| | | ATTAACAGTGGTGGAAGTAGAACAAGCTAT |
| | | GTAGACGCTGTGAAGGGCCGCTTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTGGGCGA |
| | | ACCGGGCCCAAGTTGGGGCTAGGAGACTAT |
| | | GGTATGGACTCCTGGGGCCATGGCACCTCA |
| | | CTCTTCGTGTCCTCAG |
| 334 | PMX187 | EVQLVESGGDLVKPGGSLRLSCVASGFTFS |
| | VH_AA | SYHMSWVRQAPGKGLQWVASINSGGSRTSY |
| | | VDAVKGRFTISRDNAKNTLYLQMNSLRAED |
| | | TAVYYCASGRTGPKLGLGDYGMDSWGHGT |
| | | SLFVSS |
| 335 | PMX187 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GCAGGGACCCAGCTGACCGTCCTCG |
| 336 | PMX187 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGAGTQLTVL |
| 337 | PMX187 | GFTFSSY |
| | CDR1H | |
| 338 | PMX187 | NSGGSR |
| | CDR2H | |
| 339 | PMX187 | CASGRTGPKLGLGDYGMDSW |
| | CDR3H | |
| 340 | PMX187 | GGDNIGSKSVH |
| | CDR1L | |
| 341 | PMX187 | YDSSRPT |
| | CDR2L | |
| 342 | PMX187 | CQVWDSSAKAWVF |
| | CDR3L | |
| 343 | PMX188 | GAGGTGCAGCTGGTGGAGTCTGGGGGAGAC |
| | VH_NT | CTGGTGAAGCCTGGGGGGTCCCTGAGACTG |
| | | TCCTGTGTGGCCTCTGGATTCACCTTCAGT |
| | | AGCTACCACATGAGTTGGGTCCGTCAGGCT |
| | | CCAGGGAAGGGGCTTCAATGGGTCGCATCC |
| | | ATTAACAGTGGTGGAAGTAGAACAAGCTAT |
| | | GTAGACGCTGTGAAGGGCCGCTTCACTATC |
| | | TCCAGAGACAACGCCAAGAAAACGCTGTAT |
| | | CTTCAGATGAATAGCCTGAGAGCCGAGGAC |
| | | ACGGCCGTTTATTACTGTGCGAGTGGGCGA |
| | | ACCGGGCCCAAGTTGGGACTAGGAGACTAT |
| | | GGTATGGACTCCTGGGGCCATGGCACCTCA |
| | | CTCTTCGTGTCCTCAG |
| 344 | PMX188 | EVQLVESGGDLVKPGGSLRLSCVASGFTFS |
| | VH_AA | SYHMSWVRQAPGKGLQWVASINSGGSRTSY |
| | | VDAVKGRFTISRDNAKKTLYLQMNSLRAED |
| | | TAVYYCASGRTGPKLGLGDYGMDSWGHGT |
| | | SLFVSS |
| 345 | PMX188 | TCCTATGTGCTGACACAGCTGCCATCCAAA |
| | VL_NT | AATGTCACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | AGCAGTAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 346 | PMX188 | SYVLTQLPSKNVTLKQPAHITCGGDNIGSK |
| | VL_AA | SVHWYQQKLGQAPILIIYYDSSRPTGIPER |
| | | FSGANSGNTATLTISGALAEDEADYYCQVW |
| | | DSSAKAWVFGEGTQLTVL |
| 347 | PMX188 | GFTFSSY |
| | CDR1H | |
| 348 | PMX188 | NSGGSR |
| | CDR2H | |
| 349 | PMX188 | CASGRTGPKLGLGDYGMDSW |
| | CDR3H | |
| 350 | PMX188 | GGDNIGSKSVH |
| | CDR1L | |
| 351 | PMX188 | YDSSRPT |
| | CDR2L | |
| 352 | PMX188 | CQVWDSSAKAWVF |
| | CDR3L | |
| 353 | PMX190 | GAGGTTCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCAAATGAGCTGGGTCCGCCAGGCT |
| | | CCAGGGAAGGGGCTTCAGTGGGTCGCATAC |
| | | ATTAGCAGTGGTGGCGGTAGGACAACCTAT |
| | | GCAGACGCTGTGAGGGGCCGATTAACCATC |
| | | TCCAGAGACAACGCCAAGAACACCCTGTAT |
| | | CTTCAGATGAACAGCCTGAGAGCCGAGGAC |
| | | GCGGCCGTGTATTACTGTGCGCGTGAGGGG |
| | | GGGGGGGAGATATATGGATACAAAGACTAT |
| | | GGTATGGACTACTGGGGCCATGGCACCTCA |
| | | CTCTTCGTGTCCTCAG |
| 354 | PMX190 | EVQLVESGGDLVKPGGSLRLSCVASGFTFS |
| | VH_AA | SYQMSWVRQAPGKGLQWVAYISSGGGRTTY |
| | | ADAVRGRLTISRDNAKNTLYLQMNSLRAED |
| | | AAVYYCAREGGGEIYGYKDYGMDYWGHGTS |
| | | LFVSS |
| 355 | PMX190 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTG |
| | | ACCATCAGCGGGGCCCTGGCCGAGGACGAG |
| | | GCTGACTATTACTGCCAGGTGTGGGACAGC |
| | | AGTGCTAAGGCTTGGGTTTTCGGTGAAGGG |
| | | ACCCAGCTGACCGTCCTCG |
| 356 | PMX190 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 357 | PMX190 | GFTFSSY |
| | CDR1H | |
| 358 | PMX190 | SSGGGR |
| | CDR2H | |
| 359 | PMX190 | CAREGGGEIYGYKDYGMDYW |
| | CDR3H | |
| 360 | PMX190 | GGDNIGSKSVH |
| | CDR1L | |
| 361 | PMX190 | YDSSRPT |
| | CDR2L | |
| 362 | PMX190 | CQVWDSSAKAWVF |
| | CDR3L | |
| 363 | PMX191 | GAGGTGCAACTGGTGGAGTCTGGGGGAGAC |
| | VH_NT | CTGGTGAAGCCTGCGGGGTCCCTGAGACTG |
| | | TCCTGTGTGGCCTCTGGATTCACCTTCAGT |
| | | GACTACAGTATGAGCTGGGTCCGCCAGGGT |
| | | CCTGAGAAGGGACTACAGTTGGTCGCAGGT |
| | | ATGAACAGCGATGGCAGTACCACATACTAC |
| | | ACAGACGCTGTGAGGGGCCGATTCACCATC |
| | | TCCAGAGACAACGCCAAGAACACAATGTAT |
| | | CTGCAGATGAACAGCCTGAGAGACGAGGAC |
| | | ACGGCCATTTATTACTGTGCAAAGGGGGCA |
| | | GCTGCCCCTGACTACTGGGGCCAGGGAACC |
| | | CTGGTCACCGTCTCCTCAA |
| 364 | PMX191 | EVQLVESGGDLVKPAGSLRLSCVASGFTFS |
| | VH_AA | DYSMSWVRQGPEKGLQLVAGMNSDGSTTYY |
| | | TDAVRGRFTISRDNAKNTMYLQMNSLRDED |
| | | TAIYYCAKGAAAPDYWGQGTLVTVSS |
| 365 | PMX191 | TCCTATGAGCTGACTCAGCCACCATCCGTG |
| | VL_NT | AATGTGACCCTGAGGGAGACGGCCCACATC |
| | | ACCTGTGGGGGAGACAGAATTGGAAGTAAA |
| | | TATGTTCAATGGATCCAGCAGATTCCAGGC |
| | | CAGGCCCCCGTGGTGATTATCTATAAAGAT |
| | | AACAACCGGCCGACAGGGATCCCTGAGCGA |
| | | TTCTCTGGCGCCAACTCAGGGAACACGGCT |
| | | ACCCTGACCATCAGTGGGGCCCTGGCCGAA |
| | | GACGAGGCTGACTATTACTGCCAGGTGGGG |
| | | GACAATTATACTTGGGTATTCGGTGAAGGG |
| | | ACCCAGCTGACCGTCCTCG |
| 366 | PMX191 | SYELTQPPSVNVTLRETAHITCGGDRIGSK |
| | VL_AA | YVQWIQQIPGQAPVVIIYKDNNRPTGIPER |
| | | FSGANSGNTATLTISGALAEDEADYYCQVG |
| | | DNYTWVFGEGTQLTVL |
| 367 | PMX191 | GFTFSDY |
| | CDR1H | |
| 368 | PMX191 | NSDGST |
| | CDR2H | |
| 369 | PMX191 | CAKGAAAPDYW |
| | CDR3H | |
| 370 | PMX191 | GGDRIGSKYVQ |
| | CDR1L | |
| 371 | PMX191 | KDNNRPT |
| | CDR2L | |
| 372 | PMX191 | CQVGDNYTWVF |
| | CDR3L | |
| 373 | PMX192 | GAGGTGCAGTTGGTGGAGTCTGGGGGAGAC |
| | VH_NT | CTGGTGAAGCCTGCGGGGTCCCTGAGACTG |
| | | TCCTGTGTGGCCTCTGGATTCACCTTCAGT |
| | | GACTACAGCATGAGCTGGGTCCGCCAGGCT |
| | | CCTGAGAAGGGACTGCAGTTGGTCGCAGGT |
| | | ATTAACGGCGATGGAAGTACCACATACTAC |
| | | ACAGACGCTGTGAAGGGCCGATTCACCATC |
| | | TCCAGAGACAACGCCAAGAACACAGTCTAT |
| | | CTACAGATGAACAGTCTGAGAGCCGAGGAC |
| | | ACGGCCATGTATTACTGTGCAAAGGGAGCT |
| | | GGTACTGGTGACTTTTGGGGCCAGGGAACC |
| | | CTGGTCACCGTCTCCTCAG |
| 374 | PMX192 | EVQLVESGGDLVKPAGSLRLSCVASGFTFS |
| | VH_AA | DYSMSWVRQAPEKGLQLVAGINGDGSTTYY |
| | | TDAVKGRFTISRDNAKNTVYLQMNSLRAED |
| | | TAMYYCAKGAGTGDFWGQGTLVTVSS |
| 375 | PMX192 | TCCTATGAGCTGACTCAGCCACCATCCGTG |
| | VL_NT | AATGTGACCCTGAGGGAGACGGCCCACATC |
| | | ACCTGTGGGGGAGCCAGCATTGGAAGAAAA |
| | | TATGTTCAATGGATCCAGCAGATTCCAGGC |
| | | CAGGCCCCCGTGGTGATTATCTATAAAGAT |
| | | AGCAACAGGCCGACAGGGATCCCTGAGCGA |
| | | TTCTCTGGCGCCAACTCAGGGAACACGGCT |
| | | ACCCTGACCATCAGTGGGGCCCTGGCCGAA |
| | | GACGAGGCTGACTATTACTGCCAGGTGGGG |
| | | GACAGTGGTGCTTGGGTTTTCGGTGAAGGG |
| | | ACCCAGTTGACCGTCCTCG |
| 376 | PMX192 | SYELTQPPSVNVTLRETAHITCGGASIGRK |
| | VL_AA | YVQWIQQIPGQAPVVIIYKDSNRPTGIPER |
| | | FSGANSGNTATLTISGALAEDEADYYCQVG |
| | | DSGAWVFGEGTQLTVL |
| 377 | PMX192 | GFTFSDY |
| | CDR1H | |
| 378 | PMX192 | NGDGST |
| | CDR2H | |
| 379 | PMX192 | CAKGAGTGDFW |
| | CDR3H | |
| 380 | PMX192 | GGASIGRKYVQ |
| | CDR1L | |
| 381 | PMX192 | KDSNRPT |
| | CDR2L | |
| 382 | PMX192 | CQVGDSGAWVF |
| | CDR3L | |
| 383 | PMX193 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | ACCTACCACATGAGCTGGGTCCGCCAGGCT |
| | | CCAGGGAAGGGCCTTCAGTGGGTCGCATAC |
| | | ATTAGCAGTGGTGGAAGTCGCACAAACTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | GCGGCCGTGTTTTACTGTGCGAGGCACTAC |
| | | GGTAGCTCTCTTTTTGACTACTGGGGCCAG |
| | | GGAACCCTGGTCACCGTCTCCTCAG |
| 384 | PMX193 | EVQLVESGGDLVKPGGSLRLSCVASGFTFS |
| | VH_AA | TYHMSWVRQAPGKGLQWVAYISSGGSRTNY |
| | | ADAVKGRFTISRDNAKNTLYLQMNSLRAED |
| | | AAVFYCARHYGSSLFDYWGQGTLVTVSS |
| 385 | PMX193 | GATATTGTCATGACACAGACCCCACTGTCC |
| | VL_NT | CTGTCCGTCAGCCCTGGAGAGCCGGCCTCC |
| | | ATCTCCTGCAAGGCCAGTCAGAGCCTCCTG |
| | | CACATTAATGGGAACACCTATTTGTATTGG |
| | | TTCCGACAGAAGCCAGGCCAGTCTCCACAG |
| | | CGTTTGATCTATAAGGTCTCCAACAGAGAC |
| | | CCTGGGGTCCCAGACAGGTTCAGTGGCAGC |
| | | GGGTCAGGGACAGATTTCACCCTGAGAATC |
| | | AGCAGAGTGGAGGCTGATGATGCTGGAGTT |
| | | TATTACTGCGGGCAAGGTATACAAGATCCT |
| | | ACCTTTGGCAAAGGGACACATCTGGAGATT |
| | | AAAC |
| 386 | PMX193 | DIVMTQTPLSLSVSPGEPASISCKASQSLL |
| | VL_AA | HINGNTYLYWFRQKPGQSPQRLIYKVSNRD |
| | | PGVPDRESGSGSGTDFTLRISRVEADDAGV |
| | | YYCGQGIQDPTFGKGTHLEIK |
| 387 | PMX193 | GFTFSTY |
| | CDR1H | |
| 388 | PMX193 | SSGGSR |
| | CDR2H | |
| 389 | PMX193 | CARHYGSSLFDYW |
| | CDR3H | |
| 390 | PMX193 | CKASQSLLHIN |
| | CDR1L | |
| 391 | PMX193 | PQRLIYK |
| | CDR2L | |
| 392 | PMX193 | CGQGIQDPTF |
| | CDR3L | |
| 393 | PMX194 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AACTACCACATGAGTTGGGTCCGCCAGGCT |
| | | CCAGGGAAGGGGCTTCAGTGGGTCGCATAC |
| | | AGTAACAGTGGTAGAATGAGCACAGACTAT |
| | | GCAGACGCTGTGAAGGGCCGATTCACCATC |
| | | TCCAGAGACAATGCCAAGAACACGCTGTAT |
| | | CTTCAGATGAACAGCCTGAGACCCGAGGAC |
| | | ACGGCCGTGTATTACTGTGCGACTGTCATA |
| | | TATGGCCTTTTTGACTACTGGGGCCAGGGA |
| | | ACCCTGGTCACCGTCTCCTCAG |
| 394 | PMX194 | EVQLVESGGDLVKPGGSLRLSCVASGFTFS |
| | VH_AA | NYHMSWVRQAPGKGLQWVAYSNSGRMSTDY |
| | | ADAVKGRFTISRDNAKNTLYLQMNSLRPED |
| | | TAVYYCATVIYGLFDYWGQGTLVTVSS |
| 395 | PMX194 | GATATTGTCATGACACAGACCCCACTGTCC |
| | VL_NT | CTGTCCGTCAGCCCTGGAGAGCCGGCCTCC |
| | | ATCTCCTGCAAGGCCAGTCAGAGCCTCCTG |
| | | TACAGTAATGGGAACACCTATTTGTATTGG |
| | | TTCCGACAGAAGCCAGGCCAGTCTCCACAG |
| | | CGTTTGATCTATAAGGTCTCCAAGAGAGAC |
| | | TCTGGGGTCCCAGACAGGTTCAGTGGCAGC |
| | | GGGTCAGGGACAGATTTCACCCTGAGAATC |
| | | AGAAGAGTGGAGGCTGATGATGCTGGAGTT |
| | | TATTACTGCGGGCAAGGTACACAAGATCCT |
| | | TATACTTTCAGCCAGGGAACCAAGCTGGAG |
| | | ATAAAAC |
| 396 | PMX194 | DIVMTQTPLSLSVSPGEPASISCKASQSLL |
| | VL_AA | YSNGNTYLYWFRQKPGQSPQRLIYKVSKRD |
| | | SGVPDRFSGSGSGTDFTLRIRRVEADDAGV |
| | | YYCGQGTQDPYTFSQGTKLEIK |
| 397 | PMX194 | GFTFSNY |
| | CDR1H | |
| 398 | PMX194 | NSGRMS |
| | CDR2H | |
| 399 | PMX194 | CATVIYGLFDYW |
| | CDR3H | |
| 400 | PMX194 | CKASQSLLYSN |
| | CDR1L | |
| 401 | PMX194 | PQRLIYK |
| | CDR2L | |
| 402 | PMX194 | CGQGTQDPYTF |
| | CDR3L | |
| 403 | PMX195 | GAGGTGCAGTTGGTGGAGTCTGGGGGAGAC |
| | VH_NT | CTGGTGAAGCCTGCCGGGTCCCTGAGACTG |
| | | TCCTGTATGGCCTCTGGATTCACCTTCAGT |
| | | AGTTACAGTATGAGCTGGGTCCGCCAGGCT |
| | | CCTGAGAAGGGGCTGCAGTTGGTCGCAGGT |
| | | ATTAATGGCGATGGAAGTACCACATACTAC |
| | | ACAGACGCTGTGAAGGGCCGCTTCACCATC |
| | | TCCAGAGACAACGCCAAAAACACAGTATAT |
| | | CTGCAGATGAACAACCTGAGAGTCGAGGAC |
| | | TCGGCCATGTATTATTGTGCAAAAGGGGCT |
| | | GGTACGGGAGACTACTGGGGCCAGGGAACC |
| | | CTGGTCACCGTCTCCTCAG |
| 404 | PMX195 | EVQLVESGGDLVKPAGSLRLSCMASGFTFS |
| | VH_AA | SYSMSWVRQAPEKGLQLVAGINGDGSTTYY |
| | | TDAVKGRFTISRDNAKNTVYLQMNNLRVED |
| | | SAMYYCAKGAGTGDYWGQGTLVTVSS |
| 405 | PMX195 | TCCTATGAGTTGACTCAGCCACCATCCTTG |
| | VL_NT | AATGTGACCCTGAGGGAGACGGCCCACATC |
| | | ACCTGTGGGGGAGACAGGATTGGAAGTAGA |
| | | TATGTTCAGTGGATCCAGCAGAATCCAGGC |
| | | CAGGCCCCCGTGGTGGTAATCTATAAAGAT |
| | | TCCAACAGGCCGACAGGGATCCCTGAGCGA |
| | | TTCTCTGGCGCCAACTCAGGGAACACGGCA |
| | | ACCCTGACCATCAGTGGGGCCCTGGCCGAA |
| | | GACGAGGCTGACTATTATTGCCAGGTGGGG |
| | | GACAGTGGTAATTGGGTATTCGGTGGAGGG |
| | | ACCCAGCTGACCGTCCTCG |
| 406 | PMX195 | SYELTQPPSLNVTLRETAHITCGGDRIGSR |
| | VL_AA | YVQWIQQNPGQAPVVVIYKDSNRPTGIPER |
| | | FSGANSGNTATLTISGALAEDEADYYCQVG |
| | | DSGNWVFGGGTQLTVL |
| 407 | PMX195 | GFTFSSY |
| | CDR1H | |
| 408 | PMX195 | NGDGST |
| | CDR2H | |
| 409 | PMX195 | CAKGAGTGDYW |
| | CDR3H | |
| 410 | PMX195 | GGDRIGSRYVQ |
| | CDR1L | |
| 411 | PMX195 | KDSNRPT |
| | CDR2L | |
| 412 | PMX195 | CQVGDSGNWVF |
| | CDR3L | |
| 413 | PMX196 | GAACTCACACTGCAGGAGTCAGGGCCAGGA |
| | VH_NT | CTGGTGAAGCCCTCACAGACCCTCTCTCTC |
| | | ACCTGTGTTGTGTCCGGAGGCTCCGTCACC |
| | | AGCAGTTACTACTGGAACTGGATCCGCCAG |
| | | CGCCCTGGGAGGGGACTGGAATGGATGGGT |
| | | TACTGGACAGGTAGCACAATCTACAACCCG |
| | | GCATTTCAGGGACGCATCTCCATTACTGCT |
| | | GACAC |
| | | GGCCAAGAACCAGTTCTCCCTGCAACTGAG |
| | | TTCCATGACCACCGAGGACACGGCCGTATA |
| | | TTTCTGTGTAAGAGAGAGGCGGATGGCTTT |
| | | TGGTTCCTGGGGCCAGGGCACCCTGGTCAT |
| | | TGTCTCCTCAG |
| 414 | PMX196 | ELTLQESGPGLVKPSQTLSLTCVVSGGSVT |
| | VH_AA | SSYYWNWIRQRPGRGLEWMGYWTGSTIYNP |
| | | AFQGRISITADTAKNQFSLQLSSMTTEDTA |
| | | VYFCVRERRMAFGSWGQGTLVIVSS |
| 415 | PMX196 | TCCTATGTGCTGACACAGCTGCCATCCAAA |
| | VL_NT | AGTGTGACCCTGAAGCAGCCGGCCCACATC |
| | | ACCTGTGGAGGAGACAACATTGGAAGAAAA |
| | | AGTGTTCACTGGTACCAGCAGAAGCTGGGC |
| | | CAGGCCCCTGTACTGATTATCTCTTTTGAT |
| | | AACAACAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCA |
| 416 | PMX196 | SYVLTQLPSKSVTLKQPAHITCGGDNIGRK |
| | VL_AA | SVHWYQQKLGQAPVLIISFDNNRPTGIPER |
| | | FSGANSGNTATLTISGALAEDEADYYCQVW |
| | | DSSAKASVFGGGTHLTVL |
| 417 | PMX196 | GGSVTSS |
| | CDR1H | |
| 418 | PMX196 | GYWTGS |
| | CDR2H | |
| 419 | PMX196 | CVRERRMAFGSW |
| | CDR3H | |
| 420 | PMX196 | GGDNIGRKSVH |
| | CDR1L | |
| 421 | PMX196 | FDNNRPT |
| | CDR2L | |
| 422 | PMX196 | CQVWDSSAKASVF |
| | CDR3L | |
| 423 | PMX197 | GAACTCACACTGCAGGAGTCAGGGCCAGGA |
| | VH_NT | CTGGTGAAGCCCTCACAGACCCTCTCTCTC |
| | | ACCTGTCTTGTGTCCGGAGGCTCCGTCACC |
| | | AGGAGTTATTACTGGAATTGGATCCGTCAG |
| | | CGCCCTGGGAGGGGACTGGAATGGATGGGA |
| | | TATTGGACAGGCAGCACAAATTACAATCCG |
| | | GCATTCCAGGGACGCATCTCCATCATTCCT |
| | | GACACGGCCAAGAACCAGTTCTCCCTGCAA |
| | | CTGACCTCCATGACCACCGACGACACGGCC |
| | | ATATATTTCTGTGCAAGAGAGGGGGGTCGA |
| | | ACCCTAGGTGCTTTTGCTGACTGGGGTCAG |
| | | GGCACCCTGGTCACTGTCTCCTCAG |
| 424 | PMX197 | ELTLQESGPGLVKPSQTLSLTCLVSGGSVT |
| | VH_AA | RSYYWNWIRQRPGRGLEWMGYWTGSTNYNP |
| | | AFQGRISIIPDTAKNQFSLQLTSMTTDDTA |
| | | IYFCAREGGRTLGAFADWGQGTLVTVSS |
| 425 | PMX197 | TCCTATGTGCTGACACAGCTGCCATCCAAA |
| | VL_NT | AATGTGACCCTGAAGCAGCCGGCCCACATC |
| | | ACCTGTGGGGGAGACAAAATTGGAAGTAAA |
| | | AATGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCA |
| 426 | PMX197 | SYVLTQLPSKNVTLKQPAHITCGGDKIGSK |
| | VL_AA | NVHWYQQKLGQAPVLIIYYDSSRPTGIPER |
| | | FSGANSGNTATLTISGALAEDEADYYCQVW |
| | | DSSAKASVFGGGTHLTVL |
| 427 | PMX197 | GGSVTRS |
| | CDR1H | |
| 428 | PMX197 | WTGSTN |
| | CDR2H | |
| 429 | PMX197 | CAREGGRTLGAFADW |
| | CDR3H | |
| 430 | PMX197 | GGDKIGSKNVH |
| | CDR1L | |
| 431 | PMX197 | YDSSRPT |
| | CDR2L | |
| 432 | PMX197 | CQVWDSSAKASVF |
| | CDR3L | |
| 433 | PMX198 | GAATTCACACTGCAGGAGTCAGGGCCAGGA |
| | VH_NT | CTGGTGAAGCCCTCACAGACCCTCTCTCTC |
| | | ACCTGTATTGTGTCCGGAGGCTCCGTCACC |
| | | AGCAGTTACTACTGGAACTGGATCCGCCAG |
| | | CGCCCTGGGAGGGGACTGGAATGGATGGGG |
| | | TACTGGACAGGTAGCACAAACTACAACCCG |
| | | GCATTCCAGGGACGCATCTCCATCACTCCT |
| | | GACACGGCCAAGAACCAGTTCTCCCTGCAG |
| | | CTGAGCTCCATGACCACCGAGGACACGGCC |
| | | GTGTATTATTGTGCAAGAGAGGGGGGGAGT |
| | | AGTCTTCAAGCTTTTGGTAACTGGGGCCAG |
| | | GGCACCCTGGTCACTGTCTCCTCAG |
| 434 | PMX198 | EFTLQESGPGLVKPSQTLSLTCIVSGGSVT |
| | VH_AA | SSYYWNWIRQRPGRGLEWMGYWTGSTNYNP |
| | | AFQGRISITPDTAKNQFSLQLSSMTTEDTA |
| | | VYYCAREGGSSLQAFGNWGQGTLVTVSS |
| 435 | PMX198 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGAAAA |
| | | AGTGTTCACTGGTACCAGCAGAAGCTGGGC |
| | | CAGGCCCCTATACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTAGTGTGTTCGGC |
| | | GGAGGCACCCATCTGACCGTCCTCG |
| 436 | PMX198 | SYVLTQLPSKNVTLKQPAHITCGGDNIGRK |
| | VL_AA | SVHWYQQKLGQAPILIIYYDSSRPTGIPER |
| | | FSGANSGNTATLTISGALAEDEADYYCQVW |
| | | DSSAKASVFGGGTHLTVL |
| 437 | PMX198 | GGSVTSS |
| | CDR1H | |
| 438 | PMX198 | GYWTGS |
| | CDR2H | |
| 439 | PMX198 | CAREGGSSLQAFGNW |
| | CDR3H | |
| 440 | PMX198 | GGDNIGRKSVH |
| | CDR1L | |
| 441 | PMX198 | YDSSRPT |
| | CDR2L | |
| 442 | PMX198 | CQVWDSSAKASVF |
| | CDR3L | |
| 443 | PMX200 | GAACTCACACTGCAGGAGTCAGGGCCAGGA |
| | VH_NT | CTGGTGAAGCCCTCACAGACCCTCTCTCTC |
| | | ACCTGTATTGTGTCCGGAGGCTCCGTCACC |
| | | AGCAGTTACTACTGGAACTGGATCCGCCAG |
| | | AGCCCTGGGAGGGGACTGGAATGGATGGGT |
| | | TACTGGACAGGAAATACAAACTACAACCCG |
| | | GCACTCCAGGGACGCATCTTCATCACTGCT |
| | | GACACGGCCAAGAACCAGTTCTCACTGCAG |
| | | CTGAGGTCCATGACCACCGAGGACACGGCC |
| | | GTGTATTACTGTGCAAGAGGGGGATATGGA |
| | | TACCACTATGGTATGGACTACTGGGGCCAT |
| | | GGCACGTCAGTCTTCGTGTCCTCAG |
| 444 | PMX200 | ELTLQESGPGLVKPSQTLSLTCIVSGGSVT |
| | VH_AA | SSYYWNWIRQSPGRGLEWMGYWTGNTNYNP |
| | | ALQGRIFITADTAKNQFSLQLRSMTTEDTA |
| | | VYYCARGGYGYHYGMDYWGHGTSVFVSS |
| 445 | PMX200 | TCCTATGTGCTGACACAGCTGCCGTCCAAA |
| | VL_NT | AGTGTGGCCCTGAAGCAGCCGGCCCACATC |
| | | ACCTGTGGGGGAGACAACATTGGAAGTAAA |
| | | CGTGTTCACTGGTACCAGCAGAAGCTGGGC |
| | | CAGGCCCCTGCACTGATTATCTCTTTTGAT |
| | | AATAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCACTGCTAAGGCTAGTATTTTCGGC |
| | | GGAGGCACCCATCTGACCGTCCTCG |
| 446 | PMX200 | SYVLTQLPSKSVALKQPAHITCGGDNIGSK |
| | VL_AA | RVHWYQQKLGQAPALIISFDNSRPTGIPER |
| | | FSGANSGNTATLTISGALAEDEADYYCQVW |
| | | DSTAKASIFGGGTHLTVL |
| 447 | PMX200 | GGSVTS |
| | CDR1H | |
| 448 | PMX200 | GYWTGN |
| | CDR2H | |
| 449 | PMX200 | CARGGYGYHYGMDYW |
| | CDR3H | |
| 450 | PMX200 | GGDNIGSKRVH |
| | CDR1L | |
| 451 | PMX200 | FDNSRPT |
| | CDR2L | |
| 452 | PMX200 | CQVWDSTAKASIF |
| | CDR3L | |
| 453 | PMX272 | GAGGTGCAGCTGGTGGAGTCTGGAGGCGAC |
| | VH_NT | CTGGTGAAGCCAGGAGGAAGCCTGAGGCTG |
| | | TCTTGCGTGGCTTCCGGCTTCACCTTTTCC |
| | | AGATACTATATGAACTGGGTGCGCCAGGCT |
| | | CCTGGCAAGGGACTGCAGTGGGTGGCCTAC |
| | | ATCGACAGCGACGGCTCCAGCACATCTTAT |
| | | GCCGACGCTGTGAAGGGCAGGTTCACCATC |
| | | AGCCGGGATAACGCCAAGAATACACTGTAC |
| | | CTGCAGATGAACTCCCTGAGAGCTGACGAT |
| | | ACCGCCGTGTACTATTGTACAAGCTTTGAT |
| | | TATTGGGGCCAGGGCACCCTGGTCACCGTC |
| | | TCCTCAG |
| 454 | PMX272 | EVQLVESGGDLVKPGGSLRLSCVASGFTFS |
| | VH_AA | RYYMNWVRQAPGKGLQWVAYIDSDGSSTSY |
| | | ADAVKGRFTISRDNAKNTLYLQMNSLRADD |
| | | TAVYYCTSFDYWGQGTLVTVSS |
| 455 | PMX272 | TCCTATGTGCTGACACAGCTGCCATCCAAA |
| | VL_NT | AGCGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 456 | PMX272 | SYVLTQLPSKSVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 457 | PMX272 | GFTFSRY |
| | CDR1H | |
| 458 | PMX272 | DSDGSS |
| | CDR2H | |
| 459 | PMX272 | CTSFDYW |
| | CDR3H | |
| 460 | PMX272 | GGDNIGSKSVH |
| | CDR1L | |
| 461 | PMX272 | YDSSRPT |
| | CDR2L | |
| 462 | PMX272 | CQVWDSSAKAWVF |
| | CDR3L | |
| 463 | PMX273 | GAGATGCAACTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | CGCTACCACATGAGCTGGGTCCGCCAGGCT |
| | | CCAGGGAAGGGGCTTCAGTGGGTCGCATAC |
| | | ATTGACAGCGACGGAAGTCCCACAAACTAT |
| | | GCAGACGCTGTGAAGGGCCGATTCACCATC |
| | | TCCAGAGACAACGCCAAGAACACACTATAT |
| | | CTTCAGATGAACAGCCTGAGAGCCGAGGAC |
| | | ACGGCCGTGTATTACTGTACGACCTACGAC |
| | | CAGTGGGGCCAGGGAACCCTGGTCACCGTC |
| | | TCCTCAG |
| 464 | PMX273 | EMQLVESGGDLVKPGGSLRLSCVASGFTFS |
| | VH_AA | RYHMSWVRQAPGKGLQWVAYIDSDGSPTNY |
| | | ADAVKGRFTISRDNAKNTLYLQMNSLRAED |
| | | TAVYYCTTYDQWGQGTLVTVSS |
| 465 | PMX273 | TCCTATGTGCTGACACAGCTGCCATCCAAA |
| | VL_NT | AGCGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 466 | PMX273 | SYVLTQLPSKSVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGEGTQLTVL |
| 467 | PMX273 | GFTFSRY |
| | CDR1H | |
| 468 | PMX273 | DSDGSP |
| | CDR2H | |
| 469 | PMX273 | CTTYDQW |
| | CDR3H | |
| 470 | PMX273 | GGDNIGSKSVH |
| | CDR1L | |
| 471 | PMX273 | YDSSRPT |
| | CDR2L | |
| 472 | PMX273 | CQVWDSSAKAWVF |
| | CDR3L | |
| 473 | PMX285 | GAGGTGAAGCTGGTGGAGTCTGGGGGAGAC |
| | VH_NT | CTGGTGAAGCCTGGGGGGTCCCTGAGACTT |
| | | TCCTGTGTGGCCTCTGGAGTCACCTTCAGT |
| | | AGGTATTACATGAGCTGGGTCCGCCAGGCT |
| | | CCAGGGAAGGGGCTTCAGTGGGTCGCATAC |
| | | ATTAACAGTGGTGGAAGTACCACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTGAGGGG |
| | | GTGGGGGAGATATATGGATACAAAGACTTT |
| | | GGTATGGACTACTGGGGCCATGGCACCTCA |
| | | CTCTTCGTGTCCTCAG |
| 474 | PMX285 | EVKLVESGGDLVKPGGSLRLSCVASGVTFS |
| | VH_AA | RYYMSWVRQAPGKGLQWVAYINSGGSTTSY |
| | | ADAVKGRFTISRDNAKNTLYLQMNSLRAED |
| | | TAVYYCASEGVGEIYGYKDFGMDYWGHGTS |
| | | LFVSS |
| 475 | PMX285 | TCCTATGTGCTGACACAGCTGCCATCCAAA |
| | VL_NT | AGCGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGAAGGCCGACAGGGATCCCAGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GAAGGGACCCAGCTGACCGTCCTCG |
| 476 | PMX285 | SYVLTQLPSKSVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSAKAWVFGEGTQLTVL |
| 477 | PMX285 | GVTFSRY |
| | CDR1H | |
| 478 | PMX285 | NSGGST |
| | CDR2H | |
| 479 | PMX285 | CASEGVGEIYGYKDFGMDYW |
| | CDR3H | |
| 480 | PMX285 | GGDNIGSKSVH |
| | CDR1L | |
| 481 | PMX285 | YDSRRPT |
| | CDR2L | |
| 482 | PMX285 | CQVWDSSAKAWVF |
| | CDR3L | |
| 483 | PMX286 | GAGGTGCAGCTGGTGGAGTCTGGGGGAGAC |
| | VH_NT | CTGGTGAAGCCTGGGGGGTCCCTGAGACTG |
| | | TCCTGTGTGGCCTCTGGATTCACCTTCAGT |
| | | AGCTACCACATGAGTTGGGTCCGTCAGGCT |
| | | CCAGGGAAGGGGCTTCAATGGGTCGCATCC |
| | | ATTAACAGTGGTGGAAGTAGAACAAGCTAT |
| | | GTAGACGCTGTGAAGGGCCGATTCACCATC |
| | | TCCAGAGACAACGCCAAGAAAACGCTGTAT |
| | | CTTCAGATGAATAGCCTGAGAGCCGAGGAC |
| | | ACGGCCGTTTATTACTGTGCGAGTGGGCGA |
| | | ACCGGGCCCAAGTTGGGACTAGGAGACTAT |
| | | GGTATGGACTCCTGGGGCCATGGCACCTCA |
| | | CTCTTCGTGTCCTCAG |
| 484 | PMX286 | EVQLVESGGDLVKPGGSLRLSCVASGFTFS |
| | VH_AA | SYHMSWVRQAPGKGLQWVASINSGGSRTSY |
| | | VDAVKGRFTISRDNAKKTLYLQMNSLRAED |
| | | TAVYYCASGRTGPKLGLGDYGMDSWGHGTS |
| | | LFVSS |
| 485 | PMX286 | TCCTATGTGCTGACACAGCTGCCATCCAAA |
| | VL_NT | AGCGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAAGGCTTGGGTATTCGGT |
| | | GCAGGGACCCAGCTGACCGTCCTCG |
| 486 | PMX286 | SYVLTQLPSKSVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSAKAWVFGAGTQLTVL |
| 487 | PMX286 | GFTFSSY |
| | CDR1H | |
| 488 | PMX286 | NSGGSR |
| | CDR2H | |
| 489 | PMX286 | CASGRTGPKLGLGDYGMDSW |
| | CDR3H | |
| 490 | PMX286 | GGDNIGSKSVH |
| | CDR1L | |
| 491 | PMX286 | YDSSRPT |
| | CDR2L | |
| 492 | PMX286 | CQVWDSSAKAWVF |
| | CDR3L | |

TABLE 4
Sequences of antibodies/portions thereof
that bind canine CD20

| SEQ | | |
|---|---|---|
| ID | Descrip- | |
| NO: | tion | Sequence |
| 493 | PMX227 | GAGATGCAGCTGGTGGAATCTGGGGGAGCC |
| | VH_NT | CTGGTGAAGCCTGGGGGGTCCCTGAGACTC |
| | | TCCTGTGTGGCCTCTGGATTCACCTTTAGG |
| | | AGATACCACATGAGTTGGGTCCGCCAGGCT |
| | | CCAGGACAGGGGCTTCAGTGGGTCGCATAC |
| | | ATTGACAGTGATGGAAGTCCTACAAGTTAT |
| | | GGAGACTCTGTGAAGGGCCGATTCACCATC |
| | | TCCAGAGACAACGCCAAGAATACGCTATTT |
| | | CTTCAGATGAACAGTCTGAGAGTCGACGAC |
| | | ACGGCCGTATATTACTGTACGACTCTTGAC |
| | | TACTGGGGCCGGGGAACCCTGGTCACCGTC |
| | | TCCTCAG |
| 494 | PMX227 | EMQLVESGGALVKPGGSLRLSCVASGFTFR |
| | VH_AA | RYHMSWVRQAPGQGLQWVAYIDSDGSPTSY |
| | | GDSVKGRFTISRDNAKNTLFLQMNSLRVDD |
| | | TAVYYCTTLDYWGRGTLVTVSS |
| 495 | PMX227 | TCCTATGTGCTGACACAGGTGCCATCCAAA |
| | VL_NT | AATGTGACCCTGAAGCAGCCGGCCCACATC |
| | | ACCTGTGGGGGAGACAACATTGGAGGTAAA |
| | | AGTGTTCACTGGTACCAGCAGAAGCTGGGC |
| | | CAGGCCCCTCTACTGATTATCTATTATGAT |
| | | ACCAGGAGGCCGACAGGGATCCCTGAGCGC |
| | | TTCTCCGGCGCCAACTCGGGGAACACGGCC |
| | | ACCCTGACCATCAGCAGGGCCCTGGCCGAG |
| | | GACGAGGCTGACTATTACTGCCAGGTGTGG |
| | | GACAGCAGTGGTAATGTGTTCGGCGGAGGC |
| | | ACCCATCTGACCGTCATCG |
| 496 | PMX227 | SYVLTQVPSKNVTLKQPAHITCGGDNIGGK |
| | VL_AA | SVHWYQQKLGQAPLLIIYYDTRRPTGIPER |
| | | FSGANSGNTATLTISRALAEDEADYYCQVW |
| | | DSSGNVFGGGTHLTVI |
| 497 | PMX227 | GFTFRRY |
| | CDR1H | |
| 498 | PMX227 | DSDGSP |
| | CDR2H | |
| 499 | PMX227 | CTTLDYW |
| | CDR3H | |
| 500 | PMX227 | GGDNIGGKSVH |
| | CDR1L | |
| 501 | PMX227 | YDTRRPT |
| | CDR2L | |
| 502 | PMX227 | CQVWDSSGNVF |
| | CDR3L | |
| 503 | PMX228 | GAGGTGCAGCTGGTGCAGTCTGGGGGAGAC |
| | VH_NT | CTGGTGAAGCCTGGGGGGTCCCTGAGACTT |
| | | TCCTGTGTGGCCTCTGGATACACCTTCACT |
| | | AGGTATCACATGAGTTGGGTCCGCCAGGCT |
| | | CCAGGGAAGGGGCTTCAGTGGGTCGCATTC |
| | | ATTAATAGTGATGGAACTGGTATAACCTAT |
| | | GGAGACGCTGTGAAGGGCCGATTCACCATC |
| | | TCCAGAGACAATGCCAAGAACACGCTGTAT |
| | | CTTCAGATGAACAGCCTGAGAGCCGAGGAC |
| | | ACGGCCATCTATTACTGTACGACTTTTGAC |
| | | TCCTGGGGCCAGGGAACCCTGGTCACCGTC |
| | | TCCTCAG |
| 504 | PMX228 | EVQLVQSGGDLVKPGGSLRLSCVASGYTFT |
| | VH_AA | RYHMSWVRQAPGKGLQWVAFINSDGTGITY |
| | | GDAVKGRFTISRDNAKNTLYLQMNSLRAED |
| | | TAIYYCTTFDSWGQGTLVTVSS |
| 505 | PMX228 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | AGCAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGGTAATGTGTTCGGCGGAGGC |
| | | ACCCATCTGACCGTCCTCG |
| 506 | PMX228 | SYVLTQLPSKNVTLKQPAHITCGGDNIGSK |
| | VL_AA | SVHWYQQKLGQAPILIIYYDSRRPTGIPER |
| | | FSGANSGNTATLTISGALAEDEADYYCQVW |
| | | DSSGNVFGGGTHLTVL |
| 507 | PMX228 | GYTFTRY |
| | CDR1H | |
| 508 | PMX228 | NSDGTG |
| | CDR2H | |
| 509 | PMX228 | CTTFDSW |
| | CDR3H | |
| 510 | PMX228 | GGDNIGSKSVH |
| | CDR1L | |
| 511 | PMX228 | YDSRRPT |
| | CDR2L | |
| 512 | PMX228 | CQVWDSSGNVF |
| | CDR3L | |
| 513 | PMX229 | GAGGTGCAGTTGGTGGAGTCTGGGGGAGAC |
| | VH_NT | CTGGTGAAGCCGGGGGGGTCCCTGAGACTT |
| | | TCCTGTGTGGCCTCCGGAATCACCTTCAGT |
| | | AGATATCACATGAGCTGGGTCCGCCAGGCT |
| | | CCGGGGAAGGGGCTTCAGTGGGTCGCATTC |
| | | ATTAACACTGGTAGAAGTAGCACAAACTAT |
| | | GCGGACGCTGTGGCGGGCCGATTCACCATC |
| | | TCCAGAGACAACGCCAAGAACACGCTGTAT |
| | | CTTCAAATGAACGGCCTGACAGTCGAGGAC |
| | | ACGGCCGTGTATTACTGTTCGACCCTCGAC |
| | | TACTGGGGCCAGGGAACCCTGGTCACCGTC |
| | | TCCTCAG |
| 514 | PMX229 | EVQLVESGGDLVKPGGSLRLSCVASGITFS |
| | VH_AA | RYHMSWVRQAPGKGLQWVAFINTGRSSTNY |
| | | ADAVAGRFTISRDNAKNTLYLQMNGLTVED |
| | | TAVYYCSTLDYWGQGTLVTVSS |
| 515 | PMX229 | TCCTATGTGCTGACACAGCTGCCATCCAAA |
| | VL_NT | AGTGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | AACAACAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCACGTGTGG |
| | | GACAGCAGTGTTAATGTGTTCGGCGGAGGC |
| | | ACCCATCTGACCGTCCTCG |
| 516 | PMX229 | SYVLTQLPSKSVTLKQPAHITCGGDNIGSK |
| | VL_AA | SVHWYQQKLGQAPILIIYYDNNRPTGIPER |
| | | FSGANSGNTATLTISGALAEDEADYYCHVW |
| | | DSSVNVFGGGTHLTVL |
| 517 | PMX229 | GITFSRY |
| | CDR1H | |
| 518 | PMX229 | NTGRSS |
| | CDR2H | |
| 519 | PMX229 | CSTLDYW |
| | CDR3H | |
| 520 | PMX229 | GGDNIGSKSVH |
| | CDR1L | |
| 521 | PMX229 | YDNNRPT |
| | CDR2L | |
| 522 | PMX229 | CHVWDSSVNVF |
| | CDR3L | |
| 523 | PMX230 | GAGGTGCACCTGGTGGAGTCTGGGGGAGAC |
| | VH_NT | CTGGTGAAGCCTGGGGGGTCCCTGAGACTT |
| | | TCCTGTGTGGCCTCTGGAATCACCTTCAAT |
| | | AGATATTATATGAACTGGGTCCGCCAGACT |
| | | CCAGGGAAGGGGCTTCAGTGGGTCGCATAC |
| | | ATAGACAGTGGTGGAAGTCCCACAACCTAT |
| | | GCAGACGCTGTAAAGGGCCGATTCACCATC |
| | | TCCAGAGACAACGCCAAGAACACGTTGTAT |
| | | CTTCAGATGAACAGCCTGAGAGCCGAGGAC |
| | | ACGGCCGTATATTACTGTACGAGTTTTGAC |
| | | TACTGGGGCCAGGGAACCCTGGTCACCGTC |
| | | TCCTCAG |
| 524 | PMX230 | EVHLVESGGDLVKPGGSLRLSCVASGITFN |
| | VH_AA | RYYMNWVRQTPGKGLQWVAYIDSGGSPTTY |
| | | ADAVKGRFTISRDNAKNTLYLQMNSLRAED |
| | | TAVYYCTSFDYWGQGTLVTVSS |
| 525 | PMX230 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | ACCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 526 | PMX230 | SYVLTQLPSKNVTLKQPAHITCGGDNIGSK |
| | VL_AA | SVHWYQQKLGQAPILIIYYDTSRPTGIPER |
| | | FSGANSGNTATLTISGALAEDEADYYCQVW |
| | | DSSANVFGGGTHLTVL |
| 527 | PMX230 | GITFNRY |
| | CDR1H | |
| 528 | PMX230 | DSGGSP |
| | CDR2H | |
| 529 | PMX230 | CTSFDYW |
| | CDR3H | |
| 530 | PMX230 | GGDNIGSKSVH |
| | CDR1L | |
| 531 | PMX230 | YDTSRPT |
| | CDR2L | |
| 532 | PMX230 | CQVWDSSANVF |
| | CDR3L | |
| 533 | PMX231 | GAGGTGCAACTGGTGGAGTCTGGGGGAGAC |
| | VH_NT | CTGGTGAAGCCTGGGGGGTCCCTGAGACTT |
| | | TCCTGTGTGGCCTCTGGACTCACCTTCAGT |
| | | AGGTACCACATGAGCTGGATCCGCCAGGCT |
| | | CCAGGGAAGGGGCTTCAGTGGGTCGCATAC |
| | | ATTAGCAGTGGTGGAAGTAGCACAAACTAT |
| | | GCAGGCTCTGTGAAGGGCCGATTCACCGTC |
| | | TCCAGAGACAACGCCAAGAACACTCTGTAT |
| | | CTTCAGATGAACAGTCTGAGAGCCGAAGAC |
| | | ACGGCCGTCTATTACTGTACGAGTCTTGAC |
| | | TACTGGGGCCAGGGAACCCTGGTCACCGTC |
| | | TCCTCAG |
| 534 | PMX231 | EVQLVESGGDLVKPGGSLRLSCVASGLTFS |
| | VH_AA | RYHMSWIRQAPGKGLQWVAYISSGGSSTNY |
| | | AGSVKGRFTVSRDNAKNTLYLQMNSLRAED |
| | | TAVYYCTSLDYWGQGTLVTVSS |
| 535 | PMX231 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGTAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGATAATGTGTTCGGCGGAGGC |
| | | ACCCATCTGACCGTCCTCG |
| 536 | PMX231 | SYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSDNVFGGGTHLTVL |
| 537 | PMX231 | GLTFSRY |
| | CDR1H | |
| 538 | PMX231 | SSGGSS |
| | CDR2H | |
| 539 | PMX231 | CTSLDYW |
| | CDR3H | |
| 540 | PMX231 | GGDNIGSKSVH |
| | CDR1L | |
| 541 | PMX231 | YDSRRPT |
| | CDR2L | |
| 542 | PMX231 | CQVWDSSDNVF |
| | CDR3L | |
| 543 | PMX232 | GAGATGCAGCTGGTGGAGTCTGGGGGAGAC |
| | VH_NT | CTGGTGAAGCCTGGGGAATCCCTGAGACTT |
| | | TCCTGTGTGGCCTCTGGAATCTCCTTCAAT |
| | | AACTACCACATGAGCTGGGTCCGCCAGGCT |
| | | CCAGGGAAGGGGCTTCAGTGGGTCGCATAC |
| | | ATTGACAGTGTTGGAACTAGCACAACCTAT |
| | | GCAGACGCTGTGAAGGGCCGATTCACCATC |
| | | TCCAGAGACAACGCCAAGAACACGCTGTAT |
| | | CTTCAAATGAACAGCCTGAGAGCCGAGGAC |
| | | ACGGCCGTTTATTACTGTACGACCTTTGAC |
| | | TCCTGGGGCCAGGGAACCCTGGTCACCGTC |
| | | TCCTCAG |
| 544 | PMX232 | EMQLVESGGDLVKPGESLRLSCVASGISFN |
| | VH_AA | NYHMSWVRQAPGKGLQWVAYIDSVGTSTTY |
| | | ADAVKGRFTISRDNAKNTLYLQMNSLRAED |
| | | TAVYYCTTFDSWGQGTLVTVSS |
| 545 | PMX232 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTATTGATTATCTATTATGAT |
| | | AGCAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 546 | PMX232 | SYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSANVFGGGTHLTVL |
| 547 | PMX232 | GISFNNY |
| | CDR1H | |
| 548 | PMX232 | DSVGTS |
| | CDR2H | |
| 549 | PMX232 | CTTFDSW |
| | CDR3H | |
| 550 | PMX232 | GGDNIGSKSVH |
| | CDR1L | |
| 551 | PMX232 | YDSRRPT |
| | CDR2L | |
| 552 | PMX232 | CQVWDSSANVF |
| | CDR3L | |
| 553 | PMX233 | GAAATGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGATATTACATGAACTGGGTCCGCCAGGCT |
| | | CCAGGGAAGGGACTTCAGTGGGTCGCATAT |
| | | ATTGATAGTGGTGGAAGTCCCACAAACTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | GCGGCCATATATTATTGTACGACTTTTGAC |
| | | TTCTGGGGCCAGGGAACCCTGGTCACCGTC |
| | | TCCTCAG |
| 554 | PMX233 | EMQLVESGGDLVKPGGSLRLSCVASGFTFS |
| | VH_AA | RYYMNWVRQAPGKGLQWVAYIDSGGSPTNY |
| | | ADAVKGRFTISRDNAKNTLYLQMNSLRAED |
| | | AAIYYCTTFDFWGQGTLVTVSS |
| 555 | PMX233 | TCCTATGTGGTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AACAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTCTTCGGCGGAGGC |
| | | ACCCATCTGACCGTCCTCG |
| 556 | PMX233 | SYVVTQLPSKNVTLKQPAHITCGGDNIGSK |
| | VL_AA | SVHWYQQKLGQAPVLIIYYDNRRPTGIPER |
| | | FSGANSGNTATLTISGALAEDEADYYCQVW |
| | | DSSANVFGGGTHLTVL |
| 557 | PMX233 | GFTFSRY |
| | CDR1H | |
| 558 | PMX233 | DSGGSP |
| | CDR2H | |
| 559 | PMX233 | CTTFDFW |
| | CDR3H | |
| 560 | PMX233 | GGDNIGSKSVH |
| | CDR1L | |
| 561 | PMX233 | YDNRRPT |
| | CDR2L | |
| 562 | PMX233 | CQVWDSSANVF |
| | CDR3L | |
| 563 | PMX234 | GAACTACAGCTGGTGGAGTCTGGGGGAGAC |
| | VH_NT | CTGGTGAAGCCTGGGGGGTCCCTGAGACTT |
| | | TCCTGTGTGGCCTCCGGATTCACCTTCAGT |
| | | AGATACTACTTGAACTGGGTCCGCCAGGCT |
| | | CCAGGGAAGGGGCTTCAGTGGGTCGCATTC |
| | | ATTACCAGTGGTGGCAGTAGCACAAACTAT |
| | | GCAGACGCTGTGAAGGGCCGATTCACCATC |
| | | TCCAGAGACAACGCCAAGAACACACTGTAT |
| | | CTTCAGATGAATAGCCTGAGAGCCGAGGAC |
| | | ACGGCCGTTTATTACTGTACGAGCTTCGAC |
| | | TCCTGGGGCCAGGGAACCCTGGTCACCGTC |
| | | TCCTCAG |
| 564 | PMX234 | ELQLVESGGDLVKPGGSLRLSCVASGFTFS |
| | VH_AA | RYYLNWVRQAPGKGLQWVAFITSGGSSTNY |
| | | ADAVKGRFTISRDNAKNTLYLQMNSLRAED |
| | | TAVYYCTSFDSWGQGTLVTVSS |
| 565 | PMX234 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTAATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 566 | PMX234 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 567 | PMX234 | GFTFSRY |
| | CDR1H | |
| 568 | PMX234 | TSGGSS |
| | CDR2H | |
| 569 | PMX234 | CTSFDSW |
| | CDR3H | |
| 570 | PMX234 | GGDNIGSKSVH |
| | CDR1L | |
| 571 | PMX234 | YDSSRPT |
| | CDR2L | |
| 572 | PMX234 | CQVWDSSANVF |
| | CDR3L | |
| 573 | PMX235 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGAATCACCTTCAGT |
| | | AGATATTACATGAACTGGGTCCGCCAGGCT |
| | | CCAGGGAAGGGGCTTCAGTGGGTCGCATAC |
| | | ATTAATACTGGTGAAAGTAGCACAATCTAT |
| | | TCAGACGCTGTGAAGGGCCGATTCACCATT |
| | | TCCAGAGACAACGCCAAGAACACGCTGTAT |
| | | CTTCAGATGAACAGCCTGAGAGCCGAAGAC |
| | | ACGGCCGTGTATTACTGTACGACCCTTGAG |
| | | TATTGGGGCCAGGGAACCCTGGTCACCGTC |
| | | TCCTCAG |
| 574 | PMX235 | EVQLVESGGDLVKPGGSLRLSCVASGITFS |
| | VH_AA | RYYMNWVRQAPGKGLQWVAYINTGESSTIY |
| | | SDAVKGRFTISRDNAKNTLYLQMNSLRAED |
| | | TAVYYCTTLEYWGQGTLVTVSS |
| 575 | PMX235 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGCACTGATTATCTATTATGAT |
| | | AATAGAAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGATAATGTGTTCGGCGGAGGC |
| | | ACCCACCTGACCGTCCTCG |
| 576 | PMX235 | SYVLTQLPSKNVTLKQPAHITCGGDNIGSK |
| | VL_AA | SVHWYQQKLGQAPALIIYYDNRRPTGIPER |
| | | FSGANSGNTATLTISGALAEDEADYYCQVW |
| | | DSSDNVFGGGTHLTVL |
| 577 | PMX235 | GITFSRY |
| | CDR1H | |
| 578 | PMX235 | NTGESS |
| | CDR2H | |
| 579 | PMX235 | CTTLEYW |
| | CDR3H | |
| 580 | PMX235 | GGDNIGSKSVH |
| | CDR1L | |
| 581 | PMX235 | YDNRRPT |
| | CDR2L | |
| 582 | PMX235 | CQVWDSSDNVF |
| | CDR3L | |
| 583 | PMX236 | GAGGTGCAGTTGGTGGAGTCTGGGGGAGAC |
| | VH_NT | CTGGTGAAGCCTGGGGGGTCCCTGAGACTT |
| | | TCCTGTGTGGCCTCTGGATTAACCTTCAGT |
| | | AGATATTACATGAACTGGGTCCGCCAGGCT |
| | | CCAGGGAAGGGGCTTCAGTGGGTCGCATAT |
| | | ATTGATAGTGGTGGAAGTCCCACAAACTAT |
| | | GCAGACGCTGTGAAGGGCCGATTCACCATC |
| | | TCCAGAGACAACGCCAAGAACACACTGTAT |
| | | CTTCAGATGAACAGCCTGAGAGCCGAGGAC |
| | | ACGGCCGTATATTATTGTTCGAGTTTTGAC |
| | | TACTGGGGCCAGGGAACCCTGGTCACCGTC |
| | | TCCTCAG |
| 584 | PMX236 | EVQLVESGGDLVKPGGSLRLSCVASGLTFS |
| | VH_AA | RYYMNWVRQAPGKGLQWVAYIDSGGSPTNY |
| | | ADAVKGRFTISRDNAKNTLYLQMNSLRAED |
| | | TAVYYCSSFDYWGQGTLVTVSS |
| 585 | PMX236 | TCCTATGTGGTGACACAGCTGCCATCCAAT |
| | VL_NT | ACTGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTGCTGATTATCTATTATGAT |
| | | AGCAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGTAGTGCTAATGTGTTCGGCGGAGGC |
| | | ACCCATCTGACCGTCCTCG |
| 586 | PMX236 | SYVVTQLPSNTVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSANVFGGGTHLTVL |
| 587 | PMX236 | GLTFSRY |
| | CDR1H | |
| 588 | PMX236 | DSGGSP |
| | CDR2H | |
| 589 | PMX236 | CSSFDYW |
| | CDR3H | |
| 590 | PMX236 | GGDNIGSKSVH |
| | CDR1L | |
| 591 | PMX236 | YDSRRPT |
| | CDR2L | |
| 592 | PMX236 | CQVWDSSANVF |
| | CDR3L | |
| 593 | PMX237 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGACTCACCTTCAGT |
| | | AGGTACTACATGAACTGGGTCCGCC |
| | | AGGCTCCAGGGAAGGGGCTTCAGTGGGTCG |
| | | CATACATTAACAGTGGTGGAAGTAGCACAG |
| | | ACTATGCAGACGCTGTGAAGGGCCGATTCA |
| | | CCATCTCCAGAGACAACGCCAAGAACACGC |
| | | TGTATCTTCAGATGAACAGCCTGAGAGTCG |
| | | AGGACACGGCCGTGTATTACTGTGCGAGTT |
| | | TTGACTACTGGGGCCAGGGAACCCTGGTCA |
| | | CCGTCTCCTCAG |
| 594 | PMX237 | EVQLVESGGDLVKPGGSLRLSCVASGLTFS |
| | VH_AA | RYYMNWVRQAPGKGLQWVAYINSGGSSTDY |
| | | ADAVKGRFTISRDNAKNTLYLQMNSLRVED |
| | | TAVYYCASFDYWGQGTLVTVSS |
| 595 | PMX237 | TCCTATGTGCTGACACAGCTGCCATCCAAA |
| | VL_NT | AAGGTGACCCTGAAGCAGTCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATCATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 596 | PMX237 | SYVLTQLPSKKVTLKQSAHITCGGDNIGSK |
| | VL_AA | SVHWYQQKLGQAPVLIIYHDSSRPTGIPER |
| | | FSGANSGNTATLTISGALAEDEADYYCQVW |
| | | DSSANVFGGGTHLTVL |
| 597 | PMX237 | GLTFSRY |
| | CDR1H | |
| 598 | PMX237 | NSGGSS |
| | CDR2H | |
| 599 | PMX237 | CASFDYW |
| | CDR3H | |
| 600 | PMX237 | GGDNIGSKSVH |
| | CDR1L | |
| 601 | PMX237 | HDSSRPT |
| | CDR2L | |
| 602 | PMX237 | CQVWDSSANVF |
| | CDR3L | |
| 603 | PMX238 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATTC |
| | | ATTAACACTGGTAGAAGTAGCACAAACTAT |
| | | GCAGACGCTGTGGCGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTTCGACCCTCGAC |
| | | TACTGGGGCCAGGGAACCCTGGTCACCGTC |
| | | TCCTCAG |
| 604 | PMX238 | EVQLVESGGDLVKPGGSLRLSCVASGFTFS |
| | VH_AA | RYHMSWVRQAPGKGLQWVAFINTGRSSTNY |
| | | ADAVAGRFTISRDNAKNTLYLQMNSLRAED |
| | | TAVYYCSTLDYWGQGTLVTVSS |
| 605 | PMX238 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 606 | PMX238 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 607 | PMX238 | GFTFSRY |
| | CDR1H | |
| 608 | PMX238 | NTGRSS |
| | CDR2H | |
| 609 | PMX238 | CSTLDYW |
| | CDR3H | |
| 610 | PMX238 | GGDNIGSKSVH |
| | CDR1L | |
| 611 | PMX238 | YDSSRPT |
| | CDR2L | |
| 612 | PMX238 | CQVWDSSANVF |
| | CDR3L | |
| 613 | PMX239 | GAAGTACAGCTGGTGGAGTCTGGGGGAGAC |
| | VH_NT | CTGGTGAAGCCTGGGGGGTCCCTGAGACTT |
| | | TCCTGTGTGGCCTCCGGATTCACCTTCAGT |
| | | AGATACTACATGAACTGGGTCCGCCAGGCT |
| | | CCAGGGAAGGGGCTTCAGTGGGTCGCATTC |
| | | ATTACCAGTGGTGGAAGTAGCACAAGCTAT |
| | | GGAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTACGAGCTTCGAC |
| | | TACTGGGGCCAGGGAACCCTGGTCACCGTC |
| | | TCCTCAG |
| 614 | PMX239 | EVQLVESGGDLVKPGGSLRLSCVASGFTFS |
| | VH_AA | RYYMNWVRQAPGKGLQWVAFITSGGSSTSY |
| | | GDAVKGRFTISRDNAKNTLYLQMNSLRAED |
| | | TAVYYCTSFDYWGQGTLVTVSS |
| 615 | PMX239 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGCT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 616 | PMX239 | SYVLTQLPSKNVTLKQPAHITCGGDNIGSK |
| | VL_AA | SVHWYQQKLGQAPVLIIYYASSRPTGIPER |
| | | FSGANSGNTATLTISGALAEDEADYYCQVW |
| | | DSSANVFGGGTHLTVL |
| 617 | PMX239 | GFTFSRY |
| | CDR1H | |
| 618 | PMX239 | TSGGSS |
| | CDR2H | |
| 619 | PMX239 | CTSFDYW |
| | CDR3H | |
| 620 | PMX239 | GGDNIGSKSVH |
| | CDR1L | |
| 621 | PMX239 | YASSRPT |
| | CDR2L | |
| 622 | PMX239 | CQVWDSSANVF |
| | CDR3L | |
| 623 | PMX240 | GAGGTGCAGCTGGTGGAGTCTGGGGGAGAC |
| | VH_NT | CTGGTGAAGCCGGGGGGGTCCCTGAGACTT |
| | | TCCTGTGTGGCCTCTGGACTCACCTTCAGT |
| | | AGATACCACATGAGTTGGGTCCGCCAGGCT |
| | | CCAGGGAAGGGGCTTCAGTGGGTCGCATTC |
| | | ATTAACAGTGGTAGAAGTGACACAACCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTTTATTACTGTACGACTTTTGAC |
| | | TACTGGGGCCAGGGAACCCTGGTCACCGTC |
| | | TCCTCAG |
| 624 | PMX240 | EVQLVESGGDLVKPGGSLRLSCVASGLTFS |
| | VH_AA | RYHMSWVRQAPGKGLQWVAFINSGRSDTTY |
| | | ADAVKGRFTISRDNAKNTLYLQMNSLRAED |
| | | TAVYYCTTFDYWGQGTLVTVSS |
| 625 | PMX240 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGCACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGTAGTGCTAATGTGTTCGGCGGAGGC |
| | | ACCCATCTGACCGTCCTCG |
| 626 | PMX240 | SYVLTQLPSKNVTLKQPAHITCGGDNIGSK |
| | VL_AA | SVHWYQQKLGQAPALIIYYDSSRPTGIPER |
| | | FSGANSGNTATLTISGALAEDEADYYCQVW |
| | | DSSANVFGGGTHLTVL |
| 627 | PMX240 | GLTFSRY |
| | CDR1H | |
| 628 | PMX240 | NSGRSD |
| | CDR2H | |
| 629 | PMX240 | CTTFDYW |
| | CDR3H | |
| 630 | PMX240 | GGDNIGSKSVH |
| | CDR1L | |
| 631 | PMX240 | YDSSRPT |
| | CDR2L | |
| 632 | PMX240 | CQVWDSSANVF |
| | CDR3L | |
| 633 | PMX241 | GAGGTGCAGCTGGTGGAGTCTGGGGGAGAC |
| | VH_NT | CTGGTGAAGCCGGGGGGGTCCCTGAGACTT |
| | | TCCTGTGTGGCCTCTGGAATCACCTTCAGT |
| | | GGCTACCACATGAGCTGGGTCCGCCAGGCT |
| | | CCAGGGAAGGGGCTTCAGTGGGTCGCATTC |
| | | ATTAATAGTGGTAGAAGAAGCACAAGTTAT |
| | | GCTGACGCTGTGAAGGGCCGATTCACCATT |
| | | TCCAGAGACAACGCCAAAAACACGCTGTAT |
| | | CTTCAGATGAACAGCCTGAGAGCCGAAGAT |
| | | ACGGCCATTTATTACTGTACGACCCTTGAG |
| | | TTTTGGGGCCAGGGAACCCTGGTCACCGTC |
| | | TCCTCAG |
| 634 | PMX241 | EVQLVESGGDLVKPGGSLRLSCVASGITFS |
| | VH_AA | GYHMSWVRQAPGKGLQWVAFINSGRRSTSY |
| | | ADAVKGRFTISRDNAKNTLYLQMNSLRAED |
| | | TAIYYCTTLEFWGQGTLVTVSS |
| 635 | PMX241 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCCGCAGGCCGACAGGGATCCCTGAGCGA |
| | | TTCTCCGGCGCCAACTCGGGGAACACGGCC |
| | | ACCCTGACCATCAACGGGGCCCTGGCCGAG |
| | | GACGAGGCTGACTATTACTGCCAGATGTGG |
| | | GACAGCAGTGTTAATGTGTTCGGCGGAGGC |
| | | ACCCATCTGACCGTCCTCG |
| 636 | PMX241 | SYVLTQLPSKNVTLKQPAHITCGGDNIGSK |
| | VL_AA | SVHWYQQKLGQAPVLIIYYDSRRPTGIPER |
| | | FSGANSGNTATLTINGALAEDEADYYCQMW |
| | | DSSVNVFGGGTHLTVL |
| 637 | PMX241 | GITFSGY |
| | CDR1H | |
| 638 | PMX241 | NSGRRS |
| | CDR2H | |
| 639 | PMX241 | CTTLEFW |
| | CDR3H | |
| 640 | PMX241 | GGDNIGSKSVH |
| | CDR1L | |
| 641 | PMX241 | YDSRRPT |
| | CDR2L | |
| 642 | PMX241 | CQMWDSSVNVF |
| | CDR3L | |
| 643 | PMX242 | GAGATACAACTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | CGCTACCACATGAGCTGGGTCCGCCAGGCT |
| | | CCAGGGAAGGGGCTTCAGTGGGTCGCATAT |
| | | ATTAACACTGGTGGAAGTAACACAAACTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTACGACCCTTGAC |
| | | TCCTGGGGCCAGGGAACCCTGGTCACCGTC |
| | | TCCTCAG |
| 644 | PMX242 | EIQLVESGGDLVKPGGSLRLSCVASGFTFS |
| | VH_AA | RYHMSWVRQAPGKGLQWVAYINTGGSNTNY |
| | | ADAVKGRFTISRDNAKNTLYLQMNSLRAED |
| | | TAVYYCTTLDSWGQGTLVTVSS |
| 645 | PMX242 | TCCTATGTGCTGACACAGCTGCCATTCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTATTGATTATCTATTATGAT |
| | | AGTAGAAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGTAGTGTTAATGTGTTCGGCGGAGGC |
| | | ACCCATCTGACCGTCCTCG |
| 646 | PMX242 | SYVLTQLPFKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSVNVFGGGTHLTVL |
| 647 | PMX242 | GFTFSRY |
| | CDR1H | |
| 648 | PMX242 | NTGGSN |
| | CDR2H | |
| 649 | PMX242 | CTTLDSW |
| | CDR3H | |
| 650 | PMX242 | GGDNIGSKSVH |
| | CDR1L | |
| 651 | PMX242 | YDSRRPT |
| | CDR2L | |
| 652 | PMX242 | CQVWDSSVNVF |
| | CDR3L | |
| 653 | PMX243 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTAACACAGACTAT |
| | | ACAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTTCGAGTCTGGAC |
| | | TACTGGGGCCAGGGAACCCTGGTCACCGTC |
| | | TCCTCAG |
| 654 | PMX243 | EVQLVESGGDLVKPGGSLRLSCVASGFTFS |
| | VH_AA | RYHMSWVRQAPGKGLQWVAYINSGGSNTDY |
| | | TDAVKGRFTISRDNAKNTLYLQMNSLRAED |
| | | TAVYYCSSLDYWGQGTLVTVSS |
| 655 | PMX243 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGAAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGGAGTGGTAATGTGTTCGGCGGAGGC |
| | | ACCCATCTGACCGTCCTCG |
| 656 | PMX243 | SYVLTQLPSKNVTLKQPAHITCGGDNIGSK |
| | VL_AA | SVHWYQQKLGQAPVLIIYYDRRRPTGIPER |
| | | FSGANSGNTATLTISGALAEDEADYYCQVW |
| | | DRSGNVFGGGTHLTVL |
| 657 | PMX243 | GFTFSRY |
| | CDR1H | |
| 658 | PMX243 | NSGGSN |
| | CDR2H | |
| 659 | PMX243 | CSSLDYW |
| | CDR3H | |
| 660 | PMX243 | GGDNIGSKSVH |
| | CDR1L | |
| 661 | PMX243 | YDRRRPT |
| | CDR2L | |
| 662 | PMX243 | CQVWDRSGNVF |
| | CDR3L | |
| 663 | PMX244 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTGACTCAGACTAT |
| | | GCAGACGCTGTGAAGGGCCGATTCACCATC |
| | | TCCAGAGACAACGCCAAGAATACGCTGTAT |
| | | CTTCAGATGAACAGCCTGAGAGCCGAGGAC |
| | | ACGGCCGTGTATTATTGTACGAGTCTGGAC |
| | | TACTGGGGCCAGGGAACCCTGGTCACCGTC |
| | | TCCTCAG |
| 664 | PMX244 | EVQLVESGGDLVKPGGSLRLSCVASGFTFS |
| | VH_AA | RYHMSWVRQAPGKGLQWVAYINSGGSDSDY |
| | | ADAVKGRFTISRDNAKNTLYLQMNSLRAED |
| | | TAVYYCTSLDYWGQGTLVTVSS |
| 665 | PMX244 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | AGAAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGTCAGGTGTGG |
| | | GACAGGAGTGGTAATGTGTTCGGCGGAGGC |
| | | ACCCATCTGACCGTCCTCG |
| 666 | PMX244 | SYVLTQLPSKNVTLKQPAHITCGGDNIGSK |
| | VL_AA | SVHWYQQKLGQAPILIIYYDRRRPTGIPER |
| | | FSGANSGNTATLTISGALAEDEADYYCQVW |
| | | DRSGNVFGGGTHLTVL |
| 667 | PMX244 | GFTFSRY |
| | CDR1H | |
| 668 | PMX244 | NSGGSD |
| | CDR2H | |
| 669 | PMX244 | CTSLDYW |
| | CDR3H | |
| 670 | PMX244 | GGDNIGSKSVH |
| | CDR1L | |
| 671 | PMX244 | YDRRRPT |
| | CDR2L | |
| 672 | PMX244 | CQVWDRSGNVF |
| | CDR3L | |
| 673 | PMX245 | GAGGTTCAGCTGGTGGAGTCTGGGGGAGAC |
| | VH_NT | CTGGTGAAGCCTGGGGGGTCCCTGAGACTT |
| | | TCCTGTGTGGCCTCTGGACTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGTAGTAGCACAGACTAT |
| | | GCAGACGGTGTGAAGGGCCGATTCACCATC |
| | | TCCAGAGACAACGCCAAGAACACGCTATAT |
| | | CTTCAGATGAACAGCCTGAGAGCCGAGGAC |
| | | ACGGCCGTGTATTACTGTACGAGTCTAGAA |
| | | GACTGGGGCCAGGGAACCCTGGTCACCGTC |
| | | TCCTCAG |
| 674 | PMX245 | EVQLVESGGDLVKPGGSLRLSCVASGLTFS |
| | VH_AA | RYHMSWVRQAPGKGLQWVAYINSGGSSTDY |
| | | ADGVKGRFTISRDNAKNTLYLQMNSLRAED |
| | | TAVYYCTSLEDWGQGTLVTVSS |
| 675 | PMX245 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGTTAATGTGTTCGGCGGAGGC |
| | | ACCCATCTGACCGTCCTCG |
| 676 | PMX245 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSVNVFGGGTHLTVL |
| 677 | PMX245 | GLTFSRY |
| | CDR1H | |
| 678 | PMX245 | NSGGSS |
| | CDR2H | |
| 679 | PMX245 | CTSLEDW |
| | CDR3H | |
| 680 | PMX245 | GGDNIGSKSVH |
| | CDR1L | |
| 681 | PMX245 | YDSSRPT |
| | CDR2L | |
| 682 | PMX245 | CQVWDSSVNVF |
| | CDR3L | |
| 683 | PMX246 | GAGATGCAACTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | CGCTACCACATGAGCTGGGTCCGCCAGGCT |
| | | CCAGGGAAGGGGCTTCAGTGGGTCGCATAC |
| | | ATTAATAGTGGTGGAAGTCCCACAAACTAT |
| | | GCAGACGCTGTGAAGGGCCGATTCACCATC |
| | | TCCAGAGACAACGCCAAGAACACACTATAT |
| | | CTTCAGATGAACAGCCTGAGAGCCGAGGAC |
| | | ACGGCCGTGTATTACTGTACGACCTACGAC |
| | | CAGTGGGGCCAGGGAACCCTGGTCACCGTC |
| | | TCCTCAG |
| 684 | PMX246 | EMQLVESGGDLVKPGGSLRLSCVASGFTFS |
| | VH_AA | RYHMSWVRQAPGKGLQWVAYINSGGSPTNY |
| | | ADAVKGRFTISRDNAKNTLYLQMNSLRAED |
| | | TAVYYCTTYDQWGQGTLVTVSS |
| 685 | PMX246 | TCCTATGTGCTGACACAGCTGCCATCCAAA |
| | VL_NT | AGTGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACACCAGTGCTAATGTGTTCGGCGGAGGC |
| | | ACCCATCTGACCGTCCTCG |
| 686 | PMX246 | SYVLTQLPSKSVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DTSANVFGGGTHLTVL |
| 687 | PMX246 | GFTFSRY |
| | CDR1H | |
| 688 | PMX246 | NSGGSP |
| | CDR2H | |
| 689 | PMX246 | CTTYDQW |
| | CDR3H | |
| 690 | PMX246 | GGDNIGSKSVH |
| | CDR1L | |
| 691 | PMX246 | YDSSRPT |
| | CDR2L | |
| 692 | PMX246 | CQVWDTSANVF |
| | CDR3L | |
| 693 | PMX247 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTCCCTCAAGCTAT |
| | | GCAGACGCTGTGAAGGGCCGATTCACCATC |
| | | TCCAGAGACAACGCCAAGAACACGCTGTAT |
| | | CTTCAAATGAACAGCCTGAGAGCCGAGGAC |
| | | ACGGCCGTATATTACTGTACGAGTTTTGAC |
| | | TGTTGGGGCCAGGGAACCCTGGTCACCGTC |
| | | TCCTCAG |
| 694 | PMX247 | EVQLVESGGDLVKPGGSLRLSCVASGFTFS |
| | VH_AA | RYHMSWVRQAPGKGLQWVAYINSGGSPSSY |
| | | ADAVKGRFTISRDNAKNTLYLQMNSLRAED |
| | | TAVYYCTSFDCWGQGTLVTVSS |
| 695 | PMX247 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTATTGATTATCTATTATGAT |
| | | AGCAGAAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCACCTGACCGTCCTCG |
| 696 | PMX247 | SYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSANVFGGGTHLTVL |
| 697 | PMX247 | GFTFSRY |
| | CDR1H | |
| 698 | PMX247 | NSGGSP |
| | CDR2H | |
| 699 | PMX247 | CTSFDCW |
| | CDR3H | |
| 700 | PMX247 | GGDNIGSKSVH |
| | CDR1L | |
| 701 | PMX247 | YDSRRPT |
| | CDR2L | |
| 702 | PMX247 | CQVWDSSANVF |
| | CDR3L | |
| 703 | PMX248 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGTTGGGTCCGCCAGGCT |
| | | CCAGGGAAGGGGCTTCAGTGGGTCGCATAC |
| | | ATTAACAGTGGTGGAAGTACCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCATATATTTCTGTACGAGCCTTGAC |
| | | TACTGGGGCCAGGGAACCCTGGTCACCGTC |
| | | TCCTCAG |
| 704 | PMX248 | EVQLVESGGDLVKPGGSLRLSCVASGFTFS |
| | VH_AA | RYHMSWVRQAPGKGLQWVAYINSGGSTTSY |
| | | ADAVKGRFTISRDNAKNTLYLQMNSLRAED |
| | | TAIYFCTSLDYWGQGTLVTVSS |
| 705 | PMX248 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATTCTGATTATCTATTATGAT |
| | | AGCAGAAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 706 | PMX248 | SYVLTQLPSKNVTLKQPAHITCGGDNIGSK |
| | VL_AA | SVHWYQQKLGQAPILIIYYDSRRPTGIPER |
| | | FSGANSGNTATLTISGALAEDEADYYCQVW |
| | | DSSANVFGGGTHLTVL |
| 707 | PMX248 | GFTFSRY |
| | CDR1H | |
| 708 | PMX248 | NSGGST |
| | CDR2H | |
| 709 | PMX248 | CTSLDYW |
| | CDR3H | |
| 710 | PMX248 | GGDNIGSKSVH |
| | CDR1L | |
| 711 | PMX248 | YDSRRPT |
| | CDR2L | |
| 712 | PMX248 | CQVWDSSANVF |
| | CDR3L | |
| 713 | PMX249 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGCTTTGAC |
| | | TACTGGGGCCAGGGAACCCTGGTCACCGTC |
| | | TCCTCAG |
| 714 | PMX249 | EVQLVESGGDLVKPGGSLRLSCVASGFTFS |
| | VH_AA | RYHMSWVRQAPGKGLQWVAYINSGGSSTSY |
| | | ADAVKGRFTISRDNAKNTLYLQMNSLRAED |
| | | TAVYYCASFDYWGQGTLVTVSS |
| 715 | PMX249 | CCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 716 | PMX249 | PYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSANVFGGGTHLTVL |
| 717 | PMX249 | GFTFSRY |
| | CDR1H | |
| 718 | PMX249 | NSGGSS |
| | CDR2H | |
| 719 | PMX249 | CASFDYW |
| | CDR3H | |
| 720 | PMX249 | GGDNIGSKSVH |
| | CDR1L | |
| 721 | PMX249 | YDSRRPT |
| | CDR2L | |
| 722 | PMX249 | CQVWDSSANVF |
| | CDR3L | |
| 723 | PMX250 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGACCAGCCTGAGAGCCGAGGAT |
| | | ACGGCCGTGTATTATTGTACGAGTTTTGAC |
| | | TACTGGGGCCAGGGAACCCTGGTCACCGTC |
| | | TCCTCAG |
| 724 | PMX250 | EVQLVESGGDLVKPGGSLRLSCVASGFTFS |
| | VH_AA | SYHMSWVRQAPGKGLQWVAYINSGGSSTSY |
| | | ADAVKGRFTISRDNAKNTLYLQMTSLRAED |
| | | TAVYYCTSFDYWGQGTLVTVSS |
| 725 | PMX250 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAACGTGTTCGGCGGAGGC |
| | | ACCCATCTGACCGTCCTCG |
| 726 | PMX250 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 727 | PMX250 | GFTFSSY |
| | CDR1H | |
| 728 | PMX250 | NSGGSS |
| | CDR2H | |
| 729 | PMX250 | CTSFDYW |
| | CDR3H | |
| 730 | PMX250 | GGDNIGSKSVH |
| | CDR1L | |
| 731 | PMX250 | YDSSRPT |
| | CDR2L | |
| 732 | PMX250 | CQVWDSSANVF |
| | CDR3L | |
| 733 | PMX251 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTTTTGAC |
| | | TACTGGGGCCAGGGAACCCTGGTCACCGTC |
| | | TCCTCAG |
| 734 | PMX251 | EVQLVESGGDLVKPGGSLRLSCVASGFTFS |
| | VH_AA | SYHMSWVRQAPGKGLQWVAYINSGGSSTSY |
| | | ADAVKGRFTISRDNAKNTLYLQMNSLRAED |
| | | TAVYYCASFDYWGQGTLVTVSS |
| 735 | PMX251 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 736 | PMX251 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 737 | PMX251 | GFTFSSY |
| | CDR1H | |
| 738 | PMX251 | NSGGSS |
| | CDR2H | |
| 739 | PMX251 | CASFDYW |
| | CDR3H | |
| 740 | PMX251 | GGDNIGSKSVH |
| | CDR1L | |
| 741 | PMX251 | YDSSRPT |
| | CDR2L | |
| 742 | PMX251 | CQVWDSSANVF |
| | CDR3L | |
| 743 | PMX252 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTTTAGAC |
| | | TACTGGGGCCAGGGAACCCTGGTCACCGTC |
| | | TCCTCAG |
| 744 | PMX252 | EVQLVESGGDLVKPGGSLRLSCVASGFTFS |
| | VH_AA | RYHMSWVRQAPGKGLQWVAYINSGGSSTSY |
| | | ADAVKGRFTISRDNAKNTLYLQMNSLRAED |
| | | TAVYYCASLDYWGQGTLVTVSS |
| 745 | PMX252 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACATCAGTGCTAATGTGTTCGGCGGAGGC |
| | | ACCCACCTGACCGTCCTCG |
| 746 | PMX252 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DISANVFGGGTHLTVL |
| 747 | PMX252 | GFTFSRY |
| | CDR1H | |
| 748 | PMX252 | NSGGSS |
| | CDR2H | |
| 749 | PMX252 | CASLDYW |
| | CDR3H | |
| 750 | PMX252 | GGDNIGSKSVH |
| | CDR1L | |
| 751 | PMX252 | YDSSRPT |
| | CDR2L | |
| 752 | PMX252 | CQVWDISANVF |
| | CDR3L | |
| 753 | PMX253 | GAGGTGCAGATGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTATTACATGAACTGGGTCCGCCAGGCT |
| | | CCAGGTAAGGGGCTTCAGTGGGTCGCATAC |
| | | ATTAACAGTGGTGGAGGTAGCACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTTCGAGTTTCGAC |
| | | TACTGGGGCCAGGGAACCCTGGTCACCGTC |
| | | TCCTCAG |
| 754 | PMX253 | EVQMVESGGDLVKPGGSLRLSCVASGFTFS |
| | VH_AA | RYYMNWVRQAPGKGLQWVAYINSGGGSTSY |
| | | ADAVKGRFTISRDNAKNTLYLQMNSLRAED |
| | | TAVYYCSSFDYWGQGTLVTVSS |
| 755 | PMX253 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 756 | PMX253 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 757 | PMX253 | GFTFSRY |
| | CDR1H | |
| 758 | PMX253 | NSGGGS |
| | CDR2H | |
| 759 | PMX253 | CSSFDYW |
| | CDR3H | |
| 760 | PMX253 | GGDNIGSKSVH |
| | CDR1L | |
| 761 | PMX253 | YDSSRPT |
| | CDR2L | |
| 762 | PMX253 | CQVWDSSANVF |
| | CDR3L | |
| 763 | PMX254 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGATCCGCCAGGCT |
| | | CCAGGGAAGGGACTTCAGTGGGTCGCATTC |
| | | ATTGACAGCGATGGAAGTGGTACAAGATAC |
| | | GCAGACGCTGTGAAGGGCCGATTCACCTTC |
| | | TCCAGAGACAACGCCAAGAATACGCTTTAT |
| | | CTTCAGATGAGCAGCCTGAGAGCCGAGGAC |
| | | ACGGCCGTGTATTACTGTACGAGTCTTGAG |
| | | TTCTGGGGCCAGGGAACCCTGGTCACCGTC |
| | | TCCTCAG |
| 764 | PMX254 | EVQLVESGGDLVKPGGSLRLSCVASGFTFS |
| | VH_AA | RYHMSWIRQAPGKGLQWVAFIDSDGSGTRY |
| | | ADAVKGRFTFSRDNAKNTLYLQMSSLRAED |
| | | TAVYYCTSLEFWGQGTLVTVSS |
| 765 | PMX254 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTATTGTCAGGTGTGG |
| | | GACAGCAGTGCTAATGTGTTCGGCGGAGGC |
| | | ACCCATCTGACCGTCCTCG |
| 766 | PMX254 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 767 | PMX254 | GFTFSRY |
| | CDR1H | |
| 768 | PMX254 | DSDGSG |
| | CDR2H | |
| 769 | PMX254 | CTSLEFW |
| | CDR3H | |
| 770 | PMX254 | GGDNIGSKSVH |
| | CDR1L | |
| 771 | PMX254 | YDSSRPT |
| | CDR2L | |
| 772 | PMX254 | CQVWDSSANVF |
| | CDR3L | |
| 773 | PMX255 | GAAGTGCAACTGGTGGAGTCTGGGGGAGAC |
| | VH_NT | CTGGTGAAGCCGGGGGGGTCCCTGAGACTT |
| | | TCCTGTGTGGCCTCTGGATTCACCTTCAGT |
| | | AGATATCACATGAGTTGGATCCGCCAGGCT |
| | | CCAGGGAAGGGACTTCAGTGGGTCGCATTC |
| | | ATTGACAGCGATGGAAGTGGTGCAAGATAC |
| | | GCAGACGCTGTGAAGGGCCGATTCACCTTC |
| | | TCCAGAGACAACGCCAAGAACACACTGTAT |
| | | CTTCAGATGAACAGCCTGAGAGCCGAGGAC |
| | | ACGGCCGTGTATTATTGCACGAGTCTTGAA |
| | | TACTGGGGCCAGGGAACCCTGGTCACCGTC |
| | | TCCTCAG |
| 774 | PMX255 | EVQLVESGGDLVKPGGSLRLSCVASGFTFS |
| | VH_AA | RYHMSWIRQAPGKGLQWVAFIDSDGSGARY |
| | | ADAVKGRFTFSRDNAKNTLYLQMNSLRAED |
| | | TAVYYCTSLEYWGQGTLVTVSS |
| 775 | PMX255 | TCCTATGTGCTGACACAGCTGCCATCCAAA |
| | VL_NT | AATGTGACCCTGAAGCAGCCGGCCCACATC |
| | | ACCTGTGGGGGAGACAACGTTGGAAGTAAA |
| | | AGTGTTCACTGGTACCAGCAGAAACTGGGC |
| | | CAGGCCCCTGTACTGATTATCTATTATGAT |
| | | AGCAGAAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTATTGTCAGGTGTGG |
| | | GACAGCAGTGCTAATGTGTTCGGCGGAGGC |
| | | ACCCATCTGACCGTCCTCG |
| 776 | PMX255 | SYVLTQLPSKNVTLKQPAHITCGGDNVGSK |
| | VL_AA | SVHWYQQKLGQAPVLIIYYDSRRPTGIPER |
| | | FSGANSGNTATLTISGALAEDEADYYCQVW |
| | | DSSANVFGGGTHLTVL |
| 777 | PMX255 | GFTFSRY |
| | CDR1H | |
| 778 | PMX255 | DSDGSG |
| | CDR2H | |
| 779 | PMX255 | CTSLEYW |
| | CDR3H | |
| 780 | PMX255 | GGDNVGSKSVH |
| | CDR1L | |
| 781 | PMX255 | YDSRRPT |
| | CDR2L | |
| 782 | PMX255 | CQVWDSSANVF |
| | CDR3L | |
| 783 | PMX256 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | TTTCAGATGAACAGCCTGAGAGCCGAGGAC |
| | | ACGGCCGTGTATTACTGTACGACCTTTGAC |
| | | TCCTGGGGCCAGGGAACCCTGGTCACCGTC |
| | | TCCTCAG |
| 784 | PMX256 | EVQLVESGGDLVKPGGSLRLSCVASGFTFS |
| | VH_AA | SYHMSWVRQAPGKGLQWVAYINSGGSSTSY |
| | | ADAVKGRFTISRDNAKNTLYFQMNSLRAED |
| | | TAVYYCTTFDSWGQGTLVTVSS |
| 785 | PMX256 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 786 | PMX256 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 787 | PMX256 | GFTFSSY |
| | CDR1H | |
| 788 | PMX256 | NSGGSS |
| | CDR2H | |
| 789 | PMX256 | CTTFDSW |
| | CDR3H | |
| 790 | PMX256 | GGDNIGSKSVH |
| | CDR1L | |
| 791 | PMX256 | YDSSRPT |
| | CDR2L | |
| 792 | PMX256 | CQVWDSSANVF |
| | CDR3L | |
| 793 | PMX257 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGCTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCTACAGCACGCTGTTT |
| | | CTTCAGATGAACAACCTGAGAGCCGAGGAC |
| | | ACGGCCGTTTATTACTGTACGACTCTTGAC |
| | | TCCTGGGGCCAGGGAACCCTGGTCACCGTC |
| | | TCCTCAG |
| 794 | PMX257 | EVQLVESGGDLVKPGGSLRLSCVASGFTFS |
| | VH_AA | SYHMSWVRQAPGKGLQWVAYINSGGSSTSY |
| | | ADAVKGRFTISRDNAYSTLFLQMNNLRAED |
| | | TAVYYCTTLDSWGQGTLVTVSS |
| 795 | PMX257 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 796 | PMX257 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 797 | PMX257 | GFTFSSY |
| | CDR1H | |
| 798 | PMX257 | NSGGSS |
| | CDR2H | |
| 799 | PMX257 | CTTLDSW |
| | CDR3H | |
| 800 | PMX257 | GGDNIGSKSVH |
| | CDR1L | |
| 801 | PMX257 | YDSSRPT |
| | CDR2L | |
| 802 | PMX257 | CQVWDSSANVF |
| | CDR3L | |
| 803 | PMX258 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTAACCTTCAGT |
| | | AGGTACTACATGAACTGGGTCCGCCAGGCT |
| | | CCAGGGAAGGGGCTTCAGTGGGTCGCATAC |
| | | ATTAACACTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTTTTGAC |
| | | TACTGGGGCCAGGGAACCCTGGTCACCGTC |
| | | TCCTCAG |
| 804 | PMX258 | EVQLVESGGDLVKPGGSLRLSCVASGLTFS |
| | VH_AA | RYYMNWVRQAPGKGLQWVAYINTGGSSTSY |
| | | ADAVKGRFTISRDNAKNTLYLQMNSLRAED |
| | | TAVYYCASFDYWGQGTLVTVSS |
| 805 | PMX258 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTTCTGATTATCTATTATGAT |
| | | AGTAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 806 | PMX258 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 807 | PMX258 | GLTFSRY |
| | CDR1H | |
| 808 | PMX258 | NTGGSS |
| | CDR2H | |
| 809 | PMX258 | CASFDYW |
| | CDR3H | |
| 810 | PMX258 | GGDNIGSKSVH |
| | CDR1L | |
| 811 | PMX258 | YDSSRPT |
| | CDR2L | |
| 812 | PMX258 | CQVWDSSANVF |
| | CDR3L | |
| 813 | PMX259 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTGCGAGTTTTGAC |
| | | TACTGGGGCCAGGGAACCCTGGTCACCGTC |
| | | TCCTCAG |
| 814 | PMX259 | EVQLVESGGDLVKPGGSLRLSCVASGFTFS |
| | VH_AA | RYHMSWVRQAPGKGLQWVAYINSGGSSTSY |
| | | ADAVKGRFTISRDNAKNTLYLQMNSLRAED |
| | | TAVYYCASFDYWGQGTLVTVSS |
| 815 | PMX259 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAACAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGGAAATGTGTTCGGCGGAGGC |
| | | ACCCATCTGACCGTCCTCG |
| 816 | PMX259 | SYVLTQLPSKNVTLKQPAHITCGGDNIGSK |
| | VL_AA | SVHWYQQKLGQAPVLIIYYDSNRPTGIPER |
| | | FSGANSGNTATLTISGALAEDEADYYCQVW |
| | | DSSGNVFGGGTHLTVL |
| 817 | PMX259 | GFTFSRY |
| | CDR1H | |
| 818 | PMX259 | NSGGSS |
| | CDR2H | |
| 819 | PMX259 | CASFDYW |
| | CDR3H | |
| 820 | PMX259 | GGDNIGSKSVH |
| | CDR1L | |
| 821 | PMX259 | YDSNRPT |
| | CDR2L | |
| 822 | PMX259 | CQVWDSSGNVF |
| | CDR3L | |
| 823 | PMX260 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGA |
| | | AGGTACCACATGAGCTGGGTCCGCCAGGCT |
| | | CCAGGGAAGGGGCTTCAGTGGGTCGCATAC |
| | | ATTGACAGTGGTGGAAGTGGCACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTGTATTACTGTTCGAGTCTCGAC |
| | | TACTGGGGCCAGGGAACCCTGGTCACCGTC |
| | | TCTTCAG |
| 824 | PMX260 | EVQLVESGGDLVKPGGSLRLSCVASGFTFR |
| | VH_AA | RYHMSWVRQAPGKGLQWVAYIDSGGSGTSY |
| | | ADAVKGRFTISRDNAKNTLYLQMNSLRAED |
| | | TAVYYCSSLDYWGQGTLVTVSS |
| 825 | PMX260 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGGTAATGTGTTCGGCGGAGGC |
| | | ACCCATCTGACCGTCCTCG |
| 826 | PMX260 | SYVLTQLPSKNVTLKQPAHITCGGDNIGSK |
| | VL_AA | SVHWYQQKLGQAPVLIIYYDSRRPTGIPER |
| | | FSGANSGNTATLTISGALAEDEADYYCQVW |
| | | DSSGNVFGGGTHLTVL |
| 827 | PMX260 | GFTFRRY |
| | CDR1H | |
| 828 | PMX260 | DSGGSG |
| | CDR2H | |
| 829 | PMX260 | CSSLDYW |
| | CDR3H | |
| 830 | PMX260 | GGDNIGSKSVH |
| | CDR1L | |
| 831 | PMX260 | YDSRRPT |
| | CDR2L | |
| 832 | PMX260 | CQVWDSSGNVF |
| | CDR3L | |
| 833 | PMX261 | GAAGTTCAACTGGTGGAGTCTGGGGGAGAC |
| | VH_NT | CTGGTGAAGCCTGGGGGGTCCCTGAGACTT |
| | | TCCTGTGTGGCCTCTGGAATCACCTTCAGT |
| | | AGGTACTACATGAACTGGGTCCGCCAGGCT |
| | | CCAGGGAAGGGGCTTCAGTGGGTCGCATAC |
| | | ATTGACAGTGGTGGAAGTCCCACAACCTAT |
| | | GCAGACGCTGTGAAGGGCCGATTCACCATC |
| | | TCCAGAGACAACGCCAAGAAAGCGCTGTAT |
| | | CTTCAGATGAACAGCCTGAGAGCCGAGGAC |
| | | ACGGCCGTATATTACTGTACGAGTTTTGAC |
| | | TACTGGGGCCAGGGAACCCTGGTCACCGTC |
| | | TCCTCAG |
| 834 | PMX261 | EVQLVESGGDLVKPGGSLRLSCVASGITFS |
| | VH_AA | RYYMNWVRQAPGKGLQWVAYIDSGGSPTTY |
| | | ADAVKGRFTISRDNAKKALYLQMNSLRAED |
| | | TAVYYCTSFDYWGQGTLVTVSS |
| 835 | PMX261 | TCCTATGTCCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTTCTGCCAGGTGTGG |
| | | GACAGCAGTGCTAATGTGTTCGGCGGAGGC |
| | | ACCCATCTGACCGTCCTCG |
| 836 | PMX261 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYFCQVW |
| | | DSSANVFGGGTHLTVL |
| 837 | PMX261 | GITFSRY |
| | CDR1H | |
| 838 | PMX261 | DSGGSP |
| | CDR2H | |
| 839 | PMX261 | CTSFDYW |
| | CDR3H | |
| 840 | PMX261 | GGDNIGSKSVH |
| | CDR1L | |
| 841 | PMX261 | YDSSRPT |
| | CDR2L | |
| 842 | PMX261 | CQVWDSSANVF |
| | CDR3L | |
| 843 | PMX262 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGAATCACCTTCAGT |
| | | AGATATTATATGAACTGGGTCCGCC |
| | | AGGCTCCAGGGAAGGGGCTTCAGTGGGTCG |
| | | CATACATAAACAGTGGTGGAAGTCCCACAA |
| | | CCTATGCAGACGCTGTAAAGGGCCGATTCA |
| | | CCATCTCCAGAGACAACGCCAAGAACACGT |
| | | TGTATCTTCAGATGAACAGCCTGAGAGCCG |
| | | AGGACACGGCCGTGTATTACTGTACGAGTT |
| | | TTGACTACTGGGGCCAGGGAACCCTGGTCA |
| | | CCGTCTCCTCAG |
| 844 | PMX262 | EVQLVESGGDLVKPGGSLRLSCVASGITFS |
| | VH_AA | RYYMNWVRQAPGKGLQWVAYINSGGSPTTY |
| | | ADAVKGRFTISRDNAKNTLYLQMNSLRAED |
| | | TAVYYCTSFDYWGQGTLVTVSS |
| 845 | PMX262 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | ACCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 846 | PMX262 | SYVLTQLPSKNVTLKQPAHITCGGDNIGSK |
| | VL_AA | SVHWYQQKLGQAPILIIYYDTSRPTGIPER |
| | | FSGANSGNTATLTISGALAEDEADYYCQVW |
| | | DSSANVFGGGTHLTVL |
| 847 | PMX262 | GITFSRY |
| | CDR1H | |
| 848 | PMX262 | NSGGSP |
| | CDR2H | |
| 849 | PMX262 | CTSFDYW |
| | CDR3H | |
| 850 | PMX262 | GGDNIGSKSVH |
| | CDR1L | |
| 851 | PMX262 | YDTSRPT |
| | CDR2L | |
| 852 | PMX262 | CQVWDSSANVF |
| | CDR3L | |
| 853 | PMX263 | GAGGTGCAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACTATATGAACTGGGTCCGCCAGGCT |
| | | CCAGGGAAGGGGCTTCAGTGGGTCGCATAC |
| | | ATTAACAGTGGTGGAAGTAGCACAAGCTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGACGAC |
| | | ACGGCCGTGTATTACTGTACGAGTTTTGAC |
| | | TACTGGGGCCAGGGAACCCTGGTCACCGTC |
| | | TCCTCAG |
| 854 | PMX263 | EVQLVESGGDLVKPGGSLRLSCVASGFTFS |
| | VH_AA | RYYMNWVRQAPGKGLQWVAYINSGGSSTSY |
| | | ADAVKGRFTISRDNAKNTLYLQMNSLRADD |
| | | TAVYYCTSFDYWGQGTLVTVSS |
| 855 | PMX263 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 856 | PMX263 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 857 | PMX263 | GFTFSRY |
| | CDR1H | |
| 858 | PMX263 | NSGGSS |
| | CDR2H | |
| 859 | PMX263 | CTSFDYW |
| | CDR3H | |
| 860 | PMX263 | GGDNIGSKSVH |
| | CDR1L | |
| 861 | PMX263 | YDSSRPT |
| | CDR2L | |
| 862 | PMX263 | CQVWDSSANVF |
| | CDR3L | |
| 863 | PMX264 | GAGGTGCAACTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGATACCACATGAGTTGGGTCCGCCAGGCT |
| | | CCAGGGAAGGGGCTTCAGTGGGTCGCATTC |
| | | ATTAACACTGGTAGAATGAGCACAAACTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTATATTACTGTACGAGTCTCGAC |
| | | TACTGGGGCCAGGGAACCCTTGTCACCGTC |
| | | TCCTCAG |
| 864 | PMX264 | EVQLVESGGDLVKPGGSLRLSCVASGFTFS |
| | VH_AA | RYHMSWVRQAPGKGLQWVAFINTGRMSTNY |
| | | ADAVKGRFTISRDNAKNTLYLQMNSLRAED |
| | | TAVYYCTSLDYWGQGTLVTVSS |
| 865 | PMX264 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGCTAATGTGTTCGGCGGAGGC |
| | | ACCCACCTGACCGTCCTCG |
| 866 | PMX264 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSANVFGGGTHLTVL |
| 867 | PMX264 | GFTFSRY |
| | CDR1H | |
| 868 | PMX264 | NTGRMS |
| | CDR2H | |
| 869 | PMX264 | CTSLDYW |
| | CDR3H | |
| 870 | PMX264 | GGDNIGSKSVH |
| | CDR1L | |
| 871 | PMX264 | YDSSRPT |
| | CDR2L | |
| 872 | PMX264 | CQVWDSSANVF |
| | CDR3L | |
| 873 | PMX265 | GAGGTGCAGCTGGTGCAGTCTGGGGGAGAC |
| | VH_NT | CTGGTGAAGCCTGGGGGGTCCCTGAGACTT |
| | | TCCTGTGTGGCCTCTGGATACACCTTCAGT |
| | | CGGTATCACATGAGTTGGGTCCGTCAGGCT |
| | | CCAGGGAAGGGGCTTCAGTGGGTCGCATTC |
| | | ATTAATAGTGATGGAACTGGTATAACCTAT |
| | | GGAGACGCTGTGAAGGGCCGATTCACCATC |
| | | TCCAGAGACAATGCCAAAAACACGCTGTAT |
| | | CTTCAGATGAACAGCCTGAGAGCCGAGGAC |
| | | ACGGCCATTTATTATTGTACGACTTTTGAC |
| | | TCCTGGGGCCAGGGAACCCTGGTCACCGTC |
| | | TCCTCAG |
| 874 | PMX265 | EVQLVQSGGDLVKPGGSLRLSCVASGYTFS |
| | VH_AA | RYHMSWVRQAPGKGLQWVAFINSDGTGITY |
| | | GDAVKGRFTISRDNAKNTLYLQMNSLRAED |
| | | TAIYYCTTFDSWGQGTLVTVSS |
| 875 | PMX265 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTATACTGATTATCTATTATGAT |
| | | AGTAGGAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGCAGTGGTAATGTGTTCGGCGGAGGC |
| | | ACCCATCTGACCGTCCTCG |
| 876 | PMX265 | SYVLTQLPSKNVTLKQPAHITCGGDNIGSK |
| | VL_AA | SVHWYQQKLGQAPILIIYYDSRRPTGIPER |
| | | FSGANSGNTATLTISGALAEDEADYYCQVW |
| | | DSSGNVFGGGTHLTVL |
| 877 | PMX265 | GYTFSRY |
| | CDR1H | |
| 878 | PMX265 | NSDGTG |
| | CDR2H | |
| 879 | PMX265 | CTTFDSW |
| | CDR3H | |
| 880 | PMX265 | GGDNIGSKSVH |
| | CDR1L | |
| 881 | PMX265 | YDSRRPT |
| | CDR2L | |
| 882 | PMX265 | CQVWDSSGNVF |
| | CDR3L | |
| 883 | PMX266 | GAATTACAGCTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTACCACATGAGCTGGGTCCGCCAGGCT |
| | | CCAGGGAAGGGGCTTCAGTGGGTCGCATAC |
| | | ATTAACAGTGGTGGAAGTAGTACAAGTTAT |
| | | GCAGACGCTGTGAAGGGCCGATTCACCATC |
| | | TCCAGAGACAACGCCAAGAACACGCTGTAT |
| | | CTTCAGATGAACAGCCTGAGAGCCGAGGAC |
| | | ACGGCCGTATATTACTGTACGACTTTTGAC |
| | | TCCTGGGGCCAGGGAACCCTGGTCACCGTC |
| | | TCCTCAG |
| 884 | PMX266 | ELQLVESGGDLVKPGGSLRLSCVASGFTFS |
| | VH_AA | RYHMSWVRQAPGKGLQWVAYINSGGSSTSY |
| | | ADAVKGRFTISRDNAKNTLYLQMNSLRAED |
| | | TAVYYCTTFDSWGQGTLVTVSS |
| 885 | PMX266 | TCCTATGTGCTGACACAACTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGAGCCCTGGCCGAG |
| | | GACGAGGCTGACTATTACTGCCAGGTGTGG |
| | | GACATCAGTGCTAATGTGTTCGGCGGAGGC |
| | | ACCCATCTGACCGTCCTCG |
| 886 | PMX266 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DISANVFGGGTHLTVL |
| 887 | PMX266 | GFTFSRY |
| | CDR1H | |
| 888 | PMX266 | NSGGSS |
| | CDR2H | |
| 889 | PMX266 | CTTFDSW |
| | CDR3H | |
| 890 | PMX266 | GGDNIGSKSVH |
| | CDR1L | |
| 891 | PMX266 | YDSSRPT |
| | CDR2L | |
| 892 | PMX266 | CQVWDISANVF |
| | CDR3L | |
| 893 | PMX267 | GAGATGCAACTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | CGCTACCACATGAGCTGGGTCCGCCAGGCT |
| | | CCAGGGAAGGGGCTTCAGTGGGTCGCATAC |
| | | ATTGACAGTGATGGAATTAACACAAACTAT |
| | | GCAGACGCTGTGAAGGGCCGATTCACCATC |
| | | TCCAGAGACAACGCCAAAAACACGCTGTAT |
| | | CTTCAGATGAACAGCCTGAGAGCCGAGGAC |
| | | ACGGCCCTGTATTACTGTACAACCTTTGAC |
| | | TACTGGGGCCAGGGAACCCTGGTCACCGTC |
| | | TCCTCAG |
| 894 | PMX267 | EMQLVESGGDLVKPGGSLRLSCVASGFTFS |
| | VH_AA | RYHMSWVRQAPGKGLQWVAYIDSDGINTNY |
| | | ADAVKGRFTISRDNAKNTLYLQMNSLRAED |
| | | TALYYCTTFDYWGQGTLVTVSS |
| 895 | PMX267 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCAGCAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACAGTAGTGGTAATGTGTTCGGCGGAGGC |
| | | ACCCATCTGACCGTCCTCG |
| 896 | PMX267 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DSSGNVFGGGTHLTVL |
| 89 | PMX267 | GFTFSRY |
| | CDR1H | |
| 898 | PMX267 | DSDGIN |
| | CDR2H | |
| 899 | PMX267 | CTTFDYW |
| | CDR3H | |
| 900 | PMX267 | GGDNIGSKSVH |
| | CDR1L | |
| 901 | PMX267 | YDSSRPT |
| | CDR2L | |
| 902 | PMX267 | CQVWDSSGNVF |
| | CDR3L | |
| 903 | PMX268 | GAGGTGCAGCTGGTGGAGTCTGGGGGAGAC |
| | VH_NT | CTAATGAAGCCTGGGGGGTCCCTGAGACTT |
| | | TCCTGTGTGGCCTCTGGAATCACCTTCAGT |
| | | GGCTACCACATGAGTTGGGTCCGCCAGGCT |
| | | CCAGGGAAGGGACTTCAGTGGGTCGCATTC |
| | | ATTAATAGTGGTAGAAAAAACACAGCTTAT |
| | | GCTGACGCTGTGAAGGGCCGATTCACCATC |
| | | TCCAGAGACAACGCCAAAAACACGCTGTAT |
| | | CTTCAGATGAACAGCCTGAGAGCCGAAGAT |
| | | ACGGCCATTTATTACTGTACGACCCTTGAA |
| | | TTTTGGGGCCAGGGAACCCTGGTCACCGTC |
| | | TCCTCAG |
| 904 | PMX268 | EVQLVESGGDLMKPGGSLRLSCVASGITFS |
| | VH_AA | GYHMSWVRQAPGKGLQWVAFINSGRKNTAY |
| | | ADAVKGRFTISRDNAKNTLYLQMNSLRAED |
| | | TAIYYCTTLEFWGQGTLVTVSS |
| 905 | PMX268 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AATGTTCACTGGTACCAGCAGAAGCTGGGC |
| | | CAGGCCCCTGTACTGATTATCTATTATGAT |
| | | AGCCGCAGGCCGACAGGGATCCCTGAGCGA |
| | | TTCTCCGGCGTCAACTCGGGGAACACGGCC |
| | | ACCCTGACCATCAACGGGGCCCTGGCCGAG |
| | | GACGAGGCTGACTATTACTGCCAGATGTGG |
| | | GACAGCAGTGCTAATGTGTTCGGCGGAGGC |
| | | ACCCATCTGACCGTCCTCG |
| 906 | PMX268 | SYVLTQLPSKNVTLKQPAHITCGGDNIGSK |
| | VL_AA | NVHWYQQKLGQAPVLIIYYDSRRPTGIPER |
| | | FSGVNSGNTATLTINGALAEDEADYYCQMW |
| | | DSSANVFGGGTHLTVL |
| 907 | PMX268 | GITFSGY |
| | CDR1H | |
| 908 | PMX268 | NSGRKN |
| | CDR2H | |
| 909 | PMX268 | CTTLEFW |
| | CDR3H | |
| 910 | PMX268 | GGDNIGSKNVH |
| | CDR1L | |
| 911 | PMX268 | YDSRRPT |
| | CDR2L | |
| 912 | PMX268 | CQMWDSSANVF |
| | CDR3L | |
| 913 | PMX269 | GAGGTGCGGCTGGTGGAGTCTGGGGGAGAC |
| | VH_NT | CTGGTGAAGCCGGGGGGGTCCCTGAGACTT |
| | | TCCTGTGTGGCCTCTGGACTCACCTTCAAT |
| | | AGGTACTACATGAGTTGGGTCCGCCAGGGT |
| | | CCAGGGAAGGGGCTTCAGTGGGTCGCATAC |
| | | ATTGACAGTGGTGGAAGTCCCACAACCTAT |
| | | GCAGACACTGTGAAGGGCCGATTCACCATC |
| | | TCCAGAGACAACGCCAAGAACACGCTGTAT |
| | | CTTCAGATGAACAGCCTGAGAGTCGAGGAC |
| | | ACGGCCGTGTATTACTGTACGACTCTTGAA |
| | | TACTGGGGCCAGGGAACCCTGGTCACCGTC |
| | | TCCTCAG |
| 914 | PMX269 | EVRLVESGGDLVKPGGSLRLSCVASGLTFN |
| | VH_AA | RYYMSWVRQGPGKGLQWVAYIDSGGSPTTY |
| | | ADTVKGRFTISRDNAKNTLYLQMNSLRVED |
| | | TAVYYCTTLEYWGQGTLVTVSS |
| 915 | PMX269 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGTAGTAGGCCGACAGGGATCCCTGAGCGA |
| | | TTCTCCGGCGCCAACTCGGGGAACACGGCC |
| | | ACCCTGACCATCAGCGGGGCCCTGGCCGAG |
| | | GACGAGGCTGACTATTACTGCCAGGTGTGG |
| | | GACACCAGTGCTAATGTGTTCGGCGGAGGC |
| | | ACCCATCTGACCGTCCTCG |
| 916 | PMX269 | SYVLTQLPSKNVTLKQPAHITCGGDNIGSK |
| | VL_AA | SVHWYQQKLGQAPVLIIYYDSSRPTGIPER |
| | | FSGANSGNTATLTISGALAEDEADYYCQVW |
| | | DTSANVFGGGTHLTVL |
| 917 | PMX269 | GLTFNRY |
| | CDR1H | |
| 918 | PMX269 | DSGGSP |
| | CDR2H | |
| 919 | PMX269 | CTTLEYW |
| | CDR3H | |
| 920 | PMX269 | GGDNIGSKSVH |
| | CDR1L | |
| 921 | PMX269 | YDSSRPT |
| | CDR2L | |
| 922 | PMX269 | CQVWDTSANVF |
| | CDR3L | |
| 923 | PMX270 | GAGGTGCAGTTGGTGGAGTCTGGGGGAGAC |
| | VH_NT | CTGGTGAAGCCTGGGGGGTCCCTGAGACTT |
| | | TCCTGTGTGGCCTCTGGATTCACCTTCAGT |
| | | AGGTATTACATGACCTGGGTCCGCCAGGCT |
| | | CCAGGGAAGGGGCTTCAGTGGGTCGCATAC |
| | | ATTAACAGTGATAGAAGAAGCACAACCTAT |
| | | GCAGACGCTGTGAAGGGCCGATTCACCATC |
| | | TCCAGAGACAACGCCAAAAACACGCTGTAT |
| | | CTTCAGATGAACAGCCTGAGAGCCGAGGAC |
| | | ACGGCCGTATATTACTGTACGACCCTTGAA |
| | | TATTGGGGCCAGGGAACCCTGGTCACCGTC |
| | | TCCTCAG |
| 924 | PMX270 | EVQLVESGGDLVKPGGSLRLSCVASGFTFS |
| | VH_AA | RYYMTWVRQAPGKGLQWVAYINSDRRSTTY |
| | | ADAVKGRFTISRDNAKNTLYLQMNSLRAED |
| | | TAVYYCTTLEYWGQGTLVTVSS |
| 925 | PMX270 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCCGCAGGCCGACAGGGATCCCTGAGCGA |
| | | TTCTCCGGCGCCAACTCGGGGAACACGGCC |
| | | ACCCTGACCATCAACGGGGCCCTGGCCGAG |
| | | GACGAGGCTGACTATTACTGCCAGGTGTGG |
| | | GACAGCAGTGCTAATGTGTTCGGCGGAGGC |
| | | ACCCATCTGACCGTCCTCG |
| 926 | PMX270 | SYVLTQLPSKNVTLKQPAHITCGGDNIGSK |
| | VL_AA | SVHWYQQKLGQAPVLIIYYDSRRPTGIPER |
| | | FSGANSGNTATLTINGALAEDEADYYCQVW |
| | | DSSANVFGGGTHLTVL |
| 927 | PMX270 | GFTFSRY |
| | CDR1H | |
| 928 | PMX270 | NSDRRS |
| | CDR2H | |
| 929 | PMX270 | CTTLEYW |
| | CDR3H | |
| 930 | PMX270 | GGDNIGSKSVH |
| | CDR1L | |
| 931 | PMX270 | YDSRRPT |
| | CDR2L | |
| 932 | PMX270 | CQVWDSSANVF |
| | CDR3L | |
| 933 | PMX271 | GCGGTGCAGCTGGTGGAGTCAGGGGGAGAC |
| | VH_NT | CTGGTGAAGCCTGGGGGGTCCCTGAGACTT |
| | | TCCTGTGTGGCCTCTGGAATCACCTTCAGT |
| | | AGGTACCACATGAGTTGGGTCCGCCAGGCT |
| | | CCAGGGAAGGGGCTTCAGTGGGTCGCATAC |
| | | ATTAATAGTGGTGGAAGTGCCACAAGTTAT |
| | | ACAGACGCTGTGAAGGGCCGATTCAGCATC |
| | | TCCAGAGACAACGGCAAAAACACGCTTTAT |
| | | CTTCAGATGAATAGCCTGAGAGTCGAGGAC |
| | | TCGGCCGTCTATTACTGTACGACCCTTGAC |
| | | CTCTGGGGCCAGGGAACCCTGGTCACCGTC |
| | | TCCTCAG |
| 934 | PMX271 | AVQLVESGGDLVKPGGSLRLSCVASGITFS |
| | VH_AA | RYHMSWVRQAPGKGLQWVAYINSGGSATSY |
| | | TDAVKGRFSISRDNGKNTLYLQMNSLRVED |
| | | SAVYYCTTLDLWGQGTLVTVSS |
| 935 | PMX271 | TCCTATGTGCTGACACAGCTGCCATCCAAA |
| | VL_NT | AATGTGACCCTGAAGCAGCCGGCCCACATC |
| | | ACCTGTGGGGGAGACAACATTGGAAGTAAA |
| | | AGTGTTCACTGGTACCAGCAGAAGCTGGGC |
| | | CAGGCCCCTGTACTGATTATCTATTATGAT |
| | | AGCCGCAGGCCGACAGGGATCCCTGAGCGA |
| | | TTCTCCGGCGCCAACTCGGGGAACACGGCC |
| | | ACCCTGACCATCAACGGGGCCCTGGCCGAG |
| | | GACGAGGCTGACTATTACTGTCAGATGTGG |
| | | GACAGCAGTGCTAATGTGTTCGGCGGAGGC |
| | | ACCCATCTGACCGTCCTCG |
| 936 | PMX271 | SYVLTQLPSKNVTLKQPAHITCGGDNIGSK |
| | VL_AA | SVHWYQQKLGQAPVLIIYYDSRRPTGIPER |
| | | FSGANSGNTATLTINGALAEDEADYYCQMW |
| | | DSSANVFGGGTHLTVL |
| 937 | PMX271 | GITFSRY |
| | CDR1H | |
| 938 | PMX271 | NSGGSA |
| | CDR2H | |
| 939 | PMX271 | CTTLDLW |
| | CDR3H | |
| 940 | PMX271 | GGDNIGSKSVH |
| | CDR1L | |
| 941 | PMX271 | YDSRRPT |
| | CDR2L | |
| 942 | PMX271 | CQMWDSSANVF |
| | CDR3L | |

1.-98. (canceled)

99. A bispecific canine antigen-binding molecule comprising a first antigen binding domain or antigen binding portion thereof that specifically binds canine CD3, and a second antigen binding domain that specifically binds canine CD20, wherein:
(a) the first antigen binding domain or antigen-binding portion thereof comprises (i) a first heavy chain variable region (VH) comprising VH complementarity determining region (CDR)1, VH CDR2, and VH CDR3 and (ii) a first light chain variable region (VL) comprising VL CDR1, VL CDR2, and VL CDR3, wherein: the VH CDR1 comprises the amino acid sequence SEQ ID NO: 67; the VH CDR2 comprises the amino acid sequence SEQ ID NO: 68; the VH CDR3 comprises the amino acid sequence SEQ ID NO: 69; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(b) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 87; the VH CDR2 comprises the amino acid sequence SEQ ID NO: 88; the VH CDR3 comprises the amino acid sequence SEQ ID NO: 89; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(c) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 157; the VH CDR2 comprises the amino acid sequence SEQ ID NO: 158; the VH CDR3 comprises the amino acid sequence SEQ ID NO: 159 the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(d) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 167; the VH CDR2 comprises the amino acid sequence SEQ ID NO: 168; the VH CDR3 comprises the amino acid sequence SEQ ID NO: 169; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(e) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 177; the VH CDR2 comprises the amino acid sequence SEQ ID NO: 178; the VH CDR3 comprises the amino acid sequence SEQ ID NO: 179; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(f) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 187; the VH CDR2 comprises the amino acid sequence SEQ ID NO: 188; the VH CDR3 comprises the amino acid sequence SEQ ID NO: 189; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462;
(g) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 327; the VH CDR2 comprises the amino acid sequence SEQ ID NO: 328; the VH CDR3 comprises the amino acid sequence SEQ ID NO: 329; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 480; the VL CDR2 comprises the amino acid sequence SEQ ID NO: 481; and the VL CDR3 comprises the amino acid sequence SEQ ID NO: 482;
(h) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 337; the VH CDR2 comprises the amino acid sequence SEQ ID NO: 338; the VH CDR3 comprises the amino acid sequence SEQ ID NO: 339; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 490; the VL CDR2 comprises the amino acid sequence SEQ ID NO: 491; and the VL CDR3 comprises the amino acid sequence SEQ ID NO: 492;
(i) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 357; the VH CDR2 comprises the amino acid sequence SEQ ID NO: 358; the VH CDR3 comprises the amino acid sequence SEQ ID NO: 359; the VL CDR1 comprises the amino acid sequence SEQ ID NO: 460; the VL CDR2 comprises the amino acid sequence SEQ ID NO: 461; and the VL CDR3 comprises the amino acid sequence SEQ ID NO: 462; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 460; and the VL CDR2 comprises the amino acid sequence SEQ ID NO: 461; the VL CDR3 comprises the amino acid sequence SEQ ID NO: 462;
(j) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 347; the VH CDR2 comprises the amino acid sequence SEQ ID NO: 348; the VH CDR3 comprises the amino acid sequence SEQ ID NO: 349; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 350; the VL CDR2 comprises the amino acid sequence SEQ ID NO: 351; and the VL CDR3 comprises the amino acid sequence SEQ ID NO: 352; or
(k) the first antigen binding domain or antigen-binding portion thereof comprises (i) a first VH comprising VH CDR1, VH CDR2, and VH CDR3 and (ii) a first VL comprising VL CDR1, VL CDR2, and VL CDR3, wherein: the VH CDR1 comprises the amino acid sequence SEQ ID NO: 107; the VH CDR2 comprises the amino acid sequence SEQ ID NO: 108; the VH CDR3 comprises the amino acid sequence SEQ ID NO: 109; the VL CDR1 comprises the amino acid sequence SEQ ID NO: 110; the VL CDR2 comprises the amino acid sequence SEQ ID NO: 111; and the VL CDR3 comprises the amino acid sequence SEQ ID NO: 112; and
the second antigen binding domain or antigen-binding portion thereof comprises (i) a second VH comprising VH CDR1, VH CDR2, and VH CDR3 and (ii) a second VL comprising VL CDR1, VL CDR2, and VL CDR3, wherein: the VH CDR1 comprises the amino acid sequence SEQ ID NO: 527; the VH CDR2 comprises the amino acid sequence SEQ ID NO: 528; the VH CDR3 comprises the amino acid sequence SEQ ID NO: 529; the VL CDR1 comprises the amino acid sequence SEQ ID NO: 110; the VL CDR2 comprises the amino acid sequence SEQ ID NO: 111; and the VL CDR3 comprises the amino acid sequence SEQ ID NO: 112.

100. The bispecific canine antigen-binding molecule of claim 99, wherein the molecule comprises:
(a) a first VH comprising the amino acid sequence of SEQ ID NO: 64; a second VH comprising the amino acid sequence of SEQ ID NO: 524, and a first and second VL comprising the amino acid sequence of SEQ ID NO: 456;
(b) a first VH comprising the amino acid sequence of SEQ ID NO: 84; a second VH comprising the amino acid sequence of SEQ ID NO: 524, and a first and second VL comprising the amino acid sequence of SEQ ID NO: 456;
(c) a first VH comprising the amino acid sequence of SEQ ID NO: 154; a second VH comprising the amino acid sequence of SEQ ID NO: 524, and a first and second VL comprising the amino acid sequence of SEQ ID NO: 456;
(d) a first VH comprising the amino acid sequence of SEQ ID NO: 164; a second VH comprising the amino acid sequence of SEQ ID NO: 524, and a first and second VL comprising the amino acid sequence of SEQ ID NO: 456;
(e) a first VH comprising the amino acid sequence of SEQ ID NO: 174; a second VH comprising the amino acid sequence of SEQ ID NO: 524, and a first and second VL comprising the amino acid sequence of SEQ ID NO: 456;
(f) a first VH comprising the amino acid sequence of SEQ ID NO: 184; a second VH comprising the amino acid sequence of SEQ ID NO: 524, and a first and second VL comprising the amino acid sequence of SEQ ID NO: 456;
(g) a first VH comprising the amino acid sequence of SEQ ID NO: 324; a second VH comprising the amino acid sequence of SEQ ID NO: 524, and a first and second VL comprising the amino acid sequence of SEQ ID NO: 476;
(h) a first VH comprising the amino acid sequence of SEQ ID NO: 334; a second VH comprising the amino acid sequence of SEQ ID NO: 524, and a first and second VL comprising the amino acid sequence of SEQ ID NO: 486;
(i) a first VH comprising the amino acid sequence of SEQ ID NO: 74; a second VH comprising the amino acid sequence of SEQ ID NO: 524, and a first and second VL comprising the amino acid sequence of SEQ ID NO: 456;
(j) a first VH comprising the amino acid sequence of SEQ ID NO: 344; a second VH comprising the amino acid sequence of SEQ ID NO: 524, and a first and second VL comprising the amino acid sequence of SEQ ID NO: 346; or
(k) a first VH comprising the amino acid sequence of SEQ ID NO: 104; a second VH comprising the amino acid sequence of SEQ ID NO: 524, and a first and second VL comprising the amino acid sequence of SEQ ID NO: 106.

101. The bispecific canine antigen-binding molecule according to claim 99 that binds canine CD3 with a monovalent binding dissociation equilibrium constant (KD) of 100 nM-1000 nM as measured using surface plasmon resonance.

102. The bispecific canine antigen-binding molecule of claim 99, wherein each antigen-binding domain or antigen binding portion thereof is an scFv, Fv, heavy chain or single domain antibody.

103. The bispecific canine antigen-binding molecule of claim 99, wherein each antigen-binding domain or antigen binding portion thereof is conjugated to a therapeutic moiety.

104. The bispecific canine antigen-binding molecule of claim 103, wherein the therapeutic moiety binds to a tumour antigen.

105. The bispecific canine antigen-binding molecule of claim 99, wherein the bispecific antigen-binding molecule is conjugated to a further moiety selected from a half life extending moiety, label, cytotoxin, liposome, nanoparticle or radioisotope.

106. A pharmaceutical composition comprising the bispecific canine antigen-binding molecule of claim 99.

107. A method of treating cancer or a condition mediated by B-cells in a canine subject in need thereof comprising administering to the subject an effective amount of the bispecific canine antigen-binding molecule of claim 99.

108. The method of claim 107, wherein the cancer or condition mediated by B-cells is a B cell lymphoma, leukemia or an immune mediated disease.

109. The method of claim 107 further comprising separately administering another therapeutic agent to the subject.

110. The method of claim 109, wherein the therapeutic agent is a cytotoxic agent, a radiotoxic agent, an immunosuppressant, an immunological modulating agent, such as a cytokine or a chemokine, or an antibody or antigen-binding portion thereof that binds canine CD20.

111. A kit comprising the bispecific canine antigen-binding molecule of claim 99 and instructions for use.

112. A nucleic acid sequence that encodes the bispecific canine antigen-binding molecule or a portion thereof of claim 99.

113. A vector comprising the nucleic acid sequence of claim 112.

114. A host cell comprising the nucleic acid sequence of claim 112.

115. A method for making a bispecific antigen-binding molecule comprising culturing the isolated host cell of claim 114 and recovering said antigen-binding molecule.

116. A method for detecting a CD3 protein and a CD20 protein in a biological sample from a canine subject, comprising contacting a biological sample with the bispecific antigen-binding molecule of claim 99, wherein said antigen-binding molecule is linked to a detectable label.

117. A combination therapy comprising the bispecific canine antigen-binding molecule of claim 99 and an antibody or antigen-binding portion thereof that binds canine CD20.

118. A canine antibody or antigen-binding portion thereof that binds canine CD3, wherein the antibody or antigen-binding portion comprises (i) a heavy chain variable region (VH) comprising VH complementarity determining region (CDR)1, VH CDR2, and VH CDR3;
and (ii) a light chain variable region (VL) comprising VL CDR1, VL CDR2, and VL CDR3, wherein:
(a) the VH CDR1 comprises the amino acid sequence SEQ ID NO: 47; the VH CDR2 comprises the amino acid sequence SEQ ID NO: 48; the VH CDR3 comprises the amino acid sequence SEQ ID NO: 49; the VL CDR1 comprises the amino acid sequence SEQ ID NO: 50;
the VL CDR2 comprises the amino acid sequence SEQ ID NO: 51; the VL CDR3 comprises the amino acid sequence SEQ ID NO: 52;
(b) the VH CDR1 comprises the amino acid sequence SEQ ID NO: 87; the VH CDR2 comprises the amino acid sequence SEQ ID NO: 88; the VH CDR3 comprises the amino acid sequence SEQ ID NO: 89; the VL CDR1 comprises the amino acid sequence SEQ ID NO: 90;
the VL CDR2 comprises the amino acid sequence SEQ ID NO: 91; the VL CDR3 comprises the amino acid sequence SEQ ID NO: 92;
(c) the VH CDR1 comprises the amino acid sequence SEQ ID NO: 127; the VH CDR2 comprises the amino acid sequence SEQ ID NO: 128; the VH CDR3 comprises the amino acid sequence SEQ ID NO: 129; the VL CDR1 comprises the amino acid sequence SEQ ID NO: 130;
the VL CDR2 comprises the amino acid sequence SEQ ID NO: 131; the VL CDR3 comprises the amino acid sequence SEQ ID NO: 132; or
(d) the VH CDR1 comprises the amino acid sequence SEQ ID NO: 167; the VH CDR2 comprises the amino acid sequence SEQ ID NO: 168; the VH CDR3 comprises the amino acid sequence SEQ ID NO: 169; the VL CDR1 comprises the amino acid sequence SEQ ID NO: 170;
the VL CDR2 comprises the amino acid sequence SEQ ID NO: 171; the VL CDR3 comprises the amino acid sequence SEQ ID NO: 172.

119. The canine antibody or antigen-binding portion thereof of claim 118, wherein the molecule comprises:
(a) a VH comprising the amino acid sequence of SEQ ID NO: 44; a VL comprising the amino acid sequence of SEQ ID NO: 46;
(b) a VH comprising the amino acid sequence of SEQ ID NO: 84; a VL comprising the amino acid sequence of SEQ ID NO: 86;
(c) a VH comprising the amino acid sequence of SEQ ID NO: 124; a VL comprising the amino acid sequence of SEQ ID NO: 126; or
(d) a VH comprising the amino acid sequence of SEQ ID NO: 164; a VL comprising the amino acid sequence of SEQ ID NO: 166.


SEQUENCE LISTING (946 sequences)

SEQ ID NO: 1 - DNA, 609 bp, Canis sp.
  source 1..609: mol_type = genomic DNA; note = canis familiaris
atgcagtcgaggaacctctggagaattctgggactctgtctcttatcagttggtgcttgggggcaggacgaggatttcaaagcttctgatgacttgacaagtatatctccagagaaacggtttaaggtctccatctctggaaccgaggtagtggtgacatgccctgatgtttttggatatgataatataaaatgggaaaaaaatgataaccttgtggaaggtgctagtaacagagagctatctcagaaggagttttcagaagtggacgacagtggttattatgcctgctatgcagattccataaaggagaagagctatctctacctgagagcaagagtgtgtgcaaactgcatagaggtgaatctgatggcagtggtcacaatcattgtagctgacatctgccttactctggggttgctgctgatggtgtattactggagcaagactagaaaggccaatgccaagcctgtgatgagaggaacaggtgccggcagcaggcccaggggacaaaacaaggagaagccaccacctgttcccaatccagactacgagcccatccggaaaggccagcaggacctgtattctggcctgaatcagagaggcatctga

SEQ ID NO: 2 - DNA, 516 bp, Canis sp.
  source 1..516: mol_type = genomic DNA; note = canis familiaris
atggaacactgcaggtttctggctggcctgatcctggctgtcctcctctcccgagtgagccccttcaaggtatctgtggaggaacttgaggacagagtgttcctgagttgcaataccagtgtcctaaggatagagggaactatgggaatacaactcccacacagtagaactctggacttggggaaacgcatcctggacccacgaggaatatatcggtgcaatgcgacagaagaacaatccgacaaagcaccttatatgcaagtatattatcgaatgtgtcagaactgtgtggagctgaactcagctaccctggctggcattgtcatcgctgacataatcgccactctgctccttgctttgggagtctattgctttgctggacacgagactggaaggtcctctaagtctgctgacactcaaattctgttggaaaatgaccagctctatcagccccttcgagatcggaatgatgctcagtatagtcatcttggtgaaaactggcctcggaagaagtga

SEQ ID NO: 3 - DNA, 549 bp, Canis sp.
  source 1..549: mol_type = genomic DNA; note = canis familiaris
atggagctggggaagcatctggctggcctcattttggctgtcactcttcttcaaggtgctacagcccagaaggaacaaggatttcccatgataaaagtagatggtaatcgagaagatggttcagtacttctgatttgtgactcacaaagcaaagatatcaaatggtttgaagacgggaaggaaagaactttaccaaagaaagataaaaagacgttggatctgggaagtagtatgaaggaccctcaagggatatatcagtgtcaagtagcaaagaacatttcaaagccactccaagtatattacagaatgtgtcagaactgcattgagctgaatgcaggcactataatcggctttgtcttcgctgaaatcatcagcattttcttccttgctgttggggtttacttcattgctggacaggatggagttcgccagtcaagagcctcagacaagcagactctgttgtccaatgaccagctttaccagcccctcagggatcgagaaaatgaccaatatggccaccttcaaggaaagcgactgaggaaaaattga

SEQ ID NO: 4 - protein, 202 aa, Canis sp.
  source 1..202: note = canis familiaris
MQSRNLWRILGLCLLSVGAWGQDEDFKASDDLTSISPEKRFKVSISGTEVVVTCPDVFGYDNIKWEKNDNLVEGASNRELSQKEFSEVDDSGYYACYADSIKEKSYLYLRARVCANCIEVNLMAVVTIIVADICLTLGLLLMVYYWSKTRKANAKPVMRGTGAGSRPRGQNKEKPPPVPNPDYEPIRKGQQDLYSGLNQRGI

SEQ ID NO: 5 - protein, 171 aa, Canis sp.
  source 1..171: note = canis familiaris
MEHCRFLAGLILAVLLSRVSPFKVSVEELEDRVFLSCNTSVLRIEGTMGIQLPHSRTLDLGKRILDPRGIYRCNATEEQSDKAPYMQVYYRMCQNCVELNSATLAGIVIADIIATLLLALGVYCFAGHETGRSSKSADTQILLENDQLYQPLRDRNDAQYSHLGENWPRKK

SEQ ID NO: 6 - protein, 167 aa, Canis sp.
  source 1..167: note = canis familiaris
MELGKHLAGLILAVTLLQGATAQKEQGFPMIKVDGNREDGSVLLICDSQSKDIKWFEDGKERTLPKKDKKTLDLGSSMKDPQGIYQCQVAKNISKPLQVYYRMCQNCIELNAGTIIGFVFAEIISIFFLAVGVYFIAGQDGVRQSRASDKQTLLSNDQLYQPLRDRE

SEQ ID NO: 7 - DNA, 1053 bp, Canis sp.
  source 1..1053: mol_type = genomic DNA; note = canis familiaris
atgcagtcgaggaacctctggagaattctgggactctgtctcttatcagttggtgcttgggggcaggacgaggatttcaaagcttctgatgacttgacaagtatatctccagagaaacggtttaaggtctccatctctggaaccgaggtagtggtgacatgccctgatgtttttggatatgataatataaaatgggaaaaaaatgataaccttgtggaaggtgctagtaacagagagctatctcagaaggagttttcagaagtggacgacagtggttattatgcctgctatgcagattccataaaggagaagagctatctctacctgagagcaagagtgtgtgcaaactgcatagaggtgaatgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtgcaccctgcctcccagccaggaggagatgaccaagaaccaggtgagcctgtggtgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaagtga

SEQ ID NO: 8 - DNA, 1005 bp, Canis sp.
  source 1..1005: mol_type = genomic DNA; note = canis familiaris
atggaacactgcaggtttctggctggcctgatcctggctgtcctcctctcccgagtgagccccttcaaggtatctgtggaggaacttgaggacagagtgttcctgagttgcaataccagtgtcctaaggatagagggaactatgggaatacaactcccacacagtagaactctggacttggggaaacgcatcctggacccacgaggaatatatcggtgcaatgcgacagaagaacaatccgacaaagcaccttatatgcaagtatattatcgaatgtgtcagaactgtgtggagctgaactcagctaccctggctgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccagtgcgagatgaccaagaaccaggtgagcctgagctgcgccgtgaagggattctacccttccgacatcgccgtggagtgggagtccaacggccagcccgagaacaattataagaccacccctcccgtcctcgacagcgacggatccttctttctggtgtccaggctgaccgtggataagtccaggtggcaggaaggcaacgtgttcagctgctccgtgatgcacgaggccctgcacaataggttcacccagaagtccctgagcctgtccctgggaaagtga

SEQ ID NO: 9 - protein, 350 aa, Canis sp.
  source 1..350: note = canis familiaris
MQSRNLWRILGLCLLSVGAWGQDEDFKASDDLTSISPEKRFKVSISGTEVVVTCPDVFGYDNIKWEKNDNLVEGASNRELSQKEFSEVDDSGYYACYADSIKEKSYLYLRARVCANCIEVNESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 334 aa, Canis sp.
  source 1..334: note = canis familiaris
MEHCRFLAGLILAVLLSRVSPFKVSVEELEDRVFLSCNTSVLRIEGTMGIQLPHSRTLDLGKRILDPRGIYRCNATEEQSDKAPYMQVYYRMCQNCVELNSATLAESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 11 - DNA, 984 bp, Canis sp.
  source 1..984: mol_type = genomic DNA; note = canis familiaris
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtacaccctgcctcccagccagtgcgagatgaccaagaaccaggtgagcctgagctgcgccgtgaagggattctacccttccgacatcgccgtggagtgggagtccaacggccagcccgagaacaattataagaccacccctcccgtcctcgacagcgacggatccttctttctggtgtccaggctgaccgtggataagtccaggtggcaggaaggcaacgtgttcagctgctccgtgatgcacgaggccctgcacaataggttcacccagaagtccctgagcctgtccctgggaaagtga

SEQ ID NO: 12 - protein, 327 aa, Canis sp.
  source 1..327: note = canis familiaris
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQCEMTKNQVSLSCAVKGFYPSDIAVEWESNGQPENNYKTTPPVLDSDGSFFLVSRLTVDKSRWQEGNVFSCSVMHEALHNRFTQKSLSLSLGK

SEQ ID NO: 13 - DNA, 324 bp, Canis sp.
  source 1..324: mol_type = genomic DNA; note = canis familiaris
cgtacggtggccgctccctccgtgttcatcttcccaccttccgacgagcagctgaagtccggcaccgcttctgtcgtgtgcctgctgaacaacttctacccccgcgaggccaaggtgcagtggaaggtggacaacgccctgcagtccggcaactcccaggaatccgtgaccgagcaggactccaaggacagcacctactccctgtcctccaccctgaccctgtccaaggccgactacgagaagcacaaggtgtacgcctgcgaagtgacccaccagggcctgtctagccccgtgaccaagtctttcaaccggggcgagtgttga

SEQ ID NO: 14 - protein, 107 aa, Canis sp. [sequence omitted]

SEQ ID NO: 15 - DNA, 984 bp, Canis sp.
  source 1..984: mol_type = genomic DNA; note = canis familiaris
gccagcaccaagggcccttccgtgttccccctggccccttgcagcaggagcacctccgaatccacagctgccctgggctgtctggtgaaggactactttcccgagcccgtgaccgtgagctggaacagcggcgctctgacatccggcgtccacacctttcctgccgtcctgcagtcctccggcctctactccctgtcctccgtggtgaccgtgcctagctcctccctcggcaccaagacctacacctgtaacgtggaccacaaaccctccaacaccaaggtggacaaacgggtcgagagcaagtacggccctccctgccctccttgtcctgcccccgagttcgaaggcggacccagcgtgttcctgttccctcctaagcccaaggacaccctcatgatcagccggacacccgaggtgacctgcgtggtggtggatgtgagccaggaggaccctgaggtccagttcaactggtatgtggatggcgtggaggtgcacaacgccaagacaaagccccgggaagagcagttcaactccacctacagggtggtcagcgtgctgaccgtgctgcatcaggactggctgaacggcaaggagtacaagtgcaaggtcagcaataagggactgcccagcagcatcgagaagaccatctccaaggctaaaggccagccccgggaacctcaggtgtgcaccctgcctcccagccaggaggagatgaccaagaaccaggtgagcctgtggtgcctggtgaagggattctacccttccgacatcgccgtggagtgggagtccaacggccagcccgagaacaattataagaccacccctcccgtcctcgacagcgacggatccttctttctgtactccaggctgaccgtggataagtccaggtggcaggaaggcaacgtgttcagctgctccgtgatgcacgaggccctgcacaatcactacacccagaagtccctgagcctgtccctgggaaagtga

SEQ ID NO: 16 - protein, 327 aa, Canis sp.
  source 1..327: note = canis familiaris
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVCTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - DNA, 363 bp, Canis sp.
  source 1..363: mol_type = genomic DNA; note = canis familiaris
caggttcagctgcaacagcctggcgccgagcttgtgaaacctggcgcctctgtgaagatgagctgcaaggccagcggctacaccttcaccagctacaacatgcactgggtcaagcagacccctggcagaggcctggaatggatcggagccatctatcccggcaacggcgacacctcctacaaccagaagttcaagggcaaagccacactgaccgccgacaagagcagcagcacagcctacatgcagctgagcagcctgaccagcgaagatagcgccgtgtactactgcgccagaagcacctattacggcggcgactggtacttcaacgtgtggggagctggcaccaccgtgacagtttctgct

SEQ ID NO: 18 - protein, 121 aa, Canis sp. [sequence omitted]

SEQ ID NO: 19 - DNA, 319 bp, Canis sp.
  source 1..319: mol_type = genomic DNA; note = canis familiaris
cagattgtgctgtctcagagccccgccatcctgagtgcttctccaggcgagaaagtgaccatgacctgcagagccagcagcagcgtgtcctacatccactggttccagcagaagcctggcagcagccctaagccttggatctacgccacaagcaatctggccagcggcgtgccagtcagattttctggctctggcagcggcaccagctacagcctgacaatctctagagtggaagccgaggacgccgccacctactattgtcagcagtggaccagcaatcctcctacctttggcggaggcaccaagctggaaatcaaac

SEQ ID NO: 20 - protein, 106 aa, Canis sp. [sequence omitted]

SEQ ID NO: 21 - DNA, 894 bp, Canis sp.
  source 1..894: mol_type = genomic DNA; note = canis familiaris
atgacaacacccagaaattcagtaaatgggactttcccggcagagccaatgaaaggccctattgctatgcaatctggtccaaaaccactcttcaggaggatgtcttcactggtgggccccacgcaaagcttcttcatgagggaatctaagactttgggggctgtccagattatgaatgggctcttccacattgccctggggggtcttctgatgatcccagcagggatctatgcacccatctgtgtgactgtgtggtaccctctctggggaggcattatgtatattatttccggatcactcctggcagcaacggagaaaaactccaggaagtgtttggtcaaaggaaaaatgataatgaattcattgagcctctttgctgccatttctggaatgattctttcaatcatggacatacttaatattaaaatttcccattttttaaaaatggagagtctgaattttattagagctcacacaccatatattaacatatacaactgtgaaccagctaatccctctgagaaaaactccccatctacccaatactgttacagcatacaatctctgttcttgggcattttgtcagtgatgctgatctttgccttcttccaggaacttgtaatagctggcatcgttgagaatgaatggaaaagaacgtgctccagacccaaatctaacatagttctcctgtcagcagaagaaaaaaaagaacagactattgaaataaaagaagaagtggttgggctaactgaaacatcttcccaaccaaagaatgaagaagacattgaaattattccaatccaagaagaggaagaagaagaaacagagacgaactttccagaacctccccaagatcaggaatcctcaccaatagaaaatgacagctctccttaa

SEQ ID NO: 22 - DNA, 894 bp, Canis sp.
  source 1..894: mol_type = genomic DNA; note = canis familiaris
atgacaacacccagaaattcaatgagtggaactctcccggtagatcctatgaaaagccctactgccatgtatcctgttcaaaaaataattcccaaaaggatgccttcagtggtgggccctacacaaaacttcttcatgagggaatctaagacactgggggctgtccagattatgaatgggctcttccacattgccctaggcagcctcctgatgattcacacggatgtctatgcgcccatctgtataactatgtggtaccctctctggggaggcattatgttcatcatttctggatcactcctggcagcagcggacaaaaaccccaggaagagtttggtcaaaggaaaaatgataatgaactcattgagcctctttgctgctatttctggaataatttttttgatcatggacatatttaatattaccatttcccatttttttaaaatggagaatttgaatcttattaaagctcccatgccatatgttgacatacacaactgtgacccagctaacccctctgagaaaaactctttatctatacaatattgtggcagcatacgatctgttttcttgggcgtttttgctgtgatggtgatctttacctttttccagaaacttgtgacagctggcattgttgagaatgaatggaaaaaactgtgctctaaacctaaatctgatgtagttgttctgttagctgctgaagaaaaaaaagaacagccgattgaaacaacagaagaaatggttgagctgactgaaatagcttcccaaccaaagaaagaagaagacattgaaattattccagtccaagaagaagaagaggaactggaaataaactttgcagaacctccccaggagcaggaatcttcaccaatagaaaacgacagcatcccttaa

SEQ ID NO: 23 - protein, 297 aa, Canis sp.
  source 1..297: note = canis familiaris
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSP

SEQ ID NO: 24 - protein, 297 aa, Canis sp.
  source 1..297: note = canis familiaris
MTTPRNSMSGTLPVDPMKSPTAMYPVQKIIPKRMPSVVGPTQNFFMRESKTLGAVQIMNGLFHIALGSLLMIHTDVYAPICITMWYPLWGGIMFIISGSLLAAADKNPRKSLVKGKMIMNSLSLFAAISGIIFLIMDIFNITISHFFKMENLNLIKAPMPYVDIHNCDPANPSEKNSLSIQYCGSIRSVFLGVFAVMVIFTFFQKLVTAGIVENEWKKLCSKPKSDVVVLLAAEEKKEQPIETTEEMVELTEIASQPKKEEDIEIIPVQEEEEELEINFAEPPQEQESSPIENDSIP

SEQ ID NO: 25 - DNA, 720 bp, Canis sp.
  source 1..720: mol_type = genomic DNA; note = canis familiaris
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 26 - protein, 297 aa, Canis sp.; identical to SEQ ID NO: 24
  source 1..297: note = canis familiaris

SEQ ID NO: 27 - DNA, 1895 bp, Canis sp.
  source 1..1895: mol_type = genomic DNA; note = canis familiaris
tgcagccgcagaaacacgaggccctggggcggcagagagctacgccgggcggctggtctgcagacttcctggagggccaccccaagcccgggcacctgataacctgcccggaccctcccaggagcgaggccctggtggtcacacagcctcctctcttgcagcctcccccacggccccatcggtgttcccactggcccccagctgcgggaccacatctggcgccaccgtggccctggcctgcctggtgttaggctacttccctgagccggtgaccgtgtcctggaactccggcgccctgaccagcggtgtgcacaccttcccatccgtcctgcaggcctcggggctctactctctcagcagcatggtgacagtgccctccagcaggtggctcagtgacaccttcacctgcaacgtggcccaccggcccagcagcaccaaagtggacaagaccggtaagagggtgtccactgggagacaggccaggtcagccggtccacctggaccccggggcacaggagaggcacctctgtcccccaagctcggggaggagggcctctgggcctggctgcctggtccagtgtgagcacaggctgcacagccccaccatacttcatctccactggcaggcaccagtctgggacgcccccctgacctgccctgacctagcccaccccaagagctgtccccacccctggcccccactaacctccagtctggtctctctgcagtacccaaaacagcgtctacaatagagtccaaaaccggggaaggtcccaaatgcccaggtgagtcaccagggcaccaccttgcacaccaaggcaatggcccgcatccagggactgcctggtggggggaatgttcaagaggatcccccccaaatgctgacccctgcattgtgtctcccacaccagttcctgagattcctggagcaccgtccgtcttcatcttccccccaaaacccaaggacaccctctcgatttcccggacgcccgaggtcacgtgcttggtggtggacttgggcccagatgactccaatgtccagatcacatggtttgtggataacaccgagatgcacacagccaagacgaggccgcgtgaggagcagttcaacagcacctaccgtgtggtcagtgtcctccccatcctacaccaggactggctcaaggggaaggagttcaagtgcaaggtcaacagcaaatccctcccctctgccatggagaggaccatctccaaggccaaaggtgggcaacaggacagatggggcacagggaggtcgagtggggcctggtggacccaggccagccctccactctgggagtgaccatctgtgctgacctctgaccccacaggacagccccacgagccccaggtgtacgtcctgcctccaacccaggaggagctcagcgagaacaaagtcagtgtgacctgcctgatcaaaggcttccacccgcctgacattgccgtcgagtgggagatcaccggacagccggagccagagaacaactaccagacgaccccgccccagctggacagcgacgggacctacttcctgtacagcaggctctcggtggacaggtcccactggcagaggggaaacacctacacctgctcggtgtcacacgaagctctgcacagccaccacacacagaaatccctcacccaatctccgggtaaatgagcagcgcgcccagccccccaggaggcccccgcgggctctgagcgcccacccctgtgtacatcccccccccccgggcaggcaccctgcatgaaataaagcacccagcactgccctgggacctgtgacactgtcgtgcgtctttccaaggcagagctcaggacacccgggcctctgggaggtgcgggcaggccaggctctgaggctcaggggtcaccat

SEQ ID NO: 28 - protein, 335 aa, Canis sp.
  source 1..335: note = canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPEMLGGPSVFIFPPKPKDTLLIARTPEVTCVVVDLDPEDPEVQISWFVDGKQMQTAKTQPREEQFNGTYRVVSVLPIGHQDWLKGKQFTCKVNNKALPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 29 - DNA, 1529 bp, Canis sp.
  source 1..1529: mol_type = genomic DNA; note = canis familiaris
gcctccaccacggccccctcggttttcccactggcccccagctgcgggtccacttccggctccacggtggccctggcctgcctggtgtcaggctacttccccgagcctgtaactgtgtcctggaattccggctccttgaccagcggtgtgcacaccttcccgtccgtcctgcagtcctcagggctctactccctcagcagcatggtgacagtgccctccagcaggtggcccagcgagaccttcacctgcaacgtggcccacccggccagcaaaactaaagtagacaagccaggtgagacgtcggacctcagagaggggtccactcggggacaagccagatcagctgtccctcccagacccacgtcaccggggagtcacctcagtgtccccgtcctcaaggcctcccctttctgggaaggtgtcctctgggcgtggctgcctggtccagatgaccacaggctgcattccccactgtattcccaggacccattgggtgccactgctcgagggtcccctaagttcaccccaagacctgtgcccacaccaggcccccagtaacccccagtctgctctctctgcagtgcccaaaagagaaaatggaagagttcctcgcccacctgattgtcccaaatgcccaggtgagtcagcagggccctgctctgcatcccaagccgatggtgcacacccaggcacagcctgatgggctaatgggtgttggagaagtccaccgaagtgctacctcatccttgtgtcttccattttagcccctgaaagcctgggagggccttcggtcttcatctttcccccgaaacccaaggacaccctcttgattgcccgaacacctgaggtcacatgtgtggtggtggatctgggccgcgaagaccctgaggtgcagatcagctggttcgtggacggtaagcagatgcaaacagccaagactcagcctcgtgaggagcagttcaatggcacctaccgtgtggtcagtgtcctccccattgggcaccaggactggctcaaggggaagcagttcacgtgcaaagtcaaccacattggcctcccatccccgatcgagaggaccatctccaaggccagaggtaggcagcagggcataggggctgcagggagggagagttgccctgtaaattgataccagtcctccaccctgatagtgaccatctgtgctgatcctttaccccatagggcaggcccatcaacccagtgtgtatgtcctgccgccatcccgggaggagttgagcaagaacacagtcagcttgacatgcctgatcaaagacttcttcccacctgacattgatgtggagtggcagagcaatggacagcaggagcctgagagcaagtaccgcacgaccccgccccagctggacgaggacgggtcctacttcctgtacagcaagctctctgtggacaagagccgctggcagcggggagacaccttcatatgtgcggtgatgcatgaagctctacacaaccactacacacagaaatccctctcccattctccgggtaaatga

SEQ ID NO: 30 - protein, 335 aa, Canis sp.
  source 1..335: note = canis familiaris
ASTTAPSVFPLAPSCGSTSGSTVALACLVSGYFPEPVTVSWNSGSLTSGVHTFPSVLQSSGLYSLSSMVTVPSSRWPSETFTCNVAHPASKTKVDKPVPKRENGRVPRPPDCPKCPAPESLGGPSVFIFPPKPKDTLLIARTPEVTCVVVDLGREDPEVQISWFVDGKQMQTAKTQPREEQFNGTYRVVSVLPIGHQDWLKGKQFTCKVNHIGLPSPIERTISKARGQAHQPSVYVLPPSREELSKNTVSLTCLIKDFFPPDIDVEWQSNGQQEPESKYRTTPPQLDEDGSYFLYSKLSVDKSRWQRGDTFICAVMHEALHNHYTQKSLSHSPGK

SEQ ID NO: 31 - DNA, 321 bp, Canis sp.
  source 1..321: mol_type = genomic DNA; note = canis familiaris
ggtcagcccaaggcctccccttcggtcacactcttcccgccctcctctgaggagcttggcgccaacaaggccaccctggtgtgcctcatcagcgacttctaccccagcggcgtgacagtggcctggaaggcagacggcagccccatcacccagggtgtggagaccaccaagccctccaagcagagcaacaacaagtacgcggccagcagctacctgagcctgacgcctgacaagtggaaatctcacagcagcttcagctgcctggtcacgcacgaggggagcaccgtggagaagaaggtggcccccgcagagtgctcttag

SEQ ID NO: 32 - protein, 106 aa, Canis sp.
  source 1..106: note = canis familiaris
GQPKASPSVTLFPPSSEELGANKATLVCLISDFYPSGVTVAWKADGSPITQGVETTKPSKQSNNKYAASSYLSLTPDKWKSHSSFSCLVTHEGSTVEKKVAPAECS

SEQ ID NO: 33 - DNA, 355 bp, Canis sp.
  source 1..355: mol_type = genomic DNA; note = canis familiaris
gaactcacactgcaggagtcagggccaggactggtgaagccctcacagaccctctctctcacctgtgttgtgtccggaggctccgtcaccagcagttacttctggaactggatccgccagcgccctgggaggggactggaatggatggggtactggacaggtagcacaatctacagcccggcattccagggacgcatctccatcactcctgacacggccaagaaccagttctccctgcagctgagctccatgaccaccgaggacacggccgtgtattactgttcaagagagggaggtagcaccttgggggcttttgcttactggggccagggcaccctggtcactgtctcctcag

SEQ ID NO: 34 - protein, 118 aa, Canis sp.
  source 1..118: note = canis familiaris
ELTLQESGPGLVKPSQTLSLTCVVSGGSVTSSYFWNWIRQRPGRGLEWMGYWTGSTIYSPAFQGRISITPDTAKNQFSLQLSSMTTEDTAVYYCSREGGSTLGAFAYWGQGTLVTVSS

SEQ ID NO: 35 - DNA, 325 bp, Canis sp.
  source 1..325: mol_type = genomic DNA; note = canis familiaris
tcctatgtgctgacacagttgccatccaaaaatgtgaccctgaagcagccggcccacatcacctgtgggggagacaaaattggaagtaaaagtgttcactggtaccagcagaagctgggccaggcccctgtattgattatctattatgatagcagcaggccgacagggatccctgagcgattctccggcgccaattcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 36 - protein, 108 aa, Canis sp.
  source 1..108: note = canis familiaris
SYVLTQLPSKNVTLKQPAHITCGGDKIGSKSVHWYQQKLGQAPVLIIYYDSSRPTGIPERFSGANSGNTATLTISGALAEDEADYYCQVWDSSAKASVFGGGTHLTVL

SEQ ID NO: 37 - protein, 7 aa, Canis sp.
  source 1..7: note = canis familiaris
GGSVTSS

SEQ ID NO: 38 - protein, 6 aa, Canis sp.
  source 1..6: note = canis familiaris
GYWTGS

SEQ ID NO: 39 - protein, 15 aa, Canis sp.
  source 1..15: note = canis familiaris
CSREGGSTLGAFAYW

SEQ ID NO: 40 - protein, 11 aa, Canis sp.
  source 1..11: note = canis familiaris
GGDKIGSKSVH

SEQ ID NO: 41 - protein, 7 aa, Canis sp.
  source 1..7: note = canis familiaris
YDSSRPT

SEQ ID NO: 42 - protein, 13 aa, Canis sp.
  source 1..13: note = canis familiaris
CQVWDSSAKASVF

SEQ ID NO: 43 - DNA, 355 bp, Canis sp.
  source 1..355: mol_type = genomic DNA; note = canis familiaris
gaactcacactgcaggagtcagggccaggactggtgaagccctcacagaccctctctctcacctgtgttgtgtccggaggctccgtcaccagaagttactactggaactggatccgccagcgccctgggaggggactggagtggatggggtactggacaggtaccacaaactacaatccggcatttcagggacgcatctccatcactgctgacacggccaagaaccagtgctccctgcagctgagctccatgaccaccgaggacacggccgtgtattactgtgcaagggaggggggaaagctactgggagcttttggtcactggggccagggcaccctggtcactgtctcctcag

SEQ ID NO: 44 - protein, 118 aa, Canis sp.
  source 1..118: note = canis familiaris
ELTLQESGPGLVKPSQTLSLTCVVSGGSVTRSYYWNWIRQRPGRGLEWMGYWTGTTNYNPAFQGRISITADTAKNQCSLQLSSMTTEDTAVYYCAREGGKLLGAFGHWGQGTLVTVSS

SEQ ID NO: 45 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaagattggaagtaaaagtgttcactggtaccagcagaagctgggccaggcccctgtactgattatctattatgatagcagcaggttgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 46 - protein, 108 aa, Canis sp.
  source 1..108: note = canis familiaris
SYVLTQLPSKNVTLKQPAHITCGGDKIGSKSVHWYQQKLGQAPVLIIYYDSSRLTGIPERFSGANSGNTATLTISGALAEDEADYYCQVWDSSAKASVFGGGTHLTVL

SEQ ID NO: 47 - protein, 7 aa, Canis sp.
  source 1..7: note = canis familiaris
GGSVTRS

SEQ ID NO: 48 - protein, 6 aa, Canis sp.
  source 1..6: note = canis familiaris
GYWTGT

SEQ ID NO: 49 - protein, 15 aa, Canis sp.
  source 1..15: note = canis familiaris
CAREGGKLLGAFGHW

SEQ ID NO: 50 - protein, 11 aa, Canis sp.
  source 1..11: note = canis familiaris
GGDKIGSKSVH

SEQ ID NO: 51 - protein, 7 aa, Canis sp.
  source 1..7: note = canis familiaris
YDSSRLT

SEQ ID NO: 52 - protein, 13 aa, Canis sp.
  source 1..13: note = canis familiaris
CQVWDSSAKASVF

SEQ ID NO: 53 - DNA, 355 bp, Canis sp.
  source 1..355: mol_type = genomic DNA; note = canis familiaris
gaactcacactgcaggagtcagggccaggactggtgaagccctcacagaccctctctctcacctgtgttgtgtccggaggctccgtcaccagaagttactactggaactggatccgccagcgccctgggaggggactggaatggatggggtactggacaggtagcacaatctacaacccgacattccagggacgcatctccatcactgctgacactgccaagagccagttctccctacaactgaggtccatgaccaccgaggacacggccatgtatttctgtgcaagagaaggcgggaagtcgctgggagcttttgcttactggggccagggcaccctggtcactgtctcctcag

SEQ ID NO: 54 - protein, 118 aa, Canis sp.
  source 1..118: note = canis familiaris
ELTLQESGPGLVKPSQTLSLTCVVSGGSVTRSYYWNWIRQRPGRGLEWMGYWTGSTIYNPTFQGRISITADTAKSQFSLQLRSMTTEDTAMYFCAREGGKSLGAFAYWGQGTLVTVSS

SEQ ID NO: 55 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaagattggaagtaaaaatgttcactggtaccagcagaagctgggccaggcccctgtaatgattatctattatgatagtagcaggccgacagggatccctgagcgattctccggcgccaactcggggaacacggccaccctgaccatcagcggggccctggccgaggacgaggctgactattactgccaggtgtgggacagcagtgctaaggctgctgtgttcggcggaggcacccacctgaccgtcctcg

SEQ ID NO: 56 - protein, 108 aa, Canis sp.
  source 1..108: note = canis familiaris
SYVLTQLPSKNVTLKQPAHITCGGDKIGSKNVHWYQQKLGQAPVMIIYYDSSRPTGIPERFSGANSGNTATLTISGALAEDEADYYCQVWDSSAKAAVFGGGTHLTVL

SEQ ID NO: 57 - protein, 7 aa, Canis sp.
  source 1..7: note = canis familiaris
GGSVTRS

SEQ ID NO: 58 - protein, 6 aa, Canis sp.
  source 1..6: note = canis familiaris
WTGSTI

SEQ ID NO: 59 - protein, 15 aa, Canis sp.
  source 1..15: note = canis familiaris
CAREGGKSLGAFAYW

SEQ ID NO: 60 - protein, 11 aa, Canis sp.
  source 1..11: note = canis familiaris
GGDKIGSKNVH

SEQ ID NO: 61 - protein, 7 aa, Canis sp.
  source 1..7: note = canis familiaris
YDSSRPT

SEQ ID NO: 62 - protein, 13 aa, Canis sp.
  source 1..13: note = canis familiaris
CQVWDSSAKAAVF

SEQ ID NO: 63 - DNA, 376 bp, Canis sp.
  source 1..376: mol_type = genomic DNA; note = canis familiaris
gaggtgcagatggtggagtctgggggagacctggtgaagcctggggggtccctgagattctcttgtgtggcctctggattcaccttcagtagctaccacatgagctgggtccgccaggctccagggaaggggcttcagtgggtcgcatatattaatagtggtggaagtagaacaagctatgcagacgctgtgaagggccgattcaccatctccagagacaacgccaagaacacgctgtatcttcagatgaacagcctgagagccgaagacacggccgtgtattattgtgcgagtgggagaagccccccccccctagggagttttgactatggtttggactactggggccatggcacctcactcttcgtgtcctcag

SEQ ID NO: 64 - protein, 125 aa, Canis sp.
  source 1..125: note = canis familiaris
EVQMVESGGDLVKPGGSLRFSCVASGFTFSSYHMSWVRQAPGKGLQWVAYINSGGSRTSYADAVKGRFTISRDNAKNTLYLQMNSLRAEDTAVYYCASGRSPPPLGSFDYGLDYWGHGTSLFVSS

SEQ ID NO: 65 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtattgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 66 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKAWVFGEGTQLTVL

SEQ ID NO: 67 - protein, 7 aa, Canis sp.
  source 1..7: note = canis familiaris
GFTFSSY

SEQ ID NO: 68 - protein, 6 aa, Canis sp.
  source 1..6: note = canis familiaris
NSGGSR

SEQ ID NO: 69 - protein, 20 aa, Canis sp.
  source 1..20: note = canis familiaris
CASGRSPPPLGSFDYGLDYW

SEQ ID NO: 70 - protein, 11 aa, Canis sp.
  source 1..11: note = canis familiaris
GGDNIGSKSVH

SEQ ID NO: 71 - protein, 7 aa, Canis sp.
  source 1..7: note = canis familiaris
YDSSRPT

SEQ ID NO: 72 - protein, 13 aa, Canis sp.
  source 1..13: note = canis familiaris
CQVWDSSAKAWVF

SEQ ID NO: 73 - DNA, 376 bp, Canis sp.
  source 1..376: mol_type = genomic DNA; note = canis familiaris
gaggttcagctggtggagtctgggggagacctggtgaagcctggggggtccctgagactttcctgtgtggcctctggattcaccttcagtagctaccaaatgagctgggtccgccaggctccagggaaggggcttcagtgggtcgcatacattagcagtggtggcggtaggacaacctatgcagacgctgtgaggggccgattaaccatctccagagacaacgccaagaacaccctgtatcttcagatgaacagcctgagagccgaggacgcggccgtgtattactgtgcgcgtgagggggggggggagatatatggatacaaagactatggtatggactactggggccatggcacctcactcttcgtgtcctcag

SEQ ID NO: 74 - protein, 125 aa, Canis sp.
  source 1..125: note = canis familiaris
EVQLVESGGDLVKPGGSLRLSCVASGFTFSSYQMSWVRQAPGKGLQWVAYISSGGGRTTYADAVRGRLTISRDNAKNTLYLQMNSLRAEDAAVYYCAREGGGEIYGYKDYGMDYWGHGTSLFVSS

SEQ ID NO: 75 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggttttcggtgaagggacccagctgaccgtcctcg

SEQ ID NO: 76 - protein, 108 aa, Canis sp.; identical to SEQ ID NO: 66
  source 1..108: note = canis familiaris

SEQ ID NO: 77 - protein, 7 aa, Canis sp.
  source 1..7: note = canis familiaris
GFTFSSY

SEQ ID NO: 78 - protein, 6 aa, Canis sp.
  source 1..6: note = canis familiaris
SSGGGR

SEQ ID NO: 79 - protein, 20 aa, Canis sp.
  source 1..20: note = canis familiaris
CAREGGGEIYGYKDYGMDYW

SEQ ID NO: 80 - protein, 11 aa, Canis sp.
  source 1..11: note = canis familiaris
GGDNIGSKSVH

SEQ ID NO: 81 - protein, 7 aa, Canis sp.
  source 1..7: note = canis familiaris
YDSSRPT

SEQ ID NO: 82 - protein, 13 aa, Canis sp.
  source 1..13: note = canis familiaris
CQVWDSSAKAWVF

SEQ ID NO: 83 - DNA, 379 bp, Canis sp.
  source 1..379: mol_type = genomic DNA; note = canis familiaris
gagatgcaattggtggagtctgggggagacctggtgaagcctggggggtccctgagactttcctgtgtggcctctggattcaccttcagtacttaccacatgagttgggtccgccaggctccagggaaggggcttcagtgggtcgcatacagcaatagtggtggaactaggacatattatgcagacgctgtgaagggccgattcaccatctccagagacaacgtcaagaacacgctgtatcttcagatgaacagcctgagagccgaggacacggccgtgtactactgtgcgagtgaggggagtgggggggggagatatggatttaaagactatggtatggactactggggccatggcacctcactcttcgtgtccacag

SEQ ID NO: 84 - protein, 126 aa, Canis sp.
  source 1..126: note = canis familiaris
EMQLVESGGDLVKPGGSLRLSCVASGFTFSTYHMSWVRQAPGKGLQWVAYSNSGGTRTYYADAVKGRFTISRDNVKNTLYLQMNSLRAEDTAVYYCASEGSGGGRYGFKDYGMDYWGHGTSLFVST

SEQ ID NO: 85 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 86 - protein, 108 aa, Canis sp.; identical to SEQ ID NO: 66
  source 1..108: note = canis familiaris

SEQ ID NO: 87 - protein, 7 aa, Canis sp.
  source 1..7: note = canis familiaris
GFTFSTY

SEQ ID NO: 88 - protein, 6 aa, Canis sp.
  source 1..6: note = canis familiaris
NSGGTR

SEQ ID NO: 89 - protein, 21 aa, Canis sp.
  source 1..21: note = canis familiaris
CASEGSGGGRYGFKDYGMDYW

SEQ ID NO: 90 - protein, 11 aa, Canis sp.
  source 1..11: note = canis familiaris
GGDNIGSKSVH

SEQ ID NO: 91 - protein, 7 aa, Canis sp.
  source 1..7: note = canis familiaris
YDSSRPT

SEQ ID NO: 92 - protein, 13 aa, Canis sp.
  source 1..13: note = canis familiaris
CQVWDSSAKAWVF

SEQ ID NO: 93 - DNA, 355 bp, Canis sp.
  source 1..355: mol_type = genomic DNA; note = canis familiaris
gaactcgcactgcaggagtcagggccaggactggtgaggccctcacagaccctctctctcacctgtgttgtgtccggaggctccgtcaccagaagttactactggaactggatccgccagcgccctgggaggggactggaatggatgggttactggacaggtaccacaatctacaacccggcattccagggacgcatctctatcacagttgacacggccaagaatcagttctccctgcatctgatctccatgaccaccgaggacacggccgtgtattattgtggaagagaggggggttcttccctaggtgctattgcttactggggccagggcacccgggtcactgtctcctcag

SEQ ID NO: 94 - protein, 118 aa, Canis sp.
  source 1..118: note = canis familiaris
ELALQESGPGLVRPSQTLSLTCVVSGGSVTRSYYWNWIRQRPGRGLEWMGYWTGTTIYNPAFQGRISITVDTAKNQFSLHLISMTTEDTAVYYCGREGGSSLGAIAYWGQGTRVTVSS

SEQ ID NO: 95 - DNA, 325 bp, Canis sp.
  source 1..325: mol_type = genomic DNA; note = canis familiaris
tcctatgtgttgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctgctgtgttcggcggaggcacccacctgaccgtcctcg

SEQ ID NO: 96 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKAAVFGGGTHLTVL

SEQ ID NO: 97 - protein, 7 aa, Canis sp.
  source 1..7: note = canis familiaris
GGSVTRS

SEQ ID NO: 98 - protein, 6 aa, Canis sp.
  source 1..6: note = canis familiaris
GYWTGT

SEQ ID NO: 99 - protein, 15 aa, Canis sp.
  source 1..15: note = canis familiaris
CGREGGSSLGAIAYW

SEQ ID NO: 100 - protein, 11 aa, Canis sp.
  source 1..11: note = canis familiaris
GGDNIGSKSVH

SEQ ID NO: 101 - protein, 7 aa, Canis sp.
  source 1..7: note = canis familiaris
YDSSRPT

SEQ ID NO: 102 - protein, 13 aa, Canis sp.
  source 1..13: note = canis familiaris
CQVWDSSAKAAVF

SEQ ID NO: 103 - DNA, 358 bp, Canis sp.
  source 1..358: mol_type = genomic DNA; note = canis familiaris
gaactcacactgcaggagtcagggccaggactggtgaagccctcacagaccctctctctcacctgtgttgtgtccggaggctccgtcaccaacaattactactggaactggatccgccagcgccctgggaggggactggaatggatgggttactggacaggtagcacaaagtataacccggcattccagggacgcatctccatcactgctgacacggccaacaaccagttctctctgcacctgaactccatgagttccgaggacacggccgtttattactgtgcaagagatcgagatcggcgagtcccacatgcttatggttactggggccagggcaccctggtcactgtctcctcag

SEQ ID NO: 104 - protein, 119 aa, Canis sp.
  source 1..119: note = canis familiaris
ELTLQESGPGLVKPSQTLSLTCVVSGGSVTNNYYWNWIRQRPGRGLEWMGYWTGSTKYNPAFQGRISITADTANNQFSLHLNSMSSEDTAVYYCARDRDRRVPHAYGYWGQGTLVTVSS

SEQ ID NO: 105 - DNA, 325 bp, Canis sp.
  source 1..325: mol_type = genomic DNA; note = canis familiaris
tcctatgtgctgacacagctgccatccaaaaatgtgacccttaaacagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattattgccaggtgtgggacagcagtgctaaggctagtgtgttcggcggaggcacccatctgaccgtcctcg

SEQ ID NO: 106 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKASVFGGGTHLTVL

SEQ ID NO: 107 - protein, 7 aa, Canis sp.
  source 1..7: note = canis familiaris
GGSVTNN

SEQ ID NO: 108 - protein, 6 aa, Canis sp.
  source 1..6: note = canis familiaris
GYWTGS

SEQ ID NO: 109 - protein, 16 aa, Canis sp.
  source 1..16: note = canis familiaris
CARDRDRRVPHAYGYW

SEQ ID NO: 110 - protein, 11 aa, Canis sp.
  source 1..11: note = canis familiaris
GGDNIGSKSVH

SEQ ID NO: 111 - protein, 7 aa, Canis sp.
  source 1..7: note = canis familiaris
YDSSRPT

SEQ ID NO: 112 - protein, 13 aa, Canis sp.
  source 1..13: note = canis familiaris
CQVWDSSAKASVF

SEQ ID NO: 113 - DNA, 376 bp, Canis sp.
  source 1..376: mol_type = genomic DNA; note = canis familiaris
gaggtgcagctggtggagtctgggggagacctggtgaagccgggggggtccctgagactttcctgtgtggcctctggattcaacttcagtgcctcccacatgagctgggtccgccaggctccagggaaggggcttcagtgggtcgcatacattaccaatgctggaggtagaataagttatgccgacgctgtgaagggccgattcaccatctccagagacaacgccaagaacacgctatatcttcagatgagcagcctgagagtcgaagacacggccgtgtattattgtgcgactgaaaggcccggacggcggatgggtctaaaggactatggtatggactactggggccatggcacctcactcttcgtgtcctcag

SEQ ID NO: 114 - protein, 125 aa, Canis sp.
  source 1..125: note = canis familiaris
EVQLVESGGDLVKPGGSLRLSCVASGFNFSASHMSWVRQAPGKGLQWVAYITNAGGRISYADAVKGRFTISRDNAKNTLYLQMSSLRVEDTAVYYCATERPGRRMGLKDYGMDYWGHGTSLFVSS

SEQ ID NO: 115 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 116 - protein, 108 aa, Canis sp.; identical to SEQ ID NO: 106
  source 1..108: note = canis familiaris

SEQ ID NO: 117 - protein, 7 aa, Canis sp.
  source 1..7: note = canis familiaris
GFNFSAS

SEQ ID NO: 118 - protein, 6 aa, Canis sp.
  source 1..6: note = canis familiaris
TNAGGR

SEQ ID NO: 119 - protein, 20 aa, Canis sp.
  source 1..20: note = canis familiaris
CATERPGRRMGLKDYGMDYW

SEQ ID NO: 120 - protein, 11 aa, Canis sp.
  source 1..11: note = canis familiaris
GGDNIGSKSVH

SEQ ID NO: 121 - protein, 7 aa, Canis sp.
  source 1..7: note = canis familiaris
YDSSRPT

SEQ ID NO: 122 - protein, 13 aa, Canis sp.
  source 1..13: note = canis familiaris
CQVWDSSAKASVF

SEQ ID NO: 123 - DNA, 355 bp, Canis sp.; identical to SEQ ID NO: 33
  source 1..355: mol_type = genomic DNA; note = canis familiaris

SEQ ID NO: 124 - protein, 118 aa, Canis sp.; identical to SEQ ID NO: 34
  source 1..118: note = canis familiaris

SEQ ID NO: 125 - DNA, 325 bp, Canis sp.; identical to SEQ ID NO: 115
  source 1..325: mol_type = genomic DNA; note = canis familiaris

SEQ ID NO: 126 - protein, 108 aa, Canis sp.; identical to SEQ ID NO: 106
  source 1..108: note = canis familiaris

SEQ ID NO: 127 - protein, 7 aa, Canis sp.
  source 1..7: note = canis familiaris
GGSVTSS

SEQ ID NO: 128 - protein, 6 aa, Canis sp.
  source 1..6: note = canis familiaris
GYWTGS

SEQ ID NO: 129 - protein, 15 aa, Canis sp.
  source 1..15: note = canis familiaris
CSREGGSTLGAFAYW

SEQ ID NO: 130 - protein, 11 aa, Canis sp.
  source 1..11: note = canis familiaris
GGDNIGSKSVH

SEQ ID NO: 131 - protein, 7 aa, Canis sp.
  source 1..7: note = canis familiaris
YDSSRPT

SEQ ID NO: 132 - protein, 13 aa, Canis sp.
  source 1..13: note = canis familiaris
CQVWDSSAKASVF

SEQ ID NO: 133 - DNA, 346 bp, Canis sp.
  source 1..346: mol_type = genomic DNA; note = canis familiaris
gaggtgaagttggtggagtctgggggagacctggtgaagcctggggggtccctgagactttcctgtatggcctctggattcaccctcaataattactacatgagttgggtccgccaggctccagggaaggggcttcagtgggtcgcatacattaaaagtggtggaagtgacacaagttatgtagacgctgtgaagggccgattcaccatctccagagacaatgccaagaacacgctgcatcttcagatgaacagcctgagagtcgaggacacggccgtatattattgtgcgactgacggatatatatgggcctggggccagggaaccctggtcaccgtctcctcag

SEQ ID NO: 134 - protein, 115 aa, Canis sp.
  source 1..115: note = canis familiaris
EVKLVESGGDLVKPGGSLRLSCMASGFTLNNYYMSWVRQAPGKGLQWVAYIKSGGSDTSYVDAVKGRFTISRDNAKNTLHLQMNSLRVEDTAVYYCATDGYIWAWGQGTLVTVSS

SEQ ID NO: 135 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgataacagcaggccgacagggatccctgagcgattctccggcgccaagtcggggaacacggccaccctgaccatcagcggggccctggccgaggacgaggctgactattactgccaggtgtgggacagcagttctaaggcttacgtgttcggctcaggaacccaactgaccgtcctcg

SEQ ID NO: 136 - protein, 108 aa, Canis sp.
  source 1..108: note = canis familiaris
SYVLTQLPSKNVTLKQPAHITCGGDNIGSKSVHWYQQKLGQAPVLIIYYDNSRPTGIPERFSGAKSGNTATLTISGALAEDEADYYCQVWDSSSKAYVFGSGTQLTVL

SEQ ID NO: 137 - protein, 7 aa, Canis sp.
  source 1..7: note = canis familiaris
GFTLNNY

SEQ ID NO: 138 - protein, 6 aa, Canis sp.
  source 1..6: note = canis familiaris
KSGGSD

SEQ ID NO: 139 - protein, 10 aa, Canis sp.
  source 1..10: note = canis familiaris
CATDGYIWAW

SEQ ID NO: 140 - protein, 11 aa, Canis sp.
  source 1..11: note = canis familiaris
GGDNIGSKSVH

SEQ ID NO: 141 - protein, 7 aa, Canis sp.
  source 1..7: note = canis familiaris
YDNSRPT

SEQ ID NO: 142 - protein, 13 aa, Canis sp.
  source 1..13: note = canis familiaris
CQVWDSSSKAYVF

SEQ ID NO: 143 - DNA, 355 bp, Canis sp.
  source 1..355: mol_type = genomic DNA; note = canis familiaris
gaactcacactgcaggagtcagggccaggactggtgaagccctcacagaccctctctctcacctgtgttgtgtccggaggctccgtcaccagaagttactactggaactggatccgccagcgccctgggaggggactggaatggatggggtactggacaggtaccacaaactacaatccggcatttcagggacgcatctccatcactgctgacacggccaagaaccagtgctccctgcagctgagctccatgaccaccgaggacacggccgtttattattgtgcaagggaggggggaaagctactgggagcttttggtcactggggccagggcaccctggtcactgtctcctcag

SEQ ID NO: 144 - protein, 118 aa, Canis sp.; identical to SEQ ID NO: 44
  source 1..118: note = canis familiaris

SEQ ID NO: 145 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaagattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 146 - protein, 108 aa, Canis sp.; identical to SEQ ID NO: 36
  source 1..108: note = canis familiaris

SEQ ID NO: 147 - protein, 7 aa, Canis sp.
  source 1..7: note = canis familiaris
GGSVTRS

SEQ ID NO: 148 - protein, 6 aa, Canis sp.
  source 1..6: note = canis familiaris
GYWTGT

SEQ ID NO: 149 - protein, 15 aa, Canis sp.
  source 1..15: note = canis familiaris
CAREGGKLLGAFGHW

SEQ ID NO: 150 - protein, 11 aa, Canis sp.
  source 1..11: note = canis familiaris
GGDKIGSKSVH

SEQ ID NO: 151 - protein, 7 aa, Canis sp.
  source 1..7: note = canis familiaris
YDSSRPT

SEQ ID NO: 152 - protein, 13 aa, Canis sp.
  source 1..13: note = canis familiaris
CQVWDSSAKASVF

SEQ ID NO: 153 - DNA, 376 bp, Canis sp.
  source 1..376: mol_type = genomic DNA; note = canis familiaris
gaggtgcaggtggtggagtctgggggagacctggtgaagcctggggggtccctgagactttcctgtgtgacctctggattcgccttcaataactaccacatgagctgggtccgccaggctccagggaaggggcttcagtgggtcgcatacattaacagtggtggaagtagaataagctatgcagacgctgtgaagggccgattcaccatctccagagacaacgccaagaacacgctgtatcttcagatgaacagcctgagagccgaggacacggccgtttattactgtgcgagtgagggggggggggagatatatggattcaaagactatggtatggactactggggccatggcacctcactcttcgtgtcctcag

SEQ ID NO: 154 - protein, 125 aa, Canis sp.
  source 1..125: note = canis familiaris
EVQVVESGGDLVKPGGSLRLSCVTSGFAFNNYHMSWVRQAPGKGLQWVAYINSGGSRISYADAVKGRFTISRDNAKNTLYLQMNSLRAEDTAVYYCASEGGGEIYGFKDYGMDYWGHGTSLFVSS

SEQ ID NO: 155 - DNA, 325 bp, Canis sp.; identical to SEQ ID NO: 85
  source 1..325: mol_type = genomic DNA; note = canis familiaris

SEQ ID NO: 156 - protein, 108 aa, Canis sp.; identical to SEQ ID NO: 66
  source 1..108: note = canis familiaris

SEQ ID NO: 157 - protein, 7 aa, Canis sp.
  source 1..7: note = canis familiaris
GFAFNNY

SEQ ID NO: 158 - protein, 6 aa, Canis sp.
  source 1..6: note = canis familiaris
NSGGSR

SEQ ID NO: 159 - protein, 20 aa, Canis sp.
  source 1..20: note = canis familiaris
CASEGGGEIYGFKDYGMDYW

SEQ ID NO: 160 - protein, 11 aa, Canis sp.
  source 1..11: note = canis familiaris
GGDNIGSKSVH

SEQ ID NO: 161 - protein, 7 aa, Canis sp.
  source 1..7: note = canis familiaris
YDSSRPT

SEQ ID NO: 162 - protein, 13 aa, Canis sp.
  source 1..13: note = canis familiaris
CQVWDSSAKAWVF

SEQ ID NO: 163 - DNA, 376 bp, Canis sp.
  source 1..376: mol_type = genomic DNA; note = canis familiaris
gaggtgcagttggtggagtctgggggagacctggtgaagcctggggggtccctgagactttcctgtgtggcctctggattcaccttcagtagttaccacatgagctgggtccgccaggctccagggaaggggcttcagtgggtcgcatacagtaacagtggtggaagtaggacaagttatgcagacgctgtgaagggccgattcaccatctccagagacaacgccaagaacacgctgtatcttcagatgagcagcctgagagccgaggacacggccgtatactactgtgcgagtgagggggggggggggagatatggattcaaagactatggtatggactactggggccatggcacctcactcttcgtgtcctcag

SEQ ID NO: 164 - protein, 125 aa, Canis sp.
  source 1..125: note = canis familiaris
EVQLVESGGDLVKPGGSLRLSCVASGFTFSSYHMSWVRQAPGKGLQWVAYSNSGGSRTSYADAVKGRFTISRDNAKNTLYLQMSSLRAEDTAVYYCASEGGGGRYGFKDYGMDYWGHGTSLFVSS

SEQ ID NO: 165 - DNA, 325 bp, Canis sp.; identical to SEQ ID NO: 85
  source 1..325: mol_type = genomic DNA; note = canis familiaris

SEQ ID NO: 166 - protein, 108 aa, Canis sp.; identical to SEQ ID NO: 66
  source 1..108: note = canis familiaris

SEQ ID NO: 167 - protein, 7 aa, Canis sp.
  source 1..7: note = canis familiaris
GFTFSSY

SEQ ID NO: 168 - protein, 6 aa, Canis sp.
  source 1..6: note = canis familiaris
NSGGSR

SEQ ID NO: 169 - protein, 20 aa, Canis sp.
  source 1..20: note = canis familiaris
CASEGGGGRYGFKDYGMDYW

SEQ ID NO: 170 - protein, 11 aa, Canis sp.
  source 1..11: note = canis familiaris
GGDNIGSKSVH

SEQ ID NO: 171 - protein, 7 aa, Canis sp.
  source 1..7: note = canis familiaris
YDSSRPT

SEQ ID NO: 172 - protein, 13 aa, Canis sp.
  source 1..13: note = canis familiaris
CQVWDSSAKAWVF

SEQ ID NO: 173 - DNA, 376 bp, Canis sp.
  source 1..376: mol_type = genomic DNA; note = canis familiaris
gaggtgcagctggtggagtctgggggagacctggtgaagcctggggggtccctgagactttcctgtgaggcctctggattcaccttcagtagctaccatatgagttgggtccgccaggctccagggaaggggcttcagtgggtcgcatacattaacaatggtggaagtggtacaagctatgcagacgctgtgaagggccgattcaccatctccagagacaacgccaagaacacgctctatcttcagatgaacagcctgcgagccgaggacacggccgtttatttctgtgcgagtgaggggggggggaagagatatggatacaaagactatggtatggactactggggccatggcacctcactcttcgtgtcctcag

SEQ ID NO: 174 - protein, 125 aa, Canis sp.
  source 1..125: note = canis familiaris
EVQLVESGGDLVKPGGSLRLSCEASGFTFSSYHMSWVRQAPGKGLQWVAYINNGGSGTSYADAVKGRFTISRDNAKNTLYLQMNSLRAEDTAVYFCASEGGGKRYGYKDYGMDYWGHGTSLFVSS

SEQ ID NO: 175 - DNA, 325 bp, Canis sp.; identical to SEQ ID NO: 85
  source 1..325: mol_type = genomic DNA; note = canis familiaris

SEQ ID NO: 176 - protein, 108 aa, Canis sp.; identical to SEQ ID NO: 66
  source 1..108: note = canis familiaris

SEQ ID NO: 177 - protein, 7 aa, Canis sp.
  source 1..7: note = canis familiaris
GFTFSSY

SEQ ID NO: 178 - protein, 6 aa, Canis sp.
  source 1..6: note = canis familiaris
NNGGSG

SEQ ID NO: 179 - protein, 20 aa, Canis sp.
  source 1..20: note = canis familiaris
CASEGGGKRYGYKDYGMDYW

SEQ ID NO: 180 - protein, 11 aa, Canis sp.
  source 1..11: note = canis familiaris
GGDNIGSKSVH

SEQ ID NO: 181 - protein, 7 aa, Canis sp.
  source 1..7: note = canis familiaris
YDSSRPT

SEQ ID NO: 182 - protein, 13 aa, Canis sp.
  source 1..13: note = canis familiaris
CQVWDSSAKAWVF

SEQ ID NO: 183 - DNA, 376 bp, Canis sp.
  source 1..376: mol_type = genomic DNA; note = canis familiaris
gaggtgcagttggtggagtctgggggagacctggtgaagcctggggggtccctgagactttcctgtgtggcctctggattcaccttcagtagttaccacatgagctgggtccgccaggctccagggaaggggcttcagtgggtcgcatacagtaacagtggtggaagtaggacaagttatgcagacgctgtgaagggccgattcaccatctccagagacaacgccaagaacacgctgtatcttcagatgaacagcctgagagccgaggacacggccgtgtactactgtgcgagtgagggggggggggggagatatggatacaaagactatggtatggactactggggccatggcacctcactcttcgtgtcctcag

SEQ ID NO: 184 - protein, 125 aa, Canis sp.
  source 1..125: note = canis familiaris
EVQLVESGGDLVKPGGSLRLSCVASGFTFSSYHMSWVRQAPGKGLQWVAYSNSGGSRTSYADAVKGRFTISRDNAKNTLYLQMNSLRAEDTAVYYCASEGGGGRYGYKDYGMDYWGHGTSLFVSS

SEQ ID NO: 185 - DNA, 325 bp, Canis sp.; identical to SEQ ID NO: 85
  source 1..325: mol_type = genomic DNA; note = canis familiaris

SEQ ID NO: 186 - protein, 108 aa, Canis sp.; identical to SEQ ID NO: 66
  source 1..108: note = canis familiaris

SEQ ID NO: 187 - protein, 7 aa, Canis sp.
  source 1..7: note = canis familiaris
GFTFSSY

SEQ ID NO: 188 - protein, 6 aa, Canis sp.
  source 1..6: note = canis familiaris
NSGGSR

SEQ ID NO: 189 - protein, 20 aa, Canis sp.
  source 1..20: note = canis familiaris
CASEGGGGRYGYKDYGMDYW

SEQ ID NO: 190 - protein, 11 aa, Canis sp.
  source 1..11: note = canis familiaris
GGDNIGSKSVH

SEQ ID NO: 191 - protein, 7 aa, Canis sp.
  source 1..7: note = canis familiaris
YDSSRPT

SEQ ID NO: 192 - protein, 13 aa, Canis sp.
  source 1..13: note = canis familiaris
CQVWDSSAKAWVF

SEQ ID NO: 193 - DNA, 379 bp, Canis sp.
  source 1..379: mol_type = genomic DNA; note = canis familiaris
gaggtgcaattggtggagtctgggggagacctggtgaagcctggggggtccctgagactttcctgtgtggcctctggattcaccttcagtacttaccacatgagttgggtccgccaggctccagggaaggggcttcagtgggtcgcctacagccatagtggtggaactaggacatattatgcagacgctgtgaagggccgattcaccatctccagagacaacggcaagaacacgctgtatcttcagatgaacagcctgagagccgaggacacggccgtgtactactgtgcgagtgaggggagtgggggggggagatatggatttaaagactatggtatggactactggggccatggcacctcactcttcgtgtccacag

SEQ ID NO: 194 - protein, 126 aa, Canis sp.
  source 1..126: note = canis familiaris
EVQLVESGGDLVKPGGSLRLSCVASGFTFSTYHMSWVRQAPGKGLQWVAYSHSGGTRTYYADAVKGRFTISRDNGKNTLYLQMNSLRAEDTAVYYCASEGSGGGRYGFKDYGMDYWGHGTSLFVST

SEQ ID NO: 195 - DNA, 325 bp, Canis sp.; identical to SEQ ID NO: 85
  source 1..325: mol_type = genomic DNA; note = canis familiaris

SEQ ID NO: 196 - protein, 108 aa, Canis sp.; identical to SEQ ID NO: 66
  source 1..108: note = canis familiaris

SEQ ID NO: 197 - protein, 7 aa, Canis sp.
  source 1..7: note = canis familiaris
GFTFSTY

SEQ ID NO: 198 - protein, 6 aa, Canis sp.
  source 1..6: note = canis familiaris
HSGGTR

SEQ ID NO: 199 - protein, 21 aa, Canis sp.
  source 1..21: note = canis familiaris
CASEGSGGGRYGFKDYGMDYW

SEQ ID NO: 200 - protein, 11 aa, Canis sp.
  source 1..11: note = canis familiaris
GGDNIGSKSVH

SEQ ID NO: 201 - protein, 7 aa, Canis sp.
  source 1..7: note = canis familiaris
YDSSRPT

SEQ ID NO: 202 - protein, 13 aa, Canis sp.
  source 1..13: note = canis familiaris
CQVWDSSAKAWVF

SEQ ID NO: 203 - DNA, 358 bp, Canis sp.
  source 1..358: mol_type = genomic DNA; note = canis familiaris
gaggtgcagctggtggagtctgggggagacctggtgaaggctggggtgtccctgagactttcctgtgtggcctctggattcaccttcagtaactatcacttgaactgggtccgccaggctccagggaaggggcctcagtgggtcgcatccattaacagtggtggaagtagcacatactatgcagacgctgtgaagggccgattcaccatctccagagacaacgccaagaacacgctttatcttcagatgaacagcctgagagccgaggacacggccgtgtattactgtgcgagtgaccacggtagctaccggggtatggactattggggccatggcacctcactcttcgtgtcctcag

SEQ ID NO: 204 - protein, 119 aa, Canis sp.
  source 1..119: note = canis familiaris
EVQLVESGGDLVKAGVSLRLSCVASGFTFSNYHLNWVRQAPGKGPQWVASINSGGSSTYYADAVKGRFTISRDNAKNTLYLQMNSLRAEDTAVYYCASDHGSYRGMDYWGHGTSLFVSS

SEQ ID NO: 205 - DNA, 322 bp, Canis sp.
  source 1..322: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaattcggggaacacggccaccctgaccatcagcggggccctggccgaggacgaggctgactattactgccaggtgtgggacagcagtgctaaggctgtgttcggcggaggcacccatctgaccgtcctcg

SEQ ID NO: 206 - protein, 107 aa, Canis sp.
  source 1..107: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKAVFGGGTHLTVL

SEQ ID NO: 207 - protein, 7 aa, Canis sp.
  source 1..7: note = canis familiaris
GFTFSNY

SEQ ID NO: 208 - protein, 6 aa, Canis sp.
  source 1..6: note = canis familiaris
NSGGSS

SEQ ID NO: 209 - protein, 14 aa, Canis sp.
  source 1..14: note = canis familiaris
CASDHGSYRGMDYW

SEQ ID NO: 210 - protein, 11 aa, Canis sp.
  source 1..11: note = canis familiaris
GGDNIGSKSVH

SEQ ID NO: 211 - protein, 7 aa, Canis sp.
  source 1..7: note = canis familiaris
YDSSRPT

SEQ ID NO: 212 - protein, 12 aa, Canis sp.
  source 1..12: note = canis familiaris
CQVWDSSAKAVF

SEQ ID NO: 213 - DNA, 361 bp, Canis sp.
  source 1..361: mol_type = genomic DNA; note = canis familiaris
gaactcacactgcaggagtcagggccaggactggtgaagccctcacagaccctctctctcacctgtgttgtgtccggaggctccgtcaccagtacttactactggaactggatccgccagcgccctgggaggggactggaatggatggggtactggacaggtaccacaaactacaacccggcattccagggacgcatctccatcactgctgacacggccaagaaccagttctccctgcagctgagttccatgaccaccgaggacacggccgtatattactgtgcaagagaagatttagtaacagttggttccggtgcttttaattactggggccagggcaccctggtcactgtctcctcag

SEQ ID NO: 214 - protein, 120 aa, Canis sp.
  source 1..120: note = canis familiaris
ELTLQESGPGLVKPSQTLSLTCVVSGGSVTSTYYWNWIRQRPGRGLEWMGYWTGTTNYNPAFQGRISITADTAKNQFSLQLSSMTTEDTAVYYCAREDLVTVGSGAFNYWGQGTLVTVSS

SEQ ID NO: 215 - DNA, 322 bp, Canis sp.
  source 1..322: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctacttgggtattcggtgaagggacccagctgaccgtcctcg

SEQ ID NO: 216 - protein, 107 aa, Canis sp.
  source 1..107: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSATWVFGEGTQLTVL

SEQ ID NO: 217 - protein, 7 aa, Canis sp.
  source 1..7: note = canis familiaris
GGSVTST

SEQ ID NO: 218 - protein, 6 aa, Canis sp.
  source 1..6: note = canis familiaris
GYWTGT

SEQ ID NO: 219 - protein, 17 aa, Canis sp.
  source 1..17: note = canis familiaris
CAREDLVTVGSGAFNYW

SEQ ID NO: 220 - protein, 11 aa, Canis sp.
  source 1..11: note = canis familiaris
GGDNIGSKSVH

SEQ ID NO: 221 - protein, 7 aa, Canis sp.
  source 1..7: note = canis familiaris
YDSSRPT

SEQ ID NO: 222 - protein, 12 aa, Canis sp.
  source 1..12: note = canis familiaris
CQVWDSSATWVF

SEQ ID NO: 223 - DNA, 346 bp, Canis sp.
  source 1..346: mol_type = genomic DNA; note = canis familiaris
gaggtgcagctggtggagtctgggggagacctggtgaagcctggggggtccctgagactttcctgtgtggcctctgggttcaccttcagtagctaccacatgagctgggtccgccaggctccagggaaggggcttcagtgggtcgcatacattaacagtgctggaagtaccacaagctatacagacgctgtgcagggccgattcaccatctccagagacaacgccaagaacacgctgtatcttcagatgaacagcctgagagccgaggacacggccgtgtattactgtgcgagtgagtggatatttatgccctggggccagggatccctggtcaccgtctcctcag

SEQ ID NO: 224 - protein, 115 aa, Canis sp.
  source 1..115: note = canis familiaris
EVQLVESGGDLVKPGGSLRLSCVASGFTFSSYHMSWVRQAPGKGLQWVAYINSAGSTTSYTDAVQGRFTISRDNAKNTLYLQMNSLRAEDTAVYYCASEWIFMPWGQGSLVTVSS

SEQ ID NO: 225 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagggtattcggtgaagggacccagctgaccgtcctcg

SEQ ID NO: 226 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKARVFGEGTQLTVL

SEQ ID NO: 227 - protein, 7 aa, Canis sp.
  source 1..7: note = canis familiaris
GFTFSSY

SEQ ID NO: 228 - protein, 6 aa, Canis sp.
  source 1..6: note = canis familiaris
NSAGST

SEQ ID NO: 229 - protein, 10 aa, Canis sp.
  source 1..10: note = canis familiaris
CASEWIFMPW

SEQ ID NO: 230 - protein, 11 aa, Canis sp.
  source 1..11: note = canis familiaris
GGDNIGSKSVH

SEQ ID NO: 231 - protein, 7 aa, Canis sp.
  source 1..7: note = canis familiaris
YDSSRPT

SEQ ID NO: 232 - protein, 13 aa, Canis sp.
  source 1..13: note = canis familiaris
CQVWDSSAKARVF

SEQ ID NO: 233 - DNA, 358 bp, Canis sp.
  source 1..358: mol_type = genomic DNA; note = canis familiaris
gaagtcacactgcaggagtcagggccaggactggtgaagccctcacagaccctctctctcacctgtgttgtgtccggaggctccgtcaccagcagtttttactggaactggatccgccagcgccctgggaggggactggaatggatgggatactggacaggtagcacaaattacaacccggcattccagggacgcatctccatcactgctgacacggccaagaaccagttctccctgcacctgggctccatgaccaccgatgacacggccatgtattactgtgcaagagaggagggtagttgggggagggtctattttgactactggggccagggaaccctggtcaccgtctcctcag

SEQ ID NO: 234 - protein, 119 aa, Canis sp.
  source 1..119: note = canis familiaris
EVTLQESGPGLVKPSQTLSLTCVVSGGSVTSSFYWNWIRQRPGRGLEWMGYWTGSTNYNPAFQGRISITADTAKNQFSLHLGSMTTDDTAMYYCAREEGSWGRVYFDYWGQGTLVTVSS

SEQ ID NO: 235 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtactaagacttgggtattcggtgaagggacccagctgaccgtcctcg

SEQ ID NO: 236 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSTKTWVFGEGTQLTVL

SEQ ID NO: 237 - protein, 7 aa, Canis sp.
  source 1..7: note = canis familiaris
GGSVTSS

SEQ ID NO: 238 - protein, 6 aa, Canis sp.
  source 1..6: note = canis familiaris
WTGSTN

SEQ ID NO: 239 - protein, 16 aa, Canis sp.
  source 1..16: note = canis familiaris
CAREEGSWGRVYFDYW

SEQ ID NO: 240 - protein, 11 aa, Canis sp.
  source 1..11: note = canis familiaris
GGDNIGSKSVH

SEQ ID NO: 241 - protein, 7 aa, Canis sp.
  source 1..7: note = canis familiaris
YDSSRPT

SEQ ID NO: 242 - protein, 13 aa, Canis sp.
  source 1..13: note = canis familiaris
CQVWDSSTKTWVF

SEQ ID NO: 243 - DNA, 355 bp, Canis sp.
  source 1..355: mol_type = genomic DNA; note = canis familiaris
gaactcgcactgcaggagtcagggccaggactggtgaggccctcacagaccctctctctcacctgtgttgtgtccggaggctccgtcaccagaagttactactggaactggatccgccagcgccctgggaggggactggaatggatgggttactggacaggtaccacaatctacaacccggcattccagggacgcatctccatcacagttgacacggccaagaatcagttctccctgcatctgatctccatgaccaccgaggacacggccgtgtattattgtggaagagaggggggttcttccctaggtgctattgcttactggggccagggcacccgggtcactgtctcctcag

SEQ ID NO: 244 - protein, 118 aa, Canis sp.; identical to SEQ ID NO: 94
  source 1..118: note = canis familiaris

SEQ ID NO: 245 - DNA, 325 bp, Canis sp.
  source 1..325: mol_type = genomic DNA; note = canis familiaris
tcctatgtgttgacacagctgccatccaaaaatgtgaccctgaagcagccggcccacatcacctgtgggggagacaacattggaagaaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctgctgtgttcggcggaggcacccacctgaccgtcctcg

SEQ ID NO: 246 - protein, 108 aa, Canis sp.
  source 1..108: note = canis familiaris
SYVLTQLPSKNVTLKQPAHITCGGDNIGRKSVHWYQQKLGQAPVLIIYYDSSRPTGIPERFSGANSGNTATLTISGALAEDEADYYCQVWDSSAKAAVFGGGTHLTVL

SEQ ID NO: 247 - protein, 7 aa, Canis sp.
  source 1..7: note = canis familiaris
GGSVTRS

SEQ ID NO: 248 - protein, 6 aa, Canis sp.
  source 1..6: note = canis familiaris
GYWTGT

SEQ ID NO: 249 - protein, 15 aa, Canis sp.
  source 1..15: note = canis familiaris
CGREGGSSLGAIAYW

SEQ ID NO: 250 - protein, 11 aa, Canis sp.
  source 1..11: note = canis familiaris
GGDNIGRKSVH

SEQ ID NO: 251 - protein, 7 aa, Canis sp.
  source 1..7: note = canis familiaris
YDSSRPT

SEQ ID NO: 252 - protein, 13 aa, Canis sp.
  source 1..13: note = canis familiaris
CQVWDSSAKAAVF

SEQ ID NO: 253 - DNA, 355 bp, Canis sp.; identical to SEQ ID NO: 243
  source 1..355: mol_type = genomic DNA; note = canis familiaris

SEQ ID NO: 254 - protein, 118 aa, Canis sp.; identical to SEQ ID NO: 94
  source 1..118: note = canis familiaris

SEQ ID NO: 255 - DNA, 325 bp, Canis sp.; identical to SEQ ID NO: 95
  source 1..325: mol_type = genomic DNA; note = canis familiaris

SEQ ID NO: 256 - protein, 108 aa, Canis sp.; identical to SEQ ID NO: 96
  source 1..108: note = canis familiaris

SEQ ID NO: 257 - protein, 7 aa, Canis sp.
  source 1..7: note = canis familiaris
GGSVTRS

SEQ ID NO: 258 - protein, 6 aa, Canis sp.
  source 1..6: note = canis familiaris
GYWTGT

SEQ ID NO: 259 - protein, 15 aa, Canis sp.
  source 1..15: note = canis familiaris
CGREGGSSLGAIAYW

SEQ ID NO: 260 - protein, 11 aa, Canis sp.
  source 1..11: note = canis familiaris
GGDNIGSKSVH

SEQ ID NO: 261 - protein, 7 aa, Canis sp.
  source 1..7: note = canis familiaris
YDSSRPT

SEQ ID NO: 262 - protein, 13 aa, Canis sp.
  source 1..13: note = canis familiaris
CQVWDSSAKAAVF

SEQ ID NO: 263 - DNA, 349 bp, Canis sp.
  source 1..349: mol_type = genomic DNA; note = canis familiaris
gaactcacactgcaggagtcagggccaggactggtgaagccctcacagaccctctctctcacctgtgttgtgtccggaggctccgtcaccagcaattactactggaactggatccgccagcgccctgggaggggactggaatggatggggtactggacaggtagcacaaactacaacccggcattccagggacgcatctccatcactgctgacacggccaagaagcagttctccctgcagctgaggtccatgaccattgaagacacggccgtgtattactgtgcaagaagaggaatacctatattttttgactactggggccagggaaccctggtcaccgtctcctcag

SEQ ID NO: 264 - protein, 116 aa, Canis sp.
  source 1..116: note = canis familiaris
ELTLQESGPGLVKPSQTLSLTCVVSGGSVTSNYYWNWIRQRPGRGLEWMGYWTGSTNYNPAFQGRISITADTAKKQFSLQLRSMTIEDTAVYYCARRGIPIFFDYWGQGTLVTVSS

SEQ ID NO: 265 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 266 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSASVFGGGTHLTVL

SEQ ID NO: 267 - protein, 7 aa, Canis sp.
  source 1..7: note = canis familiaris
GGSVTSN

SEQ ID NO: 268 - protein, 6 aa, Canis sp.
  source 1..6: note = canis familiaris
GYWTGS

SEQ ID NO: 269 - protein, 13 aa, Canis sp.
  source 1..13: note = canis familiaris
CARRGIPIFFDYW

SEQ ID NO: 270 - protein, 11 aa, Canis sp.
  source 1..11: note = canis familiaris
GGDNIGSKSVH

SEQ ID NO: 271 - protein, 7 aa, Canis sp.
  source 1..7: note = canis familiaris
YDSSRPT

SEQ ID NO: 272 - protein, 11 aa, Canis sp.
  source 1..11: note = canis familiaris
CQVWDSSASVF

SEQ ID NO: 273 - DNA, 349 bp, Canis sp.
  source 1..349: mol_type = genomic DNA; note = canis familiaris
gaactcacactgcaggagtcagggccaggactggtgaagccctcacagaccctctctctcacctgtattgtgtccggaggctccgtcaccaacaattactactggacctggatccgccagcgccctgggaggggactggaatggatgggatactggacaggtttcacaaactacaatccggcattccagggacgcatctccatcattgctaacacggccaagaaacaattgtccctgcagctgagctccttgaccaccgaagacacggccgtatattactgtgcaagaagggcgatacctacctattttgactactggggccagggaaccctagtcaccgtctcctccg

SEQ ID NO: 274 - protein, 116 aa, Canis sp.
  source 1..116: note = canis familiaris
ELTLQESGPGLVKPSQTLSLTCIVSGGSVTNNYYWTWIRQRPGRGLEWMGYWTGFTNYNPAFQGRISIIANTAKKQLSLQLSSLTTEDTAVYYCARRAIPTYFDYWGQGTLVTVSS

SEQ ID NO: 275 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtagaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctagtgtgttcggcggaggcacccatctgaccgtcctcg

SEQ ID NO: 276 - protein, 106 aa, Canis sp.
  source 1..106: note = canis familiaris
SYVLTQLPSKNVTLKQPAHITCGGDNIGSRSVHWYQQKLGQAPVLIIYYDSSRPTGIPERFSGANSGNTATLTISGALAEDEADYYCQVWDSSASVFGGGTHLTVL

SEQ ID NO: 277 - protein, 10 aa, Canis sp.
  source 1..10: note = canis familiaris
GGSVTNNYYW

SEQ ID NO: 278 - protein, 8 aa, Canis sp.
  source 1..8: note = canis familiaris
FHKLQSGI

SEQ ID NO: 279 - protein, 13 aa, Canis sp.
  source 1..13: note = canis familiaris
CARRAIPTYFDYW

SEQ ID NO: 280 - protein, 11 aa, Canis sp.
  source 1..11: note = canis familiaris
GGDNIGSRSVH

SEQ ID NO: 281 - protein, 7 aa, Canis sp.
  source 1..7: note = canis familiaris
YDSSRPT

SEQ ID NO: 282 - protein, 11 aa, Canis sp.
  source 1..11: note = canis familiaris
CQVWDSSASVF

SEQ ID NO: 283 - DNA, 373 bp, Canis sp.
  source 1..373: mol_type = genomic DNA; note = canis familiaris
gaggtgcagctggtggagtctgggggagacctggtgaagcctggggggtccctgagactttcctgtgtgacatctggattcaccttcagtaactatcacatgaattgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccatgtattactgtgcgagtgactgggactactgtgctgatgatacctgtgagggtatgggctactggggccatggcacctcactcttcgtgtcctcag

SEQ ID NO: 284 - protein, 124 aa, Canis sp.
  source 1..124: note = canis familiaris
EVQLVESGGDLVKPGGSLRLSCVTSGFTFSNYHMNWVRQAPGKGLQWVAYINSGGSSTSYADAVKGRFTISRDNAKNTLYLQMNSLRAEDTAMYYCASDWDYCADDTCEGMGYWGHGTSLFVSS

SEQ ID NO: 285 - DNA, 325 bp, Canis sp.
  source 1..325: mol_type = genomic DNA; note = canis familiaris
tcctatgtgctgacacagctgccatccaaaaatgtgaccctgaaacagccggcccacatcacctgtgggggagacaatattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 286 - protein, 108 aa, Canis sp.; identical to SEQ ID NO: 66
  source 1..108: note = canis familiaris

SEQ ID NO: 287 - protein, 7 aa, Canis sp.
  source 1..7: note = canis familiaris
GFTFSNY

SEQ ID NO: 288 - protein, 6 aa, Canis sp.
  source 1..6: note = canis familiaris
NSGGSS

SEQ ID NO: 289 - protein, 19 aa, Canis sp.
  source 1..19: note = canis familiaris
CASDWDYCADDTCEGMGYW

SEQ ID NO: 290 - protein, 11 aa, Canis sp.
  source 1..11: note = canis familiaris
GGDNIGSKSVH

SEQ ID NO: 291 - protein, 7 aa, Canis sp.
  source 1..7: note = canis familiaris
YDSSRPT

SEQ ID NO: 292 - protein, 13 aa, Canis sp.
  source 1..13: note = canis familiaris
CQVWDSSAKAWVF

SEQ ID NO: 293 - DNA, 358 bp, Canis sp.
  source 1..358: mol_type = genomic DNA; note = canis familiaris
gaactcacactgcaggagtcagggccaggactggtgaagccctcacagaccctctctctcacctgtgttgtgtccggaggctccgtcaccaacagttactactggaactggatccgccagcgccctgggcgggaactggaatggatggggtactggacaggtggcacaaagtacaacccggcattccagggacgcatctccatcagtgctgacacggccaagaatcagttctccctgcagctgacctccatgaccaccgaggacacggccgtgtattactgtgcaagagatcgagatcggagagtcccacatgcttatggttactggggccagggcaccctggtcactgtctcctcag

SEQ ID NO: 294 - protein, 119 aa, Canis sp.
  source 1..119: note = canis familiaris
ELTLQESGPGLVKPSQTLSLTCVVSGGSVTNSYYWNWIRQRPGRELEWMGYWTGGTKYNPAFQGRISISADTAKNQFSLQLTSMTTEDTAVYYCARDRDRRVPHAYGYWGQGTLVTVSS

SEQ ID NO: 295 - DNA, 325 bp, Canis sp.
  source 1..325: mol_type = genomic DNA; note = canis familiaris
tcctatgtactgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 296 - protein, 108 aa, Canis sp.; identical to SEQ ID NO: 106
  source 1..108: note = canis familiaris

SEQ ID NO: 297 - protein, 5 aa, Canis sp.
  source 1..5: note = canis familiaris
GGSVT

SEQ ID NO: 298 - protein, 6 aa, Canis sp.
  source 1..6: note = canis familiaris
GYWTGG

SEQ ID NO: 299 - protein, 16 aa, Canis sp.
  source 1..16: note = canis familiaris
CARDRDRRVPHAYGYW

SEQ ID NO: 300 - protein, 11 aa, Canis sp.
  source 1..11: note = canis familiaris
GGDNIGSKSVH

SEQ ID NO: 301 - protein, 7 aa, Canis sp.
  source 1..7: note = canis familiaris
YDSSRPT

SEQ ID NO: 302 - protein, 13 aa, Canis sp.
  source 1..13: note = canis familiaris
CQVWDSSAKASVF

SEQ ID NO: 303 - DNA, 355 bp, Canis sp.
  source 1..355: mol_type = genomic DNA; note = canis familiaris
gaactcacactgcaggagtcagggccaggactggtgaagccctcacagaccctctctctcacctgtggtgtgtccggaggctccgtcaccagaagtcactactggaattgggtccgccagcgccctgggaggggactggaatggatgggatactggacaggtagcacaaactacaacccggcattccagggacgcatctccatcactgctgacacggccaagaaccagttctccctgcagctgaggtccatgaccaccgaggacacggccgtatatttctgtgcaagagagactacaatacctacttttttttttgactactggggccagggaaccctggtcaccgtctcctcag

SEQ ID NO: 304 - protein, 118 aa, Canis sp.
  source 1..118: note = canis familiaris
ELTLQESGPGLVKPSQTLSLTCGVSGGSVTRSHYWNWVRQRPGRGLEWMGYWTGSTNYNPAFQGRISITADTAKNQFSLQLRSMTTEDTAVYFCARETTIPTFFFDYWGQGTLVTVSS

SEQ ID NO: 305 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcggtgctaaggcttgggttttcggtgaagggacccagctgaccgtcctcg

SEQ ID NO: 306 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSGAKAWVFGEGTQLTVL

SEQ ID NO: 307 - protein, 7 aa, Canis sp.
  source 1..7: note = canis familiaris
GGSVTRS

SEQ ID NO: 308 - protein, 6 aa, Canis sp.
  source 1..6: note = canis familiaris
GYWTGS

SEQ ID NO: 309 - protein, 15 aa, Canis sp.
  source 1..15: note = canis familiaris
CARETTIPTFFFDYW

SEQ ID NO: 310 - protein, 11 aa, Canis sp.
  source 1..11: note = canis familiaris
GGDNIGSKSVH

SEQ ID NO: 311 - protein, 7 aa, Canis sp.
  source 1..7: note = canis familiaris
YDSSRPT

SEQ ID NO: 312 - protein, 13 aa, Canis sp.
  source 1..13: note = canis familiaris
CQVWDSGAKAWVF

SEQ ID NO: 313 - DNA, 376 bp, Canis sp.
  source 1..376: mol_type = genomic DNA; note = canis familiaris
gaggtgcagctggtggagtctgggggagacctggtgaagcctggggggtccctgagactgtcctgtgtggcctctggattcaccttcagtagctaccacatgagttgggtccgtcaggctccagggaaggggcttcaatgggtcgcatccattaacagtggtggaagtagaacaagctatgtagacgctgtgaagggccgattcaccatctccagagacaacgccaagaaaacgctgtatcttcagatgaatagcctgagagccgaggacacggccgtttattactgtgcgagtgggcgaaccgggcccaagttgggactaggagactatggtatggactcctggggccatggcacctcactcttcgtgtcctcag

SEQ ID NO: 314 - protein, 125 aa, Canis sp.
  source 1..125: note = canis familiaris
EVQLVESGGDLVKPGGSLRLSCVASGFTFSSYHMSWVRQAPGKGLQWVASINSGGSRTSYVDAVKGRFTISRDNAKKTLYLQMNSLRAEDTAVYYCASGRTGPKLGLGDYGMDSWGHGTSLFVSS

SEQ ID NO: 315 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtaatgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 316 - protein, 108 aa, Canis sp.
  source 1..108: note = canis familiaris
SYVLTQLPSKNVTLKQPAHITCGGDNIGSKSVHWYQQKLGQAPVMIIYYDSSRPTGIPERFSGANSGNTATLTISGALAEDEADYYCQVWDSSAKAWVFGEGTQLTVL

SEQ ID NO: 317 - protein, 7 aa, Canis sp.
  source 1..7: note = canis familiaris
GFTFSSY

SEQ ID NO: 318 - protein, 6 aa, Canis sp.
  source 1..6: note = canis familiaris
NSGGSR

SEQ ID NO: 319 - protein, 20 aa, Canis sp.
  source 1..20: note = canis familiaris
CASGRTGPKLGLGDYGMDSW

SEQ ID NO: 320 - protein, 11 aa, Canis sp.
  source 1..11: note = canis familiaris
GGDNIGSKSVH

SEQ ID NO: 321 - protein, 7 aa, Canis sp.
  source 1..7: note = canis familiaris
YDSSRPT

SEQ ID NO: 322 - protein, 13 aa, Canis sp.
  source 1..13: note = canis familiaris
CQVWDSSAKAWVF

SEQ ID NO: 323 - DNA, 376 bp, Canis sp.
  source 1..376: mol_type = genomic DNA; note = canis familiaris
gaggtgaagctggtggagtctgggggagacctggtgaagcctggggggtccctgagactttcctgtgtggcctctggagtcaccttcagtaggtattacatgagctgggtccgccaggctccagggaaggggcttcagtgggtcgcatacattaacagtggtggaagtaccacaagttatgcagacgctgtgaagggccgattcaccatctccagagacaacgccaagaacacgctgtatcttcagatgaacagcctgagagccgaggacacggccgtgtattactgtgcgagtgagggggtgggggagatatatggatacaaagactttggtatggactactggggccatggcacctcactcttcgtgtcctcag

SEQ ID NO: 324 - protein, 125 aa, Canis sp.
  source 1..125: note = canis familiaris
EVKLVESGGDLVKPGGSLRLSCVASGVTFSRYYMSWVRQAPGKGLQWVAYINSGGSTTSYADAVKGRFTISRDNAKNTLYLQMNSLRAEDTAVYYCASEGVGEIYGYKDFGMDYWGHGTSLFVSS

SEQ ID NO: 325 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagaaggccgacagggatcccagagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 326 - protein, 108 aa, Canis sp.
  source 1..108: note = canis familiaris
SYVLTQLPSKNVTLKQPAHITCGGDNIGSKSVHWYQQKLGQAPVLIIYYDSRRPTGIPERFSGANSGNTATLTISGALAEDEADYYCQVWDSSAKAWVFGEGTQLTVL

SEQ ID NO: 327 - protein, 7 aa, Canis sp.
  source 1..7: note = canis familiaris
GVTFSRY

SEQ ID NO: 328 - protein, 6 aa, Canis sp.
  source 1..6: note = canis familiaris
NSGGST

SEQ ID NO: 329 - protein, 20 aa, Canis sp.
  source 1..20: note = canis familiaris
CASEGVGEIYGYKDFGMDYW

SEQ ID NO: 330 - protein, 11 aa, Canis sp.
  source 1..11: note = canis familiaris
GGDNIGSKSVH

SEQ ID NO: 331 - protein, 7 aa, Canis sp.
  source 1..7: note = canis familiaris
YDSRRPT

SEQ ID NO: 332 - protein, 13 aa, Canis sp.
  source 1..13: note = canis familiaris
CQVWDSSAKAWVF

SEQ ID NO: 333 - DNA, 376 bp, Canis sp.
  source 1..376: mol_type = genomic DNA; note = canis familiaris
gaggtgcagctggtggagtctgggggagacctggtgaagcctggggggtccctgagactgtcctgtgtggcctctggattcaccttcagtagctaccacatgagttgggtccgtcaggctccagggaaggggcttcagtgggtcgcatccattaacagtggtggaagtagaacaagctatgtagacgctgtgaagggccgcttcaccatctccagagacaacgccaagaacacgctgtatcttcagatgaacagcctgagagccgaggacacggccgtgtattactgtgcgagtgggcgaaccgggcccaagttggggctaggagactatggtatggactcctggggccatggcacctcactcttcgtgtcctcag

SEQ ID NO: 334 - protein, 125 aa, Canis sp.
  source 1..125: note = canis familiaris
EVQLVESGGDLVKPGGSLRLSCVASGFTFSSYHMSWVRQAPGKGLQWVASINSGGSRTSYVDAVKGRFTISRDNAKNTLYLQMNSLRAEDTAVYYCASGRTGPKLGLGDYGMDSWGHGTSLFVSS

SEQ ID NO: 335 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgcagggacccagctgaccgtcctcg

SEQ ID NO: 336 - protein, 108 aa, Canis sp.
  source 1..108: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSAKAWVFGAGTQLTVL

SEQ ID NO: 337 - protein, 7 aa, Canis sp.
  source 1..7: note = canis familiaris
GFTFSSY

SEQ ID NO: 338 - protein, 6 aa, Canis sp.
  source 1..6: note = canis familiaris
NSGGSR

SEQ ID NO: 339 - protein, 20 aa, Canis sp.
  source 1..20: note = canis familiaris
CASGRTGPKLGLGDYGMDSW

SEQ ID NO: 340 - protein, 11 aa, Canis sp.
  source 1..11: note = canis familiaris
GGDNIGSKSVH

SEQ ID NO: 341 - protein, 7 aa, Canis sp.
  source 1..7: note = canis familiaris
YDSSRPT

SEQ ID NO: 342 - protein, 13 aa, Canis sp.
  source 1..13: note = canis familiaris
CQVWDSSAKAWVF

SEQ ID NO: 343 - DNA, 376 bp, Canis sp.
  source 1..376: mol_type = genomic DNA; note = canis familiaris
gaggtgcagctggtggagtctgggggagacctggtgaagcctggggggtccctgagactgtcctgtgtggcctctggattcaccttcagtagctaccacatgagttgggtccgtcaggctccagggaaggggcttcaatgggtcgcatccattaacagtggtggaagtagaacaagctatgtagacgctgtgaagggccgcttcactatctccagagacaacgccaagaaaacgctgtatcttcagatgaatagcctgagagccgaggacacggccgtttattactgtgcgagtgggcgaaccgggcccaagttgggactaggagactatggtatggactcctggggccatggcacctcactcttcgtgtcctcag

SEQ ID NO: 344 - protein, 125 aa, Canis sp.; identical to SEQ ID NO: 314
  source 1..125: note = canis familiaris

SEQ ID NO: 345 - DNA, 325 bp, Canis sp.
  source 1..325: mol_type = genomic DNA; note = canis familiaris
tcctatgtgctgacacagctgccatccaaaaatgtcaccctgaagcagccggcccacatcacctgtgggggagacaacattggaagtaaaagtgttcactggtaccagcagaagctgggccaggcccctatactgattatctattatgatagcagtaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 346 - protein, 108 aa, Canis sp.
  source 1..108: note = canis familiaris
SYVLTQLPSKNVTLKQPAHITCGGDNIGSKSVHWYQQKLGQAPILIIYYDSSRPTGIPERFSGANSGNTATLTISGALAEDEADYYCQVWDSSAKAWVFGEGTQLTVL

SEQ ID NO: 347 - protein, 7 aa, Canis sp.
  source 1..7: note = canis familiaris
GFTFSSY

SEQ ID NO: 348 - protein, 6 aa, Canis sp.
  source 1..6: note = canis familiaris
NSGGSR

SEQ ID NO: 349 - protein, 20 aa, Canis sp.
  source 1..20: note = canis familiaris
CASGRTGPKLGLGDYGMDSW

SEQ ID NO: 350 - protein, 11 aa, Canis sp.
  source 1..11: note = canis familiaris
GGDNIGSKSVH

SEQ ID NO: 351 - protein, 7 aa, Canis sp.
  source 1..7: note = canis familiaris
YDSSRPT

SEQ ID NO: 352 - protein, 13 aa, Canis sp.
  source 1..13: note = canis familiaris
CQVWDSSAKAWVF

SEQ ID NO: 353 - DNA, 376 bp, Canis sp.; identical to SEQ ID NO: 73
  source 1..376: mol_type = genomic DNA; note = canis familiaris

SEQ ID NO: 354 - protein, 125 aa, Canis sp.; identical to SEQ ID NO: 74
  source 1..125: note = canis familiaris

SEQ ID NO: 355 - DNA, 325 bp, Canis sp.; identical to SEQ ID NO: 75
  source 1..325: mol_type = genomic DNA; note = canis familiaris

SEQ ID NO: 356 - protein, 108 aa, Canis sp.; identical to SEQ ID NO: 66
  source 1..108: note = canis familiaris

SEQ ID NO: 357 - protein, 7 aa, Canis sp.
  source 1..7: note = canis familiaris
GFTFSSY

SEQ ID NO: 358 - protein, 6 aa, Canis sp.
  source 1..6: note = canis familiaris
SSGGGR

SEQ ID NO: 359 - protein, 20 aa, Canis sp.
  source 1..20: note = canis familiaris
CAREGGGEIYGYKDYGMDYW

SEQ ID NO: 360 - protein, 11 aa, Canis sp.
  source 1..11: note = canis familiaris
GGDNIGSKSVH

SEQ ID NO: 361 - protein, 7 aa, Canis sp.
  source 1..7: note = canis familiaris
YDSSRPT

SEQ ID NO: 362 - protein, 13 aa, Canis sp.
  source 1..13: note = canis familiaris
CQVWDSSAKAWVF

SEQ ID NO: 363 - DNA, 349 bp, Canis sp.
  source 1..349: mol_type = genomic DNA; note = canis familiaris
gaggtgcaactggtggagtctgggggagacctggtgaagcctgcggggtccctgagactgtcctgtgtggcctctggattcaccttcagtgactacagtatgagctgggtccgccagggtcctgagaagggactacagttggtcgcaggtatgaacagcgatggcagtaccacatactacacagacgctgtgaggggccgattcaccatctccagagacaacgccaagaacacaatgtatctgcagatgaacagcctgagagacgaggacacggccatttattactgtgcaaagggggcagctgcccctgactactggggccagggaaccctggtcaccgtctcctcaa

SEQ ID NO: 364 - protein, 116 aa, Canis sp.
  source 1..116: note = canis familiaris
EVQLVESGGDLVKPAGSLRLSCVASGFTFSDYSMSWVRQGPEKGLQLVAGMNSDGSTTYYTDAVRGRFTISRDNAKNTMYLQMNSLRDEDTAIYYCAKGAAAPDYWGQGTLVTVSS

SEQ ID NO: 365 - DNA, 319 bp, Canis sp.
  source 1..319: mol_type = genomic DNA; note = canis familiaris
tcctatgagctgactcagccaccatccgtgaatgtgaccctgagggagacggcccacatcacctgtgggggagacagaattggaagtaaatatgttcaatggatccagcagattccaggccaggcccccgtggtgattatctataaagataacaaccggccgacagggatccctgagcgattctctggcgccaactcagggaacacggctaccctgaccatcagtggggccctggccgaagacgaggctgactattactgccaggtgggggacaattatacttgggtattcggtgaagggacccagctgaccgtcctcg

SEQ ID NO: 366 - protein, 106 aa, Canis sp.
  source 1..106: note = canis familiaris
SYELTQPPSVNVTLRETAHITCGGDRIGSKYVQWIQQIPGQAPVVIIYKDNNRPTGIPERFSGANSGNTATLTISGALAEDEADYYCQVGDNYTWVFGEGTQLTVL

SEQ ID NO: 367 - protein, 7 aa, Canis sp.
  source 1..7: note = canis familiaris
GFTFSDY

SEQ ID NO: 368 - protein, 6 aa, Canis sp.
  source 1..6: note = canis familiaris
NSDGST

SEQ ID NO: 369 - protein, 11 aa, Canis sp.
  source 1..11: note = canis familiaris
CAKGAAAPDYW

SEQ ID NO: 370 - protein, 11 aa, Canis sp.
  source 1..11: note = canis familiaris
GGDRIGSKYVQ

SEQ ID NO: 371 - protein, 7 aa, Canis sp.
  source 1..7: note = canis familiaris
KDNNRPT

SEQ ID NO: 372 - protein, 11 aa, Canis sp.
  source 1..11: note = canis familiaris
CQVGDNYTWVF

SEQ ID NO: 373 - DNA, 349 bp, Canis sp.
  source 1..349: mol_type = genomic DNA; note = canis familiaris
gaggtgcagttggtggagtctgggggagacctggtgaagcctgcggggtccctgagactgtcctgtgtggcctctggattcaccttcagtgactacagcatgagctgggtccgccaggctcctgagaagggactgcagttggtcgcaggtattaacggcgatggaagtaccacatactacacagacgctgtgaagggccgattcaccatctccagagacaacgccaagaacacagtctatctacagatgaacagtctgagagccgaggacacggccatgtattactgtgcaaagggagctggtactggtgacttttggggccagggaaccctggtcaccgtctcctcag

SEQ ID NO: 374 - protein, 116 aa, Canis sp.
  source 1..116: note = canis familiaris
EVQLVESGGDLVKPAGSLRLSCVASGFTFSDYSMSWVRQAPEKGLQLVAGINGDGSTTYYTDAVKGRFTISRDNAKNTVYLQMNSLRAEDTAMYYCAKGAGTGDFWGQGTLVTVSS

SEQ ID NO: 375 - DNA, 319 bp, Canis sp.
  source 1..319: mol_type = genomic DNA; note = canis familiaris
tcctatgagctgactcagccaccatccgtgaatgtgaccctgagggagacggcccacatcacctgtgggggagccagcattggaagaaaatatgttcaatggatccagcagattccaggccaggcccccgtggtgattatctataaagatagcaacaggccgacagggatccctgagcgattctctggcgccaactcagggaacacggctaccctgaccatcagtggggccctggccgaagacgaggctgactattactgccaggtgggggacagtggtgcttgggttttcggtgaagggacccagttgaccgtcctcg

SEQ ID NO: 376 - protein, 106 aa, Canis sp.
  source 1..106: note = canis familiaris
SYELTQPPSVNVTLRETAHITCGGASIGRKYVQWIQQIPGQAPVVIIYKDSNRPTGIPERFSGANSGNTATLTISGALAEDEADYYCQVGDSGAWVFGEGTQLTVL

SEQ ID NO: 377 - protein, 7 aa, Canis sp.
  source 1..7: note = canis familiaris
GFTFSDY

SEQ ID NO: 378 - protein, 6 aa, Canis sp.
  source 1..6: note = canis familiaris
NGDGST

SEQ ID NO: 379 - protein, 11 aa, Canis sp.
  source 1..11: note = canis familiaris
CAKGAGTGDFW

SEQ ID NO: 380 - protein, 11 aa, Canis sp.
  source 1..11: note = canis familiaris
GGASIGRKYVQ

SEQ ID NO: 381 - protein, 7 aa, Canis sp.
  source 1..7: note = canis familiaris
KDSNRPT

SEQ ID NO: 382 - protein, 11 aa, Canis sp.
  source 1..11: note = canis familiaris
CQVGDSGAWVF

SEQ ID NO: 383 - DNA, 355 bp, Canis sp.
  source 1..355: mol_type = genomic DNA; note = canis familiaris
gaggtgcagctggtggagtctgggggagacctggtgaagcctggggggtccctgagactttcctgtgtggcctctggattcaccttcagtacctaccacatgagctgggtccgccaggctccagggaagggccttcagtgggtcgcatacattagcagtggtggaagtcgcacaaactatgcagacgctgtgaagggccgattcaccatctccagagacaacgccaagaacacgctgtatcttcagatgaacagcctgagagccgaggacgcggccgtgttttactgtgcgaggcactacggtagctctctttttgactactggggccagggaaccctggtcaccgtctcctcag

SEQ ID NO: 384 - protein, 118 aa, Canis sp.
  source 1..118: note = canis familiaris
EVQLVESGGDLVKPGGSLRLSCVASGFTFSTYHMSWVRQAPGKGLQWVAYISSGGSRTNYADAVKGRFTISRDNAKNTLYLQMNSLRAEDAAVFYCARHYGSSLFDYWGQGTLVTVSS

SEQ ID NO: 385 - DNA, 334 bp, Canis sp.
  source 1..334: mol_type = genomic DNA; note = canis familiaris
gatattgtcatgacacagaccccactgtccctgtccgtcagccctggagagccggcctccatctcctgcaaggccagtcagagcctcctgcacattaatgggaacacctatttgtattggttccgacagaagccaggccagtctccacagcgtttgatctataaggtctccaacagagaccctggggtcccagacaggttcagtggcagcgggtcagggacagatttcaccctgagaatcagcagagtggaggctgatgatgctggagtttattactgcgggcaaggtatacaagatcctacctttggcaaagggacacatctggagattaaac

SEQ ID NO: 386 - protein, 111 aa, Canis sp.
  source 1..111: note = canis familiaris
DIVMTQTPLSLSVSPGEPASISCKASQSLLHINGNTYLYWFRQKPGQSPQRLIYKVSNRDPGVPDRFSGSGSGTDFTLRISRVEADDAGVYYCGQGIQDPTFGKGTHLEIK

SEQ ID NO: 387 - protein, 7 aa, Canis sp.
  source 1..7: note = canis familiaris
GFTFSTY

SEQ ID NO: 388 - protein, 6 aa, Canis sp.
  source 1..6: note = canis familiaris
SSGGSR

SEQ ID NO: 389 - protein, 13 aa, Canis sp.
  source 1..13: note = canis familiaris
CARHYGSSLFDYW

SEQ ID NO: 390 - protein, 11 aa, Canis sp.
  source 1..11: note = canis familiaris
CKASQSLLHIN

SEQ ID NO: 391 - protein, 7 aa, Canis sp.
  source 1..7: note = canis familiaris
PQRLIYK

SEQ ID NO: 392 - protein, 10 aa, Canis sp.
  source 1..10: note = canis familiaris
CGQGIQDPTF

SEQ ID NO: 393 - DNA, 352 bp, Canis sp.
  source 1..352: mol_type = genomic DNA; note = canis familiaris
gaggtgcagctggtggagtctgggggagacctggtgaagcctggggggtccctgagactttcctgtgtggcctctggattcaccttcagtaactaccacatgagttgggtccgccaggctccagggaaggggcttcagtgggtcgcatacagtaacagtggtagaatgagcacagactatgcagacgctgtgaagggccgattcaccatctccagagacaatgccaagaacacgctgtatcttcagatgaacagcctgagacccgaggacacggccgtgtattactgtgcgactgtcatatatggcctttttgactactggggccagggaaccctggtcaccgtctcctcag

SEQ ID NO: 394 - protein, 117 aa, Canis sp.
  source 1..117: note = canis familiaris
EVQLVESGGDLVKPGGSLRLSCVASGFTFSNYHMSWVRQAPGKGLQWVAYSNSGRMSTDYADAVKGRFTISRDNAKNTLYLQMNSLRPEDTAVYYCATVIYGLFDYWGQGTLVTVSS

SEQ ID NO: 395 - DNA, 337 bp, Canis sp.
  source 1..337: mol_type = genomic DNA; note = canis familiaris
gatattgtcatgacacagaccccactgtccctgtccgtcagccctggagagccggcctccatctcctgcaaggccagtcagagcctcctgtacagtaatgggaacacctatttgtattggttccgacagaagccaggccagtctccacagcgtttgatctataaggtctccaagagagactctggggtcccagacaggttcagtggcagcgggtcagggacagatttcaccctgagaatcagaagagtggaggctgatgatgctggagtttattactgcgggcaaggtacacaagatccttatactttcagccagggaaccaagctggagataaaac

SEQ ID NO: 396 - protein, 112 aa, Canis sp.
  source 1..112: note = canis familiaris
DIVMTQTPLSLSVSPGEPASISCKASQSLLYSNGNTYLYWFRQKPGQSPQRLIYKVSKRDSGVPDRFSGSGSGTDFTLRIRRVEADDAGVYYCGQGTQDPYTFSQGTKLEIK

SEQ ID NO: 397 - protein, 7 aa, Canis sp.
  source 1..7: note = canis familiaris
GFTFSNY

SEQ ID NO: 398 - protein, 6 aa, Canis sp.
  source 1..6: note = canis familiaris
NSGRMS

SEQ ID NO: 399 - protein, 12 aa, Canis sp.
  source 1..12: note = canis familiaris
CATVIYGLFDYW

SEQ ID NO: 400 - protein, 11 aa, Canis sp.
  source 1..11: note = canis familiaris
CKASQSLLYSN

SEQ ID NO: 401 - protein, 7 aa, Canis sp.
  source 1..7: note = canis familiaris
PQRLIYK

SEQ ID NO: 402 - protein, 11 aa, Canis sp.
  source 1..11: note = canis familiaris
CGQGTQDPYTF

SEQ ID NO: 403 - DNA, 349 bp, Canis sp.
  source 1..349: mol_type = genomic DNA; note = canis familiaris
gaggtgcagttggtggagtctgggggagacctggtgaagcctgccgggtccctgagactgtcctgtatggcctctggattcaccttcagtagttacagtatgagctgggtccgccaggctcctgagaaggggctgcagttggtcgcaggtattaatggcgatggaagtaccacatactacacagacgctgtgaagggccgcttcaccatctccagagacaacgccaaaaacacagtatatctgcagatgaacaacctgagagtcgaggactcggccatgtattattgtgcaaaaggggctggtacgggagactactggggccagggaaccctggtcaccgtctcctcag

SEQ ID NO: 404 - protein, 116 aa, Canis sp.
  source 1..116: note = canis familiaris
EVQLVESGGDLVKPAGSLRLSCMASGFTFSSYSMSWVRQAPEKGLQLVAGINGDGSTTYYTDAVKGRFTISRDNAKNTVYLQMNNLRVEDSAMYYCAKGAGTGDYWGQGTLVTVSS

SEQ ID NO: 405 - DNA, 319 bp, Canis sp.
  source 1..319: mol_type = genomic DNA; note = canis familiaris
tcctatgagttgactcagccaccatccttgaatgtgaccctgagggagacggcccacatcacctgtgggggagacaggattggaagtagatatgttcagtggatccagcagaatccaggccaggcccccgtggtggtaatctataaagattccaacaggccgacagggatccctgagcgattctctggcgccaactcagggaacacggcaaccctgaccatcagtggggccctggccgaagacgaggctgactattattgccaggtgggggacagtggtaattgggtattcggtggagggacccagctgaccgtcctcg

SEQ ID NO: 406 - protein, 106 aa, Canis sp.
  source 1..106: note = canis familiaris
SYELTQPPSLNVTLRETAHITCGGDRIGSRYVQWIQQNPGQAPVVVIYKDSNRPTGIPERFSGANSGNTATLTISGALAEDEADYYCQVGDSGNWVFGGGTQLTVL

SEQ ID NO: 407 - protein, 7 aa, Canis sp.
  source 1..7: note = canis familiaris
GFTFSSY

SEQ ID NO: 408 - protein, 6 aa, Canis sp.
  source 1..6: note = canis familiaris
NGDGST

SEQ ID NO: 409 - protein, 11 aa, Canis sp.
  source 1..11: note = canis familiaris
CAKGAGTGDYW

SEQ ID NO: 410 - protein, 11 aa, Canis sp.
  source 1..11: note = canis familiaris
GGDRIGSRYVQ

SEQ ID NO: 411 - protein, 7 aa, Canis sp.
  source 1..7: note = canis familiaris
KDSNRPT

SEQ ID NO: 412 - protein, 11 aa, Canis sp.
  source 1..11: note = canis familiaris
CQVGDSGNWVF

SEQ ID NO: 413 - DNA, 346 bp, Canis sp.
  source 1..346: mol_type = genomic DNA; note = canis familiaris
gaactcacactgcaggagtcagggccaggactggtgaagccctcacagaccctctctctcacctgtgttgtgtccggaggctccgtcaccagcagttactactggaactggatccgccagcgccctgggaggggactggaatggatgggttactggacaggtagcacaatctacaacccggcatttcagggacgcatctccattactgctgacacggccaagaaccagttctccctgcaactgagttccatgaccaccgaggacacggccgtatatttctgtgtaagagagaggcggatggcttttggttcctggggccagggcaccctggtcattgtctcctcag

SEQ ID NO: 414 - protein, 115 aa, Canis sp.
  source 1..115: note = canis familiaris
ELTLQESGPGLVKPSQTLSLTCVVSGGSVTSSYYWNWIRQRPGRGLEWMGYWTGSTIYNPAFQGRISITADTAKNQFSLQLSSMTTEDTAVYFCVRERRMAFGSWGQGTLVIVSS

SEQ ID NO: 415 - DNA, 325 bp, Canis sp.
  source 1..325: mol_type = genomic DNA; note = canis familiaris
tcctatgtgctgacacagctgccatccaaaagtgtgaccctgaagcagccggcccacatcacctgtggaggagacaacattggaagaaaaagtgttcactggtaccagcagaagctgggccaggcccctgtactgattatctcttttgataacaacaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctca

SEQ ID NO: 416 - protein, 108 aa, Canis sp.
  source 1..108: note = canis familiaris
SYVLTQLPSKSVTLKQPAHITCGGDNIGRKSVHWYQQKLGQAPVLIISFDNNRPTGIPERFSGANSGNTATLTISGALAEDEADYYCQVWDSSAKASVFGGGTHLTVL

SEQ ID NO: 417 - protein, 7 aa, Canis sp.
  source 1..7: note = canis familiaris
GGSVTSS

SEQ ID NO: 418 - protein, 6 aa, Canis sp.
  source 1..6: note = canis familiaris
GYWTGS

SEQ ID NO: 419 - protein, 12 aa, Canis sp.
  source 1..12: note = canis familiaris
CVRERRMAFGSW

SEQ ID NO: 420 - protein, 11 aa, Canis sp.
  source 1..11: note = canis familiaris
GGDNIGRKSVH

SEQ ID NO: 421 - protein, 7 aa, Canis sp.
  source 1..7: note = canis familiaris
FDNNRPT

SEQ ID NO: 422 - protein, 13 aa, Canis sp.
  source 1..13: note = canis familiaris
CQVWDSSAKASVF

SEQ ID NO: 423 - DNA, 355 bp, Canis sp.
  source 1..355: mol_type = genomic DNA; note = canis familiaris
gaactcacactgcaggagtcagggccaggactggtgaagccctcacagaccctctctctcacctgtcttgtgtccggaggctccgtcaccaggagttattactggaattggatccgtcagcgccctgggaggggactggaatggatgggatattggacaggcagcacaaattacaatccggcattccagggacgcatctccatcattcctgacacggccaagaaccagttctccctgcaactgacctccatgaccaccgacgacacggccatatatttctgtgcaagagaggggggtcgaaccctaggtgcttttgctgactggggtcagggcaccctggtcactgtctcctcag

SEQ ID NO: 424 - protein, 118 aa, Canis sp.
  source 1..118: note = canis familiaris
ELTLQESGPGLVKPSQTLSLTCLVSGGSVTRSYYWNWIRQRPGRGLEWMGYWTGSTNYNPAFQGRISIIPDTAKNQFSLQLTSMTTDDTAIYFCAREGGRTLGAFADWGQGTLVTVSS

SEQ ID NO: 425 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaaaattggaagtaaaaatgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctca

SEQ ID NO: 426 - protein, 108 aa, Canis sp.
  source 1..108: note = canis familiaris
SYVLTQLPSKNVTLKQPAHITCGGDKIGSKNVHWYQQKLGQAPVLIIYYDSSRPTGIPERFSGANSGNTATLTISGALAEDEADYYCQVWDSSAKASVFGGGTHLTVL

SEQ ID NO: 427 - protein, 7 aa, Canis sp.
  source 1..7: note = canis familiaris
GGSVTRS

SEQ ID NO: 428 - protein, 6 aa, Canis sp.
  source 1..6: note = canis familiaris
WTGSTN

SEQ ID NO: 429 - protein, 15 aa, Canis sp.
  source 1..15: note = canis familiaris
CAREGGRTLGAFADW

SEQ ID NO: 430 - protein, 11 aa, Canis sp.
  source 1..11: note = canis familiaris
GGDKIGSKNVH

SEQ ID NO: 431 - protein, 7 aa, Canis sp.
  source 1..7: note = canis familiaris
YDSSRPT

SEQ ID NO: 432 - protein, 13 aa, Canis sp.
  source 1..13: note = canis familiaris
CQVWDSSAKASVF

SEQ ID NO: 433 - DNA, 355 bp, Canis sp.
  source 1..355: mol_type = genomic DNA; note = canis familiaris
gaattcacactgcaggagtcagggccaggactggtgaagccctcacagaccctctctctcacctgtattgtgtccggaggctccgtcaccagcagttactactggaactggatccgccagcgccctgggaggggactggaatggatggggtactggacaggtagcacaaactacaacccggcattccagggacgcatctccatcactcctgacacggccaagaaccagttctccctgcagctgagctccatgaccaccgaggacacggccgtgtattattgtgcaagagagggggggagtagtcttcaagcttttggtaactggggccagggcaccctggtcactgtctcctcag

SEQ ID NO: 434 - protein, 118 aa, Canis sp.
  source 1..118: note = canis familiaris
EFTLQESGPGLVKPSQTLSLTCIVSGGSVTSSYYWNWIRQRPGRGLEWMGYWTGSTNYNPAFQGRISITPDTAKNQFSLQLSSMTTEDTAVYYCAREGGSSLQAFGNWGQGTLVTVSS

SEQ ID NO: 435 - DNA, 325 bp, Canis sp.
  source 1..325: mol_type = genomic DNA; note = canis familiaris
tcctatgtgctgacacagctgccatccaaaaatgtgaccctgaagcagccggcccacatcacctgtgggggagacaacattggaagaaaaagtgttcactggtaccagcagaagctgggccaggcccctatactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggctagtgtgttcggcggaggcacccatctgaccgtcctcg

SEQ ID NO: 436 - protein, 108 aa, Canis sp.
  source 1..108: note = canis familiaris
SYVLTQLPSKNVTLKQPAHITCGGDNIGRKSVHWYQQKLGQAPILIIYYDSSRPTGIPERFSGANSGNTATLTISGALAEDEADYYCQVWDSSAKASVFGGGTHLTVL

SEQ ID NO: 437 - protein, 7 aa, Canis sp.
  source 1..7: note = canis familiaris
GGSVTSS

SEQ ID NO: 438 - protein, 6 aa, Canis sp.
  source 1..6: note = canis familiaris
GYWTGS

SEQ ID NO: 439 - protein, 15 aa, Canis sp.
  source 1..15: note = canis familiaris
CAREGGSSLQAFGNW

SEQ ID NO: 440 - protein, 11 aa, Canis sp.
  source 1..11: note = canis familiaris
GGDNIGRKSVH

SEQ ID NO: 441 - protein, 7 aa, Canis sp.
  source 1..7: note = canis familiaris
YDSSRPT

SEQ ID NO: 442 - protein, 13 aa, Canis sp.
  source 1..13: note = canis familiaris
CQVWDSSAKASVF

SEQ ID NO: 443 - DNA, 355 bp, Canis sp.
  source 1..355: mol_type = genomic DNA; note = canis familiaris
gaactcacactgcaggagtcagggccaggactggtgaagccctcacagaccctctctctcacctgtattgtgtccggaggctccgtcaccagcagttactactggaactggatccgccagagccctgggaggggactggaatggatgggttactggacaggaaatacaaactacaacccggcactccagggacgcatcttcatcactgctgacacggccaagaaccagttctcactgcagctgaggtccatgaccaccgaggacacggccgtgtattactgtgcaagagggggatatggataccactatggtatggactactggggccatggcacgtcagtcttcgtgtcctcag

SEQ ID NO: 444 - protein, 118 aa, Canis sp.
  source 1..118: note = canis familiaris
ELTLQESGPGLVKPSQTLSLTCIVSGGSVTSSYYWNWIRQSPGRGLEWMGYWTGNTNYNPALQGRIFITADTAKNQFSLQLRSMTTEDTAVYYCARGGYGYHYGMDYWGHGTSVFVSS

SEQ ID NO: 445 - DNA, 325 bp, Canis sp.
  source 1..325: mol_type = genomic DNA; note = canis familiaris
tcctatgtgctgacacagctgccgtccaaaagtgtggccctgaagcagccggcccacatcacctgtgggggagacaacattggaagtaaacgtgttcactggtaccagcagaagctgggccaggcccctgcactgattatctcttttgataatagcaggccgacagggatccctgagcgattctccggcgccaactcggggaacacggccaccctgaccatcagcggggccctggccgaggacgaggctgactattactgccaggtgtgggacagcactgctaaggctagtattttcggcggaggcacccatctgaccgtcctcg

SEQ ID NO: 446 - protein, 108 aa, Canis sp.
  source 1..108: note = canis familiaris
SYVLTQLPSKSVALKQPAHITCGGDNIGSKRVHWYQQKLGQAPALIISFDNSRPTGIPERFSGANSGNTATLTISGALAEDEADYYCQVWDSTAKASIFGGGTHLTVL

SEQ ID NO: 447 - protein, 6 aa, Canis sp.
  source 1..6: note = canis familiaris
GGSVTS

SEQ ID NO: 448 - protein, 6 aa, Canis sp.
  source 1..6: note = canis familiaris
GYWTGN

SEQ ID NO: 449 - protein, 15 aa, Canis sp.
  source 1..15: note = canis familiaris
CARGGYGYHYGMDYW

SEQ ID NO: 450 - protein, 11 aa, Canis sp.
  source 1..11: note = canis familiaris
GGDNIGSKRVH

SEQ ID NO: 451 - protein, 7 aa, Canis sp.
  source 1..7: note = canis familiaris
FDNSRPT

SEQ ID NO: 452 - protein, 13 aa, Canis sp.
  source 1..13: note = canis familiaris
CQVWDSTAKASIF

SEQ ID NO: 453 - DNA, 337 bp, Canis sp.
  source 1..337: mol_type = genomic DNA; note = canis familiaris
gaggtgcagctggtggagtctggaggcgacctggtgaagccaggaggaagcctgaggctgtcttgcgtggcttccggcttcaccttttccagatactatatgaactgggtgcgccaggctcctggcaagggactgcagtgggtggcctacatcgacagcgacggctccagcacatcttatgccgacgctgtgaagggcaggttcaccatcagccgggataacgccaagaatacactgtacctgcagatgaactccctgagagctgacgataccgccgtgtactattgtacaagctttgattattggggccagggcaccctggtcaccgtctcctcag

SEQ ID NO: 454 - protein, 112 aa, Canis sp.
  source 1..112: note = canis familiaris
EVQLVESGGDLVKPGGSLRLSCVASGFTFSRYYMNWVRQAPGKGLQWVAYIDSDGSSTSYADAVKGRFTISRDNAKNTLYLQMNSLRADDTAVYYCTSFDYWGQGTLVTVSS

SEQ ID NO: 455 - DNA, 325 bp, Canis sp.
  source 1..325: mol_type = genomic DNA; note = canis familiaris
tcctatgtgctgacacagctgccatccaaaagcgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 456 - protein, 108 aa, Canis sp.
  source 1..108: note = canis familiaris
SYVLTQLPSKSVTLKQPAHITCGGDNIGSKSVHWYQQKLGQAPVLIIYYDSSRPTGIPERFSGANSGNTATLTISGALAEDEADYYCQVWDSSAKAWVFGEGTQLTVL

SEQ ID NO: 457 - protein, 7 aa, Canis sp.
  source 1..7: note = canis familiaris
GFTFSRY

SEQ ID NO: 458 - protein, 6 aa, Canis sp.
  source 1..6: note = canis familiaris
DSDGSS

SEQ ID NO: 459 - protein, 7 aa, Canis sp. [sequence omitted]

SEQ ID NO: 460 - protein, 11 aa, Canis sp.
  source 1..11: note = canis familiaris
GGDNIGSKSVH

SEQ ID NO: 461 - protein, 7 aa, Canis sp.
  source 1..7: note = canis familiaris
YDSSRPT

SEQ ID NO: 462 - protein, 13 aa, Canis sp.
  source 1..13: note = canis familiaris
CQVWDSSAKAWVF

SEQ ID NO: 463 - DNA, 337 bp, Canis sp.
  source 1..337: mol_type = genomic DNA; note = canis familiaris
gagatgcaactggtggagtctgggggagacctggtgaagcctggggggtccctgagactttcctgtgtggcctctggattcaccttcagtcgctaccacatgagctgggtccgccaggctccagggaaggggcttcagtgggtcgcatacattgacagcgacggaagtcccacaaactatgcagacgctgtgaagggccgattcaccatctccagagacaacgccaagaacacactatatcttcagatgaacagcctgagagccgaggacacggccgtgtattactgtacgacctacgaccagtggggccagggaaccctggtcaccgtctcctcag

SEQ ID NO: 464 - protein, 112 aa, Canis sp.
  source 1..112: note = canis familiaris
EMQLVESGGDLVKPGGSLRLSCVASGFTFSRYHMSWVRQAPGKGLQWVAYIDSDGSPTNYADAVKGRFTISRDNAKNTLYLQMNSLRAEDTAVYYCTTYDQWGQGTLVTVSS

SEQ ID NO: 465 - DNA, 325 bp, Canis sp.; identical to SEQ ID NO: 455
  source 1..325: mol_type = genomic DNA; note = canis familiaris

SEQ ID NO: 466 - protein, 108 aa, Canis sp.; identical to SEQ ID NO: 456
  source 1..108: note = canis familiaris

SEQ ID NO: 467 - protein, 7 aa, Canis sp.
  source 1..7: note = canis familiaris
GFTFSRY

SEQ ID NO: 468 - protein, 6 aa, Canis sp.
  source 1..6: note = canis familiaris
DSDGSP

SEQ ID NO: 469 - protein, 7 aa, Canis sp.
  source 1..7: note = canis familiaris
CTTYDQW

SEQ ID NO: 470 - protein, 11 aa, Canis sp.
  source 1..11: note = canis familiaris
GGDNIGSKSVH

SEQ ID NO: 471 - protein, 7 aa, Canis sp.
  source 1..7: note = canis familiaris
YDSSRPT

SEQ ID NO: 472 - protein, 13 aa, Canis sp.
  source 1..13: note = canis familiaris
CQVWDSSAKAWVF

SEQ ID NO: 473 - DNA, 376 bp, Canis sp.; identical to SEQ ID NO: 323
  source 1..376: mol_type = genomic DNA; note = canis familiaris

SEQ ID NO: 474 - protein, 125 aa, Canis sp.; identical to SEQ ID NO: 324
  source 1..125: note = canis familiaris

SEQ ID NO: 475 - DNA, 325 bp, Canis sp.
  source 1..325: mol_type = genomic DNA; note = canis familiaris
tcctatgtgctgacacagctgccatccaaaagcgtgaccctgaagcagccggcccacatcacctgtgggggagacaacattggaagtaaaagtgttcactggtaccagcagaagctgggccaggcccctgtactgattatctattatgatagcagaaggccgacagggatcccagagcgattctccggcgccaactcggggaacacggccaccctgaccatcagcggggccctggccgaggacgaggctgactattactgccaggtgtgggacagcagtgctaaggcttgggtattcggtgaagggacccagctgaccgtcctcg

SEQ ID NO: 476 - protein, 108 aa, Canis sp.
  source 1..108: note = canis familiaris
SYVLTQLPSKSVTLKQPAHITCGGDNIGSKSVHWYQQKLGQAPVLIIYYDSRRPTGIPERFSGANSGNTATLTISGALAEDEADYYCQVWDSSAKAWVFGEGTQLTVL

SEQ ID NO: 477 - protein, 7 aa, Canis sp.
  source 1..7: note = canis familiaris
GVTFSRY

SEQ ID NO: 478 - protein, 6 aa, Canis sp.
  source 1..6: note = canis familiaris
NSGGST

SEQ ID NO: 479 - protein, 20 aa, Canis sp.
  source 1..20: note = canis familiaris
CASEGVGEIYGYKDFGMDYW

SEQ ID NO: 480 - protein, 11 aa, Canis sp.
  source 1..11: note = canis familiaris
GGDNIGSKSVH

SEQ ID NO: 481 - protein, 7 aa, Canis sp.
  source 1..7: note = canis familiaris
YDSRRPT

SEQ ID NO: 482 - protein, 13 aa, Canis sp.
  source 1..13: note = canis familiaris
CQVWDSSAKAWVF

SEQ ID NO: 483 - DNA, 376 bp, Canis sp.; identical to SEQ ID NO: 313
  source 1..376: mol_type = genomic DNA; note = canis familiaris

SEQ ID NO: 484 - protein, 125 aa, Canis sp.; identical to SEQ ID NO: 314
  source 1..125: note = canis familiaris

SEQ ID NO: 485 - DNA, 325 bp, Canis sp.
  source 1..325: mol_type = genomic DNA; note = canis familiaris
tcctatgtgctgacacagctgccatccaaaagcgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaaggcttgggtattcggtgcagggacccagctgaccgtcctcg

SEQ ID NO: 486 - protein, 108 aa, Canis sp.
  source 1..108: note = canis familiaris
SYVLTQLPSKSVTLKQPAHITCGGDNIGSKSVHWYQQKLGQAPVLIIYYDSSRPTGIPERFSGANSGNTATLTISGALAEDEADYYCQVWDSSAKAWVFGAGTQLTVL

SEQ ID NO: 487 - protein, 7 aa, Canis sp.
  source 1..7: note = canis familiaris
GFTFSSY

SEQ ID NO: 488 - protein, 6 aa, Canis sp.
  source 1..6: note = canis familiaris
NSGGSR

SEQ ID NO: 489 - protein, 20 aa, Canis sp.
  source 1..20: note = canis familiaris
CASGRTGPKLGLGDYGMDSW

SEQ ID NO: 490 - protein, 11 aa, Canis sp.
  source 1..11: note = canis familiaris
GGDNIGSKSVH

SEQ ID NO: 491 - protein, 7 aa, Canis sp.
  source 1..7: note = canis familiaris
YDSSRPT

SEQ ID NO: 492 - protein, 13 aa, Canis sp.
  source 1..13: note = canis familiaris
CQVWDSSAKAWVF

SEQ ID NO: 493 - DNA, 337 bp, Canis sp.
  source 1..337: mol_type = genomic DNA; note = canis familiaris
gagatgcagctggtggaatctgggggagccctggtgaagcctggggggtccctgagactctcctgtgtggcctctggattcacctttaggagataccacatgagttgggtccgccaggctccaggacaggggcttcagtgggtcgcatacattgacagtgatggaagtcctacaagttatggagactctgtgaagggccgattcaccatctccagagacaacgccaagaatacgctatttcttcagatgaacagtctgagagtcgacgacacggccgtatattactgtacgactcttgactactggggccggggaaccctggtcaccgtctcctcag

SEQ ID NO: 494 - protein, 112 aa, Canis sp.
  source 1..112: note = canis familiaris
EMQLVESGGALVKPGGSLRLSCVASGFTFRRYHMSWVRQAPGQGLQWVAYIDSDGSPTSYGDSVKGRFTISRDNAKNTLFLQMNSLRVDDTAVYYCTTLDYWGRGTLVTVSS

SEQ ID NO: 495 - DNA, 319 bp, Canis sp.
  source 1..319: mol_type = genomic DNA; note = canis familiaris
tcctatgtgctgacacaggtgccatccaaaaatgtgaccctgaagcagccggcccacatcacctgtgggggagacaacattggaggtaaaagtgttcactggtaccagcagaagctgggccaggcccctctactgattatctattatgataccaggaggccgacagggatccctgagcgcttctccggcgccaactcggggaacacggccaccctgaccatcagcagggccctggccgaggacgaggctgactattactgccaggtgtgggacagcagtggtaatgtgttcggcggaggcacccatctgaccgtcatcg

SEQ ID NO: 496 - protein, 106 aa, Canis sp.
  source 1..106: note = canis familiaris
SYVLTQVPSKNVTLKQPAHITCGGDNIGGKSVHWYQQKLGQAPLLIIYYDTRRPTGIPERFSGANSGNTATLTISRALAEDEADYYCQVWDSSGNVFGGGTHLTVI

SEQ ID NO: 497 - protein, 7 aa, Canis sp.
  source 1..7: note = canis familiaris
GFTFRRY

SEQ ID NO: 498 - protein, 6 aa, Canis sp.
  source 1..6: note = canis familiaris
DSDGSP

SEQ ID NO: 499 - protein, 7 aa, Canis sp. [sequence omitted]

SEQ ID NO: 500 - protein, 11 aa, Canis sp.
  source 1..11: note = canis familiaris
GGDNIGGKSVH

SEQ ID NO: 501 - protein, 7 aa, Canis sp.
  source 1..7: note = canis familiaris
YDTRRPT

SEQ ID NO: 502 - protein, 11 aa, Canis sp.
  source 1..11: note = canis familiaris
CQVWDSSGNVF

SEQ ID NO: 503 - DNA, 337 bp, Canis sp.
  source 1..337: mol_type = genomic DNA; note = canis familiaris
gaggtgcagctggtgcagtctgggggagacctggtgaagcctggggggtccctgagactttcctgtgtggcctctggatacaccttcactaggtatcacatgagttgggtccgccaggctccagggaaggggcttcagtgggtcgcattcattaatagtgatggaactggtataacctatggagacgctgtgaagggccgattcaccatctccagagacaatgccaagaacacgctgtatcttcagatgaacagcctgagagccgaggacacggccatctattactgtacgacttttgactcctggggccagggaaccctggtcaccgtctcctcag

SEQ ID NO: 504 - protein, 112 aa, Canis sp.
  source 1..112: note = canis familiaris
EVQLVQSGGDLVKPGGSLRLSCVASGYTFTRYHMSWVRQAPGKGLQWVAFINSDGTGITYGDAVKGRFTISRDNAKNTLYLQMNSLRAEDTAIYYCTTFDSWGQGTLVTVSS

SEQ ID NO: 505 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagcaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 506 - protein, 106 aa, Canis sp.
  source 1..106: note = canis familiaris
SYVLTQLPSKNVTLKQPAHITCGGDNIGSKSVHWYQQKLGQAPILIIYYDSRRPTGIPERFSGANSGNTATLTISGALAEDEADYYCQVWDSSGNVFGGGTHLTVL

SEQ ID NO: 507 - protein, 7 aa, Canis sp.
  source 1..7: note = canis familiaris
GYTFTRY

SEQ ID NO: 508 - protein, 6 aa, Canis sp.
  source 1..6: note = canis familiaris
NSDGTG

SEQ ID NO: 509 - protein, 7 aa, Canis sp.
  source 1..7: note = canis familiaris
CTTFDSW

SEQ ID NO: 510 - protein, 11 aa, Canis sp.
  source 1..11: note = canis familiaris
GGDNIGSKSVH

SEQ ID NO: 511 - protein, 7 aa, Canis sp.
  source 1..7: note = canis familiaris
YDSRRPT

SEQ ID NO: 512 - protein, 11 aa, Canis sp.
  source 1..11: note = canis familiaris
CQVWDSSGNVF

SEQ ID NO: 513 - DNA, 337 bp, Canis sp.
  source 1..337: mol_type = genomic DNA; note = canis familiaris
gaggtgcagttggtggagtctgggggagacctggtgaagccgggggggtccctgagactttcctgtgtggcctccggaatcaccttcagtagatatcacatgagctgggtccgccaggctccggggaaggggcttcagtgggtcgcattcattaacactggtagaagtagcacaaactatgcggacgctgtggcgggccgattcaccatctccagagacaacgccaagaacacgctgtatcttcaaatgaacggcctgacagtcgaggacacggccgtgtattactgttcgaccctcgactactggggccagggaaccctggtcaccgtctcctcag

SEQ ID NO: 514 - protein, 112 aa, Canis sp.
  source 1..112: note = canis familiaris
EVQLVESGGDLVKPGGSLRLSCVASGITFSRYHMSWVRQAPGKGLQWVAFINTGRSSTNYADAVAGRFTISRDNAKNTLYLQMNGLTVEDTAVYYCSTLDYWGQGTLVTVSS

SEQ ID NO: 515 - DNA, 319 bp, Canis sp.
  source 1..319: mol_type = genomic DNA; note = canis familiaris
tcctatgtgctgacacagctgccatccaaaagtgtgaccctgaagcagccggcccacatcacctgtgggggagacaacattggaagtaaaagtgttcactggtaccagcagaagctgggccaggcccctatactgattatctattatgataacaacaggccgacagggatccctgagcgattctccggcgccaactcggggaacacggccaccctgaccatcagcggggccctggccgaggacgaggctgactattactgccacgtgtgggacagcagtgttaatgtgttcggcggaggcacccatctgaccgtcctcg

SEQ ID NO: 516 - protein, 106 aa, Canis sp.
  source 1..106: note = canis familiaris
SYVLTQLPSKSVTLKQPAHITCGGDNIGSKSVHWYQQKLGQAPILIIYYDNNRPTGIPERFSGANSGNTATLTISGALAEDEADYYCHVWDSSVNVFGGGTHLTVL

SEQ ID NO: 517 - protein, 7 aa, Canis sp.
  source 1..7: note = canis familiaris
GITFSRY

SEQ ID NO: 518 - protein, 6 aa, Canis sp.
  source 1..6: note = canis familiaris
NTGRSS

SEQ ID NO: 519 - protein, 7 aa, Canis sp.
  source 1..7: note = canis familiaris
CSTLDYW

SEQ ID NO: 520 - protein, 11 aa, Canis sp.
  source 1..11: note = canis familiaris
GGDNIGSKSVH

SEQ ID NO: 521 - protein, 7 aa, Canis sp.
  source 1..7: note = canis familiaris
YDNNRPT

SEQ ID NO: 522 - protein, 11 aa, Canis sp.
  source 1..11: note = canis familiaris
CHVWDSSVNVF

SEQ ID NO: 523 - DNA, 337 bp, Canis sp.
  source 1..337: mol_type = genomic DNA; note = canis familiaris
gaggtgcacctggtggagtctgggggagacctggtgaagcctggggggtccctgagactttcctgtgtggcctctggaatcaccttcaatagatattatatgaactgggtccgccagactccagggaaggggcttcagtgggtcgcatacatagacagtggtggaagtcccacaacctatgcagacgctgtaaagggccgattcaccatctccagagacaacgccaagaacacgttgtatcttcagatgaacagcctgagagccgaggacacggccgtatattactgtacgagttttgactactggggccagggaaccctggtcaccgtctcctcag

SEQ ID NO: 524 - protein, 112 aa, Canis sp.
  source 1..112: note = canis familiaris
EVHLVESGGDLVKPGGSLRLSCVASGITFNRYYMNWVRQTPGKGLQWVAYIDSGGSPTTYADAVKGRFTISRDNAKNTLYLQMNSLRAEDTAVYYCTSFDYWGQGTLVTVSS

SEQ ID NO: 525 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgataccagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 526 - protein, 106 aa, Canis sp.
  source 1..106: note = canis familiaris
SYVLTQLPSKNVTLKQPAHITCGGDNIGSKSVHWYQQKLGQAPILIIYYDTSRPTGIPERFSGANSGNTATLTISGALAEDEADYYCQVWDSSANVFGGGTHLTVL

SEQ ID NO: 527 - protein, 7 aa, Canis sp.
  source 1..7: note = canis familiaris
GITFNRY

SEQ ID NO: 528 - protein, 6 aa, Canis sp.
  source 1..6: note = canis familiaris
DSGGSP

SEQ ID NO: 529 - protein, 7 aa, Canis sp. [sequence omitted]

SEQ ID NO: 530 - protein, 11 aa, Canis sp.
  source 1..11: note = canis familiaris
GGDNIGSKSVH

SEQ ID NO: 531 - protein, 7 aa, Canis sp.
  source 1..7: note = canis familiaris
YDTSRPT

SEQ ID NO: 532 - protein, 11 aa, Canis sp.
  source 1..11: note = canis familiaris
CQVWDSSANVF

SEQ ID NO: 533 - DNA, 337 bp, Canis sp.
  source 1..337: mol_type = genomic DNA; note = canis familiaris
gaggtgcaactggtggagtctgggggagacctggtgaagcctggggggtccctgagactttcctgtgtggcctctggactcaccttcagtaggtaccacatgagctggatccgccaggctccagggaaggggcttcagtgggtcgcatacattagcagtggtggaagtagcacaaactatgcaggctctgtgaagggccgattcaccgtctccagagacaacgccaagaacactctgtatcttcagatgaacagtctgagagccgaagacacggccgtctattactgtacgagtcttgactactggggccagggaaccctggtcaccgtctcctcag

SEQ ID NO: 534 - protein, 112 aa, Canis sp.
  source 1..112: note = canis familiaris
EVQLVESGGDLVKPGGSLRLSCVASGLTFSRYHMSWIRQAPGKGLQWVAYISSGGSSTNYAGSVKGRFTVSRDNAKNTLYLQMNSLRAEDTAVYYCTSLDYWGQGTLVTVSS

SEQ ID NO: 535 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagtaggaggccgacagggatccctgagcgattctccggcgccaactcggggaacacggccaccctgaccatcagcggggccctggccgaggacgaggctgactattactgccaggtgtgggacagcagtgataatgtgttcggcggaggcacccatctgaccgtcctcg

SEQ ID NO: 536 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTISGALAEDEADYYCQVWDSSDNVFGGGTHLTVL

SEQ ID NO: 537 - protein, 7 aa, Canis sp.
  source 1..7: note = canis familiaris
GLTFSRY

SEQ ID NO: 538 - protein, 6 aa, Canis sp.
  source 1..6: note = canis familiaris
SSGGSS

SEQ ID NO: 539 - protein, 7 aa, Canis sp.
  source 1..7: note = canis familiaris
CTSLDYW

SEQ ID NO: 540 - protein, 11 aa, Canis sp.
  source 1..11: note = canis familiaris
GGDNIGSKSVH

SEQ ID NO: 541 - protein, 7 aa, Canis sp.
  source 1..7: note = canis familiaris
YDSRRPT

SEQ ID NO: 542 - protein, 11 aa, Canis sp.
  source 1..11: note = canis familiaris
CQVWDSSDNVF

SEQ ID NO: 543 - DNA, 337 bp, Canis sp.
  source 1..337: mol_type = genomic DNA; note = canis familiaris
gagatgcagctggtggagtctgggggagacctggtgaagcctggggaatccctgagactttcctgtgtggcctctggaatctccttcaataactaccacatgagctgggtccgccaggctccagggaaggggcttcagtgggtcgcatacattgacagtgttggaactagcacaacctatgcagacgctgtgaagggccgattcaccatctccagagacaacgccaagaacacgctgtatcttcaaatgaacagcctgagagccgaggacacggccgtttattactgtacgacctttgactcctggggccagggaaccctggtcaccgtctcctcag

SEQ ID NO: 544 - protein, 112 aa, Canis sp.
  source 1..112: note = canis familiaris
EMQLVESGGDLVKPGESLRLSCVASGISFNNYHMSWVRQAPGKGLQWVAYIDSVGTSTTYADAVKGRFTISRDNAKNTLYLQMNSLRAEDTAVYYCTTFDSWGQGTLVTVSS

SEQ ID NO: 545 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtattgattatctattatgatagcaggaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 546 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 547 - protein, 7 aa, Canis sp.
  source 1..7: note = canis familiaris
GISFNNY

SEQ ID NO: 548 - protein, 6 aa, Canis sp.
  source 1..6: note = canis familiaris
DSVGTS

SEQ ID NO: 549 - protein, 7 aa, Canis sp.
  source 1..7: note = canis familiaris
CTTFDSW

SEQ ID NO: 550 - protein, 11 aa, Canis sp.
  source 1..11: note = canis familiaris
GGDNIGSKSVH

SEQ ID NO: 551 - protein, 7 aa, Canis sp.
  source 1..7: note = canis familiaris
YDSRRPT

SEQ ID NO: 552 - protein, 11 aa, Canis sp.
  source 1..11: note = canis familiaris
CQVWDSSANVF

SEQ ID NO: 553 - DNA, 337 bp, Canis sp.
  source 1..337: mol_type = genomic DNA; note = canis familiaris
gaaatgcagctggtggagtctgggggagacctggtgaagcctggggggtccctgagactttcctgtgtggcctctggattcaccttcagtagatattacatgaactgggtccgccaggctccagggaagggacttcagtgggtcgcatatattgatagtggtggaagtcccacaaactatgcagacgctgtgaagggccgattcaccatctccagagacaacgccaagaacacgctgtatcttcagatgaacagcctgagagccgaggacgcggccatatattattgtacgacttttgacttctggggccagggaaccctggtcaccgtctcctcag

SEQ ID NO: 554 - protein, 112 aa, Canis sp.
  source 1..112: note = canis familiaris
EMQLVESGGDLVKPGGSLRLSCVASGFTFSRYYMNWVRQAPGKGLQWVAYIDSGGSPTNYADAVKGRFTISRDNAKNTLYLQMNSLRAEDAAIYYCTTFDFWGQGTLVTVSS

SEQ ID NO: 555 - DNA, 319 bp, Canis sp.
  source 1..319: mol_type = genomic DNA; note = canis familiaris
tcctatgtggtgacacagctgccatccaaaaatgtgaccctgaagcagccggcccacatcacctgtgggggagacaacattggaagtaaaagtgttcactggtaccagcagaagctgggccaggcccctgtactgattatctattatgataacaggaggccgacagggatccctgagcgattctccggcgccaactcggggaacacggccaccctgaccatcagcggggccctggccgaggacgaggctgactattactgccaggtgtgggacagcagtgctaatgtcttcggcggaggcacccatctgaccgtcctcg

SEQ ID NO: 556 - protein, 106 aa, Canis sp.
  source 1..106: note = canis familiaris
SYVVTQLPSKNVTLKQPAHITCGGDNIGSKSVHWYQQKLGQAPVLIIYYDNRRPTGIPERFSGANSGNTATLTISGALAEDEADYYCQVWDSSANVFGGGTHLTVL

SEQ ID NO: 557 - protein, 7 aa, Canis sp.
  source 1..7: note = canis familiaris
GFTFSRY

SEQ ID NO: 558 - protein, 6 aa, Canis sp.
  source 1..6: note = canis familiaris
DSGGSP

SEQ ID NO: 559 - protein, 7 aa, Canis sp.
  source 1..7: note = canis familiaris
CTTFDFW

SEQ ID NO: 560 - protein, 11 aa, Canis sp.
  source 1..11: note = canis familiaris
GGDNIGSKSVH

SEQ ID NO: 561 - protein, 7 aa, Canis sp.
  source 1..7: note = canis familiaris
YDNRRPT

SEQ ID NO: 562 - protein, 11 aa, Canis sp.
  source 1..11: note = canis familiaris
CQVWDSSANVF

SEQ ID NO: 563 - DNA, 337 bp, Canis sp.
  source 1..337: mol_type = genomic DNA; note = canis familiaris
gaactacagctggtggagtctgggggagacctggtgaagcctggggggtccctgagactttcctgtgtggcctccggattcaccttcagtagatactacttgaactgggtccgccaggctccagggaaggggcttcagtgggtcgcattcattaccagtggtggcagtagcacaaactatgcagacgctgtgaagggccgattcaccatctccagagacaacgccaagaacacactgtatcttcagatgaatagcctgagagccgaggacacggccgtttattactgtacgagcttcgactcctggggccagggaaccctggtcaccgtctcctcag

SEQ ID NO: 564 - protein, 112 aa, Canis sp.
  source 1..112: note = canis familiaris
ELQLVESGGDLVKPGGSLRLSCVASGFTFSRYYLNWVRQAPGKGLQWVAFITSGGSSTNYADAVKGRFTISRDNAKNTLYLQMNSLRAEDTAVYYCTSFDSWGQGTLVTVSS

SEQ ID NO: 565 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactaattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 566 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSANVFGGGTHLTVL

SEQ ID NO: 567 - protein, 7 aa, Canis sp.
  source 1..7: note = canis familiaris
GFTFSRY

SEQ ID NO: 568 - protein, 6 aa, Canis sp.
  source 1..6: note = canis familiaris
TSGGSS

SEQ ID NO: 569 - protein, 7 aa, Canis sp.
  source 1..7: note = canis familiaris
CTSFDSW

SEQ ID NO: 570 - protein, 11 aa, Canis sp.
  source 1..11: note = canis familiaris
GGDNIGSKSVH

SEQ ID NO: 571 - protein, 7 aa, Canis sp.
  source 1..7: note = canis familiaris
YDSSRPT

SEQ ID NO: 572 - protein, 11 aa, Canis sp.
  source 1..11: note = canis familiaris
CQVWDSSANVF

SEQ ID NO: 573 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggaatcaccttcagtagatattacatgaactgggtccgccaggctccagggaaggggcttcagtgggtcgcatacattaatactggtgaaagtagcacaatctattcagacgctgtgaagggccgattcaccatttccagagacaacgccaagaacacgctgtatcttcagatgaacagcctgagagccgaagacacggccgtgtattactgtacgacccttgagtattggggccagggaaccctggtcaccgtctcctcag

SEQ ID NO: 574 - protein, 112 aa, Canis sp.
  source 1..112: note = canis familiaris
EVQLVESGGDLVKPGGSLRLSCVASGITFSRYYMNWVRQAPGKGLQWVAYINTGESSTIYSDAVKGRFTISRDNAKNTLYLQMNSLRAEDTAVYYCTTLEYWGQGTLVTVSS

SEQ ID NO: 575 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgcactgattatctattatgataatagaaggccgacagggatccctgagcgattctccggcgccaactcggggaacacggccaccctgaccatcagcggggccctggccgaggacgaggctgactattactgccaggtgtgggacagcagtgataatgtgttcggcggaggcacccacctgaccgtcctcg

SEQ ID NO: 576 - protein, 106 aa, Canis sp.
  source 1..106: note = canis familiaris
SYVLTQLPSKNVTLKQPAHITCGGDNIGSKSVHWYQQKLGQAPALIIYYDNRRPTGIPERFSGANSGNTATLTISGALAEDEADYYCQVWDSSDNVFGGGTHLTVL

SEQ ID NO: 577 - protein, 7 aa, Canis sp.
  source 1..7: note = canis familiaris
GITFSRY

SEQ ID NO: 578 - protein, 6 aa, Canis sp.
  source 1..6: note = canis familiaris
NTGESS

SEQ ID NO: 579 - protein, 7 aa, Canis sp.
  source 1..7: note = canis familiaris
CTTLEYW

SEQ ID NO: 580 - protein, 11 aa, Canis sp.
  source 1..11: note = canis familiaris
GGDNIGSKSVH

SEQ ID NO: 581 - protein, 7 aa, Canis sp.
  source 1..7: note = canis familiaris
YDNRRPT

SEQ ID NO: 582 - protein, 11 aa, Canis sp.
  source 1..11: note = canis familiaris
CQVWDSSDNVF

SEQ ID NO: 583 - DNA, 337 bp, Canis sp.
  source 1..337: mol_type = genomic DNA; note = canis familiaris
gaggtgcagttggtggagtctgggggagacctggtgaagcctggggggtccctgagactttcctgtgtggcctctggattaaccttcagtagatattacatgaactgggtccgccaggctccagggaaggggcttcagtgggtcgcatatattgatagtggtggaagtcccacaaactatgcagacgctgtgaagggccgattcaccatctccagagacaacgccaagaacacactgtatcttcagatgaacagcctgagagccgaggacacggccgtatattattgttcgagttttgactactggggccagggaaccctggtcaccgtctcctcag

SEQ ID NO: 584 - protein, 112 aa, Canis sp.
  source 1..112: note = canis familiaris
EVQLVESGGDLVKPGGSLRLSCVASGLTFSRYYMNWVRQAPGKGLQWVAYIDSGGSPTNYADAVKGRFTISRDNAKNTLYLQMNSLRAEDTAVYYCSSFDYWGQGTLVTVSS

SEQ ID NO: 585 - DNA, 319 bp, Canis sp.
  source 1..319: mol_type = genomic DNA; note = canis familiaris
tcctatgtggtgacacagctgccatccaatactgtgaccctgaagcagccggcccacatcacctgtgggggagacaacattggaagtaaaagtgttcactggtaccagcagaagctgggccaggcccctgtgctgattatctattatgatagcaggaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 586 - protein, 106 aa, Canis sp.
  source 1..106: note = canis familiaris
SYVVTQLPSNTVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 587 - protein, 7 aa, Canis sp.
  source 1..7: note = canis familiaris
GLTFSRY

SEQ ID NO: 588 - protein, 6 aa, Canis sp.
  source 1..6: note = canis familiaris
DSGGSP

SEQ ID NO: 589 - protein, 7 aa, Canis sp.
  source 1..7: note = canis familiaris
CSSFDYW

SEQ ID NO: 590 - protein, 11 aa, Canis sp.
  source 1..11: note = canis familiaris
GGDNIGSKSVH

SEQ ID NO: 591 - protein, 7 aa, Canis sp.
  source 1..7: note = canis familiaris
YDSRRPT

SEQ ID NO: 592 - protein, 11 aa, Canis sp.
  source 1..11: note = canis familiaris
CQVWDSSANVF

SEQ ID NO: 593 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggactcaccttcagtaggtactacatgaactgggtccgccaggctccagggaaggggcttcagtgggtcgcatacattaacagtggtggaagtagcacagactatgcagacgctgtgaagggccgattcaccatctccagagacaacgccaagaacacgctgtatcttcagatgaacagcctgagagtcgaggacacggccgtgtattactgtgcgagttttgactactggggccagggaaccctggtcaccgtctcctcag

SEQ ID NO: 594 - protein, 112 aa, Canis sp.
  source 1..112: note = canis familiaris
EVQLVESGGDLVKPGGSLRLSCVASGLTFSRYYMNWVRQAPGKGLQWVAYINSGGSSTDYADAVKGRFTISRDNAKNTLYLQMNSLRVEDTAVYYCASFDYWGQGTLVTVSS

SEQ ID NO: 595 - DNA, 319 bp, Canis sp.
  source 1..319: mol_type = genomic DNA; note = canis familiaris
tcctatgtgctgacacagctgccatccaaaaaggtgaccctgaagcagtcggcccacatcacctgtgggggagacaacattggaagtaaaagtgttcactggtaccagcagaagctgggccaggcccctgtactgattatctatcatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 596 - protein, 106 aa, Canis sp.
  source 1..106: note = canis familiaris
SYVLTQLPSKKVTLKQSAHITCGGDNIGSKSVHWYQQKLGQAPVLIIYHDSSRPTGIPERFSGANSGNTATLTISGALAEDEADYYCQVWDSSANVFGGGTHLTVL

SEQ ID NO: 597 - protein, 7 aa, Canis sp.
  source 1..7: note = canis familiaris
GLTFSRY

SEQ ID NO: 598 - protein, 6 aa, Canis sp.
  source 1..6: note = canis familiaris
NSGGSS

SEQ ID NO: 599 - protein, 7 aa, Canis sp. [sequence omitted]

SEQ ID NO: 600 - protein, 11 aa, Canis sp.
  source 1..11: note = canis familiaris
GGDNIGSKSVH

SEQ ID NO: 601 - protein, 7 aa, Canis sp.
  source 1..7: note = canis familiaris
HDSSRPT

SEQ ID NO: 602 - protein, 11 aa, Canis sp.
  source 1..11: note = canis familiaris
CQVWDSSANVF

SEQ ID NO: 603 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcattcattaacactggtagaagtagcacaaactatgcagacgctgtggcgggccgattcaccatctccagagacaacgccaagaacacgctgtatcttcagatgaacagcctgagagccgaggacacggccgtgtattactgttcgaccctcgactactggggccagggaaccctggtcaccgtctcctcag

SEQ ID NO: 604 - protein, 112 aa, Canis sp.
  source 1..112: note = canis familiaris
EVQLVESGGDLVKPGGSLRLSCVASGFTFSRYHMSWVRQAPGKGLQWVAFINTGRSSTNYADAVAGRFTISRDNAKNTLYLQMNSLRAEDTAVYYCSTLDYWGQGTLVTVSS

SEQ ID NO: 605 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 606 - protein, 106 aa, Canis sp.; identical to SEQ ID NO: 566
  source 1..106: note = canis familiaris

SEQ ID NO: 607 - protein, 7 aa, Canis sp.
  source 1..7: note = canis familiaris
GFTFSRY

SEQ ID NO: 608 - protein, 6 aa, Canis sp.
  source 1..6: note = canis familiaris
NTGRSS

SEQ ID NO: 609 - protein, 7 aa, Canis sp.
  source 1..7: note = canis familiaris
CSTLDYW

SEQ ID NO: 610 - protein, 11 aa, Canis sp.
  source 1..11: note = canis familiaris
GGDNIGSKSVH

SEQ ID NO: 611 - protein, 7 aa, Canis sp.
  source 1..7: note = canis familiaris
YDSSRPT

SEQ ID NO: 612 - protein, 11 aa, Canis sp.
  source 1..11: note = canis familiaris
CQVWDSSANVF

SEQ ID NO: 613 - DNA, 337 bp, Canis sp.
  source 1..337: mol_type = genomic DNA; note = canis familiaris
gaagtacagctggtggagtctgggggagacctggtgaagcctggggggtccctgagactttcctgtgtggcctccggattcaccttcagtagatactacatgaactgggtccgccaggctccagggaaggggcttcagtgggtcgcattcattaccagtggtggaagtagcacaagctatggagacgctgtgaagggccgattcaccatctccagagacaacgccaagaacacgctgtatcttcagatgaacagcctgagagccgaggacacggccgtgtattactgtacgagcttcgactactggggccagggaaccctggtcaccgtctcctcag

SEQ ID NO: 614 - protein, 112 aa, Canis sp.
  source 1..112: note = canis familiaris
EVQLVESGGDLVKPGGSLRLSCVASGFTFSRYYMNWVRQAPGKGLQWVAFITSGGSSTSYGDAVKGRFTISRDNAKNTLYLQMNSLRAEDTAVYYCTSFDYWGQGTLVTVSS

SEQ ID NO: 615 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgctagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 616 - protein, 106 aa, Canis sp.
  source 1..106: note = canis familiaris
SYVLTQLPSKNVTLKQPAHITCGGDNIGSKSVHWYQQKLGQAPVLIIYYASSRPTGIPERFSGANSGNTATLTISGALAEDEADYYCQVWDSSANVFGGGTHLTVL

SEQ ID NO: 617 - protein, 7 aa, Canis sp.
  source 1..7: note = canis familiaris
GFTFSRY

SEQ ID NO: 618 - protein, 6 aa, Canis sp.
  source 1..6: note = canis familiaris
TSGGSS

SEQ ID NO: 619 - protein, 7 aa, Canis sp. [sequence omitted]

SEQ ID NO: 620 - protein, 11 aa, Canis sp.
  source 1..11: note = canis familiaris
GGDNIGSKSVH

SEQ ID NO: 621 - protein, 7 aa, Canis sp.
  source 1..7: note = canis familiaris
YASSRPT

SEQ ID NO: 622 - protein, 11 aa, Canis sp.
  source 1..11: note = canis familiaris
CQVWDSSANVF

SEQ ID NO: 623 - DNA, 337 bp, Canis sp.
  source 1..337: mol_type = genomic DNA; note = canis familiaris
gaggtgcagctggtggagtctgggggagacctggtgaagccgggggggtccctgagactttcctgtgtggcctctggactcaccttcagtagataccacatgagttgggtccgccaggctccagggaaggggcttcagtgggtcgcattcattaacagtggtagaagtgacacaacctatgcagacgctgtgaagggccgattcaccatctccagagacaacgccaagaacacgctgtatcttcagatgaacagcctgagagccgaggacacggccgtttattactgtacgacttttgactactggggccagggaaccctggtcaccgtctcctcag

SEQ ID NO: 624 - protein, 112 aa, Canis sp.
  source 1..112: note = canis familiaris
EVQLVESGGDLVKPGGSLRLSCVASGLTFSRYHMSWVRQAPGKGLQWVAFINSGRSDTTYADAVKGRFTISRDNAKNTLYLQMNSLRAEDTAVYYCTTFDYWGQGTLVTVSS

SEQ ID NO: 625 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgcactgattatctattatgatagcagcaggccgacagggatccctgagcgattctccggcgccaactcggggaacacggccaccctgaccatcagcggggccctggccgaggacgaggctgactattactgccaggtgtgggacagtagtgctaatgtgttcggcggaggcacccatctgaccgtcctcg

SEQ ID NO: 626 - protein, 106 aa, Canis sp.
  source 1..106: note = canis familiaris
SYVLTQLPSKNVTLKQPAHITCGGDNIGSKSVHWYQQKLGQAPALIIYYDSSRPTGIPERFSGANSGNTATLTISGALAEDEADYYCQVWDSSANVFGGGTHLTVL

SEQ ID NO: 627 - protein, 7 aa, Canis sp.
  source 1..7: note = canis familiaris
GLTFSRY

SEQ ID NO: 628 - protein, 6 aa, Canis sp.
  source 1..6: note = canis familiaris
NSGRSD

SEQ ID NO: 629 - protein, 7 aa, Canis sp. [sequence omitted]

SEQ ID NO: 630 - protein, 11 aa, Canis sp.
  source 1..11: note = canis familiaris
GGDNIGSKSVH

SEQ ID NO: 631 - protein, 7 aa, Canis sp.
  source 1..7: note = canis familiaris
YDSSRPT

SEQ ID NO: 632 - protein, 11 aa, Canis sp.
  source 1..11: note = canis familiaris
CQVWDSSANVF

SEQ ID NO: 633 - DNA, 337 bp, Canis sp.
  source 1..337: mol_type = genomic DNA; note = canis familiaris
gaggtgcagctggtggagtctgggggagacctggtgaagccgggggggtccctgagactttcctgtgtggcctctggaatcaccttcagtggctaccacatgagctgggtccgccaggctccagggaaggggcttcagtgggtcgcattcattaatagtggtagaagaagcacaagttatgctgacgctgtgaagggccgattcaccatttccagagacaacgccaaaaacacgctgtatcttcagatgaacagcctgagagccgaagatacggccatttattactgtacgacccttgagttttggggccagggaaccctggtcaccgtctcctcag

SEQ ID NO: 634 - protein, 112 aa, Canis sp.
  source 1..112: note = canis familiaris
EVQLVESGGDLVKPGGSLRLSCVASGITFSGYHMSWVRQAPGKGLQWVAFINSGRRSTSYADAVKGRFTISRDNAKNTLYLQMNSLRAEDTAIYYCTTLEFWGQGTLVTVSS

SEQ ID NO: 635 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgccagatgtgggacagcagtgttaatgtgttcggcggaggcacccatctgaccgtcctcg

SEQ ID NO: 636 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTINGALAEDEADYYCQMWDSSVNVFGGGTHLTVL

SEQ ID NO: 637 - protein, 7 aa, Canis sp.
  source 1..7: note = canis familiaris
GITFSGY

SEQ ID NO: 638 - protein, 6 aa, Canis sp.
  source 1..6: note = canis familiaris
NSGRRS

SEQ ID NO: 639 - protein, 7 aa, Canis sp.
  source 1..7: note = canis familiaris
CTTLEFW

SEQ ID NO: 640 - protein, 11 aa, Canis sp.
  source 1..11: note = canis familiaris
GGDNIGSKSVH

SEQ ID NO: 641 - protein, 7 aa, Canis sp.
  source 1..7: note = canis familiaris
YDSRRPT

SEQ ID NO: 642 - protein, 11 aa, Canis sp.
  source 1..11: note = canis familiaris
CQMWDSSVNVF

SEQ ID NO: 643 - DNA, 337 bp, Canis sp.
  source 1..337: mol_type = genomic DNA; note = canis familiaris
gagatacaactggtggagtctgggggagacctggtgaagcctggggggtccctgagactttcctgtgtggcctctggattcaccttcagtcgctaccacatgagctgggtccgccaggctccagggaaggggcttcagtgggtcgcatatattaacactggtggaagtaacacaaactatgcagacgctgtgaagggccgattcaccatctccagagacaacgccaagaacacgctgtatcttcagatgaacagcctgagagccgaggacacggccgtgtattactgtacgacccttgactcctggggccagggaaccctggtcaccgtctcctcag

SEQ ID NO: 644 - protein, 112 aa, Canis sp.
  source 1..112: note = canis familiaris
EIQLVESGGDLVKPGGSLRLSCVASGFTFSRYHMSWVRQAPGKGLQWVAYINTGGSNTNYADAVKGRFTISRDNAKNTLYLQMNSLRAEDTAVYYCTTLDSWGQGTLVTVSS

SEQ ID NO: 645 - DNA, 319 bp, Canis sp.
  source 1..319: mol_type = genomic DNA; note = canis familiaris
tcctatgtgctgacacagctgccattcaaaaatgtgaccctgaagcagccggcccacatcacctgtgggggagacaacattggaagtaaaagtgttcactggtaccagcagaagctgggccaggcccctgtattgattatctattatgatagtagaaggccgacagggatccctgagcgattctccggcgccaactcggggaacacggccaccctgaccatcagcggggccctggccgaggacgaggctgactattactgccaggtgtgggacagtagtgttaatgtgttcggcggaggcacccatctgaccgtcctcg

SEQ ID NO: 646 - protein, 106 aa, Canis sp.
  source 1..106: note = canis familiaris
SYVLTQLPFKNVTLKQPAHITCGGDNIGSKSVHWYQQKLGQAPVLIIYYDSRRPTGIPERFSGANSGNTATLTISGALAEDEADYYCQVWDSSVNVFGGGTHLTVL

SEQ ID NO: 647 - protein, 7 aa, Canis sp.
  source 1..7: note = canis familiaris
GFTFSRY

SEQ ID NO: 648 - protein, 6 aa, Canis sp.
  source 1..6: note = canis familiaris
NTGGSN

SEQ ID NO: 649 - protein, 7 aa, Canis sp.
  source 1..7: note = canis familiaris
CTTLDSW

SEQ ID NO: 650 - protein, 11 aa, Canis sp.
  source 1..11: note = canis familiaris
GGDNIGSKSVH

SEQ ID NO: 651 - protein, 7 aa, Canis sp.
  source 1..7: note = canis familiaris
YDSRRPT

SEQ ID NO: 652 - protein, 11 aa, Canis sp.
  source 1..11: note = canis familiaris
CQVWDSSVNVF

SEQ ID NO: 653 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtaacacagactatacagacgctgtgaagggccgattcaccatctccagagacaacgccaagaacacgctgtatcttcagatgaacagcctgagagccgaggacacggccgtgtattactgttcgagtctggactactggggccagggaaccctggtcaccgtctcctcag

SEQ ID NO: 654 - protein, 112 aa, Canis sp.
  source 1..112: note = canis familiaris
EVQLVESGGDLVKPGGSLRLSCVASGFTFSRYHMSWVRQAPGKGLQWVAYINSGGSNTDYTDAVKGRFTISRDNAKNTLYLQMNSLRAEDTAVYYCSSLDYWGQGTLVTVSS

SEQ ID NO: 655 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagaaggaggccgacagggatccctgagcgattctccggcgccaactcggggaacacggccaccctgaccatcagcggggccctggccgaggacgaggctgactattactgccaggtgtgggacaggagtggtaatgtgttcggcggaggcacccatctgaccgtcctcg

SEQ ID NO: 656 - protein, 106 aa, Canis sp.
  source 1..106: note = canis familiaris
SYVLTQLPSKNVTLKQPAHITCGGDNIGSKSVHWYQQKLGQAPVLIIYYDRRRPTGIPERFSGANSGNTATLTISGALAEDEADYYCQVWDRSGNVFGGGTHLTVL

SEQ ID NO: 657 - protein, 7 aa, Canis sp.
  source 1..7: note = canis familiaris
GFTFSRY

SEQ ID NO: 658 - protein, 6 aa, Canis sp.
  source 1..6: note = canis familiaris
NSGGSN

SEQ ID NO: 659 - protein, 7 aa, Canis sp. [sequence omitted]

SEQ ID NO: 660 - protein, 11 aa, Canis sp.
  source 1..11: note = canis familiaris
GGDNIGSKSVH

SEQ ID NO: 661 - protein, 7 aa, Canis sp.
  source 1..7: note = canis familiaris
YDRRRPT

SEQ ID NO: 662 - protein, 11 aa, Canis sp.
  source 1..11: note = canis familiaris
CQVWDRSGNVF

SEQ ID NO: 663 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtgactcagactatgcagacgctgtgaagggccgattcaccatctccagagacaacgccaagaatacgctgtatcttcagatgaacagcctgagagccgaggacacggccgtgtattattgtacgagtctggactactggggccagggaaccctggtcaccgtctcctcag

SEQ ID NO: 664 - protein, 112 aa, Canis sp.
  source 1..112: note = canis familiaris
EVQLVESGGDLVKPGGSLRLSCVASGFTFSRYHMSWVRQAPGKGLQWVAYINSGGSDSDYADAVKGRFTISRDNAKNTLYLQMNSLRAEDTAVYYCTSLDYWGQGTLVTVSS

SEQ ID NO: 665 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagaaggaggccgacagggatccctgagcgattctccggcgccaactcggggaacacggccaccctgaccatcagcggggccctggccgaggacgaggctgactattactgtcaggtgtgggacaggagtggtaatgtgttcggcggaggcacccatctgaccgtcctcg

SEQ ID NO: 666 - protein, 106 aa, Canis sp.
  source 1..106: note = canis familiaris
SYVLTQLPSKNVTLKQPAHITCGGDNIGSKSVHWYQQKLGQAPILIIYYDRRRPTGIPERFSGANSGNTATLTISGALAEDEADYYCQVWDRSGNVFGGGTHLTVL

SEQ ID NO: 667 - protein, 7 aa, Canis sp.
  source 1..7: note = canis familiaris
GFTFSRY

SEQ ID NO: 668 - protein, 6 aa, Canis sp.
  source 1..6: note = canis familiaris
NSGGSD

SEQ ID NO: 669 - protein, 7 aa, Canis sp.
  source 1..7: note = canis familiaris
CTSLDYW

SEQ ID NO: 670 - protein, 11 aa, Canis sp.
  source 1..11: note = canis familiaris
GGDNIGSKSVH

SEQ ID NO: 671 - protein, 7 aa, Canis sp.
  source 1..7: note = canis familiaris
YDRRRPT

SEQ ID NO: 672 - protein, 11 aa, Canis sp.
  source 1..11: note = canis familiaris
CQVWDRSGNVF

SEQ ID NO: 673 - DNA, 337 bp, Canis sp.
  source 1..337: mol_type = genomic DNA; note = canis familiaris
gaggttcagctggtggagtctgggggagacctggtgaagcctggggggtccctgagactttcctgtgtggcctctggactcaccttcagtaggtaccacatgagctgggtccgccaggctccagggaaggggcttcagtgggtcgcatacattaacagtggtggtagtagcacagactatgcagacggtgtgaagggccgattcaccatctccagagacaacgccaagaacacgctatatcttcagatgaacagcctgagagccgaggacacggccgtgtattactgtacgagtctagaagactggggccagggaaccctggtcaccgtctcctcag

SEQ ID NO: 674 - protein, 112 aa, Canis sp.
  source 1..112: note = canis familiaris
EVQLVESGGDLVKPGGSLRLSCVASGLTFSRYHMSWVRQAPGKGLQWVAYINSGGSSTDYADGVKGRFTISRDNAKNTLYLQMNSLRAEDTAVYYCTSLEDWGQGTLVTVSS

SEQ ID NO: 675 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgttaatgtgttcggcggaggcacccatctgaccgtcctcg

SEQ ID NO: 676 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSVNVFGGGTHLTVL

SEQ ID NO: 677 - protein, 7 aa, Canis sp.
  source 1..7: note = canis familiaris
GLTFSRY

SEQ ID NO: 678 - protein, 6 aa, Canis sp.
  source 1..6: note = canis familiaris
NSGGSS

SEQ ID NO: 679 - protein, 7 aa, Canis sp.
  source 1..7: note = canis familiaris
CTSLEDW

SEQ ID NO: 680 - protein, 11 aa, Canis sp.
  source 1..11: note = canis familiaris
GGDNIGSKSVH

SEQ ID NO: 681 - protein, 7 aa, Canis sp.
  source 1..7: note = canis familiaris
YDSSRPT

SEQ ID NO: 682 - protein, 11 aa, Canis sp.
  source 1..11: note = canis familiaris
CQVWDSSVNVF

SEQ ID NO: 683 - DNA, 337 bp, Canis sp.
  source 1..337: mol_type = genomic DNA; note = canis familiaris
gagatgcaactggtggagtctgggggagacctggtgaagcctggggggtccctgagactttcctgtgtggcctctggattcaccttcagtcgctaccacatgagctgggtccgccaggctccagggaaggggcttcagtgggtcgcatacattaatagtggtggaagtcccacaaactatgcagacgctgtgaagggccgattcaccatctccagagacaacgccaagaacacactatatcttcagatgaacagcctgagagccgaggacacggccgtgtattactgtacgacctacgaccagtggggccagggaaccctggtcaccgtctcctcag

SEQ ID NO: 684 - protein, 112 aa, Canis sp.
  source 1..112: note = canis familiaris
EMQLVESGGDLVKPGGSLRLSCVASGFTFSRYHMSWVRQAPGKGLQWVAYINSGGSPTNYADAVKGRFTISRDNAKNTLYLQMNSLRAEDTAVYYCTTYDQWGQGTLVTVSS

SEQ ID NO: 685 - DNA, 319 bp, Canis sp.
  source 1..319: mol_type = genomic DNA; note = canis familiaris
tcctatgtgctgacacagctgccatccaaaagtgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacaccagtgctaatgtgttcggcggaggcacccatctgaccgtcctcg

SEQ ID NO: 686 - protein, 106 aa, Canis sp.
  source 1..106: note = canis familiaris
SYVLTQLPSKSVTLKQPAHITCGGDNIGSKSVHWYQQKLGQAPVLIIYYDSSRPTGIPERFSGANSGNTATLTISGALAEDEADYYCQVWDTSANVFGGGTHLTVL

SEQ ID NO: 687 - protein, 7 aa, Canis sp.
  source 1..7: note = canis familiaris
GFTFSRY

SEQ ID NO: 688 - protein, 6 aa, Canis sp.
  source 1..6: note = canis familiaris
NSGGSP

SEQ ID NO: 689 - protein, 7 aa, Canis sp.
  source 1..7: note = canis familiaris
CTTYDQW

SEQ ID NO: 690 - protein, 11 aa, Canis sp.
  source 1..11: note = canis familiaris
GGDNIGSKSVH

SEQ ID NO: 691 - protein, 7 aa, Canis sp.
  source 1..7: note = canis familiaris
YDSSRPT

SEQ ID NO: 692 - protein, 11 aa, Canis sp.
  source 1..11: note = canis familiaris
CQVWDTSANVF

SEQ ID NO: 693 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtccctcaagctatgcagacgctgtgaagggccgattcaccatctccagagacaacgccaagaacacgctgtatcttcaaatgaacagcctgagagccgaggacacggccgtatattactgtacgagttttgactgttggggccagggaaccctggtcaccgtctcctcag

SEQ ID NO: 694 - protein, 112 aa, Canis sp.
  source 1..112: note = canis familiaris
EVQLVESGGDLVKPGGSLRLSCVASGFTFSRYHMSWVRQAPGKGLQWVAYINSGGSPSSYADAVKGRFTISRDNAKNTLYLQMNSLRAEDTAVYYCTSFDCWGQGTLVTVSS

SEQ ID NO: 695 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtattgattatctattatgatagcagaaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccacctgaccgtcctcg

SEQ ID NO: 696 - protein, 106 aa, Canis sp.; identical to SEQ ID NO: 546
  source 1..106: note = canis familiaris

SEQ ID NO: 697 - protein, 7 aa, Canis sp.
  source 1..7: note = canis familiaris
GFTFSRY

SEQ ID NO: 698 - protein, 6 aa, Canis sp.
  source 1..6: note = canis familiaris
NSGGSP

SEQ ID NO: 699 - protein, 7 aa, Canis sp.
  source 1..7: note = canis familiaris
CTSFDCW

SEQ ID NO: 700 - protein, 11 aa, Canis sp.
  source 1..11: note = canis familiaris
GGDNIGSKSVH

SEQ ID NO: 701 - protein, 7 aa, Canis sp.
  source 1..7: note = canis familiaris
YDSRRPT

SEQ ID NO: 702 - protein, 11 aa, Canis sp.
  source 1..11: note = canis familiaris
CQVWDSSANVF

SEQ ID NO: 703 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagttgggtccgccaggctccagggaaggggcttcagtgggtcgcatacattaacagtggtggaagtaccacaagctatgcagacgctgtgaagggccgattcaccatctccagagacaacgccaagaacacgctgtatcttcagatgaacagcctgagagccgaggacacggccatatatttctgtacgagccttgactactggggccagggaaccctggtcaccgtctcctcag

SEQ ID NO: 704 - protein, 112 aa, Canis sp.
  source 1..112: note = canis familiaris
EVQLVESGGDLVKPGGSLRLSCVASGFTFSRYHMSWVRQAPGKGLQWVAYINSGGSTTSYADAVKGRFTISRDNAKNTLYLQMNSLRAEDTAIYFCTSLDYWGQGTLVTVSS

SEQ ID NO: 705 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctattctgattatctattatgatagcagaaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 706 - protein, 106 aa, Canis sp.
  source 1..106: note = canis familiaris
SYVLTQLPSKNVTLKQPAHITCGGDNIGSKSVHWYQQKLGQAPILIIYYDSRRPTGIPERFSGANSGNTATLTISGALAEDEADYYCQVWDSSANVFGGGTHLTVL

SEQ ID NO: 707 - protein, 7 aa, Canis sp.
  source 1..7: note = canis familiaris
GFTFSRY

SEQ ID NO: 708 - protein, 6 aa, Canis sp.
  source 1..6: note = canis familiaris
NSGGST

SEQ ID NO: 709 - protein, 7 aa, Canis sp.
  source 1..7: note = canis familiaris
CTSLDYW

SEQ ID NO: 710 - protein, 11 aa, Canis sp.
  source 1..11: note = canis familiaris
GGDNIGSKSVH

SEQ ID NO: 711 - protein, 7 aa, Canis sp.
  source 1..7: note = canis familiaris
YDSRRPT

SEQ ID NO: 712 - protein, 11 aa, Canis sp.
  source 1..11: note = canis familiaris
CQVWDSSANVF

SEQ ID NO: 713 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagctttgactactggggccagggaaccctggtcaccgtctcctcag

SEQ ID NO: 714 - protein, 112 aa, Canis sp.
  source 1..112: note = canis familiaris
EVQLVESGGDLVKPGGSLRLSCVASGFTFSRYHMSWVRQAPGKGLQWVAYINSGGSSTSYADAVKGRFTISRDNAKNTLYLQMNSLRAEDTAVYYCASFDYWGQGTLVTVSS

SEQ ID NO: 715 - DNA, 319 bp, Canis sp.
  source 1..319: mol_type = genomic DNA; note = canis familiaris
ccctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcaggaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 716 - protein, 106 aa, Canis sp.
  source 1..106: note = canis familiaris
PYVLTQLPSKNVTLKQPAHITCGGDNIGSKSVHWYQQKLGQAPVLIIYYDSRRPTGIPERFSGANSGNTATLTISGALAEDEADYYCQVWDSSANVFGGGTHLTVL

SEQ ID NO: 717 - protein, 7 aa, Canis sp.
  source 1..7: note = canis familiaris
GFTFSRY

SEQ ID NO: 718 - protein, 6 aa, Canis sp.
  source 1..6: note = canis familiaris
NSGGSS

SEQ ID NO: 719 - protein, 7 aa, Canis sp. [sequence omitted]

SEQ ID NO: 720 - protein, 11 aa, Canis sp.
  source 1..11: note = canis familiaris
GGDNIGSKSVH

SEQ ID NO: 721 - protein, 7 aa, Canis sp.
  source 1..7: note = canis familiaris
YDSRRPT

SEQ ID NO: 722 - protein, 11 aa, Canis sp.
  source 1..11: note = canis familiaris
CQVWDSSANVF

SEQ ID NO: 723 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaccagcctgagagccgaggatacggccgtgtattattgtacgagttttgactactggggccagggaaccctggtcaccgtctcctcag

SEQ ID NO: 724 - protein, 112 aa, Canis sp.
  source 1..112: note = canis familiaris
EVQLVESGGDLVKPGGSLRLSCVASGFTFSSYHMSWVRQAPGKGLQWVAYINSGGSSTSYADAVKGRFTISRDNAKNTLYLQMTSLRAEDTAVYYCTSFDYWGQGTLVTVSS

SEQ ID NO: 725 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaacgtgttcggcggaggcacccatctgaccgtcctcg

SEQ ID NO: 726 - protein, 106 aa, Canis sp.; identical to SEQ ID NO: 566
  source 1..106: note = canis familiaris

SEQ ID NO: 727 - protein, 7 aa, Canis sp.
  source 1..7: note = canis familiaris
GFTFSSY

SEQ ID NO: 728 - protein, 6 aa, Canis sp.
  source 1..6: note = canis familiaris
NSGGSS

SEQ ID NO: 729 - protein, 7 aa, Canis sp. [sequence omitted]

SEQ ID NO: 730 - protein, 11 aa, Canis sp.
  source 1..11: note = canis familiaris
GGDNIGSKSVH

SEQ ID NO: 731 - protein, 7 aa, Canis sp.
  source 1..7: note = canis familiaris
YDSSRPT

SEQ ID NO: 732 - protein, 11 aa, Canis sp.
  source 1..11: note = canis familiaris
CQVWDSSANVF

SEQ ID NO: 733 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagttttgactactggggccagggaaccctggtcaccgtctcctcag

SEQ ID NO: 734 - protein, 112 aa, Canis sp.
  source 1..112: note = canis familiaris
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCASFDYWGQGTLVTVSS

SEQ ID NO: 735 - DNA, 319 bp, Canis sp.; identical to SEQ ID NO: 605
  source 1..319: mol_type = genomic DNA; note = canis familiaris

SEQ ID NO: 736 - protein, 106 aa, Canis sp.; identical to SEQ ID NO: 566
  source 1..106: note = canis familiaris

SEQ ID NO: 737 - protein, 7 aa, Canis sp.
  source 1..7: note = canis familiaris
GFTFSSY

SEQ ID NO: 738 - protein, 6 aa, Canis sp.
  source 1..6: note = canis familiaris
NSGGSS

SEQ ID NO: 739 - protein, 7 aa, Canis sp. [sequence omitted]

SEQ ID NO: 740 - protein, 11 aa, Canis sp.
  source 1..11: note = canis familiaris
GGDNIGSKSVH

SEQ ID NO: 741 - protein, 7 aa, Canis sp.
  source 1..7: note = canis familiaris
YDSSRPT

SEQ ID NO: 742 - protein, 11 aa, Canis sp.
  source 1..11: note = canis familiaris
CQVWDSSANVF

SEQ ID NO: 743 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagtttagactactggggccagggaaccctggtcaccgtctcctcag

SEQ ID NO: 744 - protein, 112 aa, Canis sp.
  source 1..112: note = canis familiaris
EVQLVESGGDLVKPGGSLRLSCVASGFTFSRYHMSWVRQAPGKGLQWVAYINSGGSSTSYADAVKGRFTISRDNAKNTLYLQMNSLRAEDTAVYYCASLDYWGQGTLVTVSS

SEQ ID NO: 745 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacatcagtgctaatgtgttcggcggaggcacccacctgaccgtcctcg

SEQ ID NO: 746 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDISANVFGGGTHLTVL

SEQ ID NO: 747 - protein, 7 aa, Canis sp.
  source 1..7: note = canis familiaris
GFTFSRY

SEQ ID NO: 748 - protein, 6 aa, Canis sp.
  source 1..6: note = canis familiaris
NSGGSS

SEQ ID NO: 749 - protein, 7 aa, Canis sp. [sequence omitted]

SEQ ID NO: 750 - protein, 11 aa, Canis sp.
  source 1..11: note = canis familiaris
GGDNIGSKSVH

SEQ ID NO: 751 - protein, 7 aa, Canis sp.
  source 1..7: note = canis familiaris
YDSSRPT

SEQ ID NO: 752 - protein, 11 aa, Canis sp.
  source 1..11: note = canis familiaris
CQVWDISANVF

SEQ ID NO: 753 - DNA, 337 bp, Canis sp.
  source 1..337: mol_type = genomic DNA; note = canis familiaris
gaggtgcagatggtggagtctgggggagacctggtgaagcctggggggtccctgagactttcctgtgtggcctctggattcaccttcagtaggtattacatgaactgggtccgccaggctccaggtaaggggcttcagtgggtcgcatacattaacagtggtggaggtagcacaagttatgcagacgctgtgaagggccgattcaccatctccagagacaacgccaagaacacgctgtatcttcagatgaacagcctgagagccgaggacacggccgtgtattactgttcgagtttcgactactggggccagggaaccctggtcaccgtctcctcag

SEQ ID NO: 754 - protein, 112 aa, Canis sp.
  source 1..112: note = canis familiaris
EVQMVESGGDLVKPGGSLRLSCVASGFTFSRYYMNWVRQAPGKGLQWVAYINSGGGSTSYADAVKGRFTISRDNAKNTLYLQMNSLRAEDTAVYYCSSFDYWGQGTLVTVSS

SEQ ID NO: 755 - DNA, 319 bp, Canis sp.; identical to SEQ ID NO: 605
  source 1..319: mol_type = genomic DNA; note = canis familiaris

SEQ ID NO: 756 - protein, 106 aa, Canis sp.; identical to SEQ ID NO: 566
  source 1..106: note = canis familiaris

SEQ ID NO: 757 - protein, 7 aa, Canis sp.
  source 1..7: note = canis familiaris
GFTFSRY

SEQ ID NO: 758 - protein, 6 aa, Canis sp.
  source 1..6: note = canis familiaris
NSGGGS

SEQ ID NO: 759 - protein, 7 aa, Canis sp.
  source 1..7: note = canis familiaris
CSSFDYW

SEQ ID NO: 760 - protein, 11 aa, Canis sp.
  source 1..11: note = canis familiaris
GGDNIGSKSVH

SEQ ID NO: 761 - protein, 7 aa, Canis sp.
  source 1..7: note = canis familiaris
YDSSRPT

SEQ ID NO: 762 - protein, 11 aa, Canis sp.
  source 1..11: note = canis familiaris
CQVWDSSANVF

SEQ ID NO: 763 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctggatccgccaggctccagggaagggacttcagtgggtcgcattcattgacagcgatggaagtggtacaagatacgcagacgctgtgaagggccgattcaccttctccagagacaacgccaagaatacgctttatcttcagatgagcagcctgagagccgaggacacggccgtgtattactgtacgagtcttgagttctggggccagggaaccctggtcaccgtctcctcag

SEQ ID NO: 764 - protein, 112 aa, Canis sp.
  source 1..112: note = canis familiaris
EVQLVESGGDLVKPGGSLRLSCVASGFTFSRYHMSWIRQAPGKGLQWVAFIDSDGSGTRYADAVKGRFTFSRDNAKNTLYLQMSSLRAEDTAVYYCTSLEFWGQGTLVTVSS

SEQ ID NO: 765 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattattgtcaggtgtgggacagcagtgctaatgtgttcggcggaggcacccatctgaccgtcctcg

SEQ ID NO: 766 - protein, 106 aa, Canis sp.; identical to SEQ ID NO: 566
  source 1..106: note = canis familiaris

SEQ ID NO: 767 - protein, 7 aa, Canis sp.
  source 1..7: note = canis familiaris
GFTFSRY

SEQ ID NO: 768 - protein, 6 aa, Canis sp.
  source 1..6: note = canis familiaris
DSDGSG

SEQ ID NO: 769 - protein, 7 aa, Canis sp.
  source 1..7: note = canis familiaris
CTSLEFW

SEQ ID NO: 770 - protein, 11 aa, Canis sp.
  source 1..11: note = canis familiaris
GGDNIGSKSVH

SEQ ID NO: 771 - protein, 7 aa, Canis sp.
  source 1..7: note = canis familiaris
YDSSRPT

SEQ ID NO: 772 - protein, 11 aa, Canis sp.
  source 1..11: note = canis familiaris
CQVWDSSANVF

SEQ ID NO: 773 - DNA, 337 bp, Canis sp.
  source 1..337: mol_type = genomic DNA; note = canis familiaris
gaagtgcaactggtggagtctgggggagacctggtgaagccgggggggtccctgagactttcctgtgtggcctctggattcaccttcagtagatatcacatgagttggatccgccaggctccagggaagggacttcagtgggtcgcattcattgacagcgatggaagtggtgcaagatacgcagacgctgtgaagggccgattcaccttctccagagacaacgccaagaacacactgtatcttcagatgaacagcctgagagccgaggacacggccgtgtattattgcacgagtcttgaatactggggccagggaaccctggtcaccgtctcctcag

SEQ ID NO: 774 - protein, 112 aa, Canis sp.
  source 1..112: note = canis familiaris
EVQLVESGGDLVKPGGSLRLSCVASGFTFSRYHMSWIRQAPGKGLQWVAFIDSDGSGARYADAVKGRFTFSRDNAKNTLYLQMNSLRAEDTAVYYCTSLEYWGQGTLVTVSS

SEQ ID NO: 775 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacgttggaagtaaaagtgttcactggtaccagcagaaactgggccaggcccctgtactgattatctattatgatagcagaaggccgacagggatccctgagcgattctccggcgccaactcggggaacacggccaccctgaccatcagcggggccctggccgaggacgaggctgactattattgtcaggtgtgggacagcagtgctaatgtgttcggcggaggcacccatctgaccgtcctcg

SEQ ID NO: 776 - protein, 106 aa, Canis sp.
  source 1..106: note = canis familiaris
SYVLTQLPSKNVTLKQPAHITCGGDNVGSKSVHWYQQKLGQAPVLIIYYDSRRPTGIPERFSGANSGNTATLTISGALAEDEADYYCQVWDSSANVFGGGTHLTVL

SEQ ID NO: 777 - protein, 7 aa, Canis sp.
  source 1..7: note = canis familiaris
GFTFSRY

SEQ ID NO: 778 - protein, 6 aa, Canis sp.
  source 1..6: note = canis familiaris
DSDGSG

SEQ ID NO: 779 - protein, 7 aa, Canis sp.
  source 1..7: note = canis familiaris
CTSLEYW

SEQ ID NO: 780 - protein, 11 aa, Canis sp.
  source 1..11: note = canis familiaris
GGDNVGSKSVH

SEQ ID NO: 781 - protein, 7 aa, Canis sp.
  source 1..7: note = canis familiaris
YDSRRPT

SEQ ID NO: 782 - protein, 11 aa, Canis sp.
  source 1..11: note = canis familiaris
CQVWDSSANVF

SEQ ID NO: 783 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtattttcagatgaacagcctgagagccgaggacacggccgtgtattactgtacgacctttgactcctggggccagggaaccctggtcaccgtctcctcag

SEQ ID NO: 784 - protein, 112 aa, Canis sp.
  source 1..112: note = canis familiaris
EVQLVESGGDLVKPGGSLRLSCVASGFTFSSYHMSWVRQAPGKGLQWVAYINSGGSSTSYADAVKGRFTISRDNAKNTLYFQMNSLRAEDTAVYYCTTFDSWGQGTLVTVSS

SEQ ID NO: 785 - DNA, 319 bp, Canis sp.; identical to SEQ ID NO: 605
  source 1..319: mol_type = genomic DNA; note = canis familiaris

SEQ ID NO: 786 - protein, 106 aa, Canis sp.; identical to SEQ ID NO: 566
  source 1..106: note = canis familiaris

SEQ ID NO: 787 - protein, 7 aa, Canis sp.
  source 1..7: note = canis familiaris
GFTFSSY

SEQ ID NO: 788 - protein, 6 aa, Canis sp.
  source 1..6: note = canis familiaris
NSGGSS

SEQ ID NO: 789 - protein, 7 aa, Canis sp.
  source 1..7: note = canis familiaris
CTTFDSW

SEQ ID NO: 790 - protein, 11 aa, Canis sp.
  source 1..11: note = canis familiaris
GGDNIGSKSVH

SEQ ID NO: 791 - protein, 7 aa, Canis sp.
  source 1..7: note = canis familiaris
YDSSRPT

SEQ ID NO: 792 - protein, 11 aa, Canis sp.
  source 1..11: note = canis familiaris
CQVWDSSANVF

SEQ ID NO: 793 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtagctaccacatgagctgggtccgccaggctccagggaaggggcttcagtgggtcgcatacattaacagtggtggaagtagcacaagctatgcagacgctgtgaagggccgattcaccatctccagagacaacgcctacagcacgctgtttcttcagatgaacaacctgagagccgaggacacggccgtttattactgtacgactcttgactcctggggccagggaaccctggtcaccgtctcctcag

SEQ ID NO: 794 - protein, 112 aa, Canis sp.
  source 1..112: note = canis familiaris
EVQLVESGGDLVKPGGSLRLSCVASGFTFSSYHMSWVRQAPGKGLQWVAYINSGGSSTSYADAVKGRFTISRDNAYSTLFLQMNNLRAEDTAVYYCTTLDSWGQGTLVTVSS

SEQ ID NO: 795 - DNA, 319 bp, Canis sp.; identical to SEQ ID NO: 605
  source 1..319: mol_type = genomic DNA; note = canis familiaris

SEQ ID NO: 796 - protein, 106 aa, Canis sp.; identical to SEQ ID NO: 566
  source 1..106: note = canis familiaris

SEQ ID NO: 797 - protein, 7 aa, Canis sp.
  source 1..7: note = canis familiaris
GFTFSSY

SEQ ID NO: 798 - protein, 6 aa, Canis sp.
  source 1..6: note = canis familiaris
NSGGSS

SEQ ID NO: 799 - protein, 7 aa, Canis sp.
  source 1..7: note = canis familiaris
CTTLDSW

SEQ ID NO: 800 - protein, 11 aa, Canis sp.
  source 1..11: note = canis familiaris
GGDNIGSKSVH

SEQ ID NO: 801 - protein, 7 aa, Canis sp.
  source 1..7: note = canis familiaris
YDSSRPT

SEQ ID NO: 802 - protein, 11 aa, Canis sp.
  source 1..11: note = canis familiaris
CQVWDSSANVF

SEQ ID NO: 803 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattaaccttcagtaggtactacatgaactgggtccgccaggctccagggaaggggcttcagtgggtcgcatacattaacactggtggaagtagcacaagctatgcagacgctgtgaagggccgattcaccatctccagagacaacgccaagaacacgctgtatcttcagatgaacagcctgagagccgaggacacggccgtgtattactgtgcgagttttgactactggggccagggaaccctggtcaccgtctcctcag

SEQ ID NO: 804 - protein, 112 aa, Canis sp.
  source 1..112: note = canis familiaris
EVQLVESGGDLVKPGGSLRLSCVASGLTFSRYYMNWVRQAPGKGLQWVAYINTGGSSTSYADAVKGRFTISRDNAKNTLYLQMNSLRAEDTAVYYCASFDYWGQGTLVTVSS

SEQ ID NO: 805 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgttctgattatctattatgatagtagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccatctgaccgtcctcg

SEQ ID NO: 806 - protein, 106 aa, Canis sp.; identical to SEQ ID NO: 566
  source 1..106: note = canis familiaris

SEQ ID NO: 807 - protein, 7 aa, Canis sp.
  source 1..7: note = canis familiaris
GLTFSRY

SEQ ID NO: 808 - protein, 6 aa, Canis sp.
  source 1..6: note = canis familiaris
NTGGSS

SEQ ID NO: 809 - protein, 7 aa, Canis sp. [sequence omitted]

SEQ ID NO: 810 - protein, 11 aa, Canis sp.
  source 1..11: note = canis familiaris
GGDNIGSKSVH

SEQ ID NO: 811 - protein, 7 aa, Canis sp.
  source 1..7: note = canis familiaris
YDSSRPT

SEQ ID NO: 812 - protein, 11 aa, Canis sp.
  source 1..11: note = canis familiaris
CQVWDSSANVF

SEQ ID NO: 813 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgaggacacggccgtgtattactgtgcgagttttgactactggggccagggaaccctggtcaccgtctcctcag

SEQ ID NO: 814 - protein, 112 aa, Canis sp.; identical to SEQ ID NO: 714
  source 1..112: note = canis familiaris

SEQ ID NO: 815 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcaacaggccgacagggatccctgagcgattctccggcgccaactcggggaacacggccaccctgaccatcagcggggccctggccgaggacgaggctgactattactgccaggtgtgggacagcagtggaaatgtgttcggcggaggcacccatctgaccgtcctcg

SEQ ID NO: 816 - protein, 106 aa, Canis sp.
  source 1..106: note = canis familiaris
SYVLTQLPSKNVTLKQPAHITCGGDNIGSKSVHWYQQKLGQAPVLIIYYDSNRPTGIPERFSGANSGNTATLTISGALAEDEADYYCQVWDSSGNVFGGGTHLTVL

SEQ ID NO: 817 - protein, 7 aa, Canis sp.
  source 1..7: note = canis familiaris
GFTFSRY

SEQ ID NO: 818 - protein, 6 aa, Canis sp.
  source 1..6: note = canis familiaris
NSGGSS

SEQ ID NO: 819 - protein, 7 aa, Canis sp. [sequence omitted]

SEQ ID NO: 820 - protein, 11 aa, Canis sp.
  source 1..11: note = canis familiaris
GGDNIGSKSVH

SEQ ID NO: 821 - protein, 7 aa, Canis sp.
  source 1..7: note = canis familiaris
YDSNRPT

SEQ ID NO: 822 - protein, 11 aa, Canis sp.
  source 1..11: note = canis familiaris
CQVWDSSGNVF

SEQ ID NO: 823 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagaaggtaccacatgagctgggtccgccaggctccagggaaggggcttcagtgggtcgcatacattgacagtggtggaagtggcacaagttatgcagacgctgtgaagggccgattcaccatctccagagacaacgccaagaacacgctgtatcttcagatgaacagcctgagagccgaggacacggccgtgtattactgttcgagtctcgactactggggccagggaaccctggtcaccgtctcttcag

SEQ ID NO: 824 - protein, 112 aa, Canis sp.
  source 1..112: note = canis familiaris
EVQLVESGGDLVKPGGSLRLSCVASGFTFRRYHMSWVRQAPGKGLQWVAYIDSGGSGTSYADAVKGRFTISRDNAKNTLYLQMNSLRAEDTAVYYCSSLDYWGQGTLVTVSS

SEQ ID NO: 825 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 826 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTISGALAEDEADYYCQVWDSSGNVFGGGTHLTVL

SEQ ID NO: 827 - protein, 7 aa, Canis sp.
  source 1..7: note = canis familiaris
GFTFRRY

SEQ ID NO: 828 - protein, 6 aa, Canis sp.
  source 1..6: note = canis familiaris
DSGGSG

SEQ ID NO: 829 - protein, 7 aa, Canis sp. [sequence omitted]

SEQ ID NO: 830 - protein, 11 aa, Canis sp.
  source 1..11: note = canis familiaris
GGDNIGSKSVH

SEQ ID NO: 831 - protein, 7 aa, Canis sp.
  source 1..7: note = canis familiaris
YDSRRPT

SEQ ID NO: 832 - protein, 11 aa, Canis sp.
  source 1..11: note = canis familiaris
CQVWDSSGNVF

SEQ ID NO: 833 - DNA, 337 bp, Canis sp.
  source 1..337: mol_type = genomic DNA; note = canis familiaris
gaagttcaactggtggagtctgggggagacctggtgaagcctggggggtccctgagactttcctgtgtggcctctggaatcaccttcagtaggtactacatgaactgggtccgccaggctccagggaaggggcttcagtgggtcgcatacattgacagtggtggaagtcccacaacctatgcagacgctgtgaagggccgattcaccatctccagagacaacgccaagaaagcgctgtatcttcagatgaacagcctgagagccgaggacacggccgtatattactgtacgagttttgactactggggccagggaaccctggtcaccgtctcctcag

SEQ ID NO: 834 - protein, 112 aa, Canis sp.
  source 1..112: note = canis familiaris
EVQLVESGGDLVKPGGSLRLSCVASGITFSRYYMNWVRQAPGKGLQWVAYIDSGGSPTTYADAVKGRFTISRDNAKKALYLQMNSLRAEDTAVYYCTSFDYWGQGTLVTVSS

SEQ ID NO: 835 - DNA, 319 bp, Canis sp.
  source 1..319: mol_type = genomic DNA; note = canis familiaris
tcctatgtcctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactatttctgccaggtgtgggacagcagtgctaatgtgttcggcggaggcacccatctgaccgtcctcg

SEQ ID NO: 836 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYFCQVWDSSANVFGGGTHLTVL

SEQ ID NO: 837 - protein, 7 aa, Canis sp.
  source 1..7: note = canis familiaris
GITFSRY

SEQ ID NO: 838 - protein, 6 aa, Canis sp.
  source 1..6: note = canis familiaris
DSGGSP

SEQ ID NO: 839 - protein, 7 aa, Canis sp. [sequence omitted]

SEQ ID NO: 840 - protein, 11 aa, Canis sp.
  source 1..11: note = canis familiaris
GGDNIGSKSVH

SEQ ID NO: 841 - protein, 7 aa, Canis sp.
  source 1..7: note = canis familiaris
YDSSRPT

SEQ ID NO: 842 - protein, 11 aa, Canis sp.
  source 1..11: note = canis familiaris
CQVWDSSANVF

SEQ ID NO: 843 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggaatcaccttcagtagatattatatgaactgggtccgccaggctccagggaaggggcttcagtgggtcgcatacataaacagtggtggaagtcccacaacctatgcagacgctgtaaagggccgattcaccatctccagagacaacgccaagaacacgttgtatcttcagatgaacagcctgagagccgaggacacggccgtgtattactgtacgagttttgactactggggccagggaaccctggtcaccgtctcctcag

SEQ ID NO: 844 - protein, 112 aa, Canis sp.
  source 1..112: note = canis familiaris
EVQLVESGGDLVKPGGSLRLSCVASGITFSRYYMNWVRQAPGKGLQWVAYINSGGSPTTYADAVKGRFTISRDNAKNTLYLQMNSLRAEDTAVYYCTSFDYWGQGTLVTVSS

SEQ ID NO: 845 - DNA, 319 bp, Canis sp.; identical to SEQ ID NO: 525
  source 1..319: mol_type = genomic DNA; note = canis familiaris

SEQ ID NO: 846 - protein, 106 aa, Canis sp.; identical to SEQ ID NO: 526
  source 1..106: note = canis familiaris

SEQ ID NO: 847 - protein, 7 aa, Canis sp.
  source 1..7: note = canis familiaris
GITFSRY

SEQ ID NO: 848 - protein, 6 aa, Canis sp.
  source 1..6: note = canis familiaris
NSGGSP

SEQ ID NO: 849 - protein, 7 aa, Canis sp. [sequence omitted]

SEQ ID NO: 850 - protein, 11 aa, Canis sp.
  source 1..11: note = canis familiaris
GGDNIGSKSVH

SEQ ID NO: 851 - protein, 7 aa, Canis sp.
  source 1..7: note = canis familiaris
YDTSRPT

SEQ ID NO: 852 - protein, 11 aa, Canis sp.
  source 1..11: note = canis familiaris
CQVWDSSANVF

SEQ ID NO: 853 - DNA, 337 bp, Canis sp.
  source 1..337: mol_type = genomic DNA; note = canis familiaris
gaggtgcagctggtggagtctgggggagacctggtgaagcctggggggtccctgagactttcctgtgtggcctctggattcaccttcagtaggtactatatgaactgggtccgccaggctccagggaaggggcttcagtgggtcgcatacattaacagtggtggaagtagcacaagctatgcagacgctgtgaagggccgattcaccatctccagagacaacgccaagaacacgctgtatcttcagatgaacagcctgagagccgacgacacggccgtgtattactgtacgagttttgactactggggccagggaaccctggtcaccgtctcctcag

SEQ ID NO: 854 - protein, 112 aa, Canis sp.
  source 1..112: note = canis familiaris
EVQLVESGGDLVKPGGSLRLSCVASGFTFSRYYMNWVRQAPGKGLQWVAYINSGGSSTSYADAVKGRFTISRDNAKNTLYLQMNSLRADDTAVYYCTSFDYWGQGTLVTVSS

SEQ ID NO: 855 - DNA, 319 bp, Canis sp.; identical to SEQ ID NO: 605
  source 1..319: mol_type = genomic DNA; note = canis familiaris

SEQ ID NO: 856 - protein, 106 aa, Canis sp.; identical to SEQ ID NO: 566
  source 1..106: note = canis familiaris

SEQ ID NO: 857 - protein, 7 aa, Canis sp.
  source 1..7: note = canis familiaris
GFTFSRY

SEQ ID NO: 858 - protein, 6 aa, Canis sp.
  source 1..6: note = canis familiaris
NSGGSS

SEQ ID NO: 859 - protein, 7 aa, Canis sp. [sequence omitted]

SEQ ID NO: 860 - protein, 11 aa, Canis sp.
  source 1..11: note = canis familiaris
GGDNIGSKSVH

SEQ ID NO: 861 - protein, 7 aa, Canis sp.
  source 1..7: note = canis familiaris
YDSSRPT

SEQ ID NO: 862 - protein, 11 aa, Canis sp.
  source 1..11: note = canis familiaris
CQVWDSSANVF

SEQ ID NO: 863 - DNA, 337 bp, Canis sp.
  source 1..337: mol_type = genomic DNA; note = canis familiaris
gaggtgcaactggtggagtctgggggagacctggtgaagcctggggggtccctgagactttcctgtgtggcctctggattcaccttcagtagataccacatgagttgggtccgccaggctccagggaaggggcttcagtgggtcgcattcattaacactggtagaatgagcacaaactatgcagacgctgtgaagggccgattcaccatctccagagacaacgccaagaacacgctgtatcttcagatgaacagcctgagagccgaggacacggccgtatattactgtacgagtctcgactactggggccagggaacccttgtcaccgtctcctcag

SEQ ID NO: 864 - protein, 112 aa, Canis sp.
  source 1..112: note = canis familiaris
EVQLVESGGDLVKPGGSLRLSCVASGFTFSRYHMSWVRQAPGKGLQWVAFINTGRMSTNYADAVKGRFTISRDNAKNTLYLQMNSLRAEDTAVYYCTSLDYWGQGTLVTVSS

SEQ ID NO: 865 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagcagtgctaatgtgttcggcggaggcacccacctgaccgtcctcg

SEQ ID NO: 866 - protein, 106 aa, Canis sp.; identical to SEQ ID NO: 566
  source 1..106: note = canis familiaris

SEQ ID NO: 867 - protein, 7 aa, Canis sp.
  source 1..7: note = canis familiaris
GFTFSRY

SEQ ID NO: 868 - protein, 6 aa, Canis sp.
  source 1..6: note = canis familiaris
NTGRMS

SEQ ID NO: 869 - protein, 7 aa, Canis sp.
  source 1..7: note = canis familiaris
CTSLDYW

SEQ ID NO: 870 - protein, 11 aa, Canis sp.
  source 1..11: note = canis familiaris
GGDNIGSKSVH

SEQ ID NO: 871 - protein, 7 aa, Canis sp.
  source 1..7: note = canis familiaris
YDSSRPT

SEQ ID NO: 872 - protein, 11 aa, Canis sp.
  source 1..11: note = canis familiaris
CQVWDSSANVF

SEQ ID NO: 873 - DNA, 337 bp, Canis sp.
  source 1..337: mol_type = genomic DNA; note = canis familiaris
gaggtgcagctggtgcagtctgggggagacctggtgaagcctggggggtccctgagactttcctgtgtggcctctggatacaccttcagtcggtatcacatgagttgggtccgtcaggctccagggaaggggcttcagtgggtcgcattcattaatagtgatggaactggtataacctatggagacgctgtgaagggccgattcaccatctccagagacaatgccaaaaacacgctgtatcttcagatgaacagcctgagagccgaggacacggccatttattattgtacgacttttgactcctggggccagggaaccctggtcaccgtctcctcag

SEQ ID NO: 874 - protein, 112 aa, Canis sp.
  source 1..112: note = canis familiaris
EVQLVQSGGDLVKPGGSLRLSCVASGYTFSRYHMSWVRQAPGKGLQWVAFINSDGTGITYGDAVKGRFTISRDNAKNTLYLQMNSLRAEDTAIYYCTTFDSWGQGTLVTVSS

SEQ ID NO: 875 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctatactgattatctattatgatagtaggaggccgacagggatccctgagcgattctccggcgccaactcggggaacacggccaccctgaccatcagcggggccctggccgaggacgaggctgactattactgccaggtgtgggacagcagtggtaatgtgttcggcggaggcacccatctgaccgtcctcg

SEQ ID NO: 876 - protein, 106 aa, Canis sp.; identical to SEQ ID NO: 506
  source 1..106: note = canis familiaris

SEQ ID NO: 877 - protein, 7 aa, Canis sp.
  source 1..7: note = canis familiaris
GYTFSRY

SEQ ID NO: 878 - protein, 6 aa, Canis sp.
  source 1..6: note = canis familiaris
NSDGTG

SEQ ID NO: 879 - protein, 7 aa, Canis sp.
  source 1..7: note = canis familiaris
CTTFDSW

SEQ ID NO: 880 - protein, 11 aa, Canis sp.
  source 1..11: note = canis familiaris
GGDNIGSKSVH

SEQ ID NO: 881 - protein, 7 aa, Canis sp.
  source 1..7: note = canis familiaris
YDSRRPT

SEQ ID NO: 882 - protein, 11 aa, Canis sp.
  source 1..11: note = canis familiaris
CQVWDSSGNVF

SEQ ID NO: 883 - DNA, 337 bp, Canis sp.
  source 1..337: mol_type = genomic DNA; note = canis familiaris
gaattacagctggtggagtctgggggagacctggtgaagcctggggggtccctgagactttcctgtgtggcctctggattcaccttcagtaggtaccacatgagctgggtccgccaggctccagggaaggggcttcagtgggtcgcatacattaacagtggtggaagtagtacaagttatgcagacgctgtgaagggccgattcaccatctccagagacaacgccaagaacacgctgtatcttcagatgaacagcctgagagccgaggacacggccgtatattactgtacgacttttgactcctggggccagggaaccctggtcaccgtctcctcag

SEQ ID NO: 884 - protein, 112 aa, Canis sp.
  source 1..112: note = canis familiaris
ELQLVESGGDLVKPGGSLRLSCVASGFTFSRYHMSWVRQAPGKGLQWVAYINSGGSSTSYADAVKGRFTISRDNAKNTLYLQMNSLRAEDTAVYYCTTFDSWGQGTLVTVSS

SEQ ID NO: 885 - DNA, 319 bp, Canis sp.
  source 1..319: mol_type = genomic DNA; note = canis familiaris
tcctatgtgctgacacaactgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggagccctggccgaggacgaggctgactattactgccaggtgtgggacatcagtgctaatgtgttcggcggaggcacccatctgaccgtcctcg

SEQ ID NO: 886 - protein, 106 aa, Canis sp.; identical to SEQ ID NO: 746
  source 1..106: note = canis familiaris

SEQ ID NO: 887 - protein, 7 aa, Canis sp.
  source 1..7: note = canis familiaris
GFTFSRY

SEQ ID NO: 888 - protein, 6 aa, Canis sp.
  source 1..6: note = canis familiaris
NSGGSS

SEQ ID NO: 889 - protein, 7 aa, Canis sp.
  source 1..7: note = canis familiaris
CTTFDSW

SEQ ID NO: 890 - protein, 11 aa, Canis sp.
  source 1..11: note = canis familiaris
GGDNIGSKSVH

SEQ ID NO: 891 - protein, 7 aa, Canis sp.
  source 1..7: note = canis familiaris
YDSSRPT

SEQ ID NO: 892 - protein, 11 aa, Canis sp.
  source 1..11: note = canis familiaris
CQVWDISANVF

SEQ ID NO: 893 - DNA, 337 bp, Canis sp.
  source 1..337: mol_type = genomic DNA; note = canis familiaris
gagatgcaactggtggagtctgggggagacctggtgaagcctggggggtccctgagactttcctgtgtggcctctggattcaccttcagtcgctaccacatgagctgggtccgccaggctccagggaaggggcttcagtgggtcgcatacattgacagtgatggaattaacacaaactatgcagacgctgtgaagggccgattcaccatctccagagacaacgccaaaaacacgctgtatcttcagatgaacagcctgagagccgaggacacggccctgtattactgtacaacctttgactactggggccagggaaccctggtcaccgtctcctcag

SEQ ID NO: 894 - protein, 112 aa, Canis sp.
  source 1..112: note = canis familiaris
EMQLVESGGDLVKPGGSLRLSCVASGFTFSRYHMSWVRQAPGKGLQWVAYIDSDGINTNYADAVKGRFTISRDNAKNTLYLQMNSLRAEDTALYYCTTFDYWGQGTLVTVSS

SEQ ID NO: 895 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagcagcaggccgacagggatccctgagcgattctccggcgccaactcggggaacacggccaccctgaccatcagcggggccctggccgaggacgaggctgactattactgccaggtgtgggacagtagtggtaatgtgttcggcggaggcacccatctgaccgtcctcg

SEQ ID NO: 896 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDSSGNVFGGGTHLTVL

SEQ ID NO: 897 - protein, 7 aa, Canis sp.
  source 1..7: note = canis familiaris
GFTFSRY

SEQ ID NO: 898 - protein, 6 aa, Canis sp.
  source 1..6: note = canis familiaris
DSDGIN

SEQ ID NO: 899 - protein, 7 aa, Canis sp. [sequence omitted]

SEQ ID NO: 900 - protein, 11 aa, Canis sp.
  source 1..11: note = canis familiaris
GGDNIGSKSVH

SEQ ID NO: 901 - protein, 7 aa, Canis sp.
  source 1..7: note = canis familiaris
YDSSRPT

SEQ ID NO: 902 - protein, 11 aa, Canis sp.
  source 1..11: note = canis familiaris
CQVWDSSGNVF

SEQ ID NO: 903 - DNA, 337 bp, Canis sp.
  source 1..337: mol_type = genomic DNA; note = canis familiaris
gaggtgcagctggtggagtctgggggagacctaatgaagcctggggggtccctgagactttcctgtgtggcctctggaatcaccttcagtggctaccacatgagttgggtccgccaggctccagggaagggacttcagtgggtcgcattcattaatagtggtagaaaaaacacagcttatgctgacgctgtgaagggccgattcaccatctccagagacaacgccaaaaacacgctgtatcttcagatgaacagcctgagagccgaagatacggccatttattactgtacgacccttgaattttggggccagggaaccctggtcaccgtctcctcag

SEQ ID NO: 904 - protein, 112 aa, Canis sp.
  source 1..112: note = canis familiaris
EVQLVESGGDLMKPGGSLRLSCVASGITFSGYHMSWVRQAPGKGLQWVAFINSGRKNTAYADAVKGRFTISRDNAKNTLYLQMNSLRAEDTAIYYCTTLEFWGQGTLVTVSS

SEQ ID NO: 905 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaaatgttcactggtaccagcagaagctgggccaggcccctgtactgattatctattatgatagccgcaggccgacagggatccctgagcgattctccggcgtcaactcggggaacacggccaccctgaccatcaacggggccctggccgaggacgaggctgactattactgccagatgtgggacagcagtgctaatgtgttcggcggaggcacccatctgaccgtcctcg

SEQ ID NO: 906 - protein, 106 aa, Canis sp.
  source 1..106: note = canis familiaris
SYVLTQLPSKNVTLKQPAHITCGGDNIGSKNVHWYQQKLGQAPVLIIYYDSRRPTGIPERFSGVNSGNTATLTINGALAEDEADYYCQMWDSSANVFGGGTHLTVL

SEQ ID NO: 907 - protein, 7 aa, Canis sp.
  source 1..7: note = canis familiaris
GITFSGY

SEQ ID NO: 908 - protein, 6 aa, Canis sp.
  source 1..6: note = canis familiaris
NSGRKN

SEQ ID NO: 909 - protein, 7 aa, Canis sp.
  source 1..7: note = canis familiaris
CTTLEFW

SEQ ID NO: 910 - protein, 11 aa, Canis sp.
  source 1..11: note = canis familiaris
GGDNIGSKNVH

SEQ ID NO: 911 - protein, 7 aa, Canis sp.
  source 1..7: note = canis familiaris
YDSRRPT

SEQ ID NO: 912 - protein, 11 aa, Canis sp.
  source 1..11: note = canis familiaris
CQMWDSSANVF

SEQ ID NO: 913 - DNA, 337 bp, Canis sp.
  source 1..337: mol_type = genomic DNA; note = canis familiaris
gaggtgcggctggtggagtctgggggagacctggtgaagccgggggggtccctgagactttcctgtgtggcctctggactcaccttcaataggtactacatgagttgggtccgccagggtccagggaaggggcttcagtgggtcgcatacattgacagtggtggaagtcccacaacctatgcagacactgtgaagggccgattcaccatctccagagacaacgccaagaacacgctgtatcttcagatgaacagcctgagagtcgaggacacggccgtgtattactgtacgactcttgaatactggggccagggaaccctggtcaccgtctcctcag

SEQ ID NO: 914 - protein, 112 aa, Canis sp.
  source 1..112: note = canis familiaris
EVRLVESGGDLVKPGGSLRLSCVASGLTFNRYYMSWVRQGPGKGLQWVAYIDSGGSPTTYADTVKGRFTISRDNAKNTLYLQMNSLRVEDTAVYYCTTLEYWGQGTLVTVSS

SEQ ID NO: 915 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagtagtaggccgacagggatccctgagcgattctccggcgccaactcggggaacacggccaccctgaccatcagcggggccctggccgaggacgaggctgactattactgccaggtgtgggacaccagtgctaatgtgttcggcggaggcacccatctgaccgtcctcg

SEQ ID NO: 916 - protein, 106 aa, Canis sp.
  source 1..106: note = canis familiaris
SYVLTQLPSKNVTLKQPAHITCGGDNIGSKSVHWYQQKLGQAPVLIIYYDSSRPTGIPERFSGANSGNTATLTISGALAEDEADYYCQVWDTSANVFGGGTHLTVL

SEQ ID NO: 917 - protein, 7 aa, Canis sp.
  source 1..7: note = canis familiaris
GLTFNRY

SEQ ID NO: 918 - protein, 6 aa, Canis sp.
  source 1..6: note = canis familiaris
DSGGSP

SEQ ID NO: 919 - protein, 7 aa, Canis sp.
  source 1..7: note = canis familiaris
CTTLEYW

SEQ ID NO: 920 - protein, 11 aa, Canis sp.
  source 1..11: note = canis familiaris
GGDNIGSKSVH

SEQ ID NO: 921 - protein, 7 aa, Canis sp.
  source 1..7: note = canis familiaris
YDSSRPT

SEQ ID NO: 922 - protein, 11 aa, Canis sp.
  source 1..11: note = canis familiaris
CQVWDTSANVF

SEQ ID NO: 923 - DNA, 337 bp, Canis sp.
  source 1..337: mol_type = genomic DNA; note = canis familiaris
gaggtgcagttggtggagtctgggggagacctggtgaagcctggggggtccctgagactttcctgtgtggcctctggattcaccttcagtaggtattacatgacctgggtccgccaggctccagggaaggggcttcagtgggtcgcatacattaacagtgatagaagaagcacaacctatgcagacgctgtgaagggccgattcaccatctccagagacaacgccaaaaacacgctgtatcttcagatgaacagcctgagagccgaggacacggccgtatattactgtacgacccttgaatattggggccagggaaccctggtcaccgtctcctcag

SEQ ID NO: 924 - protein, 112 aa, Canis sp.
  source 1..112: note = canis familiaris
EVQLVESGGDLVKPGGSLRLSCVASGFTFSRYYMTWVRQAPGKGLQWVAYINSDRRSTTYADAVKGRFTISRDNAKNTLYLQMNSLRAEDTAVYYCTTLEYWGQGTLVTVSS

SEQ ID NO: 925 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgccaggtgtgggacagcagtgctaatgtgttcggcggaggcacccatctgaccgtcctcg

SEQ ID NO: 926 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTINGALAEDEADYYCQVWDSSANVFGGGTHLTVL

SEQ ID NO: 927 - protein, 7 aa, Canis sp.
  source 1..7: note = canis familiaris
GFTFSRY

SEQ ID NO: 928 - protein, 6 aa, Canis sp.
  source 1..6: note = canis familiaris
NSDRRS

SEQ ID NO: 929 - protein, 7 aa, Canis sp.
  source 1..7: note = canis familiaris
CTTLEYW

SEQ ID NO: 930 - protein, 11 aa, Canis sp.
  source 1..11: note = canis familiaris
GGDNIGSKSVH

SEQ ID NO: 931 - protein, 7 aa, Canis sp.
  source 1..7: note = canis familiaris
YDSRRPT

SEQ ID NO: 932 - protein, 11 aa, Canis sp.
  source 1..11: note = canis familiaris
CQVWDSSANVF

SEQ ID NO: 933 - DNA, 337 bp, Canis sp.
  source 1..337: mol_type = genomic DNA; note = canis familiaris
gcggtgcagctggtggagtcagggggagacctggtgaagcctggggggtccctgagactttcctgtgtggcctctggaatcaccttcagtaggtaccacatgagttgggtccgccaggctccagggaaggggcttcagtgggtcgcatacattaatagtggtggaagtgccacaagttatacagacgctgtgaagggccgattcagcatctccagagacaacggcaaaaacacgctttatcttcagatgaatagcctgagagtcgaggactcggccgtctattactgtacgacccttgacctctggggccagggaaccctggtcaccgtctcctcag

SEQ ID NO: 934 - protein, 112 aa, Canis sp.
  source 1..112: note = canis familiaris
AVQLVESGGDLVKPGGSLRLSCVASGITFSRYHMSWVRQAPGKGLQWVAYINSGGSATSYTDAVKGRFSISRDNGKNTLYLQMNSLRVEDSAVYYCTTLDLWGQGTLVTVSS

SEQ ID NO: 935 - DNA, 319 bp, Canis sp.
  source 1..319: mol_type = genomic DNA; note = canis familiaris
tcctatgtgctgacacagctgccatccaaaaatgtgaccctgaagcagccggcccacatcacctgtgggggagacaacattggaagtaaaagtgttcactggtaccagcagaagctgggccaggcccctgtactgattatctattatgatagccgcaggccgacagggatccctgagcgattctccggcgccaactcggggaacacggccaccctgaccatcaacggggccctggccgaggacgaggctgactattactgtcagatgtgggacagcagtgctaatgtgttcggcggaggcacccatctgaccgtcctcg

SEQ ID NO: 936 - protein, 106 aa, Canis sp.
  source 1..106: note = canis familiaris
SYVLTQLPSKNVTLKQPAHITCGGDNIGSKSVHWYQQKLGQAPVLIIYYDSRRPTGIPERFSGANSGNTATLTINGALAEDEADYYCQMWDSSANVFGGGTHLTVL

SEQ ID NO: 937 - protein, 7 aa, Canis sp.
  source 1..7: note = canis familiaris
GITFSRY

SEQ ID NO: 938 - protein, 6 aa, Canis sp.
  source 1..6: note = canis familiaris
NSGGSA

SEQ ID NO: 939 - protein, 7 aa, Canis sp.
  source 1..7: note = canis familiaris
CTTLDLW

SEQ ID NO: 940 - protein, 11 aa, Canis sp.
  source 1..11: note = canis familiaris
GGDNIGSKSVH

SEQ ID NO: 941 - protein, 7 aa, Canis sp.
  source 1..7: note = canis familiaris
YDSRRPT

SEQ ID NO: 942 - protein, 11 aa, Canis sp.
  source 1..11: note = canis familiaris
CQMWDSSANVF

SEQ ID NO: 943 - protein, 19 aa, Canis sp.
  source 1..19: note = canis familiaris
MESALSWVFLVTILKGVQG

SEQ ID NO: 944 - protein, 19 aa, Canis sp.
  source 1..19: note = canis familiaris
MAWTHLLLSLLALCTGSVA

SEQ ID NO: 945 - protein, 19 aa, Canis sp.
  source 1..19: note = canis familiaris
MGWSCIILFLVATATGVHS

SEQ ID NO: 946 - protein, 98 aa, Canis sp.
  source 1..98: note = canis familiaris
EVQLVESGGDLVKPGGSLRLSCVASGFTFSSYHMSWVRQAPGKGLQWVAYINSGGSSTSYADAVKGRFTISRDNAKNTLYLQMNSLRAEDTAVYYCAS